(19) United States
(12) Patent Application Publication
(10) Pub. No.: US 2020/0231580 A1
(43) Pub. Date: Jul. 23, 2020

(54) ORGANIC COMPOUND AND ORGANIC ELECTROLUMINESCENT ELEMENT INCLUDING SAME

(71) Applicant: DOOSAN CORPORATION, Seoul (KR)

(72) Inventor: Tae Jin CHOI, Yongin-si (KR)

(73) Assignee: DOOSAN CORPORATION, Seoul (KR)

(21) Appl. No.: 16/630,168

(22) PCT Filed: Jul. 9, 2018
(86) PCT No.: PCT/KR2018/007734

§ 371 Date: Jan. 10, 2020

(30) Foreign Application Priority Data
Jul. 13, 2017 (KR) 10-2017-0089032

(51) Int. Cl. C07D 409/14; C07D 405/04; C07D 405/10; C07D 405/14; H01L 51/00; C07D 333/10

(52) U.S. Cl. CPC C07D 409/14; C07D 405/04; C07D 405/10; C07D 405/14; H01L 51/0073; H01L 51/0074; H01L 51/5056; H01L 51/0068; H01L 51/0067; H01L 51/006; C07D 333/10; H01L 51/0061; H01L 51/0072; H01L 51/0065

(57) ABSTRACT

The present invention relate to a novel organic compound and an organic electroluminescent device including the same, and in particular, to a novel dibenzofuran-based compound having excellent thermal stability, carrier transport ability, light emitting ability and the like, and an organic electroluminescent device having low driving voltage, and enhanced luminous efficiency, lifetime properties and the like by including the novel dibenzofuran-based compound as a material of one or more organic material layers.

### TECHNICAL FIELD

The present invention relate to a novel organic compound and an organic electroluminescent device including the same, and in particular, to a novel dibenzofuran-based compound having excellent thermal stability, carrier transport ability, light emitting ability and the like, and an organic electroluminescent device having low driving voltage, and enhanced luminous efficiency, lifetime properties and the like by including the novel dibenzofuran-based compound as a material of one or more organic material layers.

### BACKGROUND ART

With the observation of organic thin film light emission made by Bemanose in 1950s as a start, studies on organic electroluminescent (EL) devices (hereinafter, simply referred to as ‘organic EL device’) leading to blue electroluminescence using a single anthracene crystal in 1965 have been continued, and in 1987, an organic EL device having a two-layer laminated structure formed with a hole layer (NPB) and a light emitting layer (Alq3) has been proposed by Tang.

After that, in order for an organic EL device to obtain properties of high efficiency and long lifetime required for commercialization, a form of a multilayer laminated structure providing each of distinguishing and subdivided functions such as an organic layer responsible for hole injection and transfer, an organic layer responsible for electron injection and transfer, an organic layer inducing electroluminescence by bonding holes and electrons, and the like in the device has been proposed.

When a voltage is applied between the two electrodes in an organic electroluminescent device, holes and electrons are injected to an organic material layer from the anode and the cathode, respectively. When the injected holes and electrons meet, excitons are formed, and light emits when these excitons fall back to the ground state. Materials included in the organic material layer may be divided into a light emitting material, a hole injection material, a hole transport material, an electron transport material, an electron injection material and the like depending on the function.

As for an electron spin of excitons formed by recombination of electrons and holes, singlet excitons and triplet excitons are produced in a ratio of 25% and 75%, respectively. Herein, depending on the electron spin type of formed excitons, an organic EL device may be divided into a fluorescent EL device in which singlet excitons contribute to light emission, and a phosphorescent EL device in which triplet excitons contribute to light emission.

In the fluorescent EL device having light emission by singlet excitons, internal quantum efficiency may not theoretically exceed 25% by the ratio of generation, and external quantum efficiency is accepted to have a limit of 5%.

In the phosphorescent EL device having light emission by triplet excitons, luminous efficiency may be enhanced by up to 4 times compared to fluorescence when using a metal complex compound including a transition metal heavy atom such as Ir or Pt as a phosphorescent dopant.

As described above, the phosphorescent EL device theoretically exhibits higher efficiency than fluorescence in terms of luminous efficiency. However, unlike green and red phosphorescent devices, a blue phosphorescent device has not been commercialized due to insignificant development levels for color purity of deep blue color, highly efficient phosphorescent dopant, and a host with a wide energy gap, and blue fluorescent device products have been used instead.

As performance of an organic EL device has been enhanced to a commercializable level by introducing a multilayer laminated structure, there have been attempts to expand its application to, beginning with radio display products for vehicles in 1997, portable information display devices and display devices for TV.

In addition, with a recent trend toward large and high resolution displays, development of an organic EL device having high efficiency and long lifetime has been required. Particularly, high resolution of a display is obtained when more pixels are formed in the same area. The organic EL pixels have a reduced light emitting area by such high resolution, which eventually shortens a lifetime, and this has become the most important challenge that an organic EL device needs to overcome.

However, existing organic EL device materials have a low glass transition temperature and thereby have decreased thermal stability, and a satisfactory level has not been obtained in terms of a lifetime of an organic electroluminescent device and an improvement has also been required in terms of light emission properties.

### DISCLOSURE

### Technical Problem

The present invention is directed to providing a novel compound capable of being used as a light emitting layer material, a hole transport layer material, a light emitting auxiliary layer material, a hole blocking layer material or the like by having excellent thermal stability, carrier transport ability, light emitting ability and the like.

The present invention is also directed to providing an organic electroluminescent device having low driving voltage, high luminous efficiency, and enhanced lifetime properties by including the novel compound.

### Technical Solution

One embodiment of the present invention provides a compound represented by the following Chemical Formula 1:

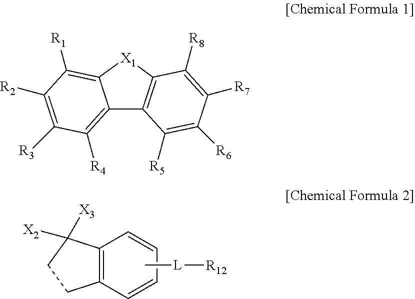

wherein,

X₁ is selected from the group consisting of O, S, C(R₉)(R₁₀) and N(R₁₁),

at least one of R₁ and R₂, R₂ and R₃, R₃ and R₄, R₄ and R₅, R₅ and R₆, R₆ and R₇, and R₇ and R₈ is fused with the ring represented by Chemical Formula 2 to form a fused ring,

R₁, R₂, R₃, R₄, R₅, R₆, R₇ and R₈ not forming a fused ring with the ring represented by Chemical Formula 2 are the same as or different from each other, and each independently selected from the group consisting of hydrogen, deuterium, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group, or may bond to adjacent groups to form a fused ring,

a dotted line of Chemical Formula 2 is a part fused to Chemical Formula 1,

X₂ and X₃ are the same as or different from each other, and each independently selected from the group consisting of hydrogen, deuterium, a C₁˜C₂ alkyl group and a C₆˜C₆₀ aryl group,

L is selected from the group consisting of a single bond, a C₆˜C₁₈ arylene group and a heteroarylene group having 5 to 18 nuclear atoms,

R₉ to R₁₁ are the same as or different from each other, and each independently selected from the group consisting of hydrogen, deuterium, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group,

R₁₂ is selected from the group consisting of a C₆˜C₆₀ acyl group, a heteroaryl group having 5 to 60 nuclear atoms, a C₃˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylamine group, and

the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the arylphosphine group, the arylphosphine oxide group, the mono or diarylphosphinyl group and the arylamine group of R₁ to R₁₂, X₂ and X₃ are each independently unsubstituted or substituted with one or more types of substituents selected from the group consisting of a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylamine group, and when substituted with a plurality of the substituents, these may be the same as or different from each other.

Another embodiment of the present invention provides an organic electroluminescent device including an anode, a cathode, and one or more organic material layers provided between the anode and the cathode, wherein at least one of the one or more organic material layers includes the compound of Chemical Formula 1.

The “alkyl” in the present invention means a monovalent substituent derived from linear or branched saturated hydrocarbon having 1 to 40 carbon atoms. Examples thereof may include methyl, ethyl, propyl, isobutyl, sec-butyl, pentyl, iso-amyl, hexyl and the like, but are not limited thereto.

The “alkenyl” in the present invention means a monovalent substituent derived from linear or branched unsaturated hydrocarbon having one or more carbon-carbon double bonds and having 2 to 40 carbon atoms. Examples thereof may include vinyl, allyl, isopropenyl, 2-butenyl and the like, but are not limited thereto.

The “alkynyl” in the present invention means a monovalent substituent derived from linear or branched unsaturated hydrocarbon having one or more carbon-carbon triple bonds and having 2 to 40 carbon atoms. Examples thereof may include ethynyl, 2-propynyl and the like, but are not limited thereto.

The “aryl” in the present invention means a monovalent substituent derived from aromatic hydrocarbon having a single ring or two or more rings combined and having 6 to 60 carbon atoms. In addition, a form of two or more rings simply attached (pendant) or fused with each other may also be included. Examples of such awl may include phenyl, naphthyl, phenanthryl, anthryl and the like, but are not limited thereto.

The “heteroaryl” in the present invention means a monovalent substituent derived from monoheterocyclic or polyheterocyclic aromatic hydrocarbon having 5 to 40 nuclear atoms. Herein, one or more carbons, preferably 1 to 3 carbons, in the ring are substituted with a heteroatom such as N, O, S or Se. In addition, a form of two or more rings simply attached (pendant) or fused with each other may also be included, and furthermore, a form fused with an aryl group may also be included. Examples of such heteroaryl may include 6-membered monocyclic rings such as pyridyl, pyrazinyl, pyrimidinyl, pyridazinyl or triazinyl, polycyclic rings such as phenoxathienyl, indolizinyl, indolyl, purinyl, quinolyl, benzothiazole or carbazolyl, 2-furanyl, N-imidazolyl, 2-isoxazolyl, 2-pyridinyl, 2-pyrimidinyl and the like, but are not limited thereto.

The “aryloxy” in the present invention is a monovalent substituent represented by RO-, and R means aryl having 6 to 60 carbon atoms. Examples of such aryloxy may include phenyloxy, naphthyloxy, diphenyloxy and the like, but are not limited thereto.

The “alkyloxy” in the present invention is a monovalent substituent represented by R′O-, and R′ means alkyl having 1 to 40 carbon atoms and may include a linear, branched or cyclic structure. Examples of the alkyloxy may include methoxy, ethoxy, n-propoxy, 1-propoxy, t-butoxy, n-butoxy, pentoxy and the like, but are not limited thereto.

The “arylamine” in the present invention means amine substituted with aryl having 6 to 60 carbon atoms.

The “cycloalkyl” in the present invention means a monovalent substituent derived from monocyclic or polycyclic non-aromatic hydrocarbon having 3 to 40 carbon atoms. Examples of such cycloalkyl may include cyclopropyl, cyclopentyl, cyclohexyl, norbomyl, adamantine and the like, but are not limited thereto.

The “heterocycloalkyl” in the present invention means a monovalent substituent derived from non-aromatic hydrocarbon having 3 to 40 nuclear atoms, and one or more carbons, preferably 1 to 3 carbons, in the ring are substituted with a heteroatom such as N, O, S or Se. Examples of such heterocycloalkyl may include morpholine, piperazine and the like, but are not limited thereto.

The “alkylsilyl” in the present invention means silyl substituted with alkyl having 1 to 40 carbon atoms, and the “arylsilyl” means silyl substituted with aryl having 6 to 60 carbon atoms.

The “fused ring” in the present invention means a fused aliphatic ring, a fused aromatic ring, a fused heteroaliphatic ring, a fused heteroaromatic ring, or a combined form thereof.

### Advantageous Effects

A compound provided in the present invention has excellent thermal stability, carrier transport ability, light emitting ability and the like, and therefore, is useful as an organic material layer material of an organic electroluminescent device.

In addition, an organic electroluminescent device including the compound of the present invention in an organic material layer has properties of excellent light emitting performance, low driving voltage, high efficiency and long lifetime, and can be effectively used in a full color display panel and the like.

### MODE FOR DISCLOSURE

Hereinafter, the present invention will be described.

1. Novel Organic Compound

One embodiment of the present invention provides a novel compound having excellent thermal stability, carrier transport ability, light emitting ability and the like.

Specifically, the novel organic compound according to the present invention has a structure in which an indene moiety or the like is fused to a hetero compound to form a basic skeleton, and various substituents bond or are fused to such a basic skeleton.

Preferably, the novel organic compound of the present invention may be represented by the following Chemical Formula 1:

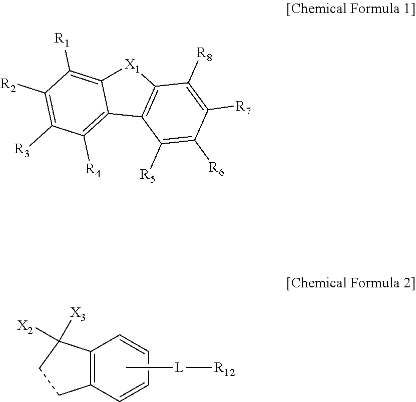

wherein,

X₁ is selected from the group consisting of O, S, C(R₉)(R₁₀) and N(R₁₁),

at least one of R₁ and R₂, R₂ and R₃, R₃ and R₄, R₄ and R₅, R₅ and R₆, R₆ and R₇, and R₇ and R₈ is fused with the ring represented by Chemical Formula 2 to form a fused ring,

R₁, R₂, R₃, R₄, R₅, R₆, R₇ and R₈ not forming a fused ring with the ring represented by Chemical Formula 2 are the same as or different from each other, and each independently selected from the group consisting of hydrogen, deuterium, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group, or may bond to adjacent groups to form a fused ring,

a dotted line of Chemical Formula 2 is a part fused to Chemical Formula 1,

X₂ and X₃ are the same as or different from each other, and each independently selected from the group consisting of hydrogen, deuterium, a C₁˜C₂ alkyl group and a C₆˜C₆₀ aryl group,

L is selected from the group consisting of a single bond, a C₆˜C₁₈ arylene group and a heteroarylene group having 5 to 18 nuclear atoms,

R₉ to R₁₁ are the same as or different from each other, and each independently selected from the group consisting of hydrogen, deuterium, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group,

R₁₂ is selected from the group consisting of a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylamine group, and

the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the arylphosphine group, the arylphosphine oxide group, the mono or diarylphosphinyl group and the arylamine group of R₁ to R₁₂, X₂ and X₃ are each independently unsubstituted or substituted with one or more types of substituents selected from the group consisting of a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylamine group, and when substituted with a plurality of the substituents, these may be the same as or different from each other.

More specifically, the novel organic compound according to the present invention has a structure in which an indene moiety or the like is fused to a compound formed with dibenzofuran, dibenzothiophene, fluorene and carbazole to form a basic skeleton, preferably, an indene moiety or the like is fused to dibenzofuran to form a basic skeleton, and various substituents bond or are fused thereto.

According to preferred one embodiment of the present invention, Chemical Formula 1 may be represented by the following Chemical Formulae 3 to 8:

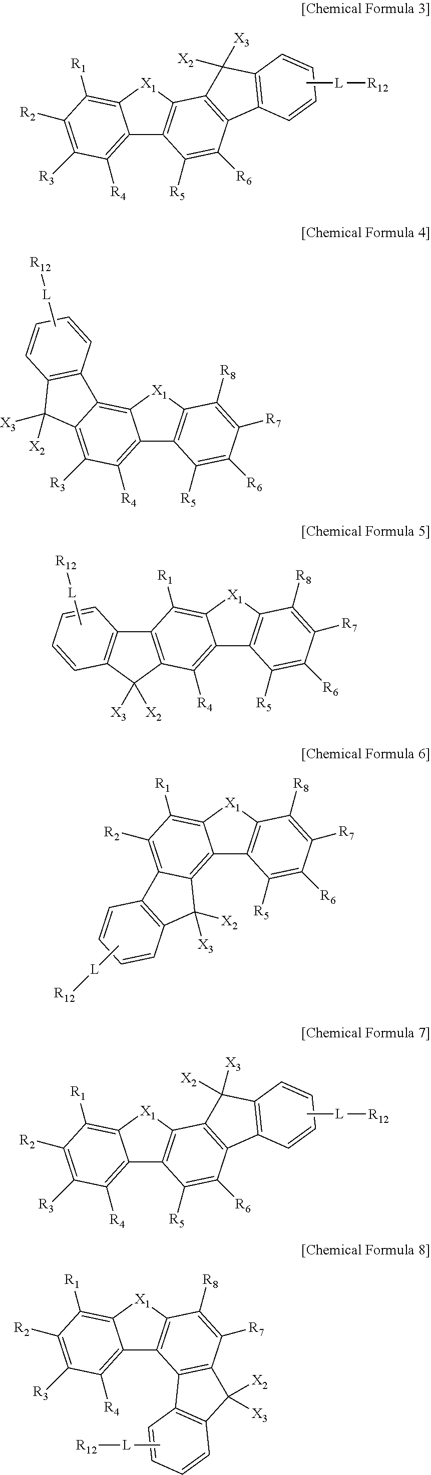

wherein,

X₁ to X₃, L and R₁ to R₈ each have the same definition as in Chemical Formula 1.

According to preferred one embodiment of the present invention, X₁ of Chemical Formula 1 to Chemical Formula 8 may be O.

According to preferred one embodiment of the present invention, X₂ and X₃ of Chemical Formula 1 to Chemical Formula 8 may be a methyl group.

According to preferred one embodiment of the present invention, R₁₂ of Chemical Formula 2 may be a substituent represented by any one of the following Chemical Formula 9 to Chemical Formula 11:

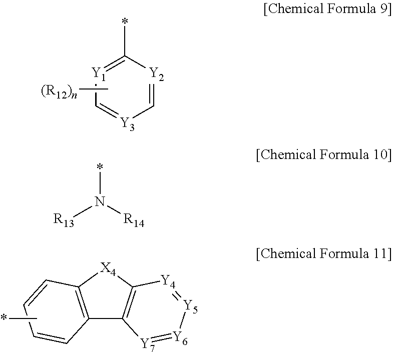

wherein,

* means a part forming a bond,

n is an integer of 0 to 2,

Y₁ to Y₃ are the same as or different from each other and each independently C(R₁₅) or N, and at least one or more are N,

Y₄ to Y₇ are the same as or different from each other, and each independently C(R₁₆) or N,

X₄ is selected from the group consisting of O, S, C(R₁₇)(R₁₈), Si(R₁₉)(R₂₀) and N(R₂₁),

R₁₂ to R₁₄ are the same as or different from each other, and each independently selected from the group consisting of hydrogen, deuterium, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₆˜C₆₀ alkylboron group, a C₆˜C₆₀ arylboron group, a C₆˜C₆₀ arylphosphine group, a C₆˜C₆₀ arylphosphine oxide group and a C₆˜C₆₀ arylamine group,

R₁₅ and R₁₆ are the same as or different from each other, and each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group, or may bond to adjacent groups to form a fused ring,

R₁₇ to R₂₁ are the same as or different from each other, and each independently selected from the group consisting of a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group, or may bond to adjacent groups to form a fused ring, and

the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the arylphosphine group, the arylphosphine oxide group, the mono or diarylphosphinyl group and the arylamine group of R₁₂ to R₂₁ are each independently unsubstituted or substituted with one or more types of substituents selected from the group consisting of a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylamine group, and when substituted with a plurality of the substituents, these may be the same as or different from each other.

According to preferred one embodiment of the present invention, L may be a single bond, a phenylene group or a biphenylene group.

According to preferred one embodiment of the present invention, the compound represented by Chemical Formula 1 may be selected from the group consisting of the following compounds.

The compound represented by Chemical Formula 1 is a compound selected from the group consisting of the following compounds:

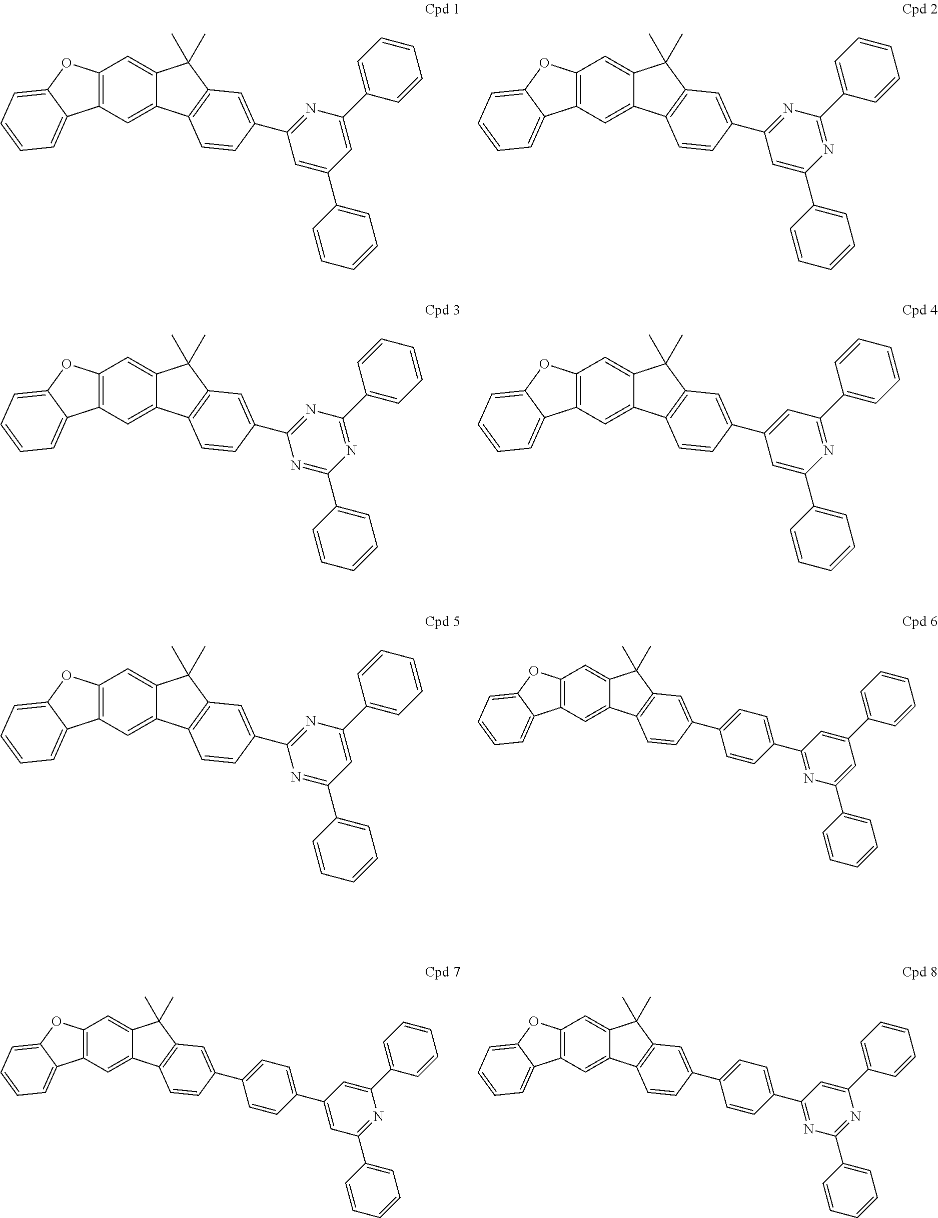

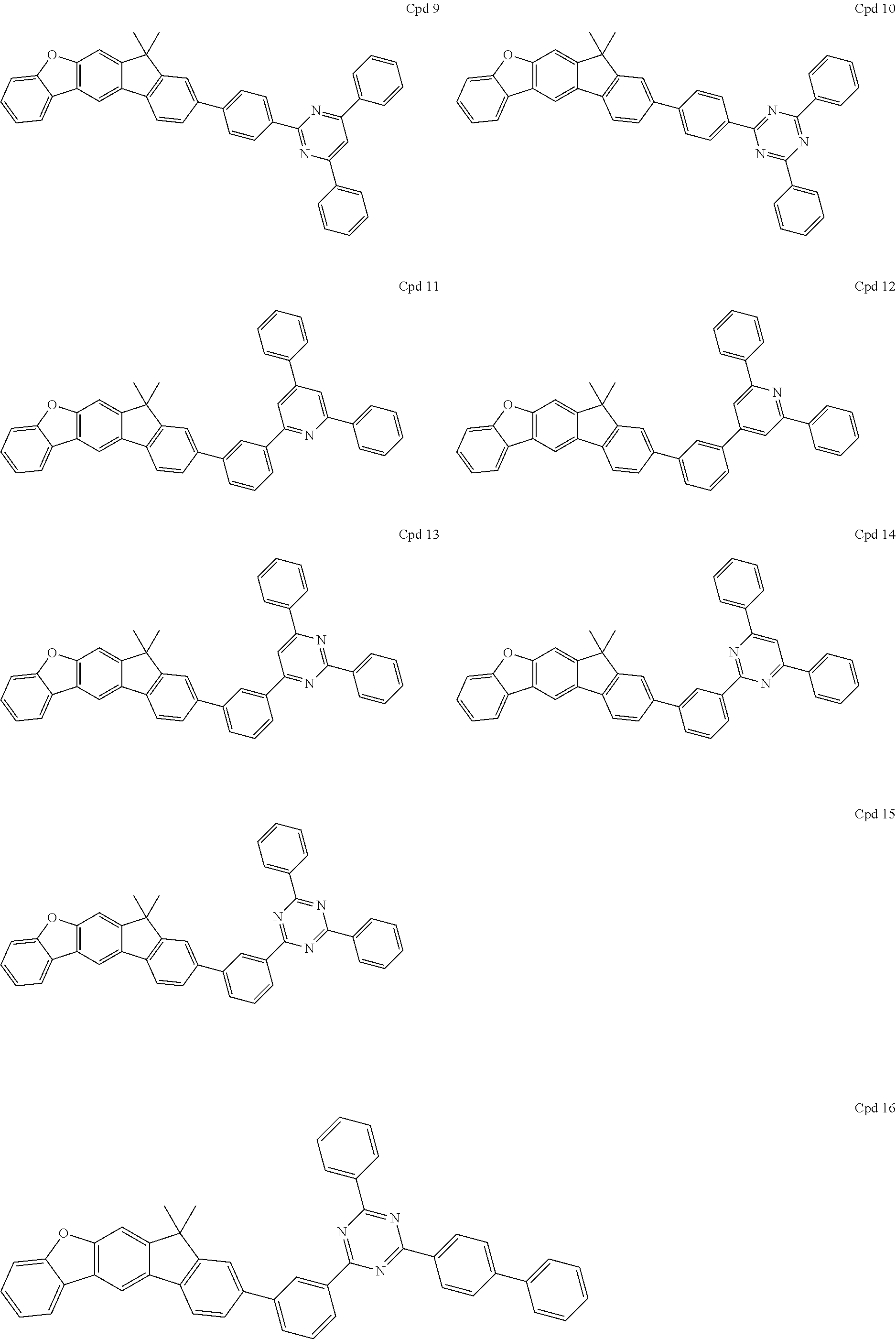

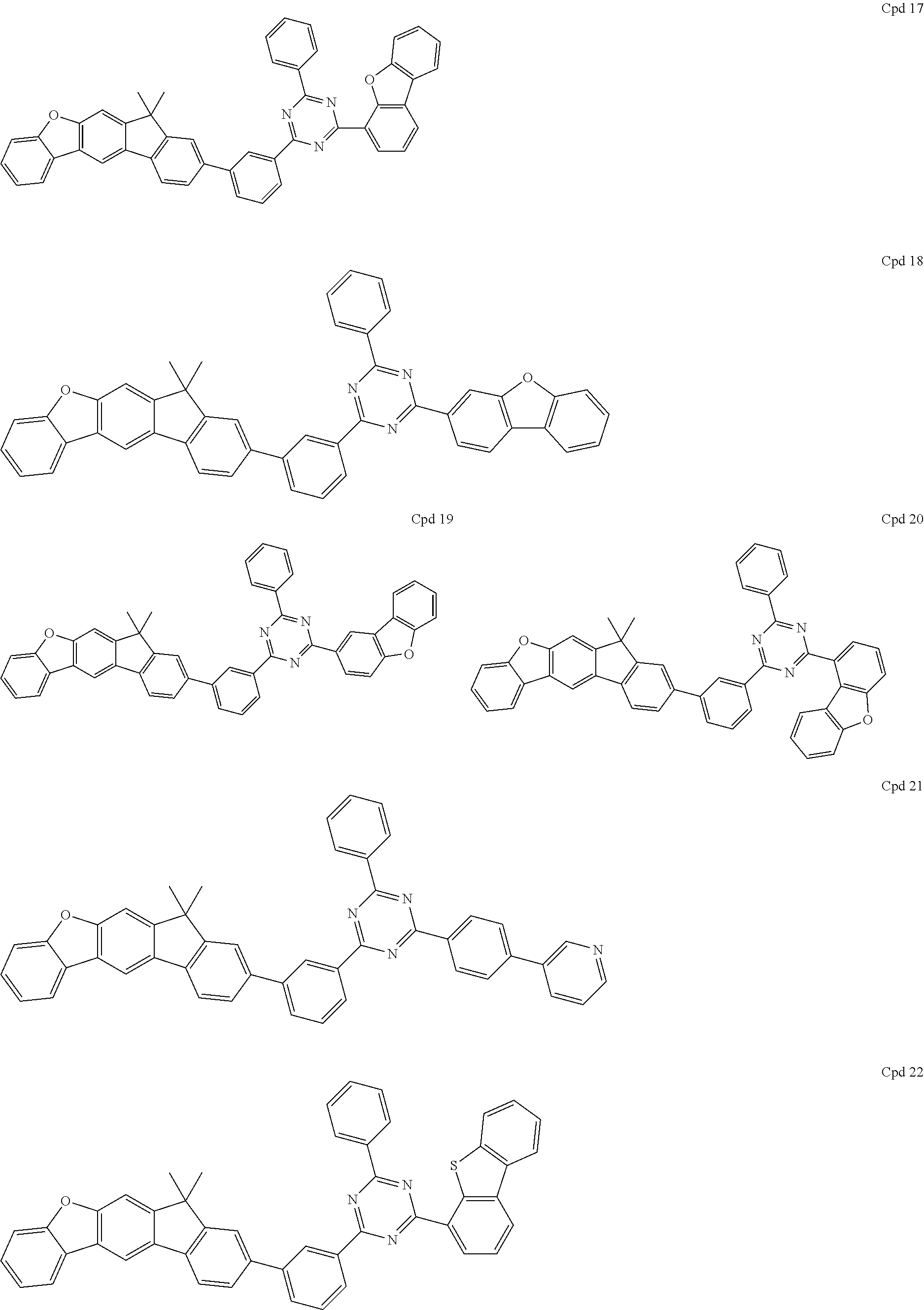

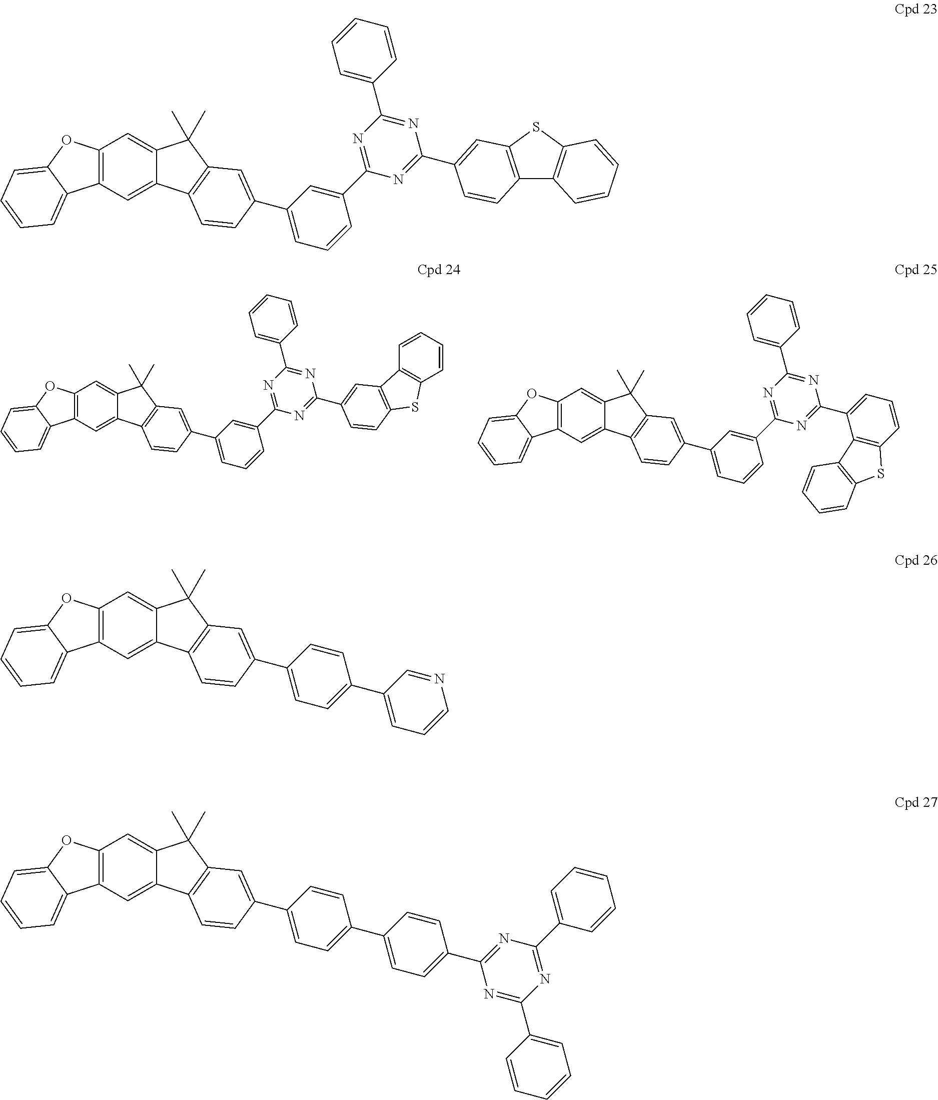 [SMILES: CC1(C)c2cc(-c3cccc(-c4nc(-c5ccccc5)nc(-c5ccc6c(c5)sc5ccccc56)n4)c3)ccc2-c2cc3c(cc21)oc1ccccc13 ; CC1(C)c2cc(-c3cccc(-c4nc(-c5ccccc5)nc(-c5ccc6sc7ccccc7c6c5)n4)c3)ccc2-c2cc3c(cc21)oc1ccccc13 ; CC1(C)c2cc(-c3cccc(-c4nc(-c5ccccc5)nc(-c5cccc6sc7ccccc7c56)n4)c3)ccc2-c2cc3c(cc21)oc1ccccc13 ; CC1(C)c2cc(-c3ccc(-c4cccnc4)cc3)ccc2-c2cc3c(cc21)oc1ccccc13 ; CC1(C)c2cc(-c3ccc(-c4ccc(-c5nc(-c6ccccc6)nc(-c6ccccc6)n5)cc4)cc3)ccc2-c2cc3c(cc21)oc1ccccc13]

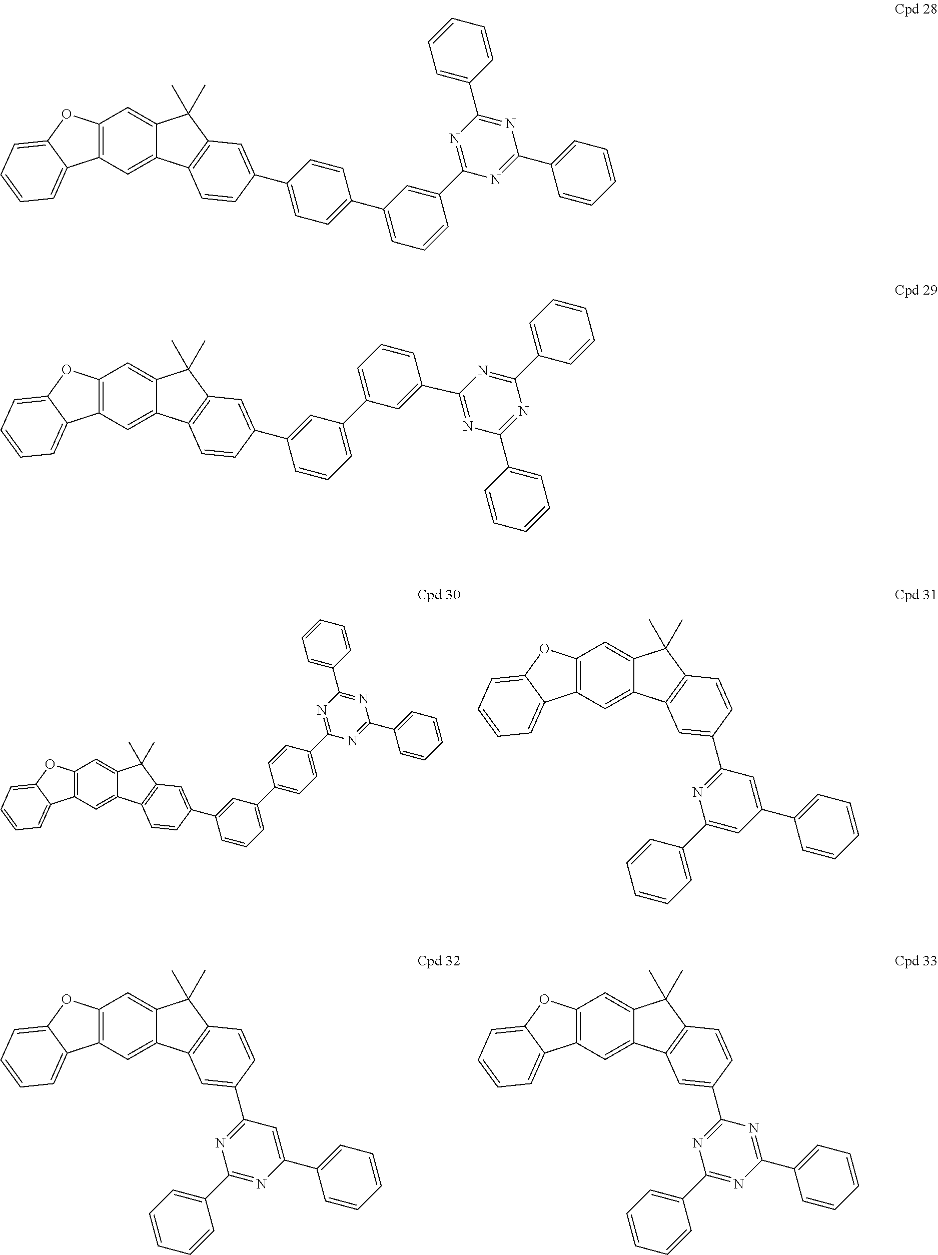

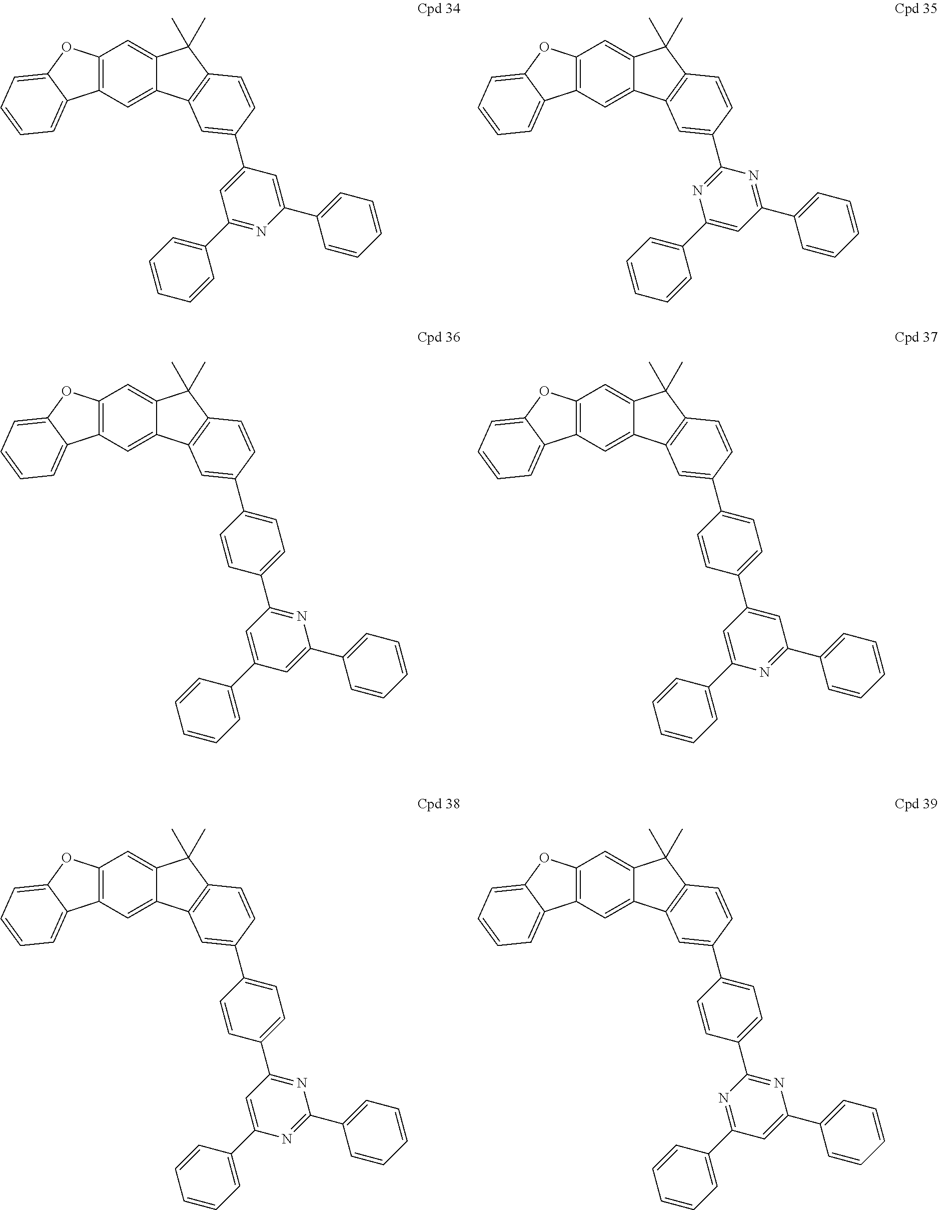 [SMILES: CC1(C)c2ccc(-c3cc(-c4ccccc4)nc(-c4ccccc4)c3)cc2-c2cc3c(cc21)oc1ccccc13 ; CC1(C)c2ccc(-c3nc(-c4ccccc4)cc(-c4ccccc4)n3)cc2-c2cc3c(cc21)oc1ccccc13 ; CC1(C)c2ccc(-c3ccc(-c4cc(-c5ccccc5)cc(-c5ccccc5)n4)cc3)cc2-c2cc3c(cc21)oc1ccccc13 ; CC1(C)c2ccc(-c3ccc(-c4cc(-c5ccccc5)nc(-c5ccccc5)c4)cc3)cc2-c2cc3c(cc21)oc1ccccc13 ; CC1(C)c2ccc(-c3ccc(-c4cc(-c5ccccc5)nc(-c5ccccc5)n4)cc3)cc2-c2cc3c(cc21)oc1ccccc13 ; CC1(C)c2ccc(-c3ccc(-c4nc(-c5ccccc5)cc(-c5ccccc5)n4)cc3)cc2-c2cc3c(cc21)oc1ccccc13]

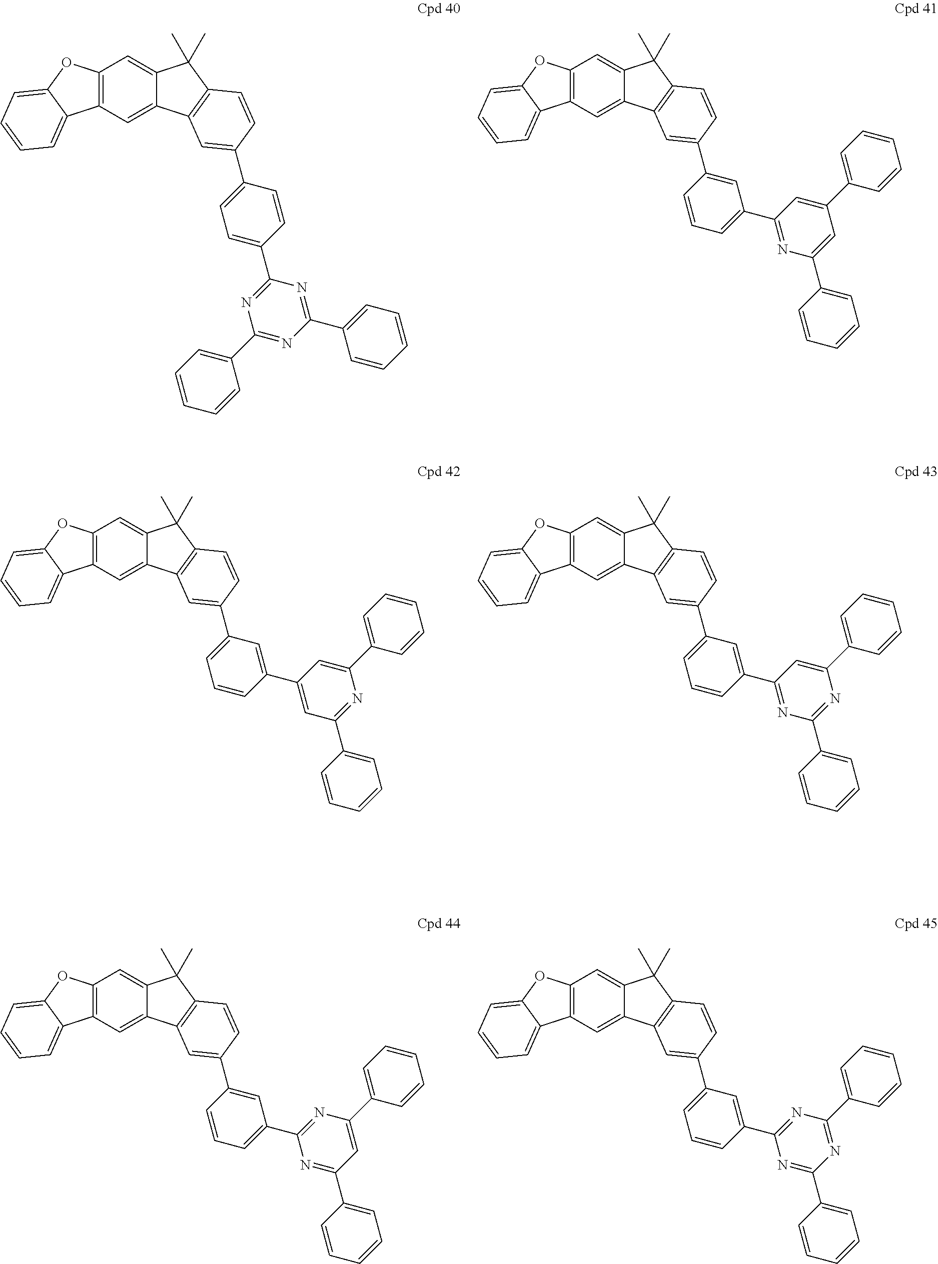

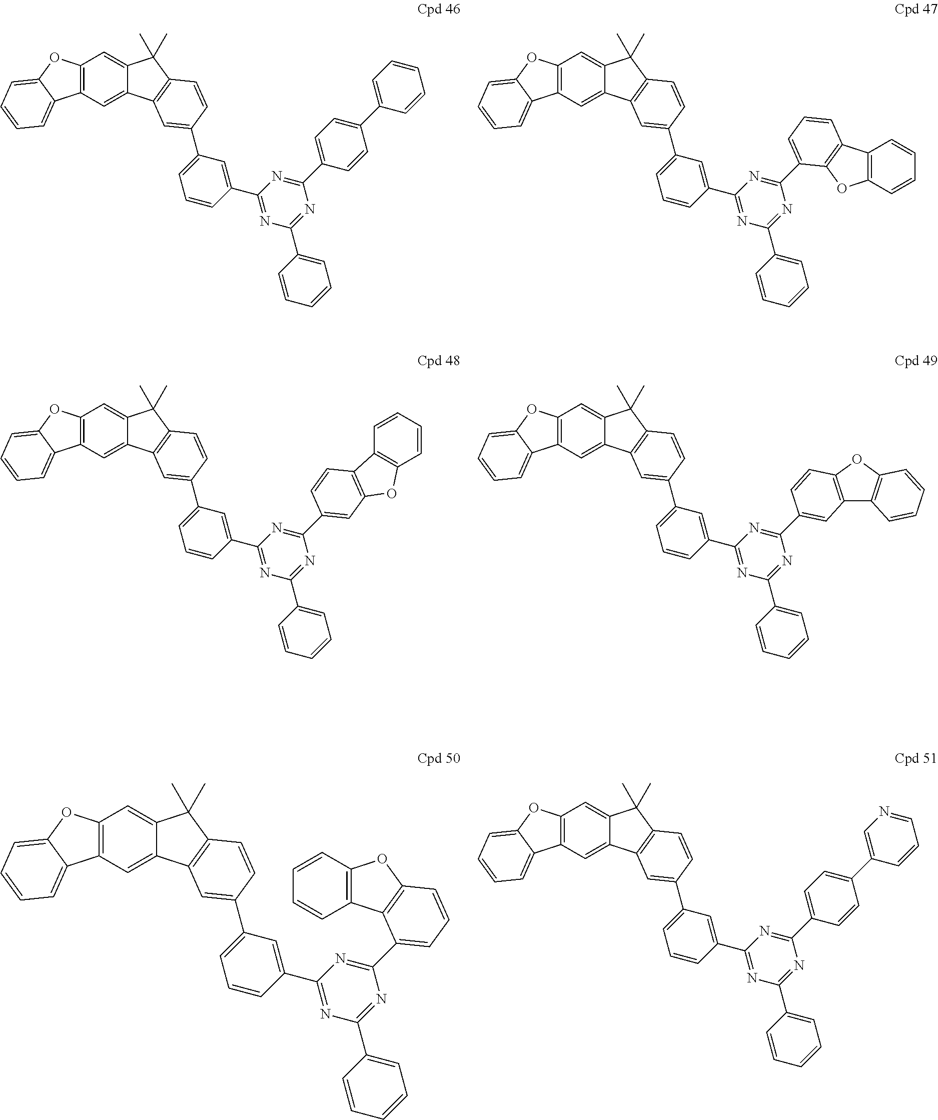

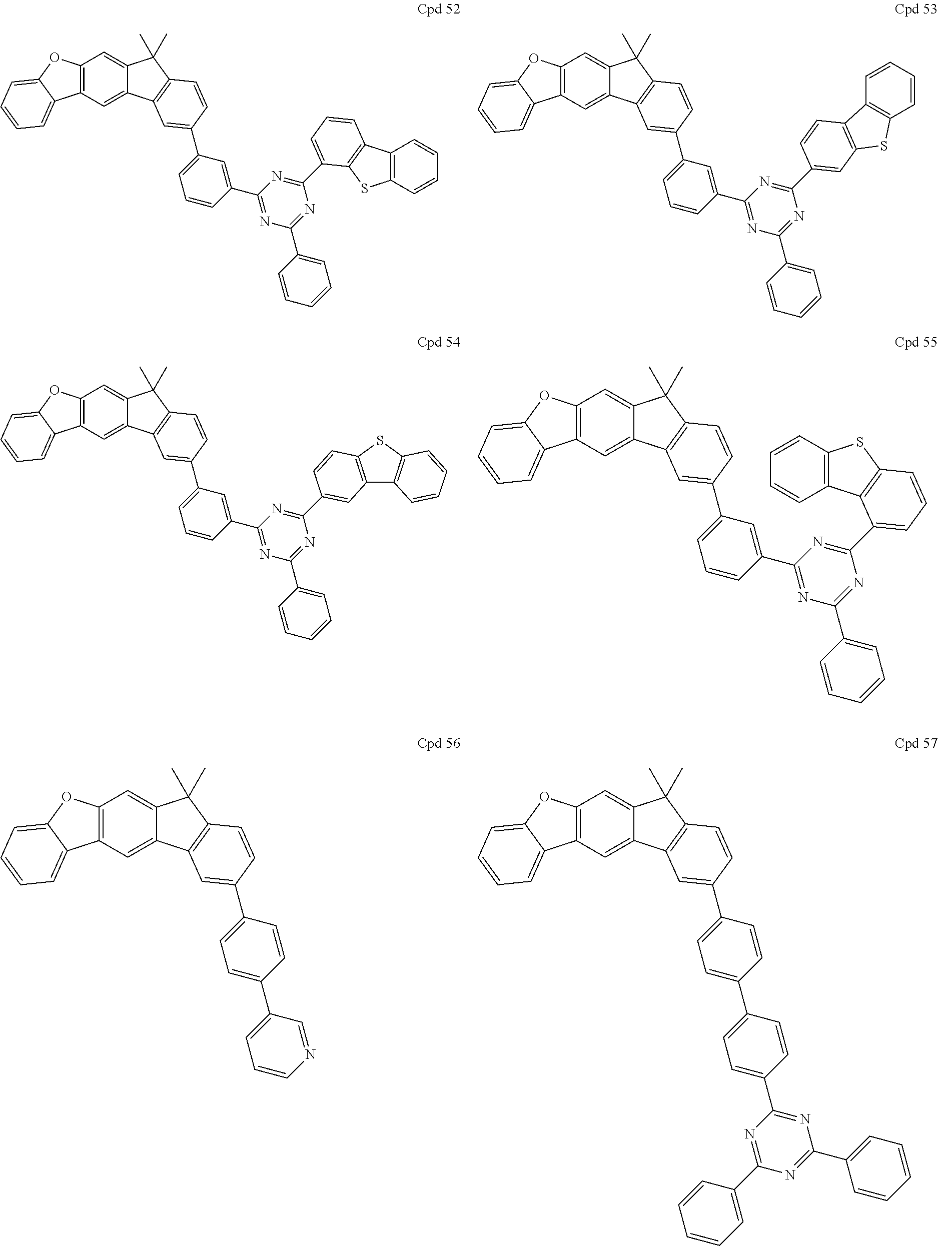

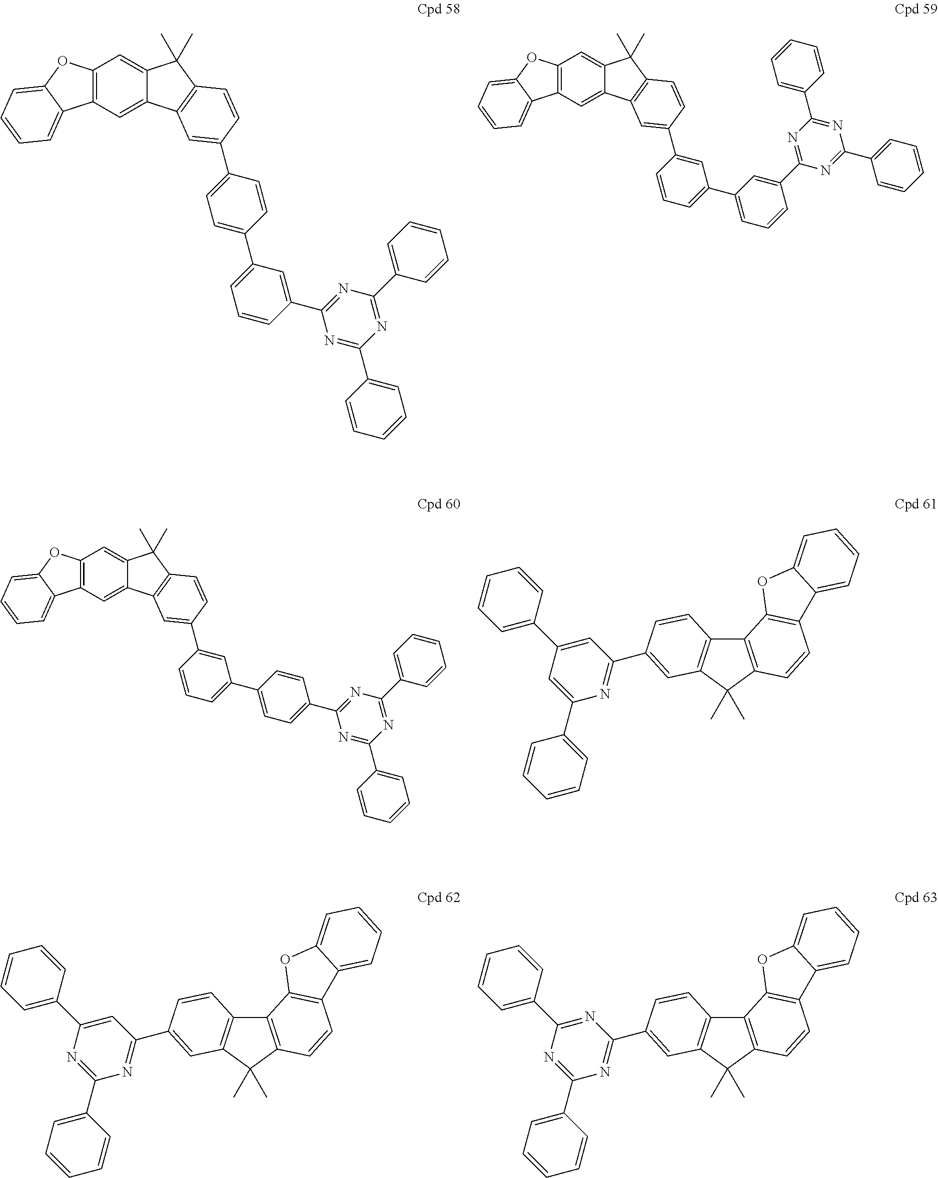 [SMILES: CC1(C)c2ccc(-c3ccc(-c4cccc(-c5nc(-c6ccccc6)nc(-c6ccccc6)n5)c4)cc3)cc2-c2cc3c(cc21)oc1ccccc13 ; CC1(C)c2ccc(-c3cccc(-c4cccc(-c5nc(-c6ccccc6)nc(-c6ccccc6)n5)c4)c3)cc2-c2cc3c(cc21)oc1ccccc13 ; CC1(C)c2ccc(-c3cccc(-c4ccc(-c5nc(-c6ccccc6)nc(-c6ccccc6)n5)cc4)c3)cc2-c2cc3c(cc21)oc1ccccc13 ; CC1(C)c2cc(-c3cc(-c4ccccc4)cc(-c4ccccc4)n3)ccc2-c2c1ccc1c2oc2ccccc21 ; CC1(C)c2cc(-c3cc(-c4ccccc4)nc(-c4ccccc4)n3)ccc2-c2c1ccc1c2oc2ccccc21 ; CC1(C)c2cc(-c3nc(-c4ccccc4)nc(-c4ccccc4)n3)ccc2-c2c1ccc1c2oc2ccccc21]

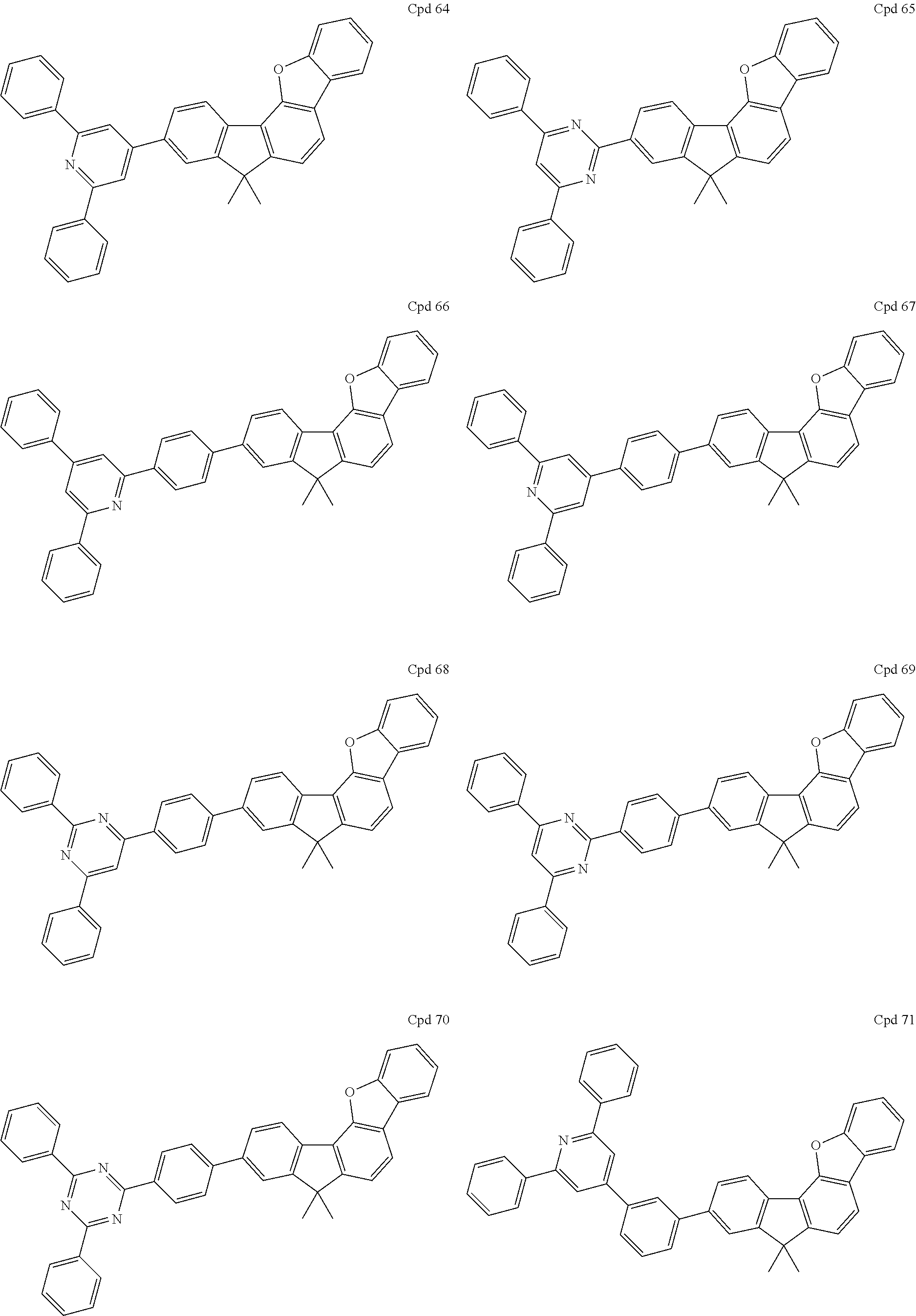

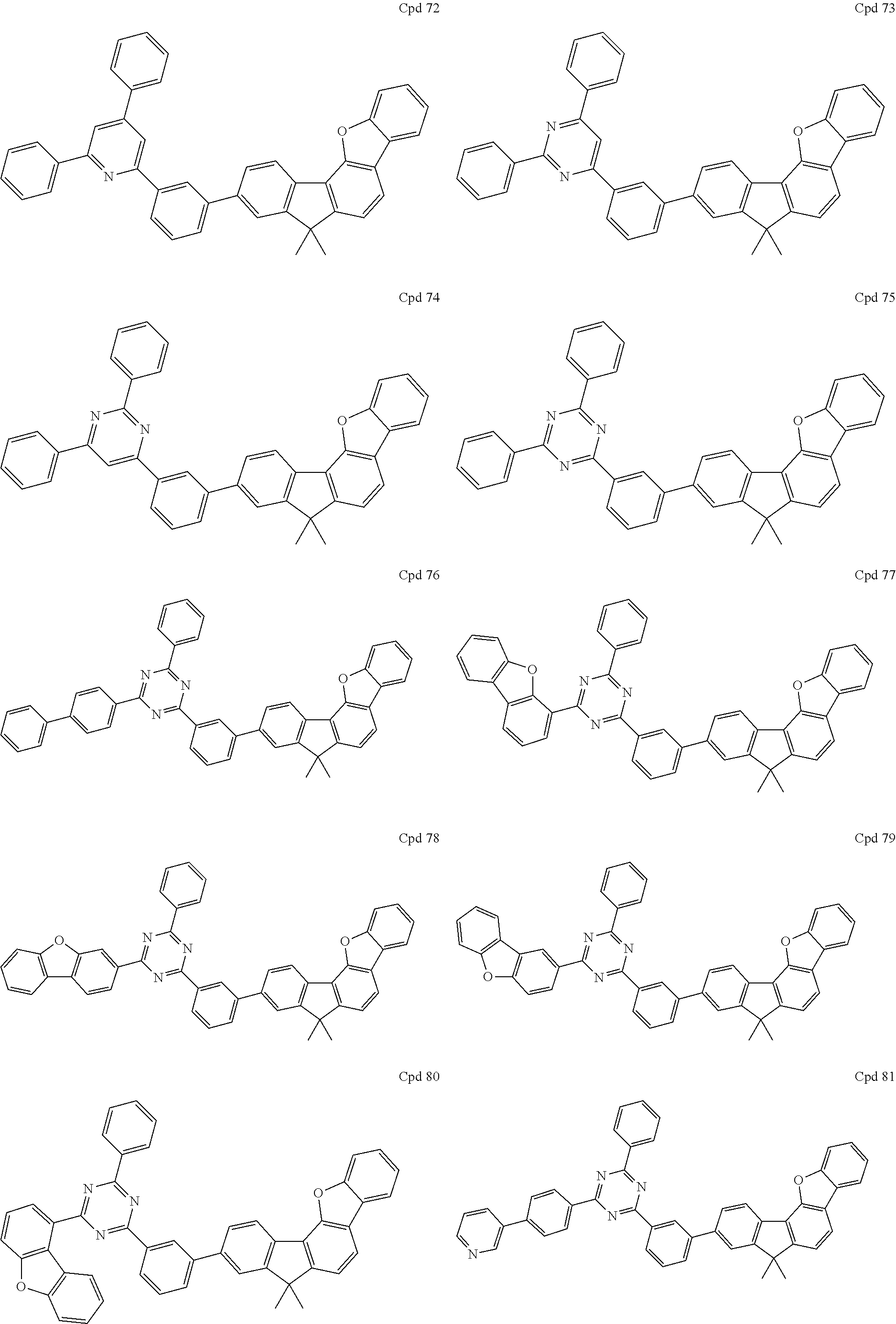

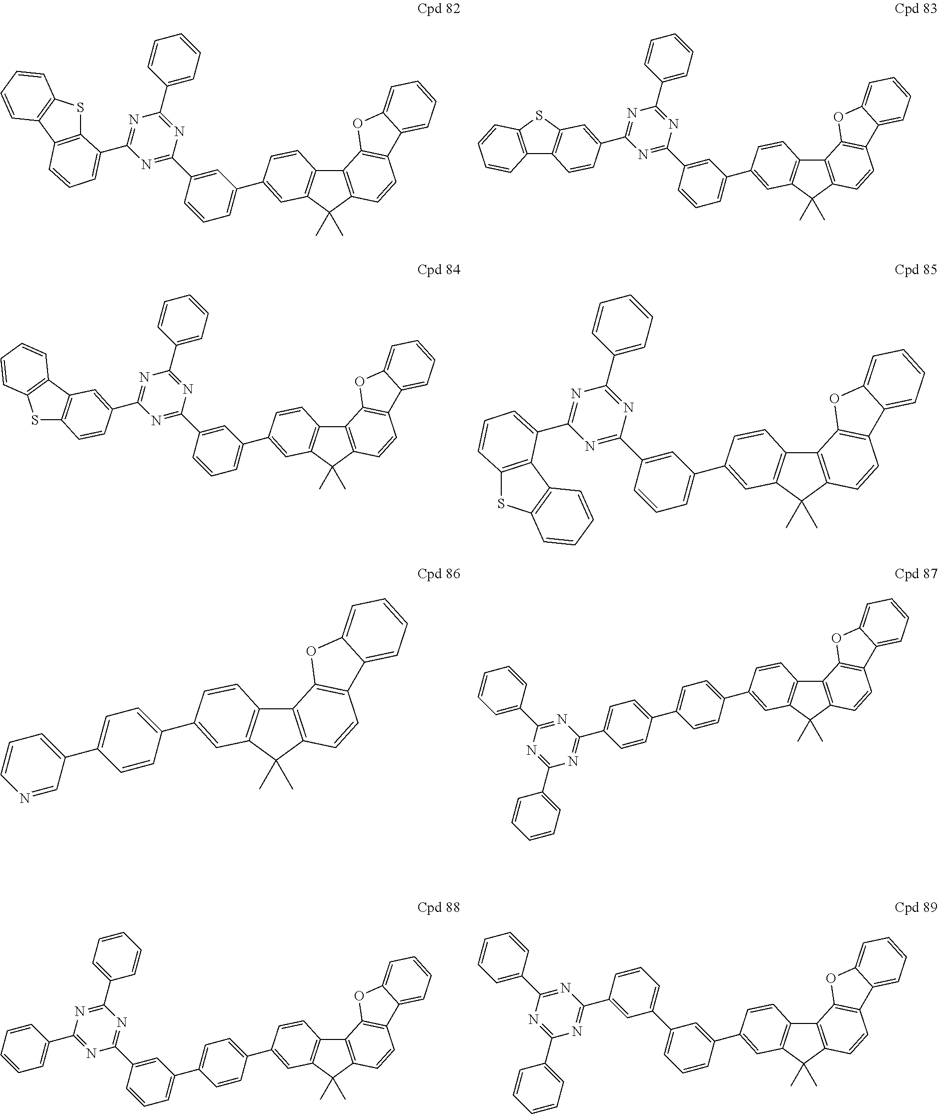

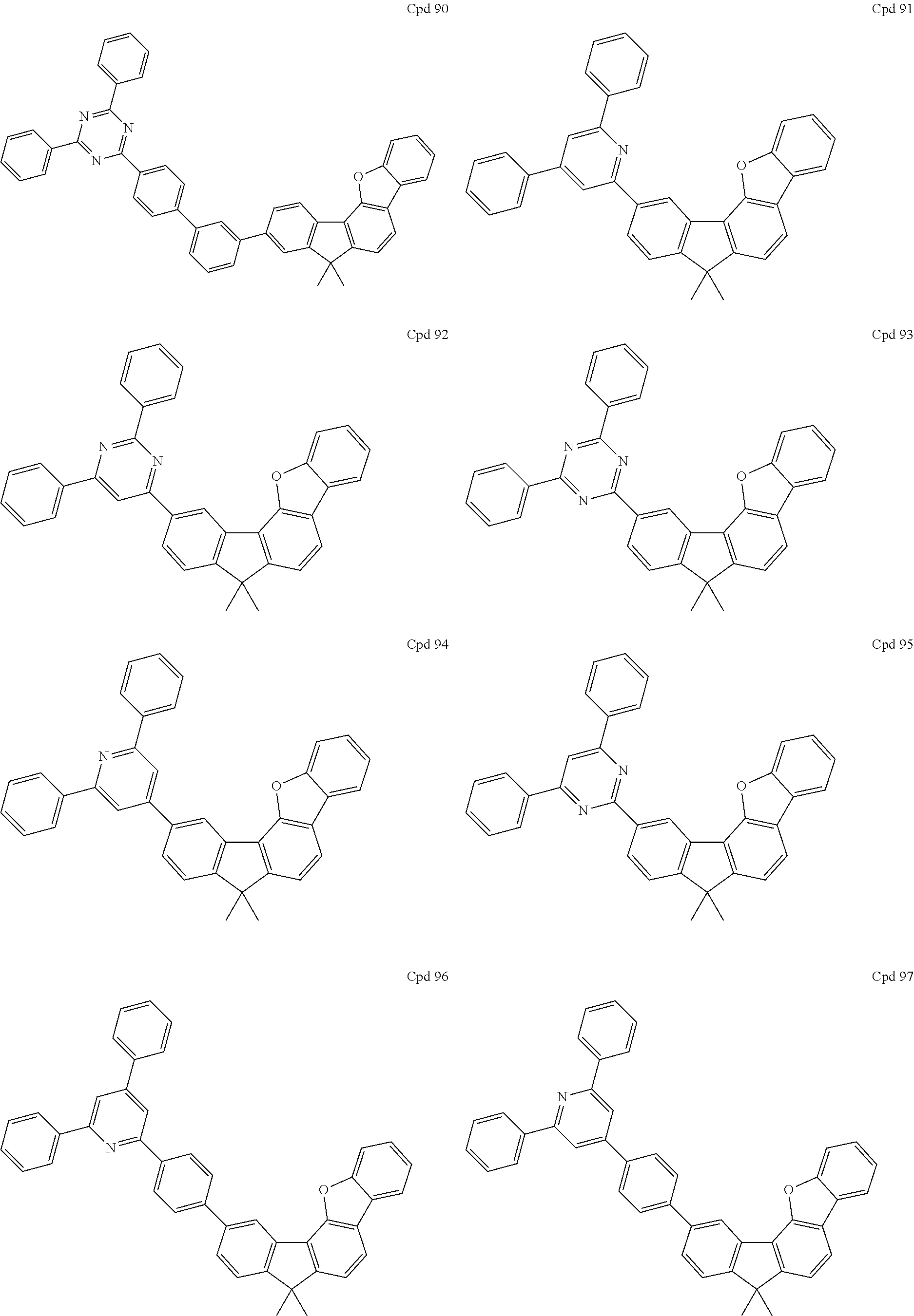

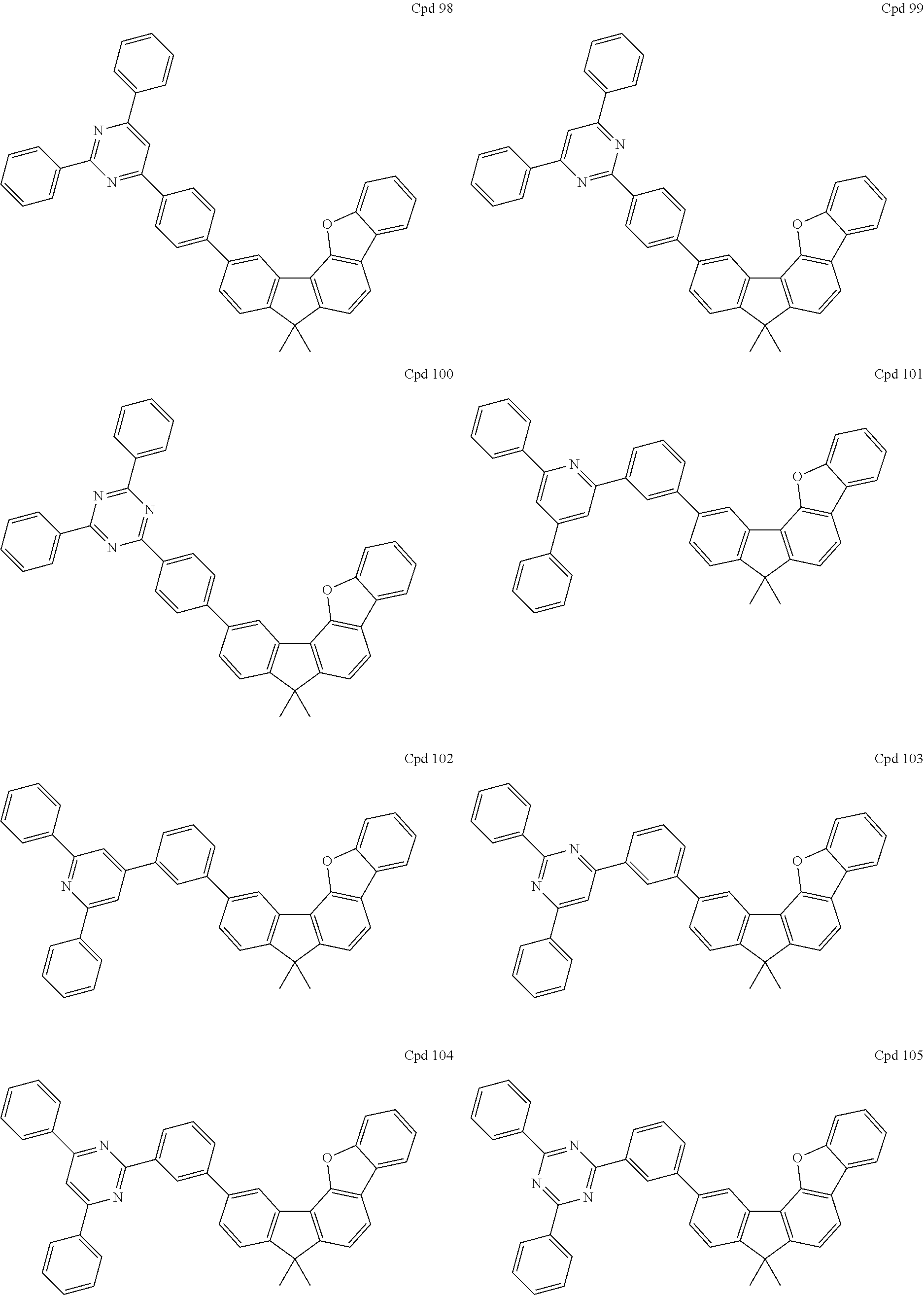

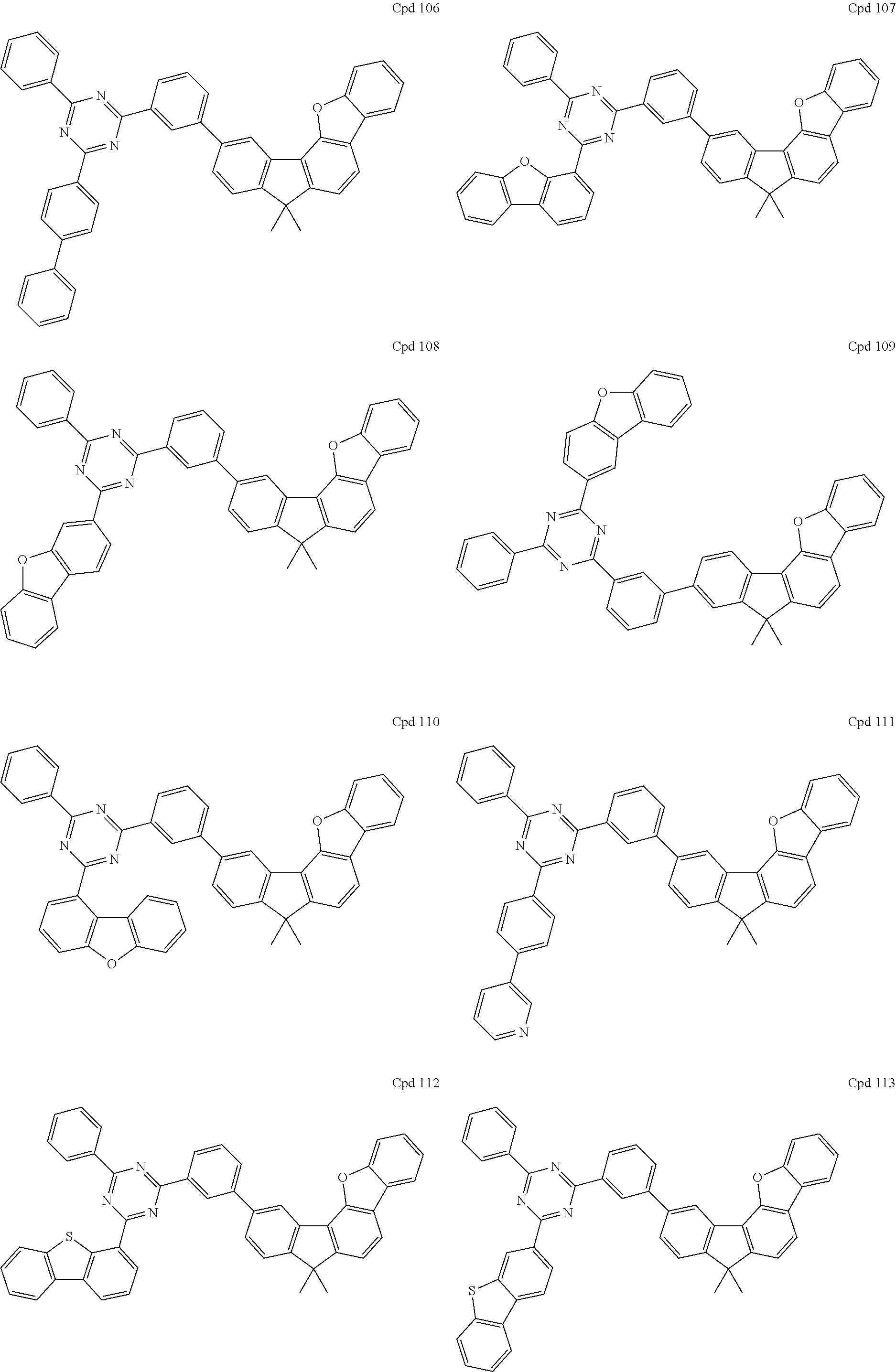

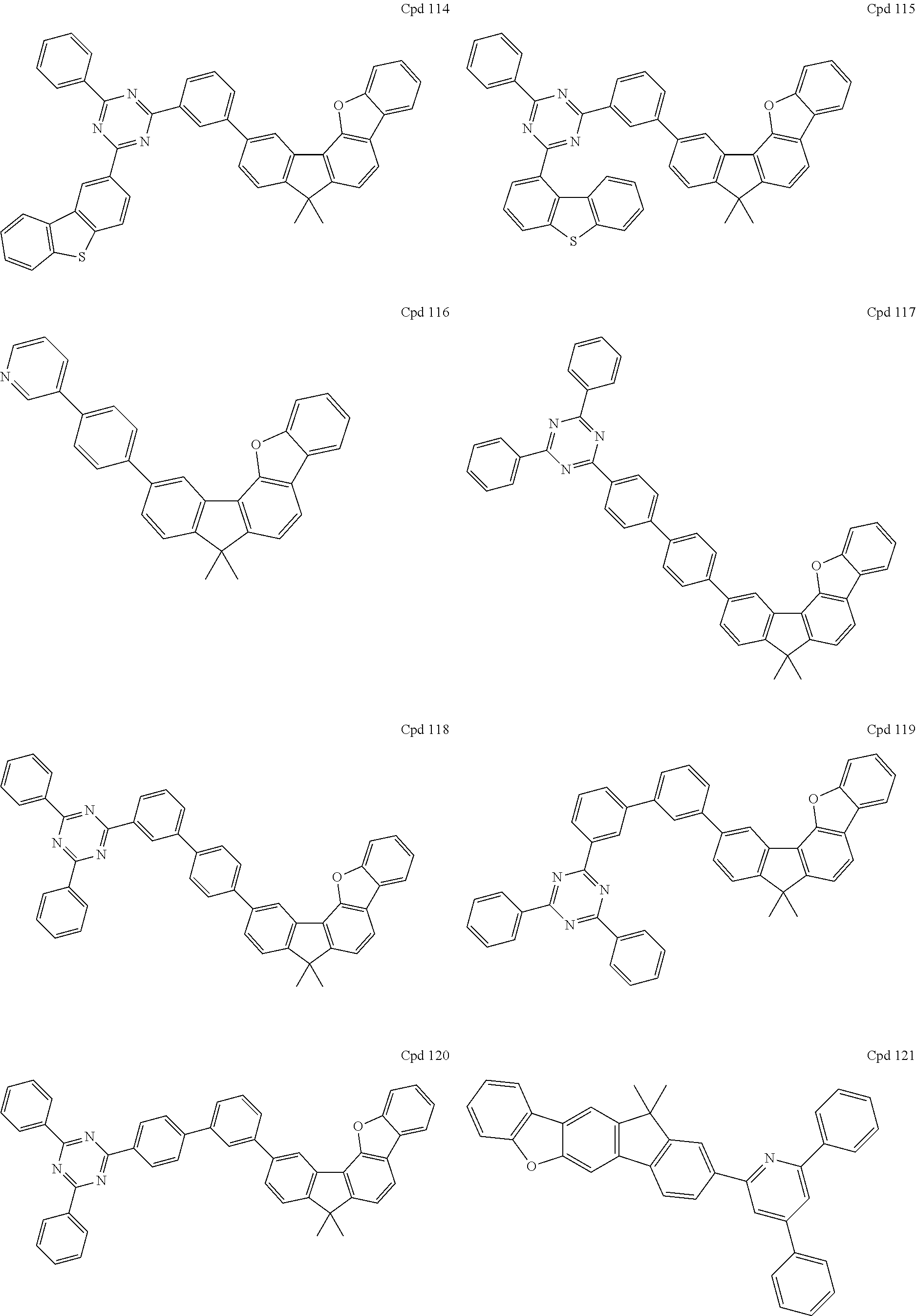

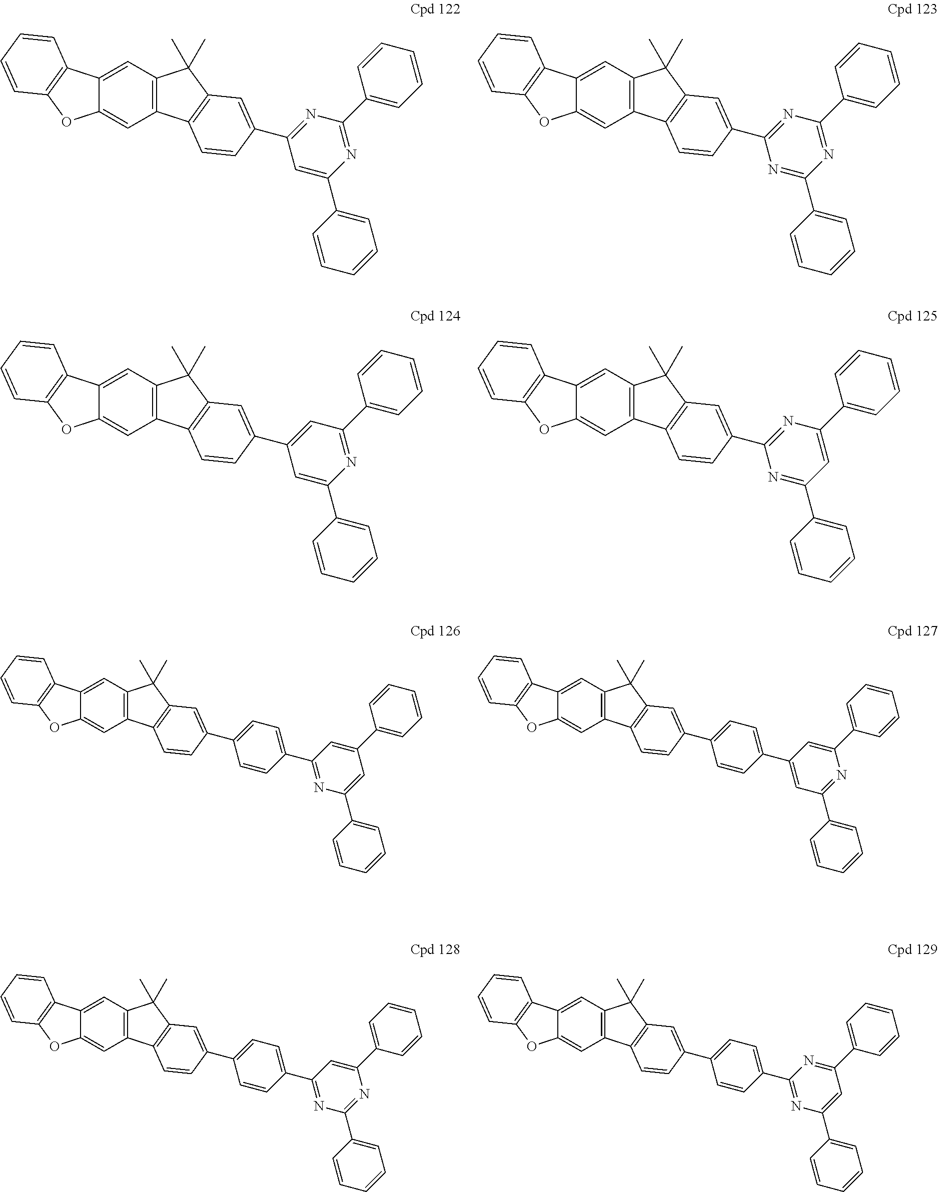

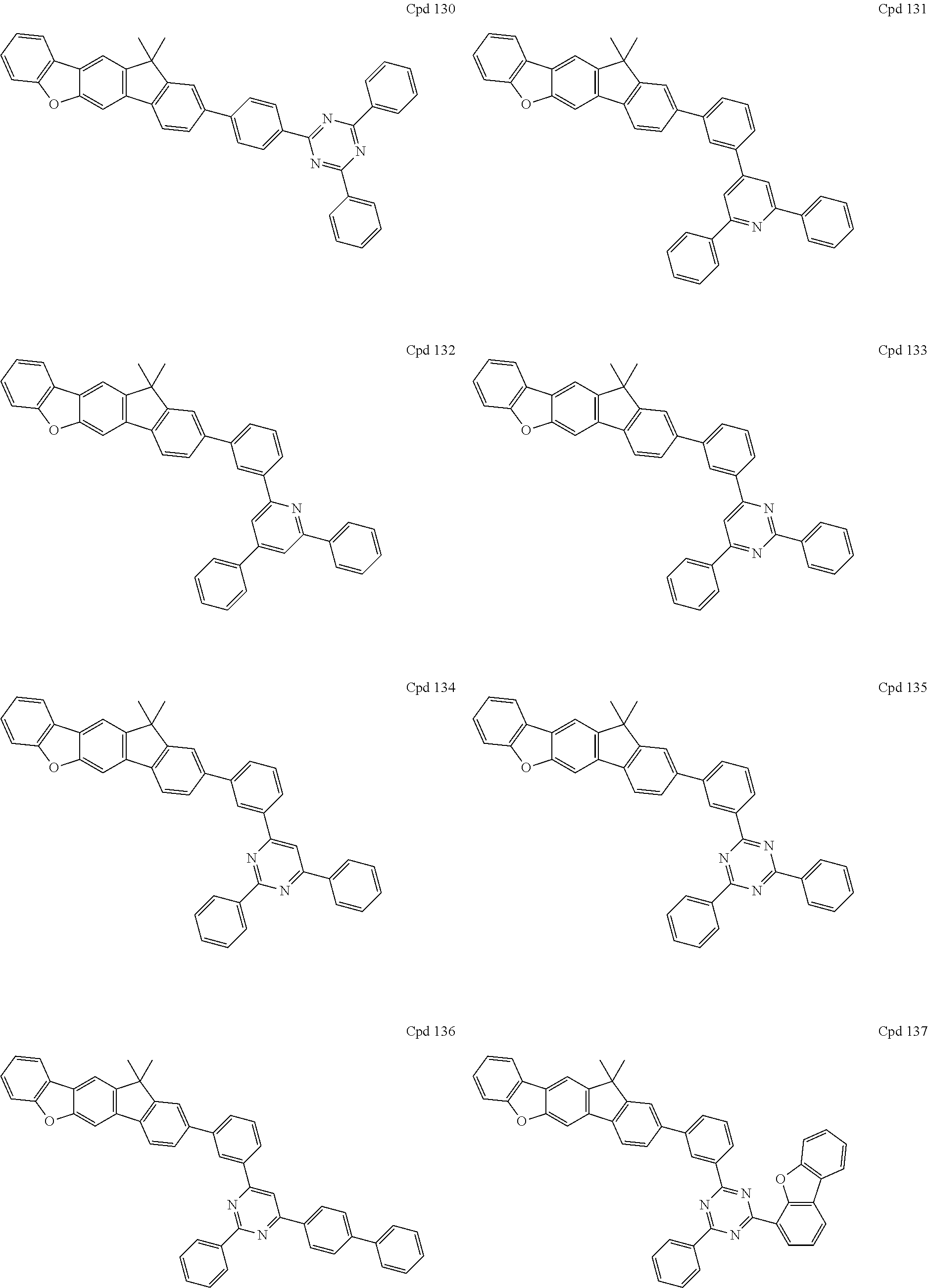

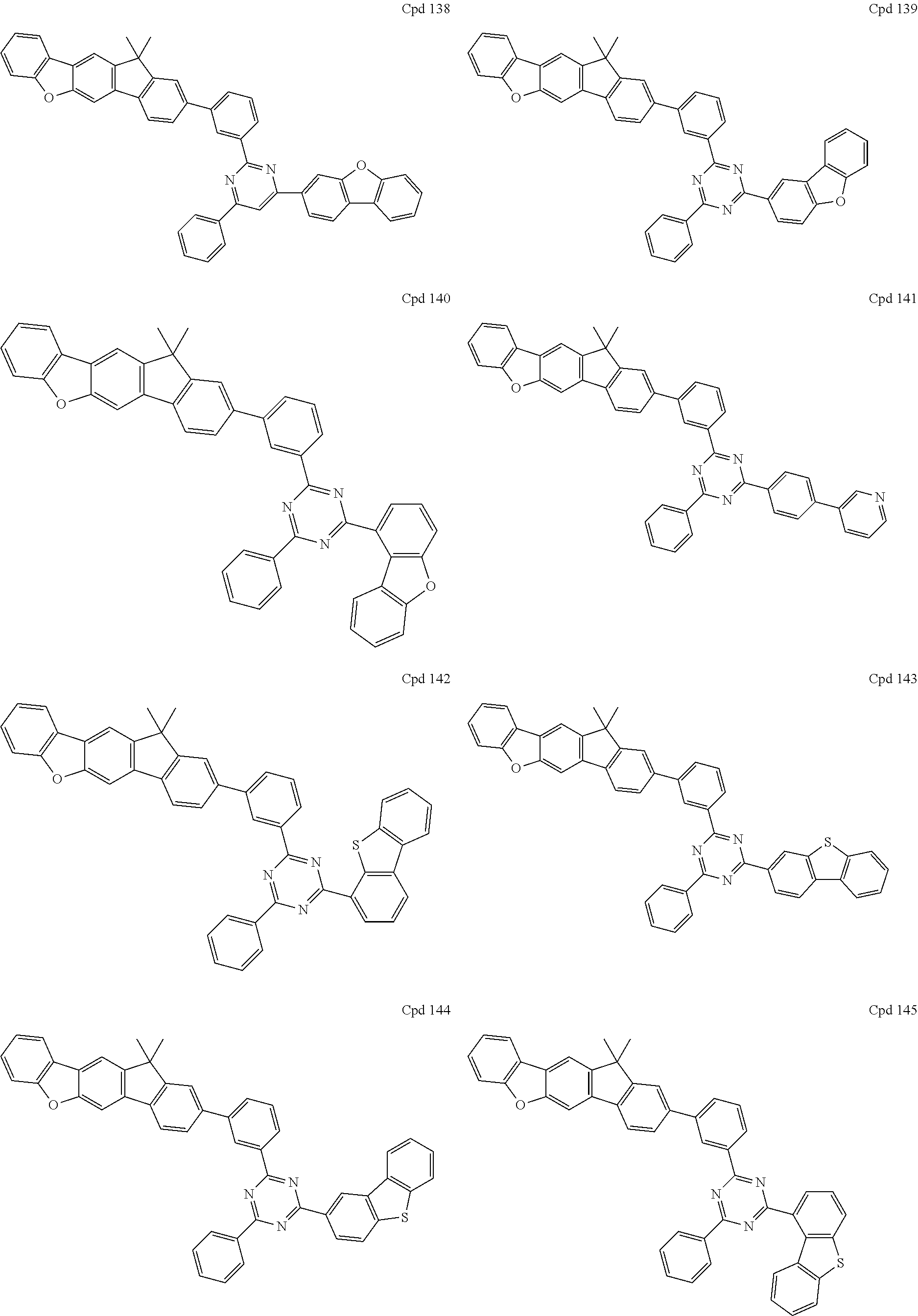

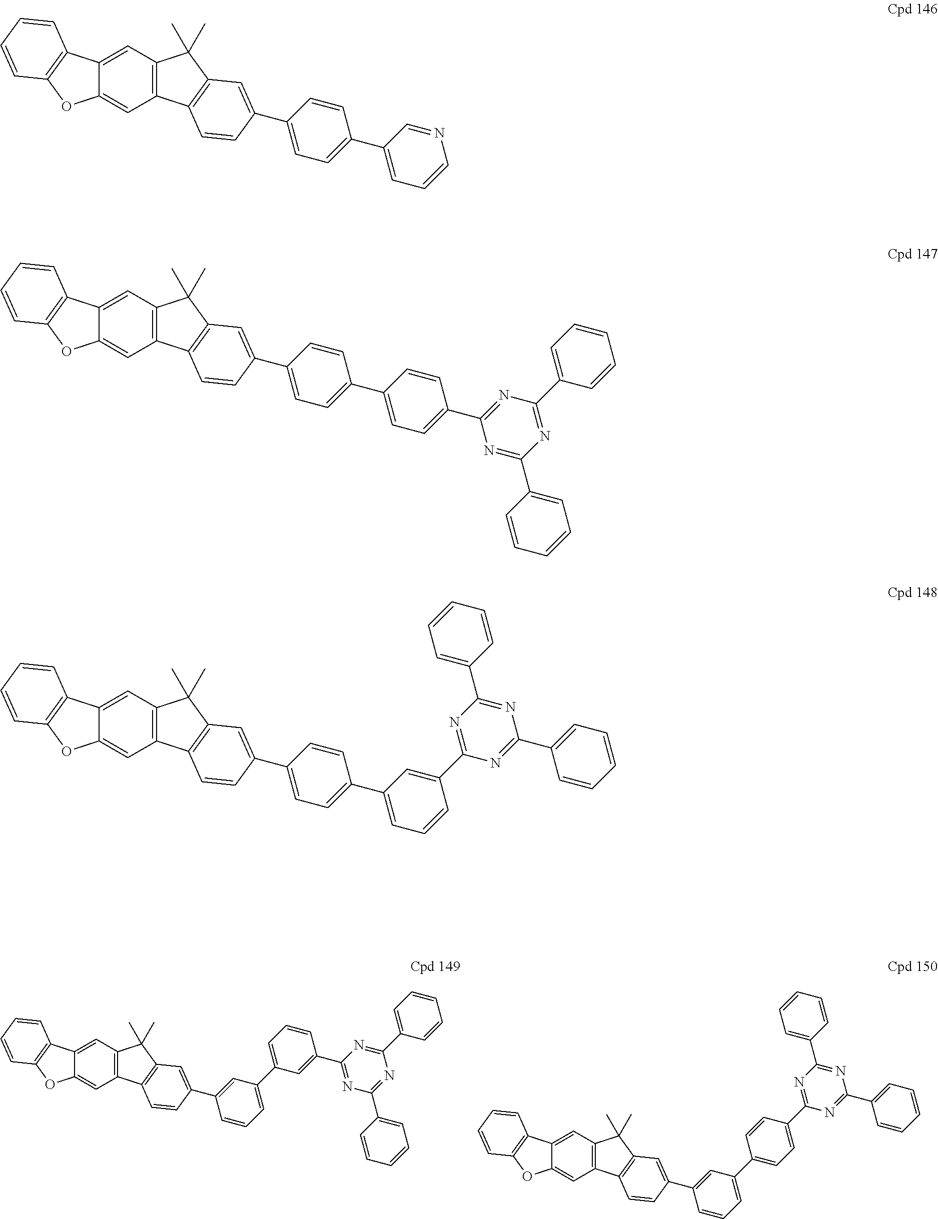 [SMILES: CC1(C)c2cc(-c3ccc(-c4cccnc4)cc3)ccc2-c2cc3oc4ccccc4c3cc21 ; CC1(C)c2cc(-c3ccc(-c4ccc(-c5nc(-c6ccccc6)nc(-c6ccccc6)n5)cc4)cc3)ccc2-c2cc3oc4ccccc4c3cc21 ; CC1(C)c2cc(-c3ccc(-c4cccc(-c5nc(-c6ccccc6)nc(-c6ccccc6)n5)c4)cc3)ccc2-c2cc3oc4ccccc4c3cc21 ; CC1(C)c2cc(-c3cccc(-c4cccc(-c5nc(-c6ccccc6)nc(-c6ccccc6)n5)c4)c3)ccc2-c2cc3oc4ccccc4c3cc21 ; CC1(C)c2cc(-c3cccc(-c4ccc(-c5nc(-c6ccccc6)nc(-c6ccccc6)n5)cc4)c3)ccc2-c2cc3oc4ccccc4c3cc21]

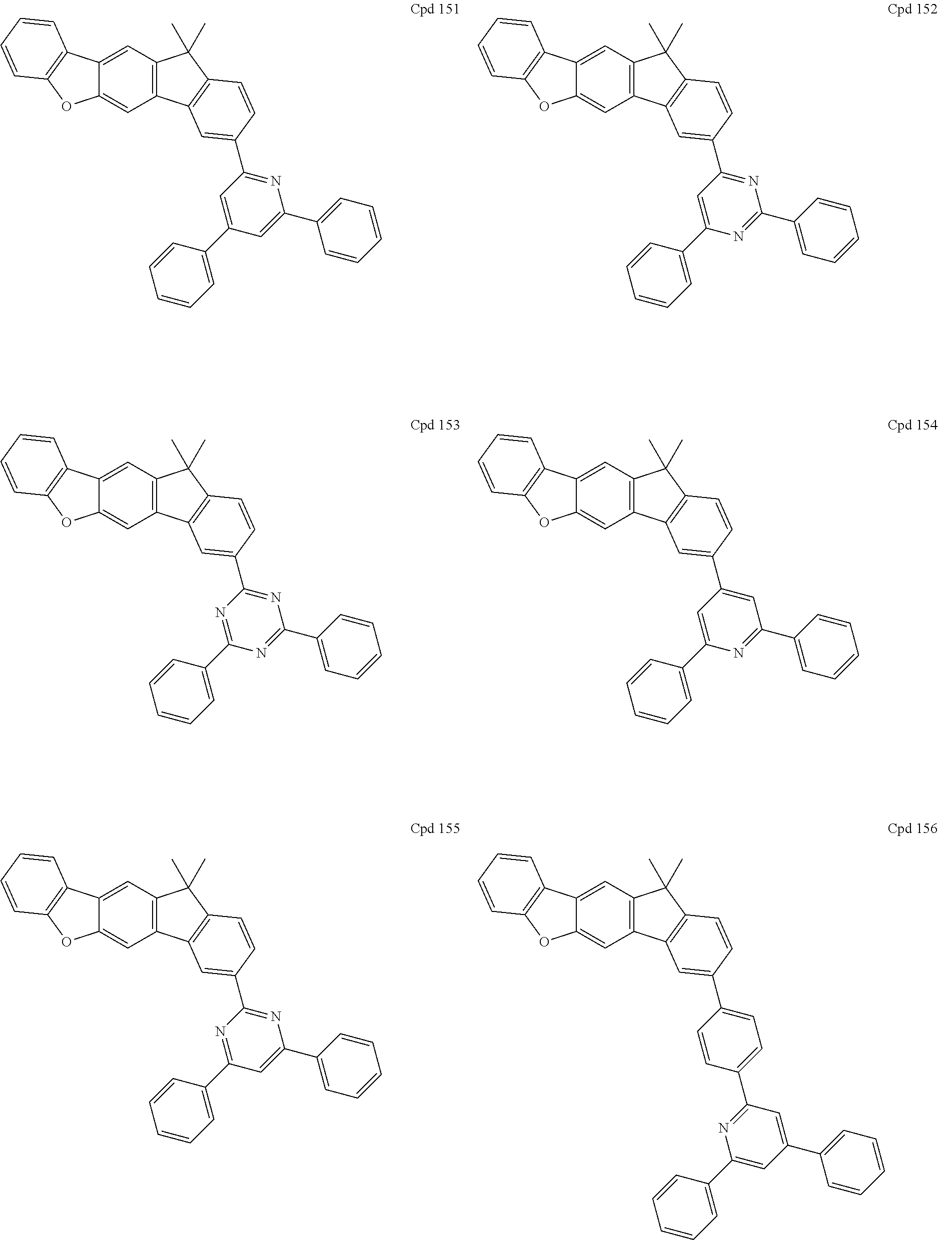 [SMILES: CC1(C)c2ccc(-c3cc(-c4ccccc4)cc(-c4ccccc4)n3)cc2-c2cc3oc4ccccc4c3cc21 ; CC1(C)c2ccc(-c3cc(-c4ccccc4)nc(-c4ccccc4)n3)cc2-c2cc3oc4ccccc4c3cc21 ; CC1(C)c2ccc(-c3nc(-c4ccccc4)nc(-c4ccccc4)n3)cc2-c2cc3oc4ccccc4c3cc21 ; CC1(C)c2ccc(-c3cc(-c4ccccc4)nc(-c4ccccc4)c3)cc2-c2cc3oc4ccccc4c3cc21 ; CC1(C)c2ccc(-c3nc(-c4ccccc4)cc(-c4ccccc4)n3)cc2-c2cc3oc4ccccc4c3cc21 ; CC1(C)c2ccc(-c3ccc(-c4cc(-c5ccccc5)cc(-c5ccccc5)n4)cc3)cc2-c2cc3oc4ccccc4c3cc21]

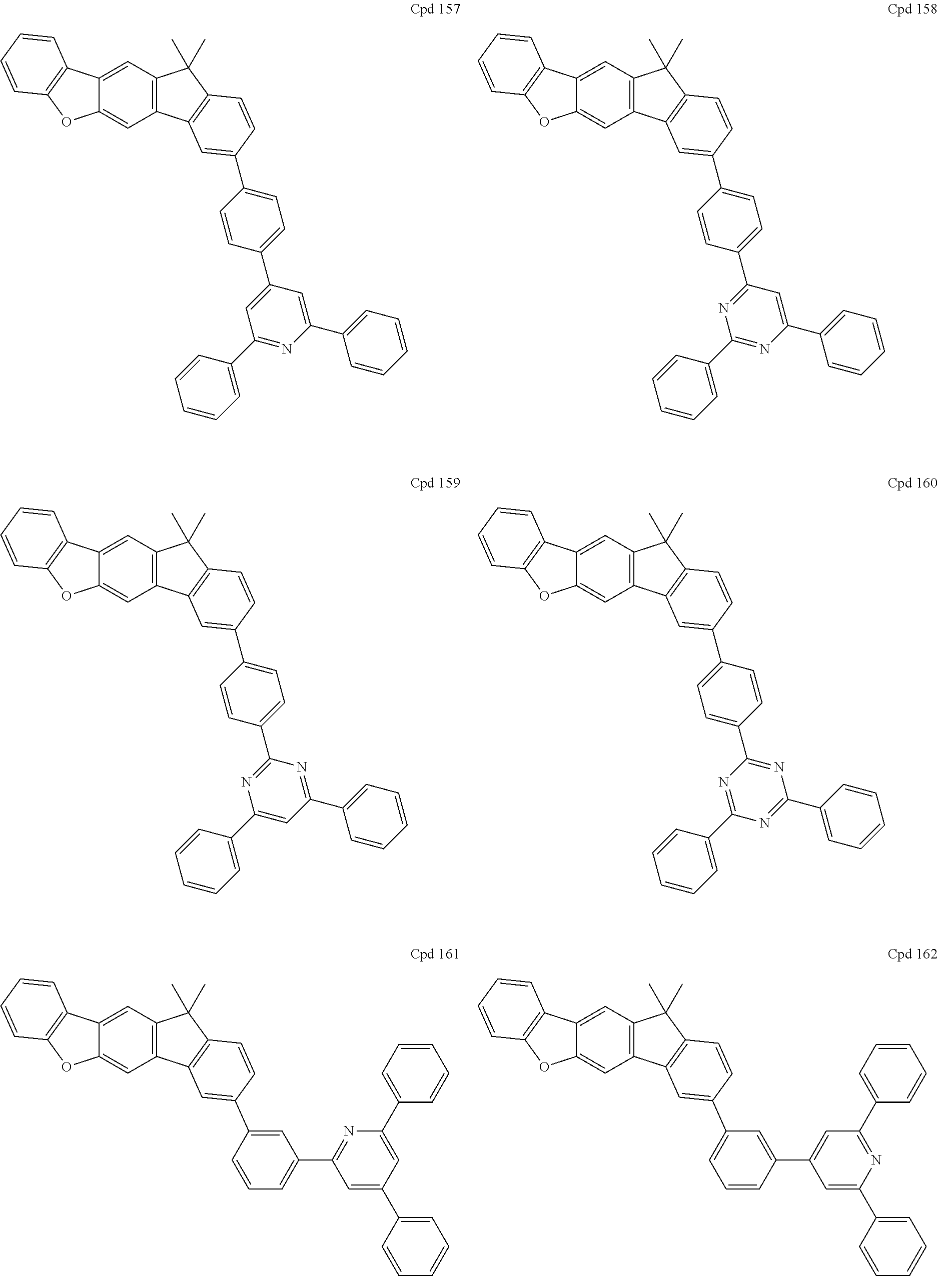 [SMILES: CC1(C)c2ccc(-c3ccc(-c4cc(-c5ccccc5)nc(-c5ccccc5)c4)cc3)cc2-c2cc3oc4ccccc4c3cc21 ; CC1(C)c2ccc(-c3ccc(-c4cc(-c5ccccc5)nc(-c5ccccc5)n4)cc3)cc2-c2cc3oc4ccccc4c3cc21 ; CC1(C)c2ccc(-c3ccc(-c4nc(-c5ccccc5)cc(-c5ccccc5)n4)cc3)cc2-c2cc3oc4ccccc4c3cc21 ; CC1(C)c2ccc(-c3ccc(-c4nc(-c5ccccc5)nc(-c5ccccc5)n4)cc3)cc2-c2cc3oc4ccccc4c3cc21 ; CC1(C)c2ccc(-c3cccc(-c4cc(-c5ccccc5)cc(-c5ccccc5)n4)c3)cc2-c2cc3oc4ccccc4c3cc21 ; CC1(C)c2ccc(-c3cccc(-c4cc(-c5ccccc5)nc(-c5ccccc5)c4)c3)cc2-c2cc3oc4ccccc4c3cc21]

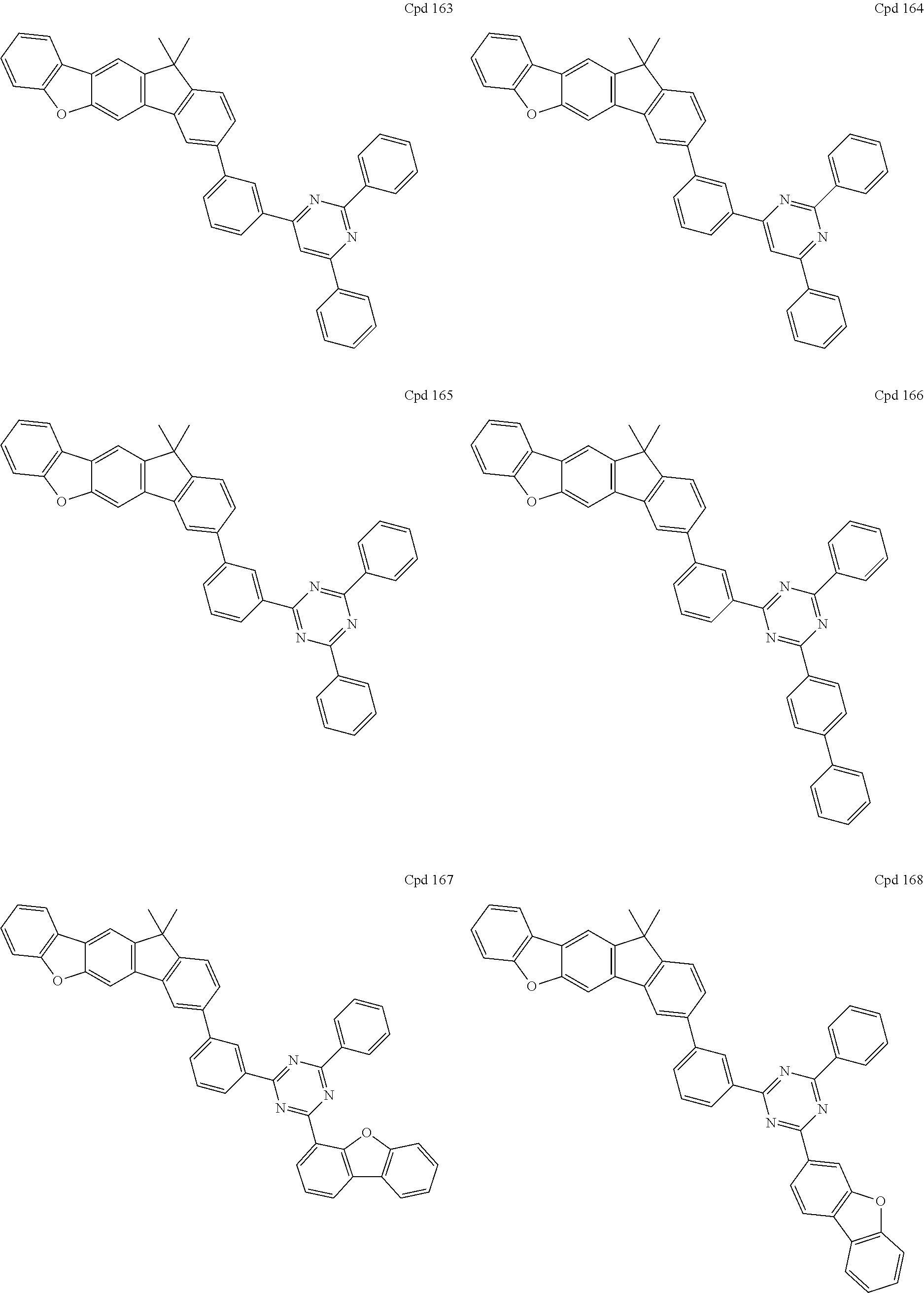

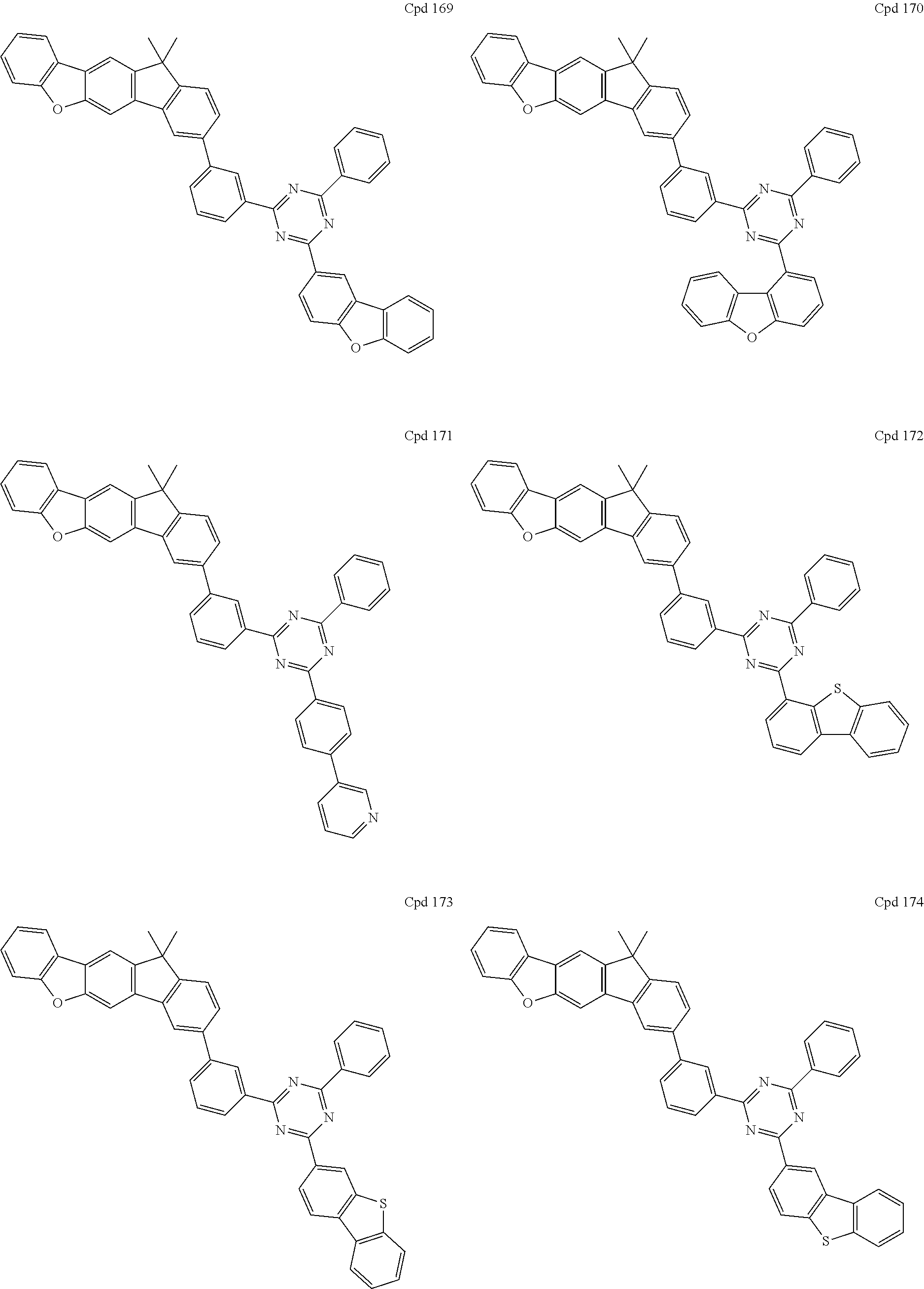

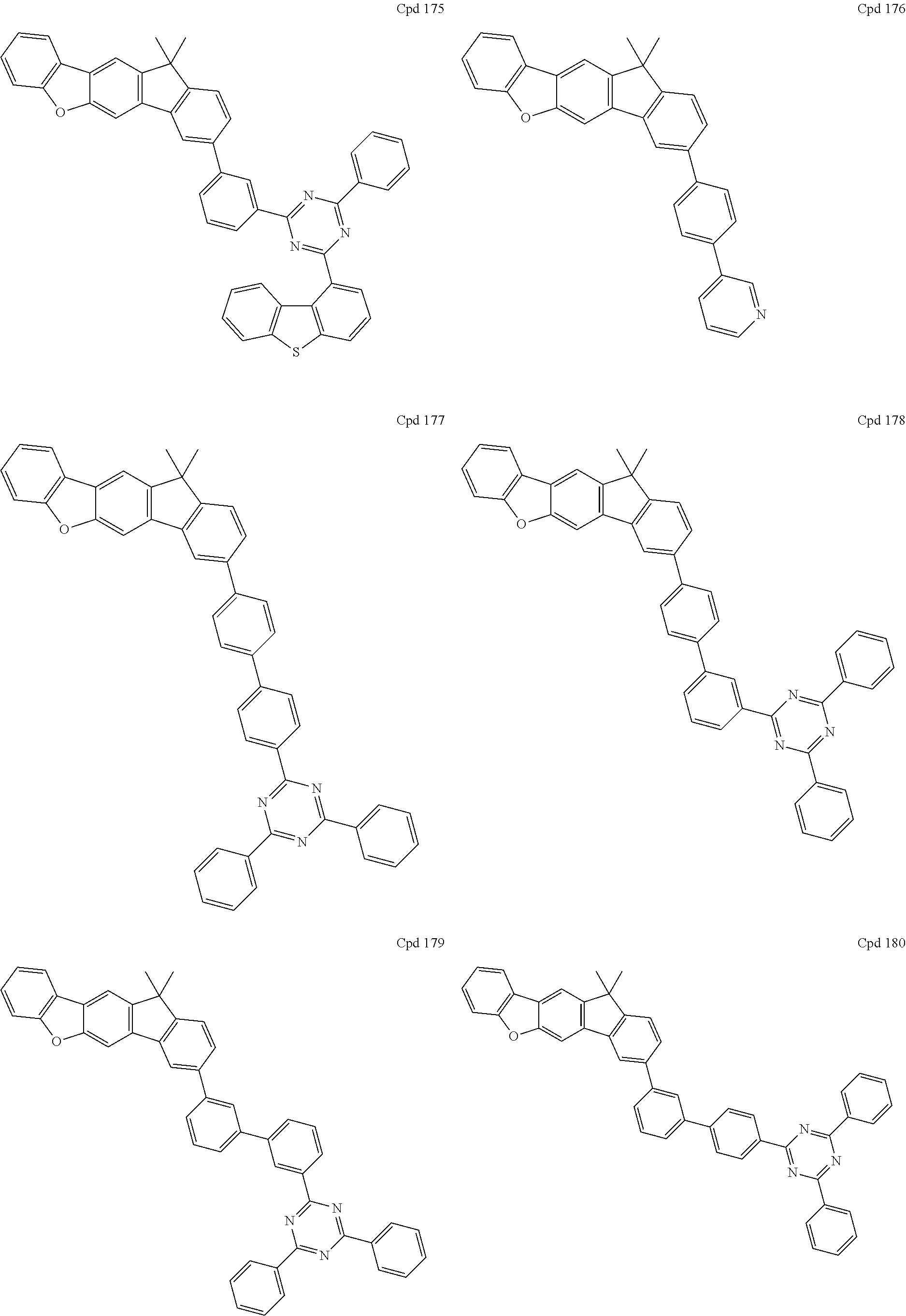

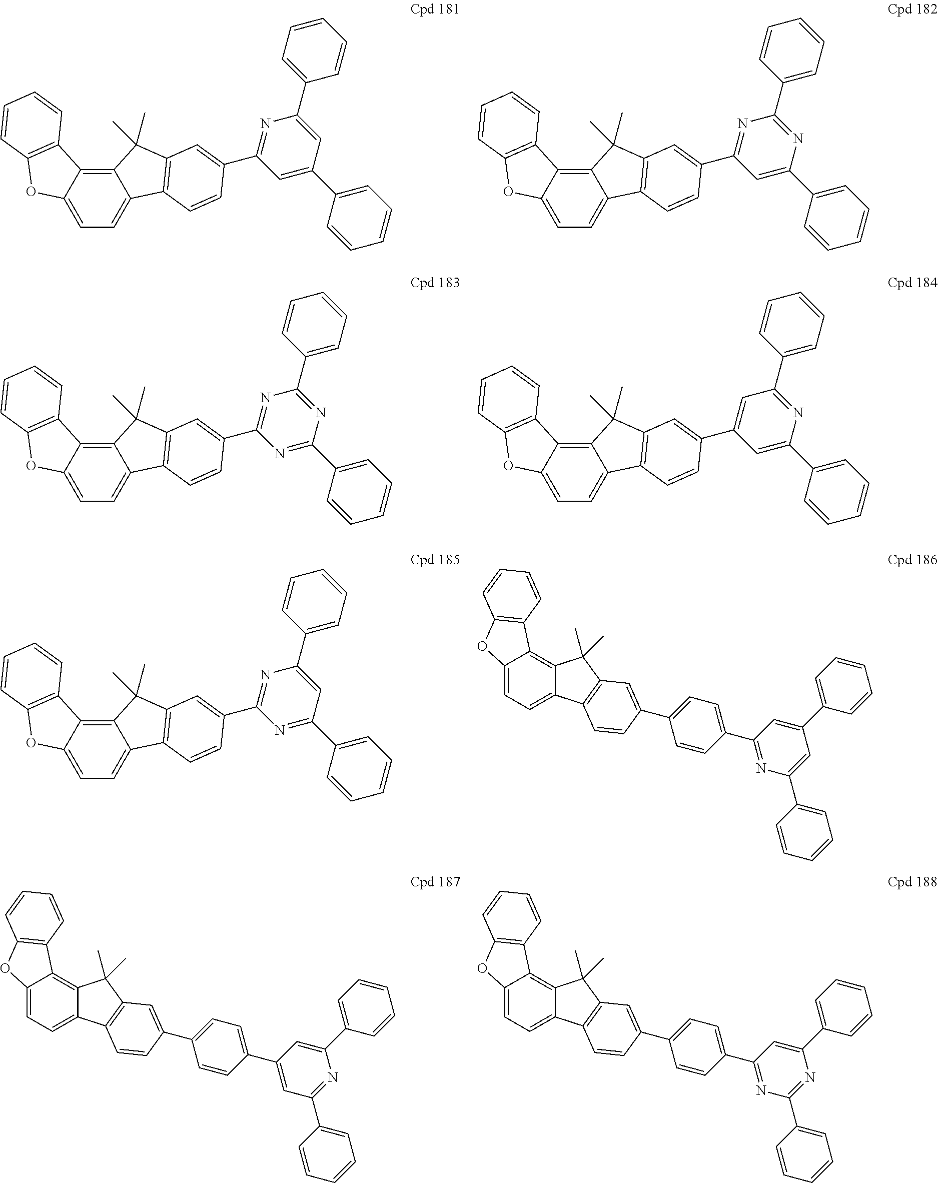

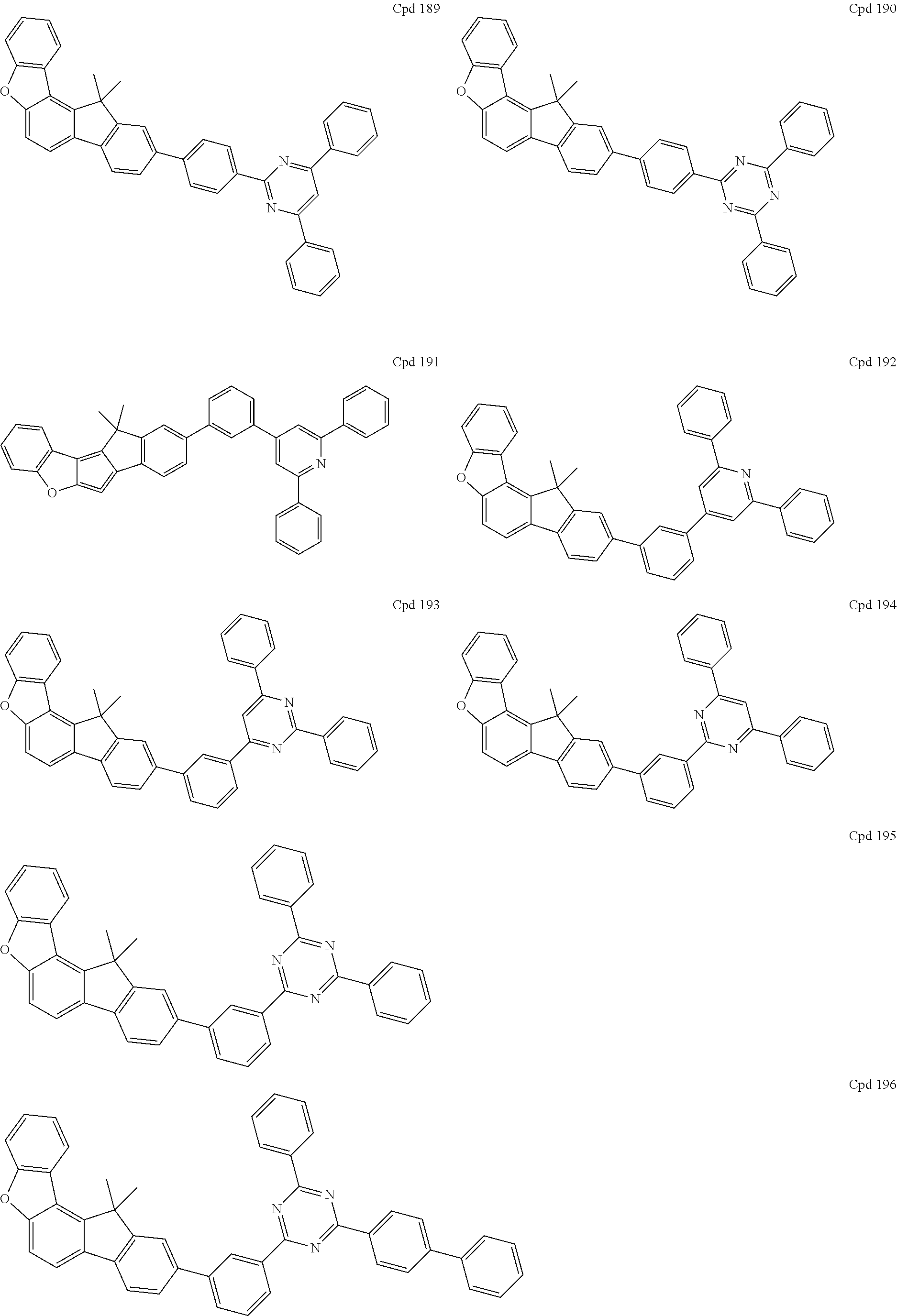

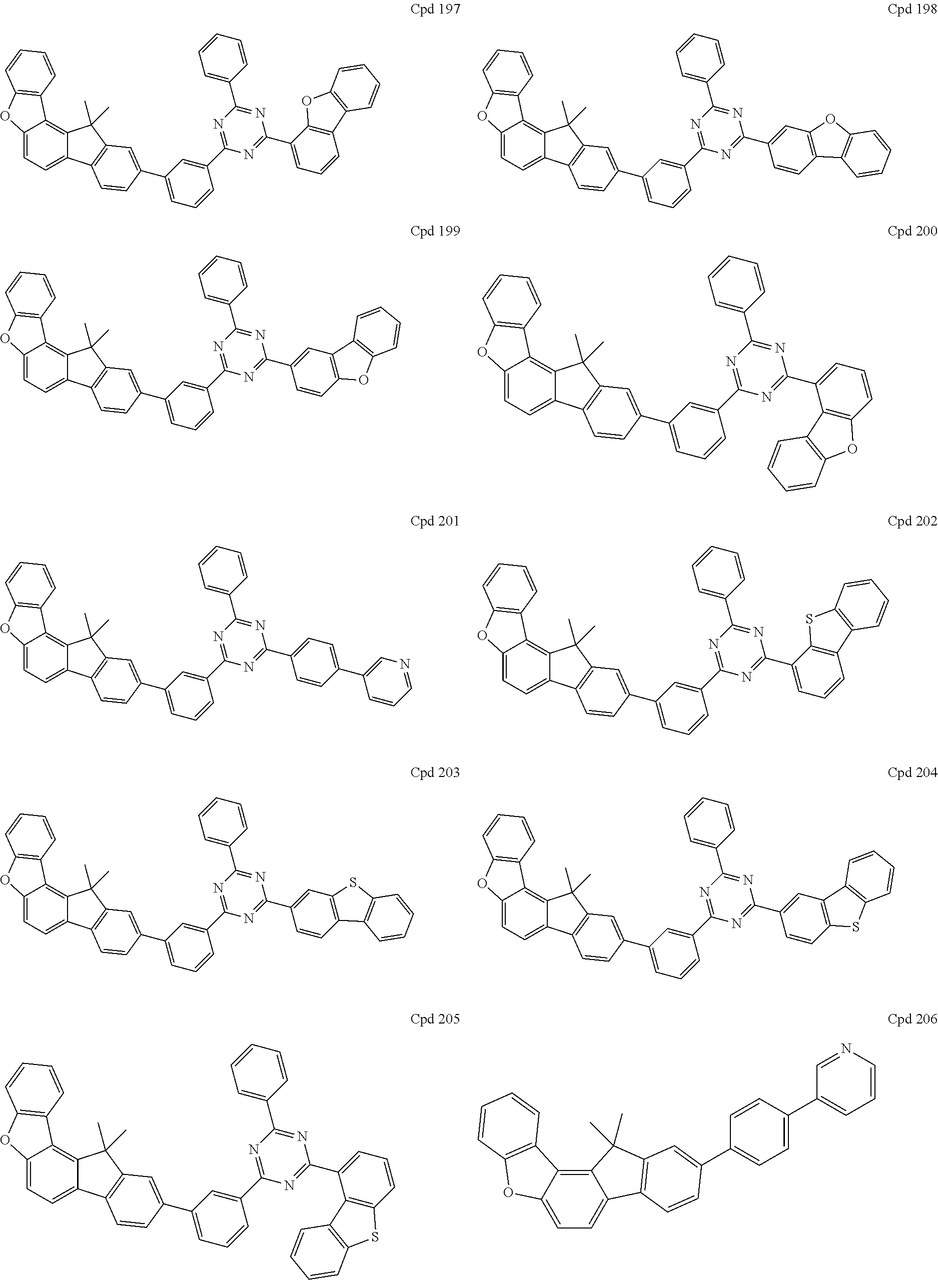

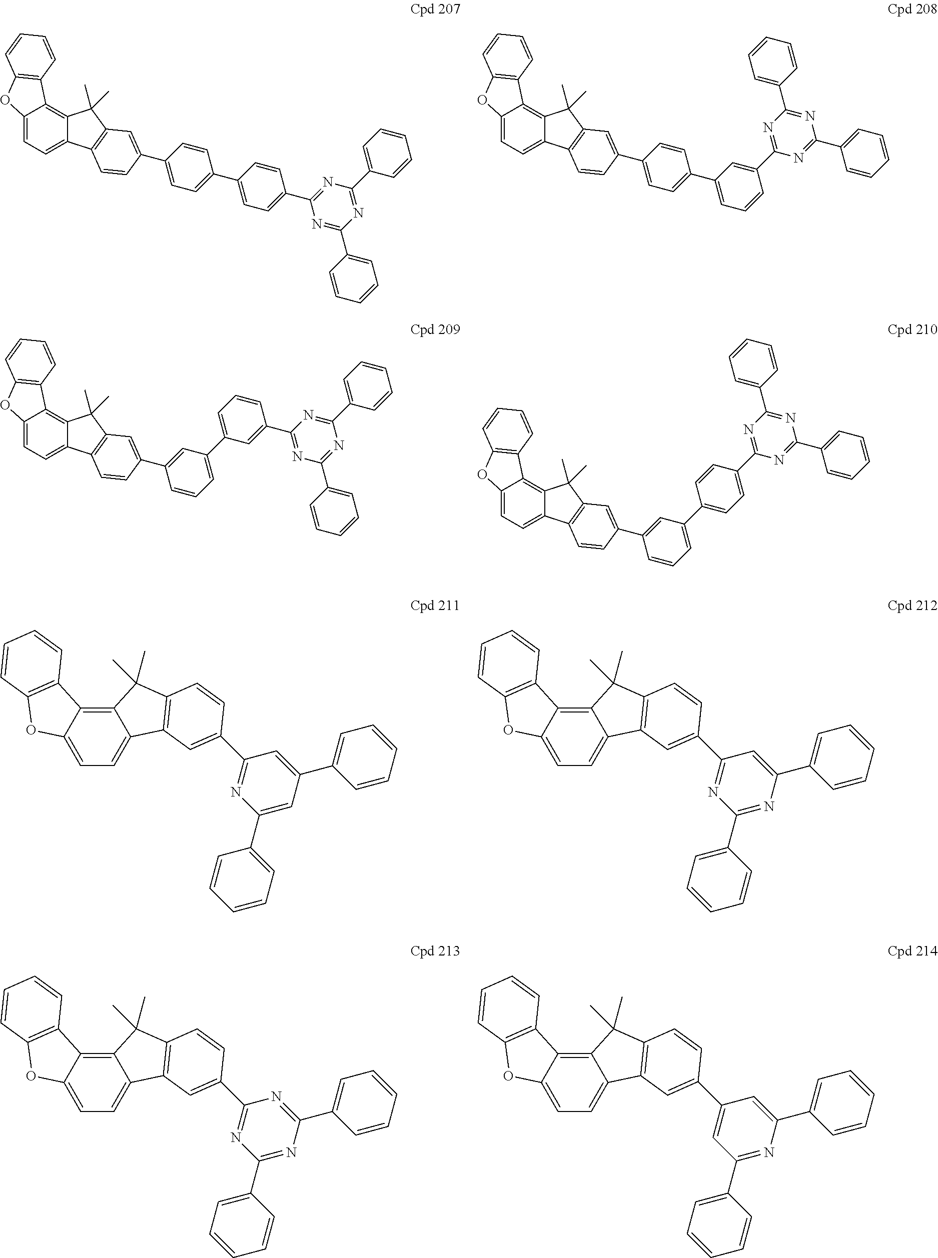

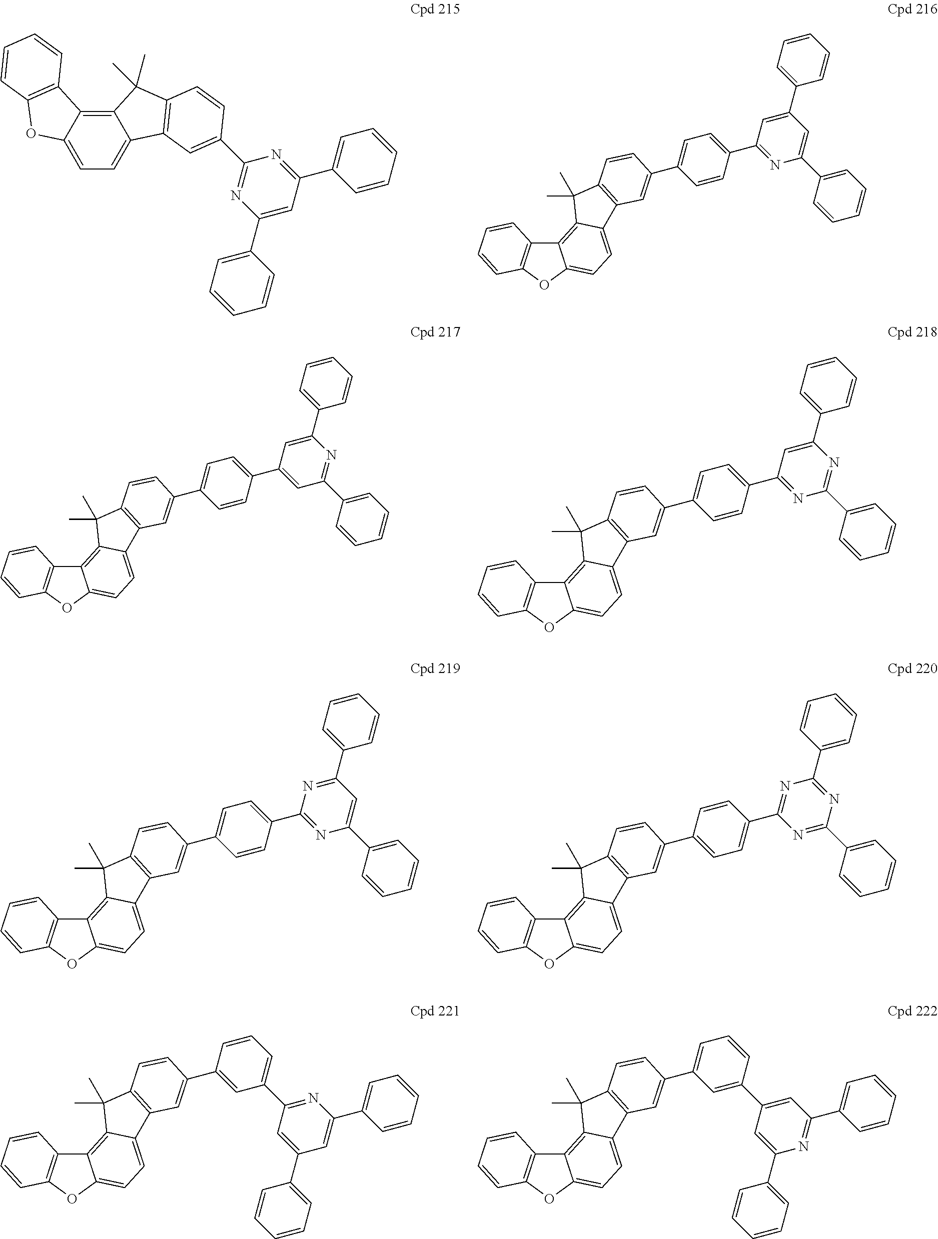

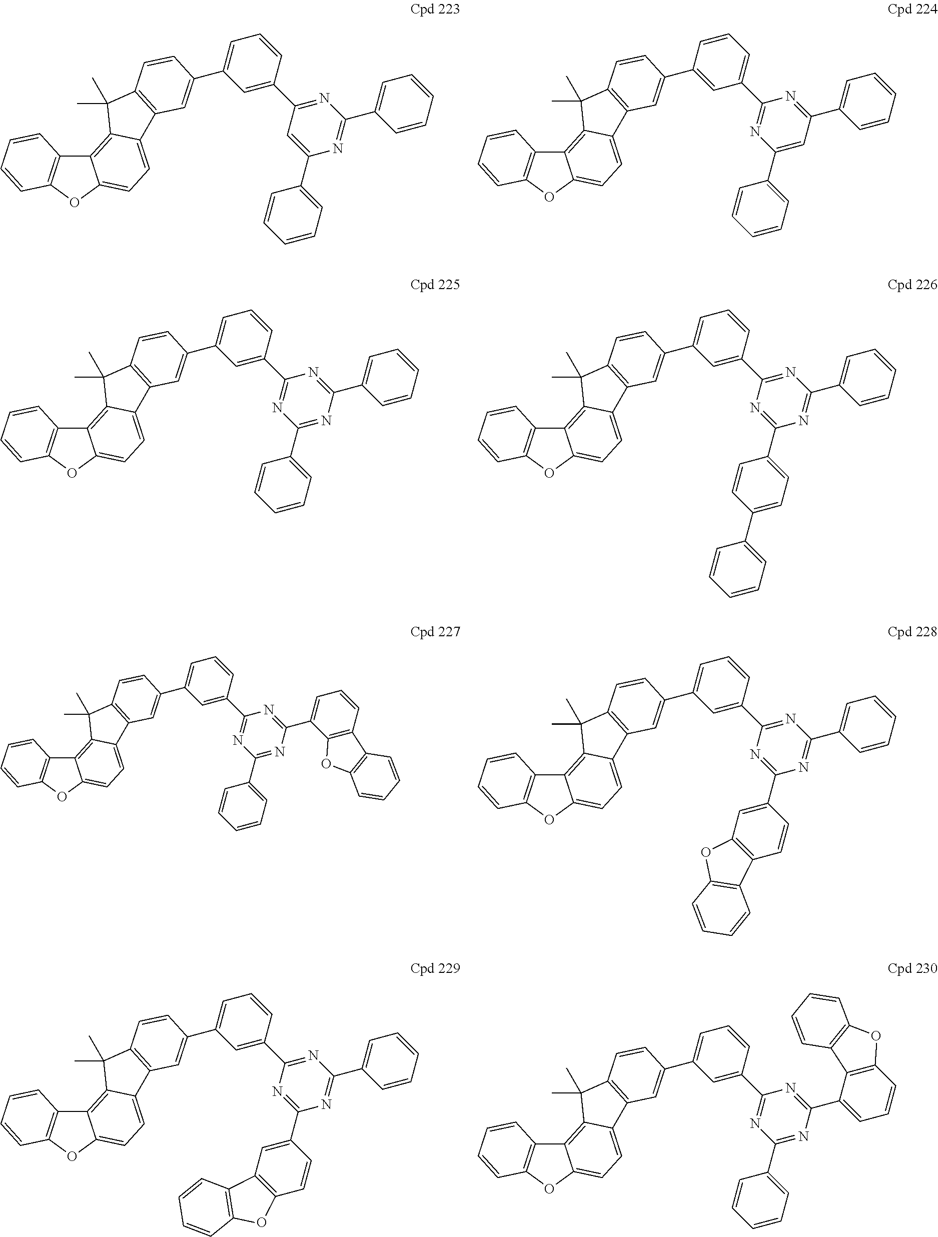

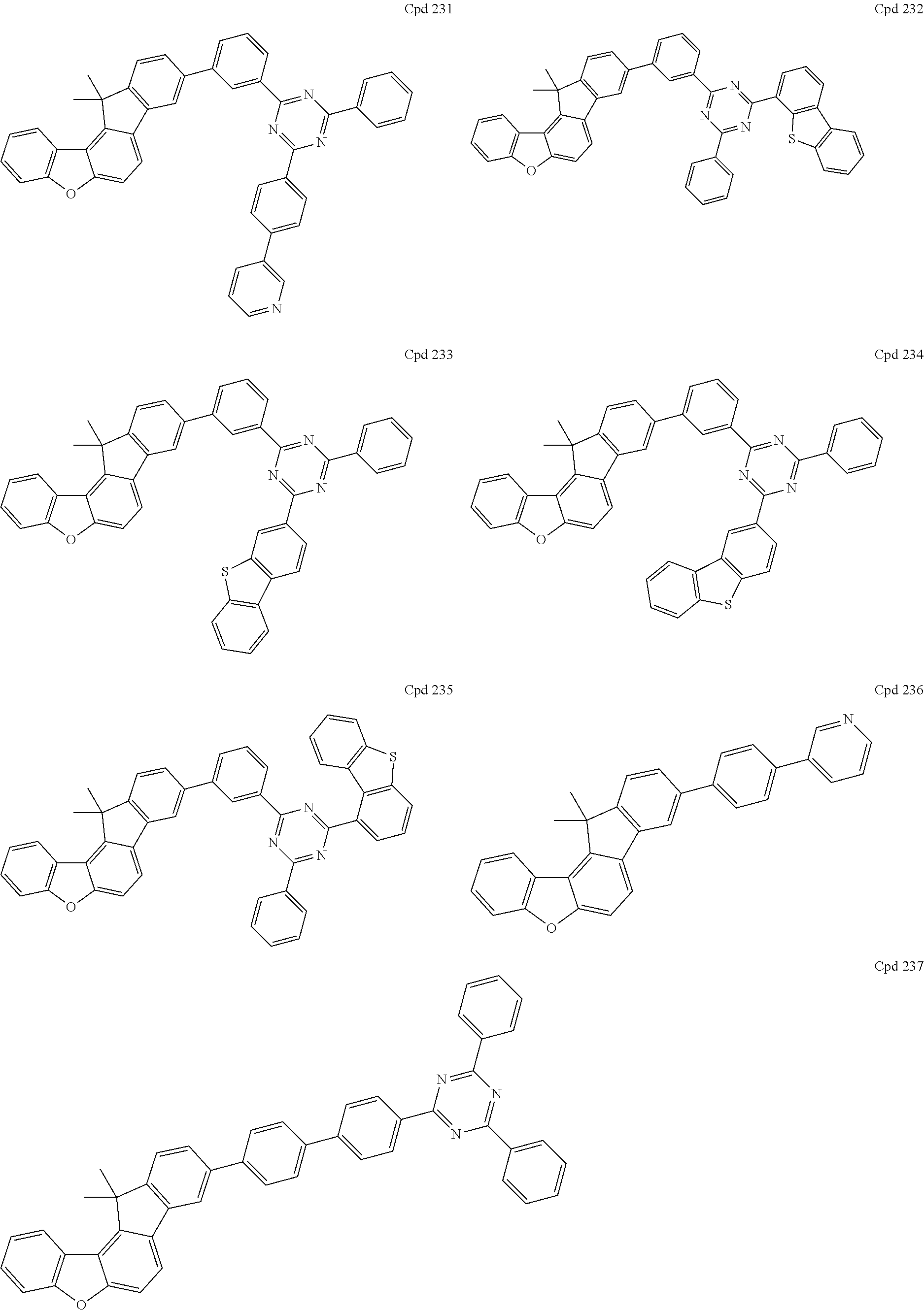

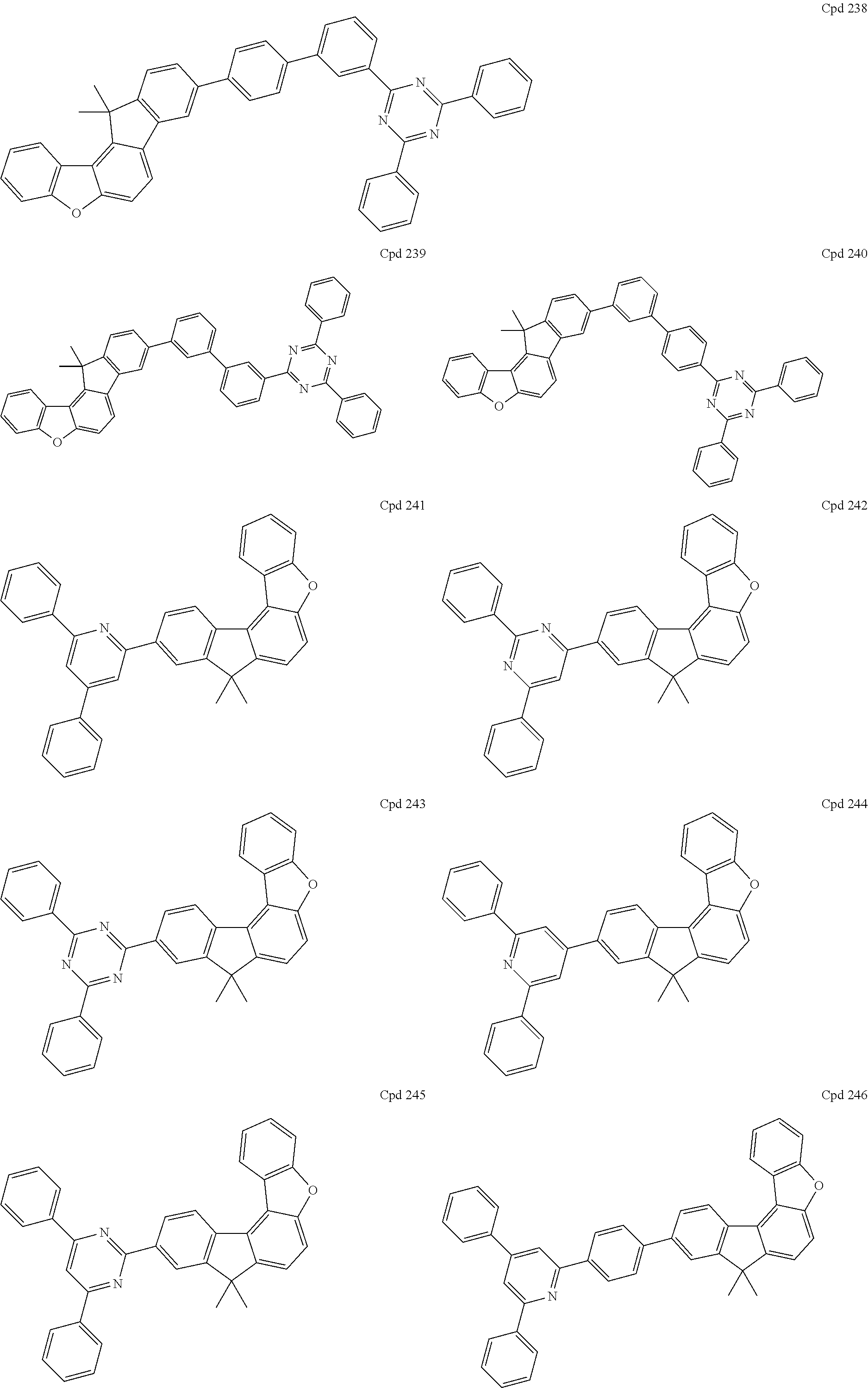

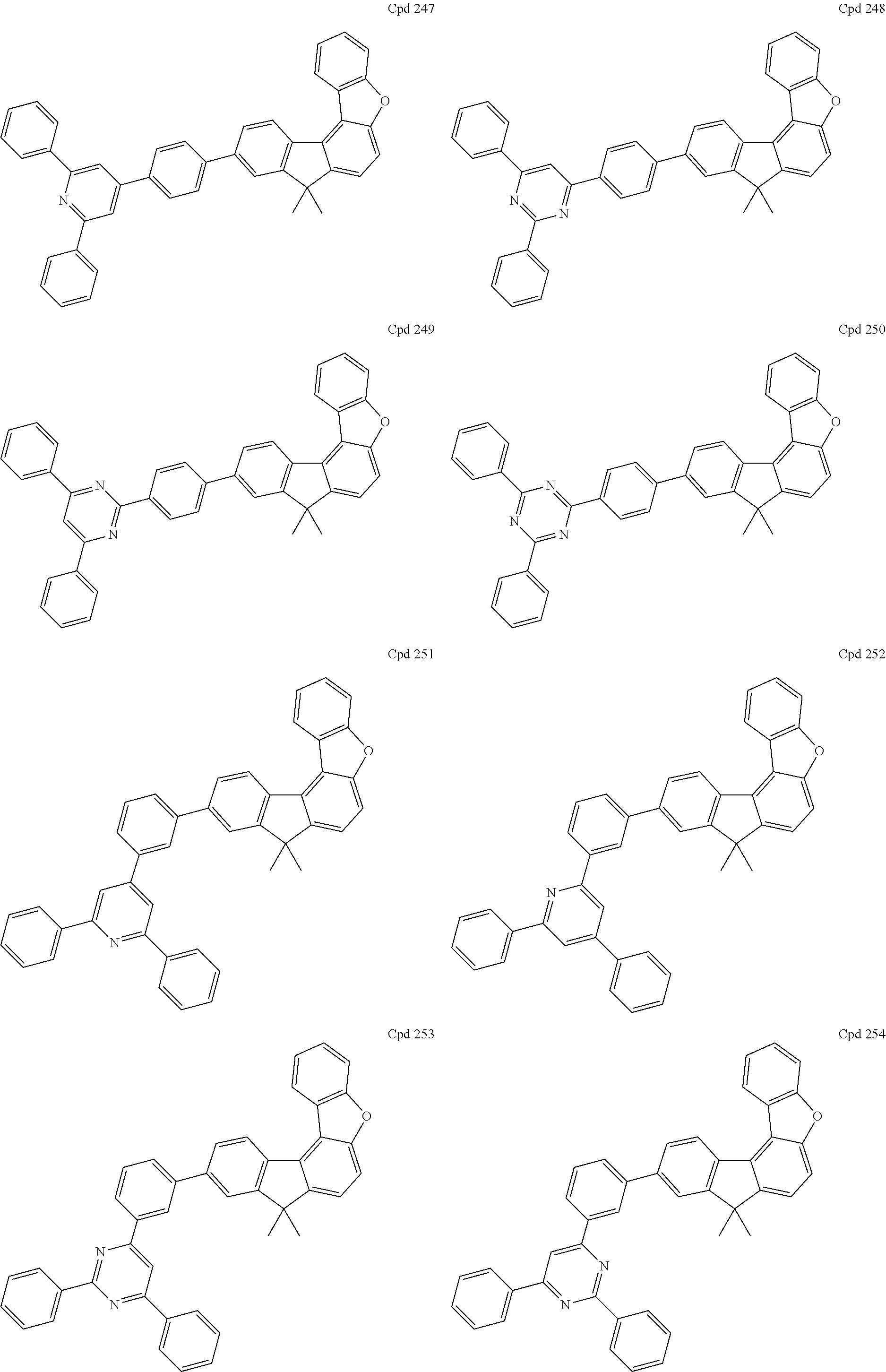

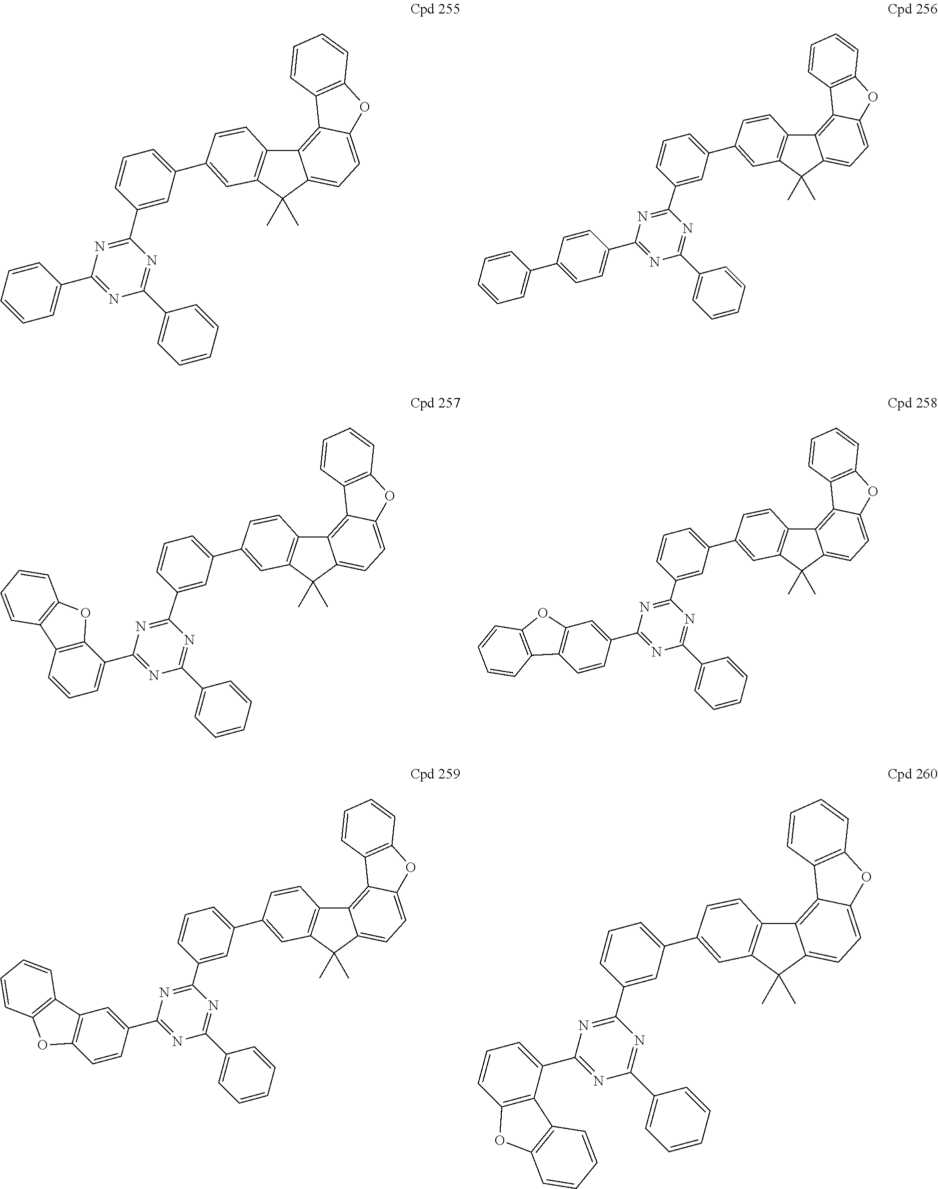

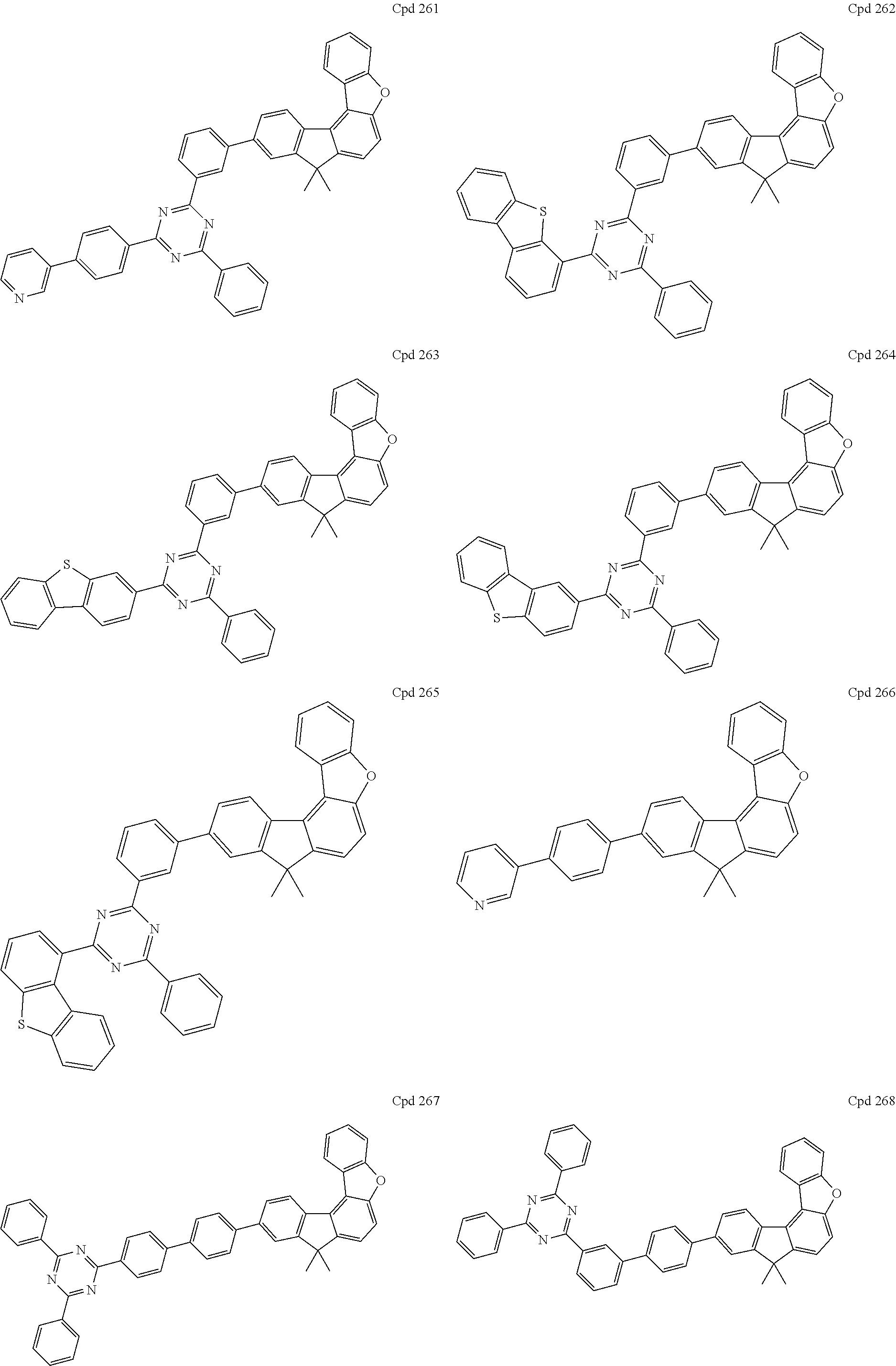

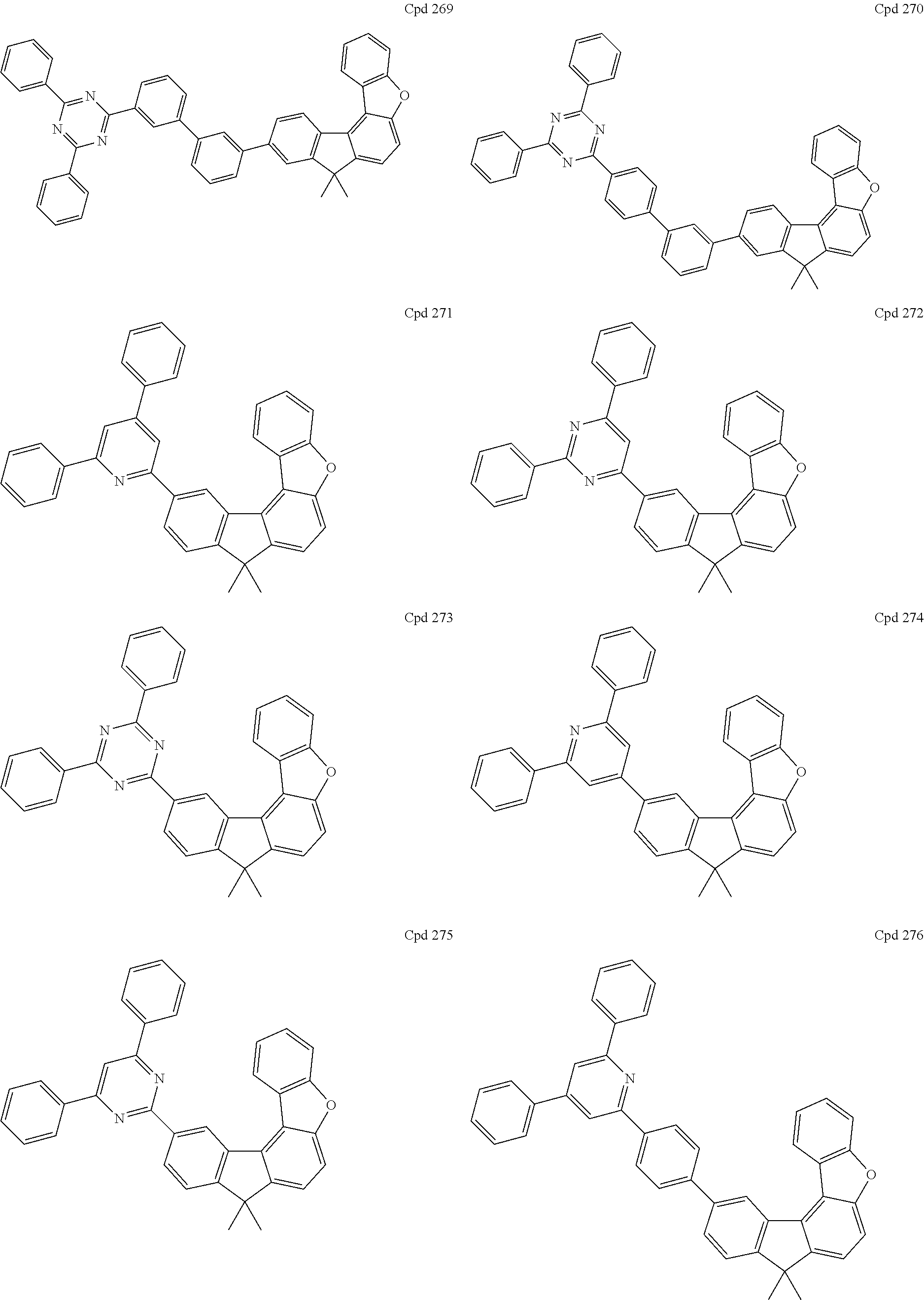

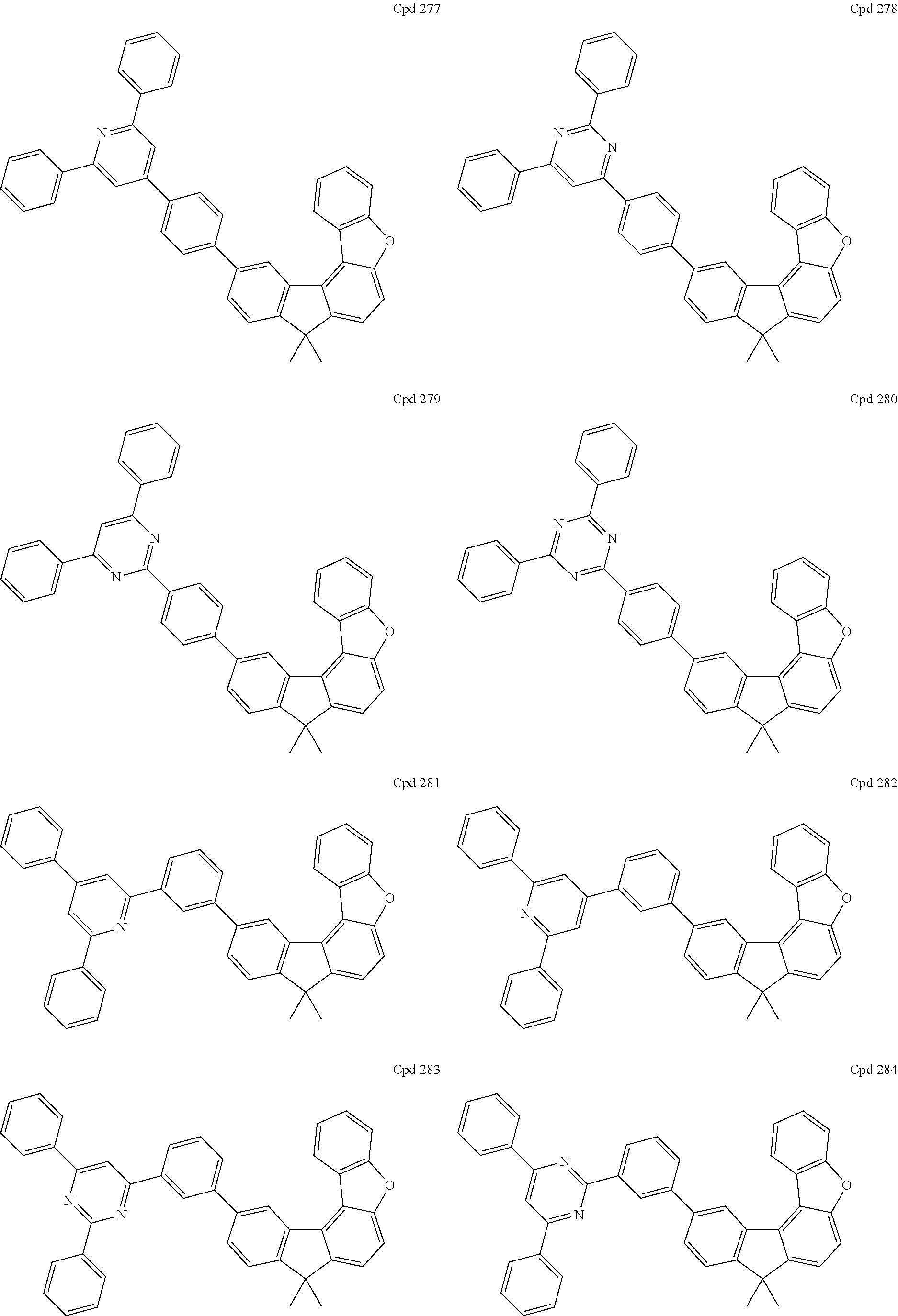

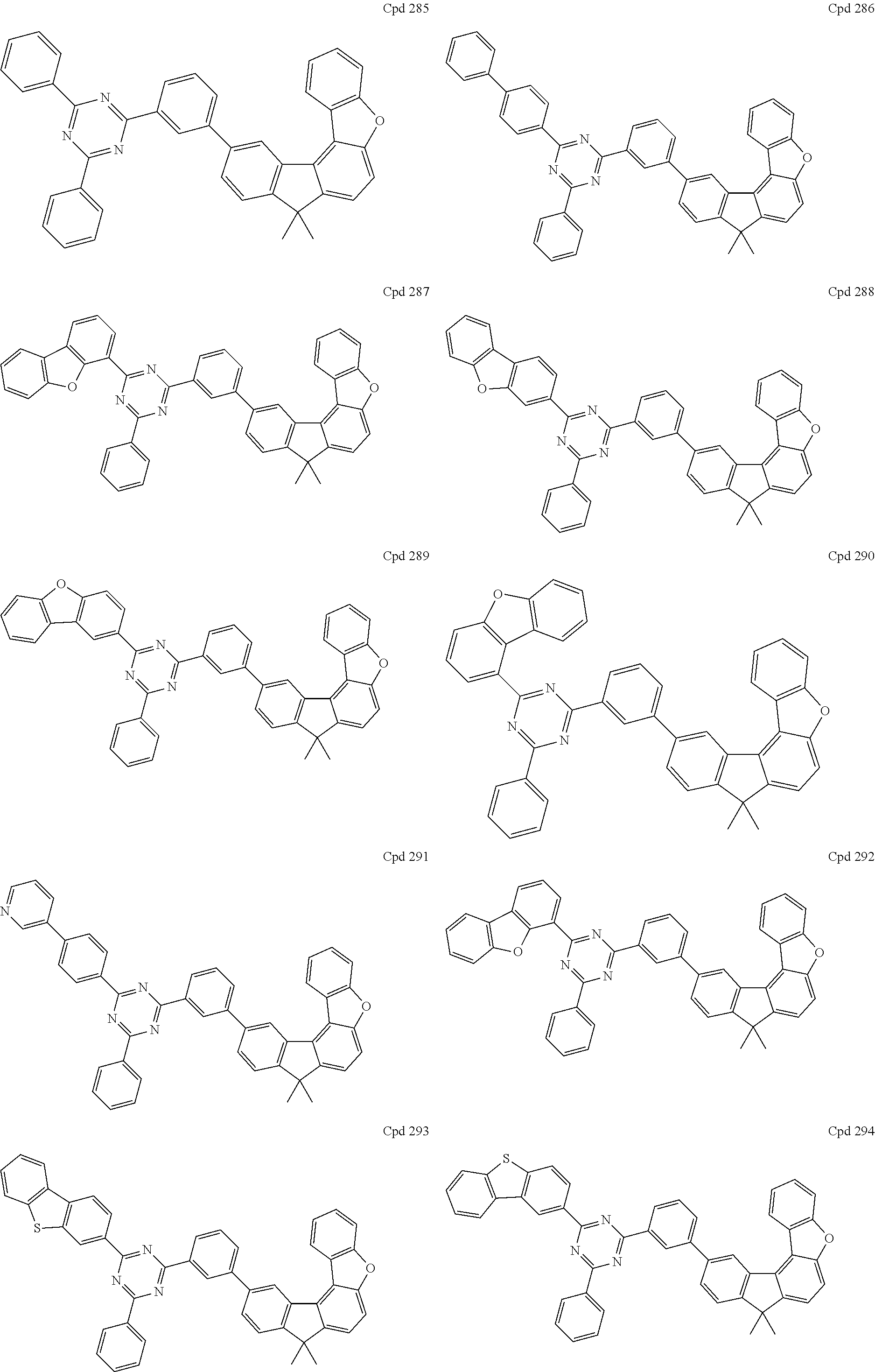

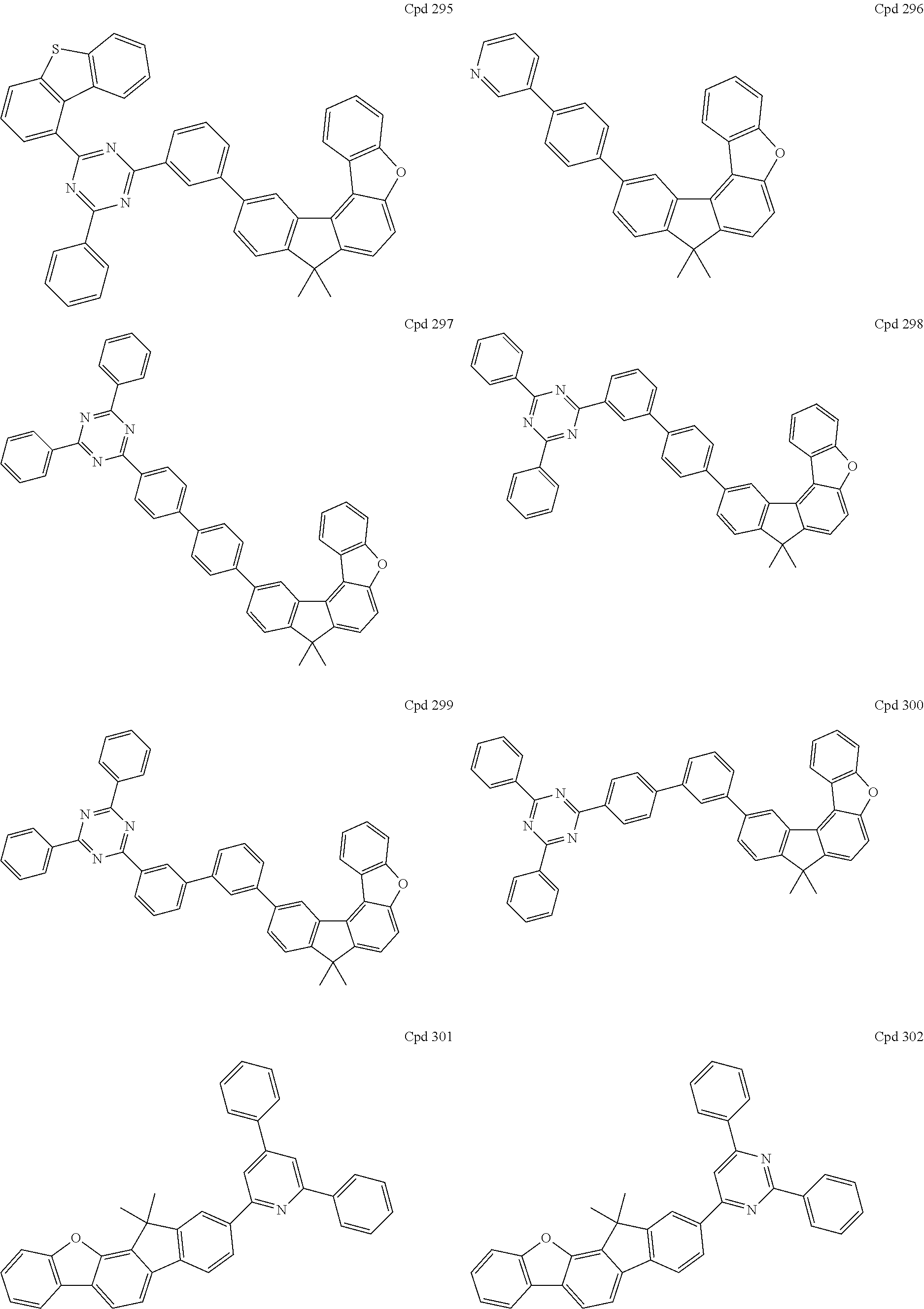

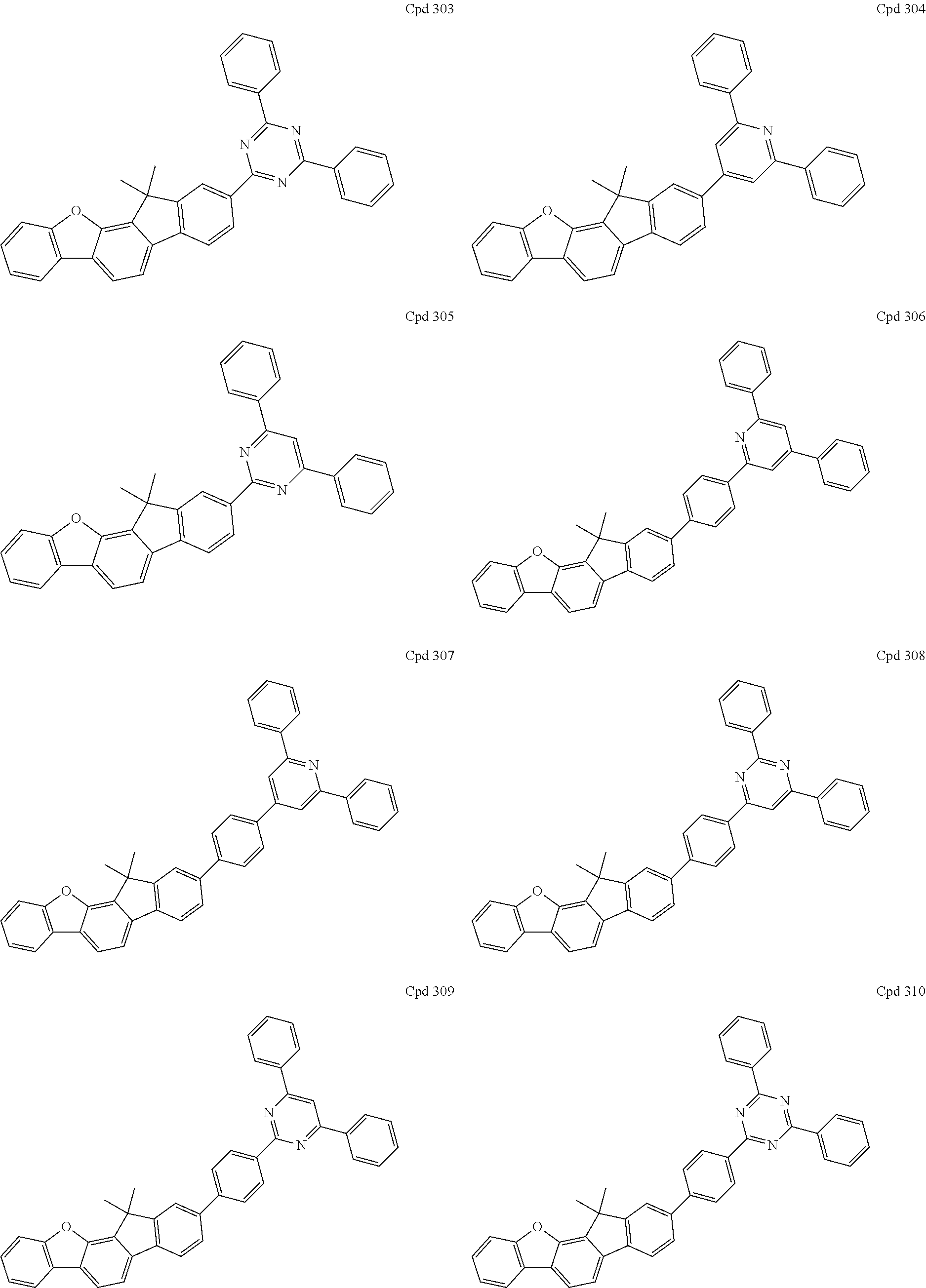

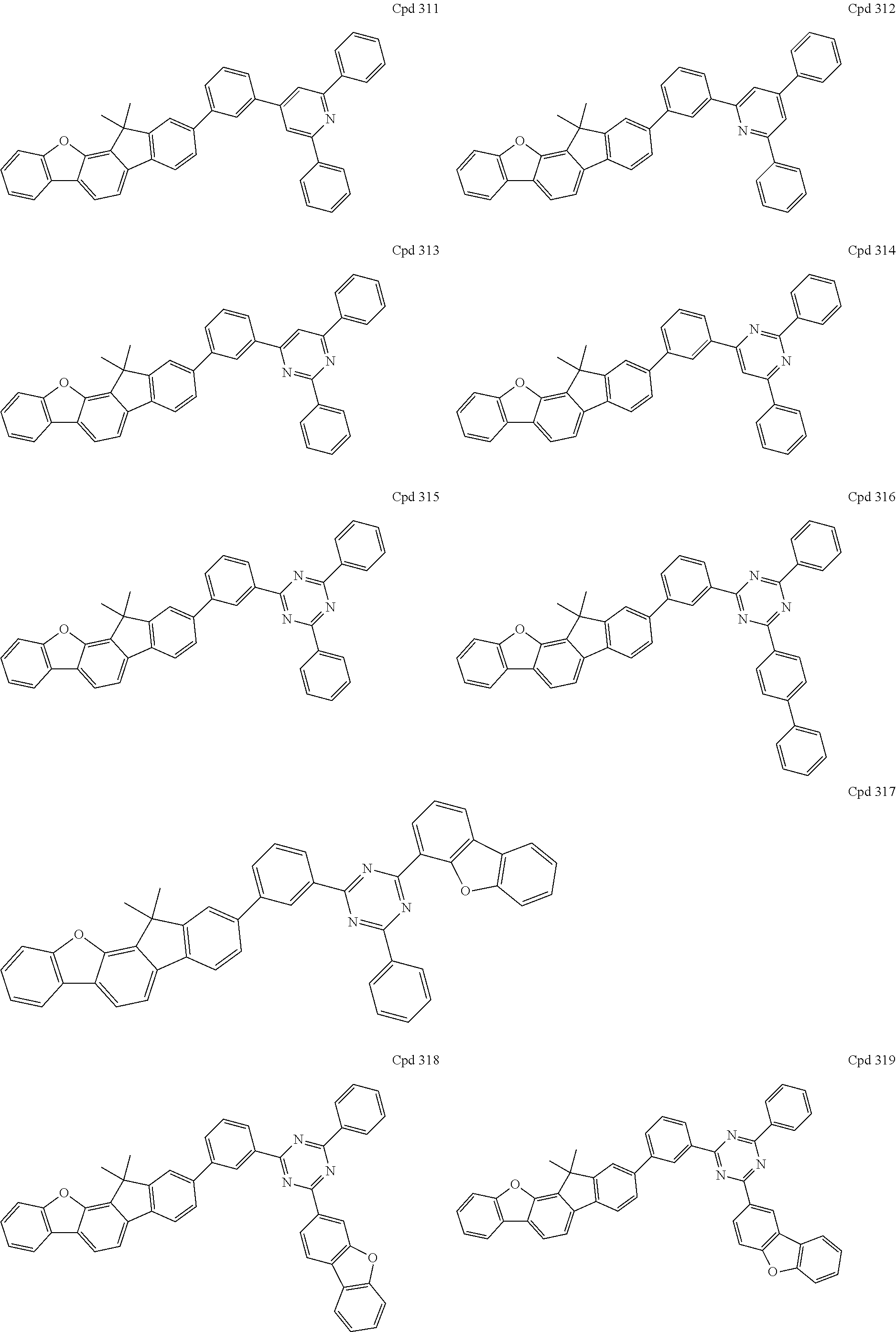

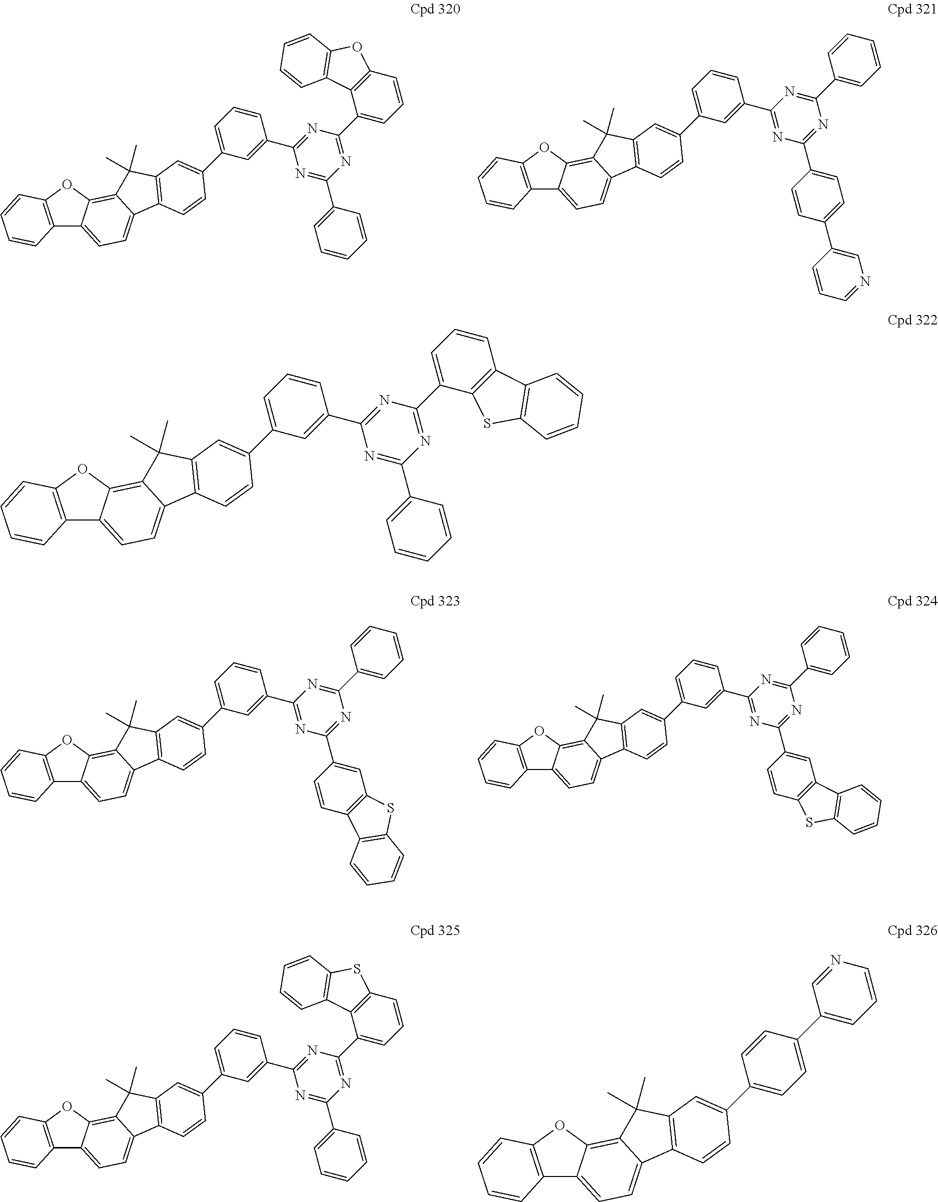

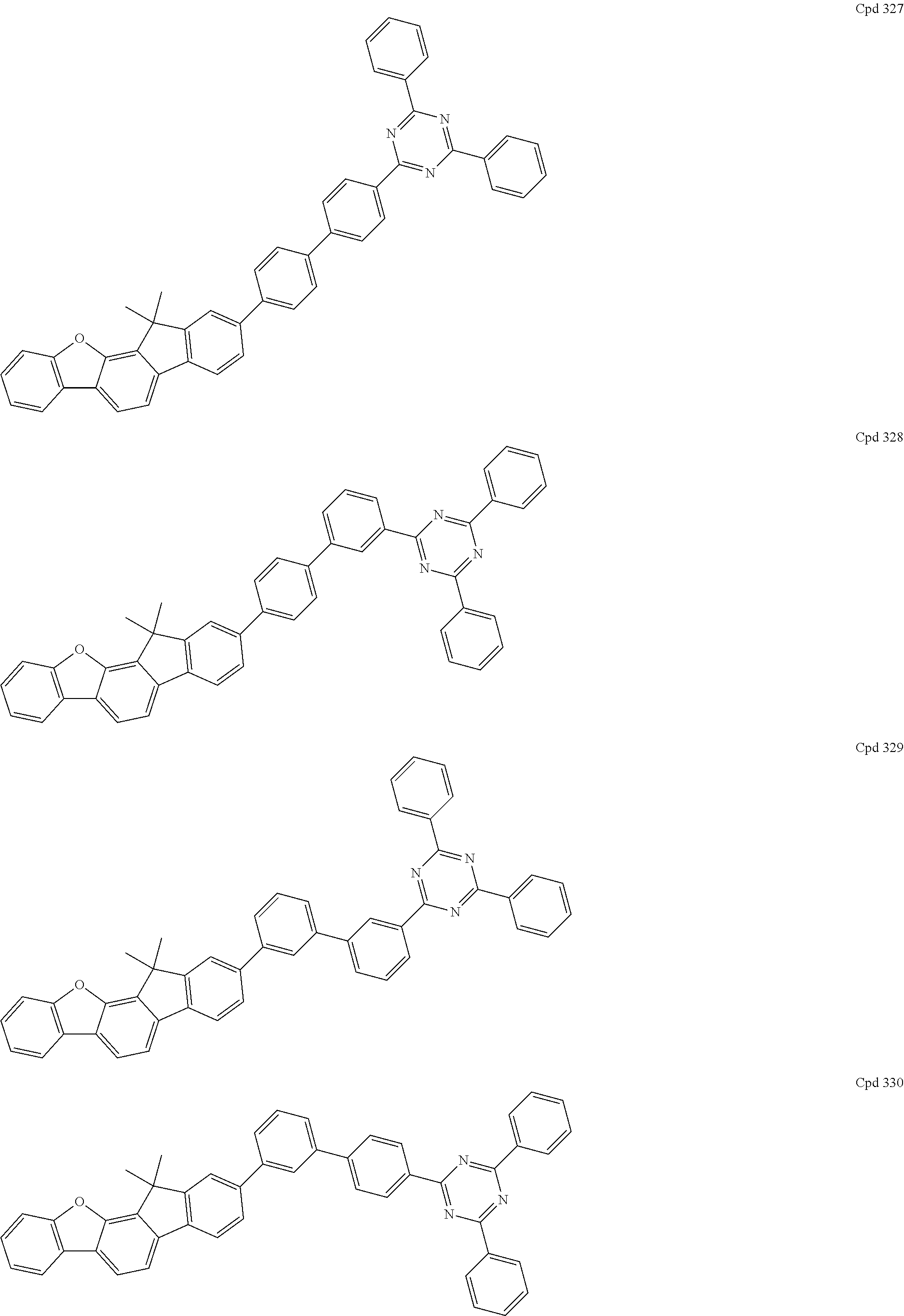 [SMILES: CC1(C)c2cc(-c3ccc(-c4ccc(-c5nc(-c6ccccc6)nc(-c6ccccc6)n5)cc4)cc3)ccc2-c2ccc3c(oc4ccccc43)c21 ; CC1(C)c2cc(-c3ccc(-c4cccc(-c5nc(-c6ccccc6)nc(-c6ccccc6)n5)c4)cc3)ccc2-c2ccc3c(oc4ccccc43)c21 ; CC1(C)c2cc(-c3cccc(-c4cccc(-c5nc(-c6ccccc6)nc(-c6ccccc6)n5)c4)c3)ccc2-c2ccc3c(oc4ccccc43)c21 ; CC1(C)c2cc(-c3cccc(-c4ccc(-c5nc(-c6ccccc6)nc(-c6ccccc6)n5)cc4)c3)ccc2-c2ccc3c(oc4ccccc43)c21]

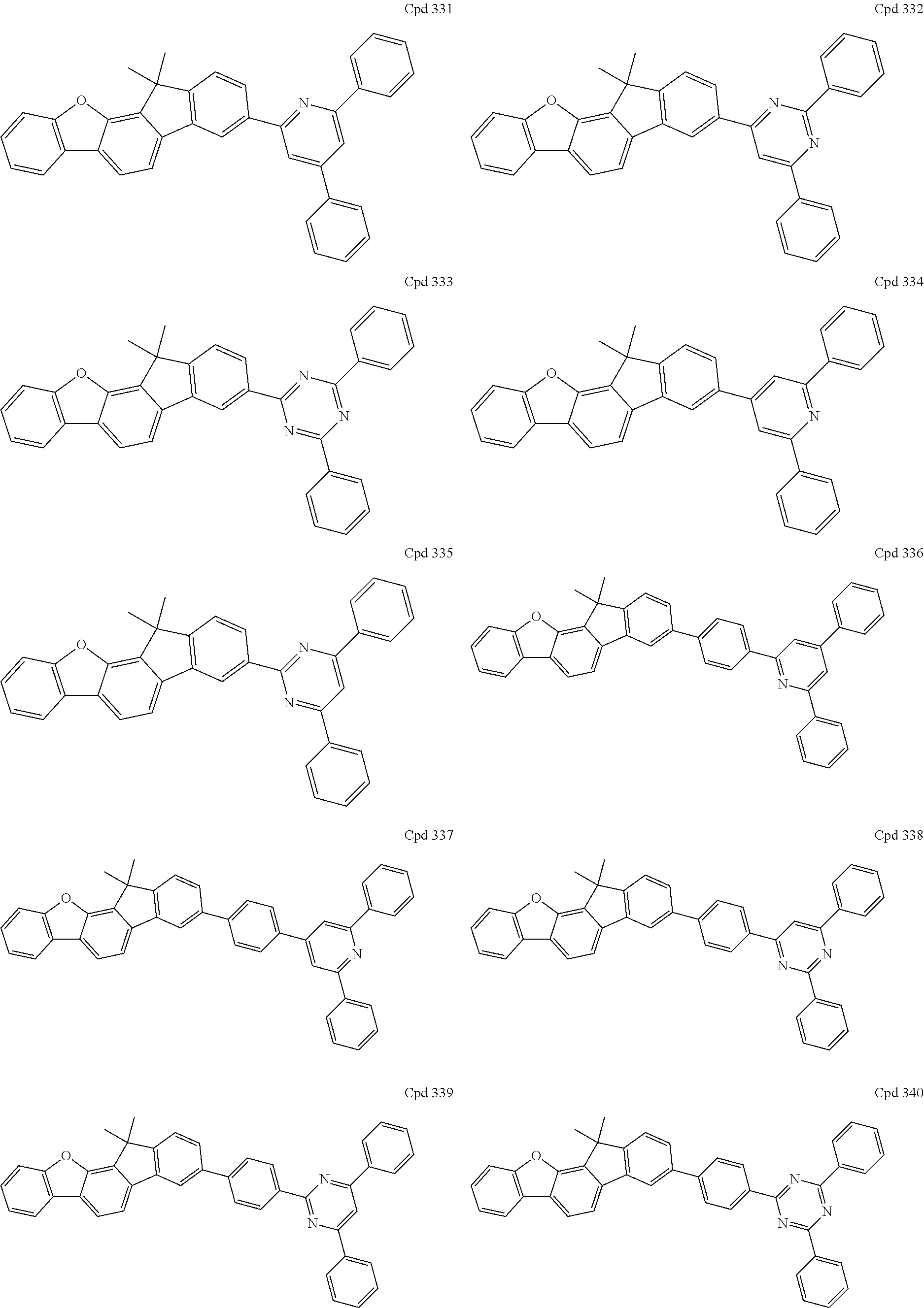

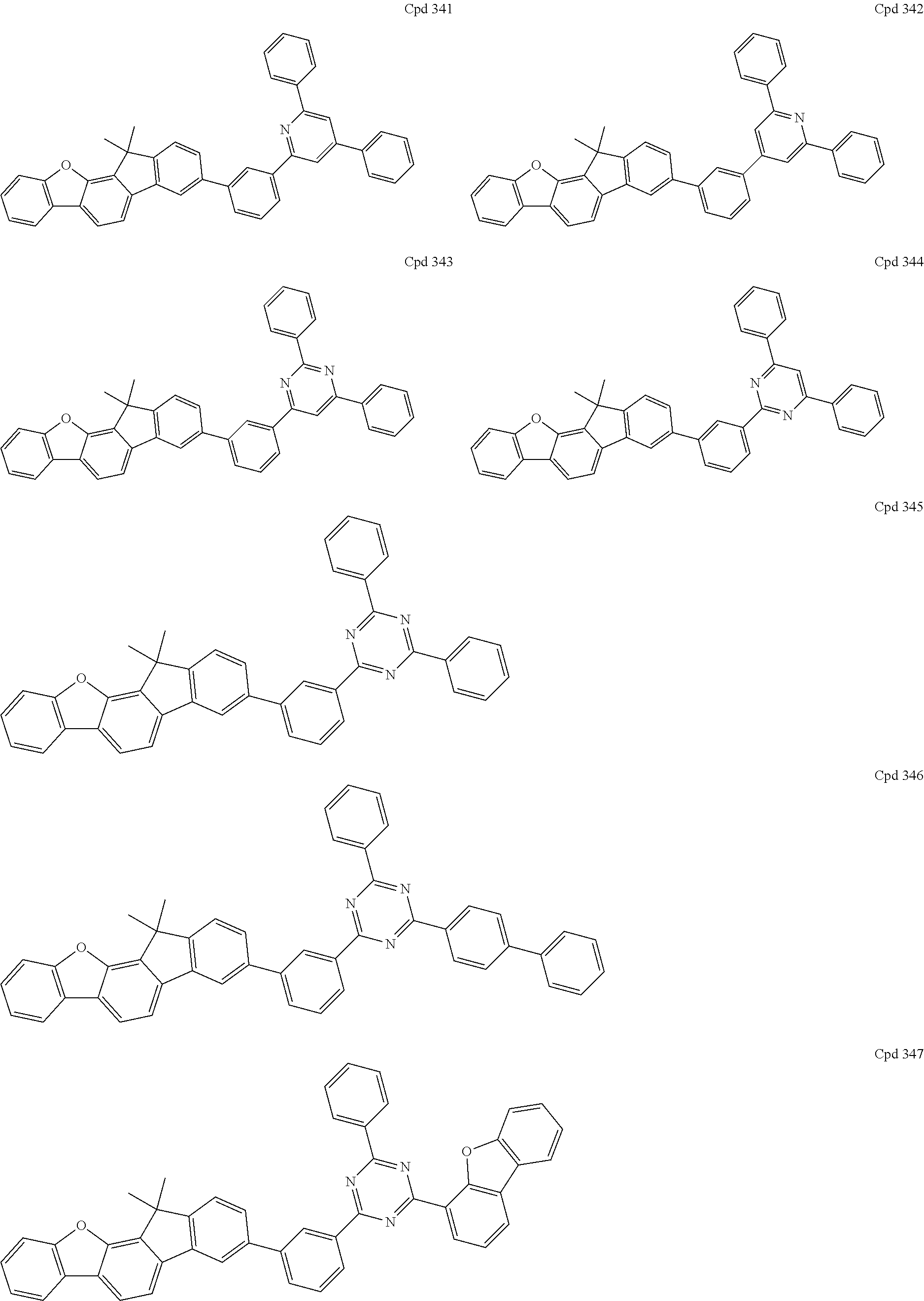

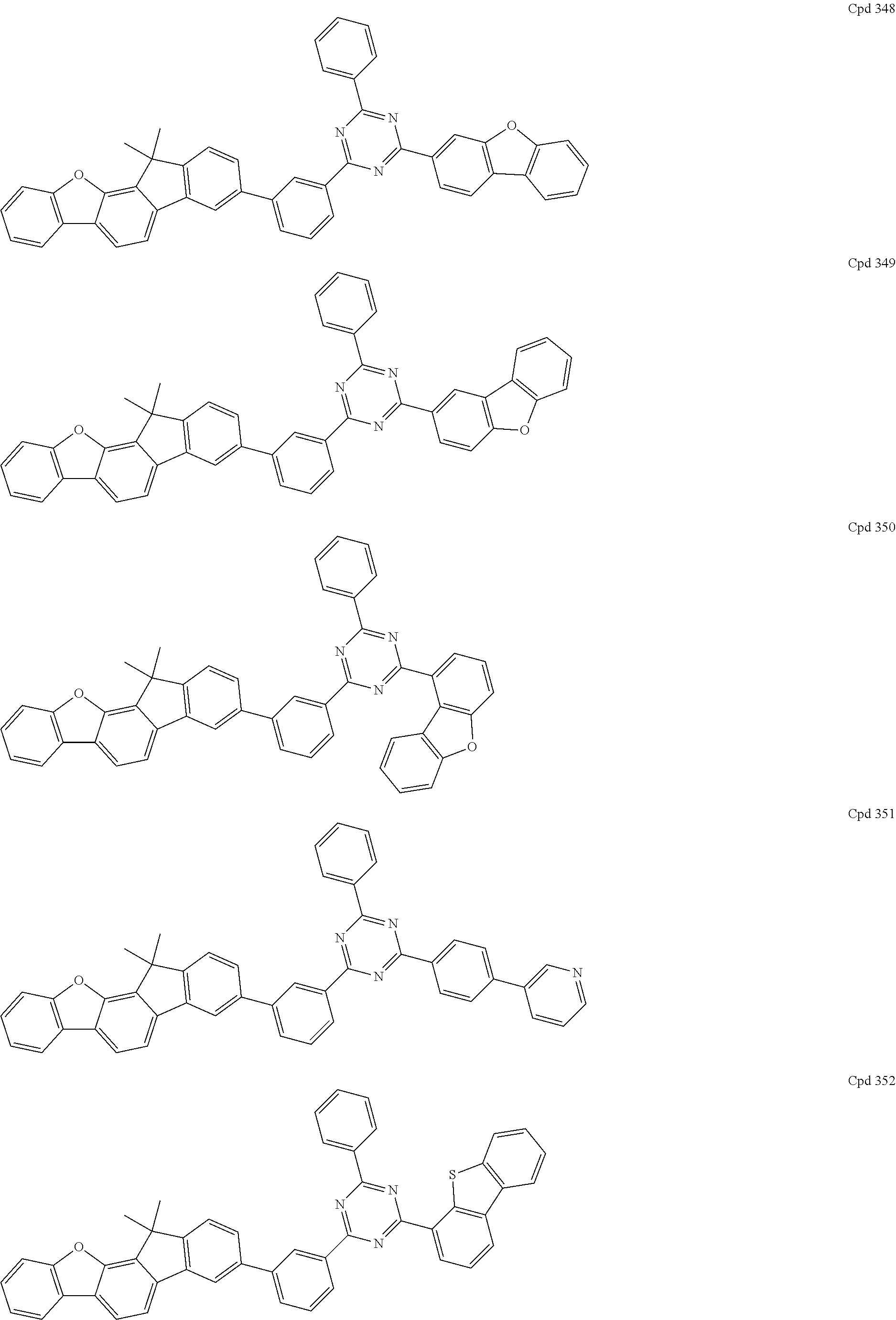

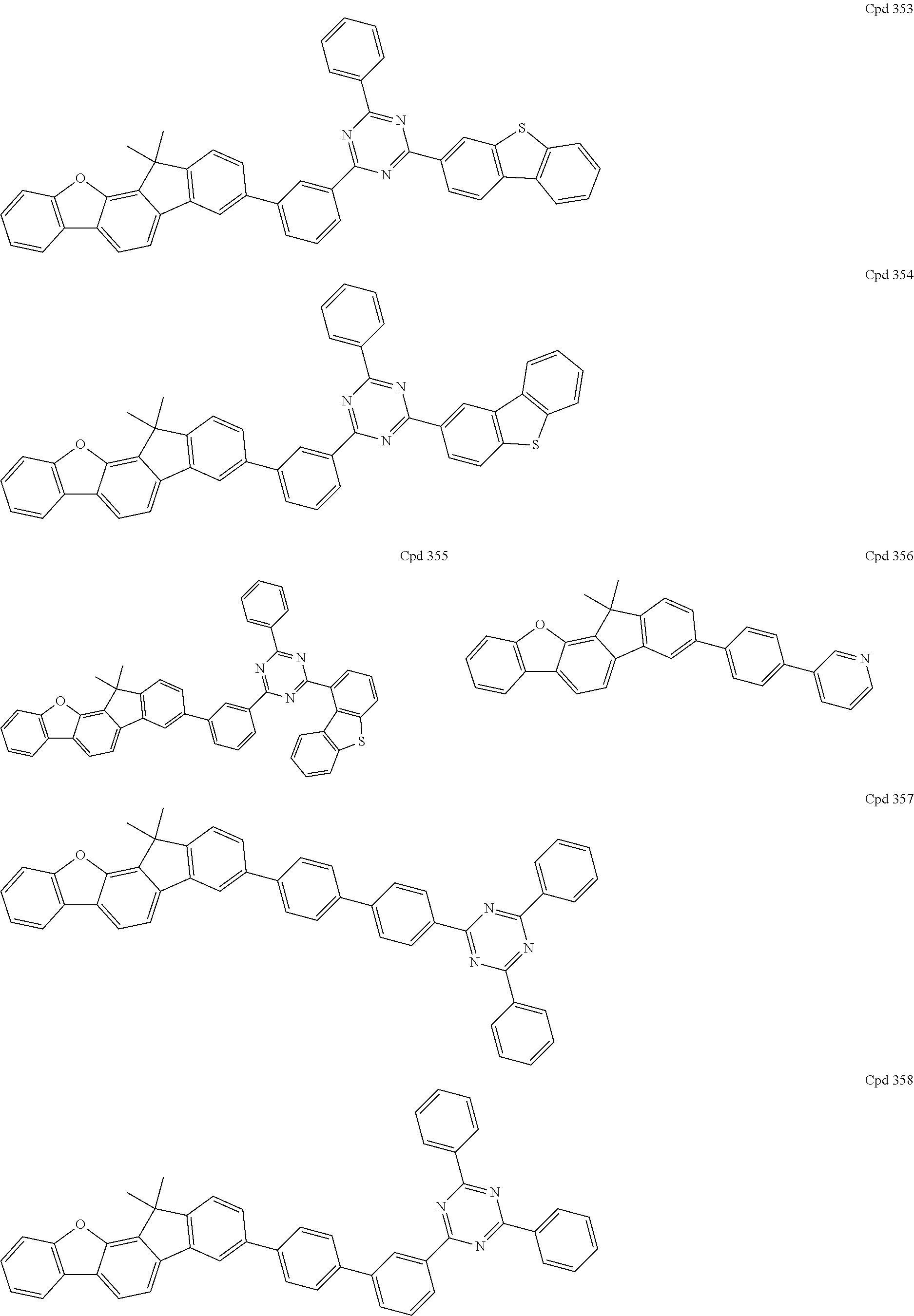

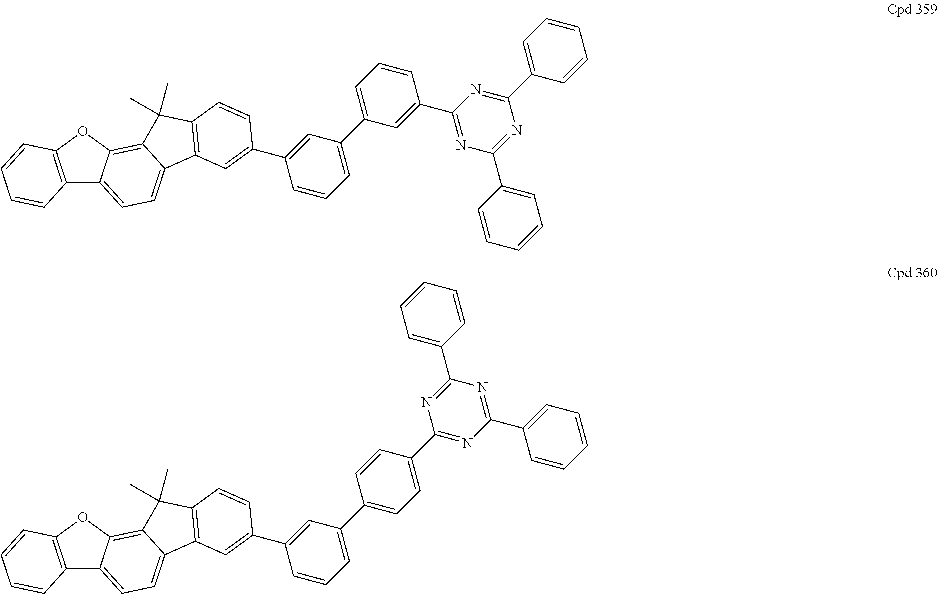 [SMILES: CC1(C)c2ccc(-c3cccc(-c4cccc(-c5nc(-c6ccccc6)nc(-c6ccccc6)n5)c4)c3)cc2-c2ccc3c(oc4ccccc43)c21 ; CC1(C)c2ccc(-c3cccc(-c4ccc(-c5nc(-c6ccccc6)nc(-c6ccccc6)n5)cc4)c3)cc2-c2ccc3c(oc4ccccc43)c21]

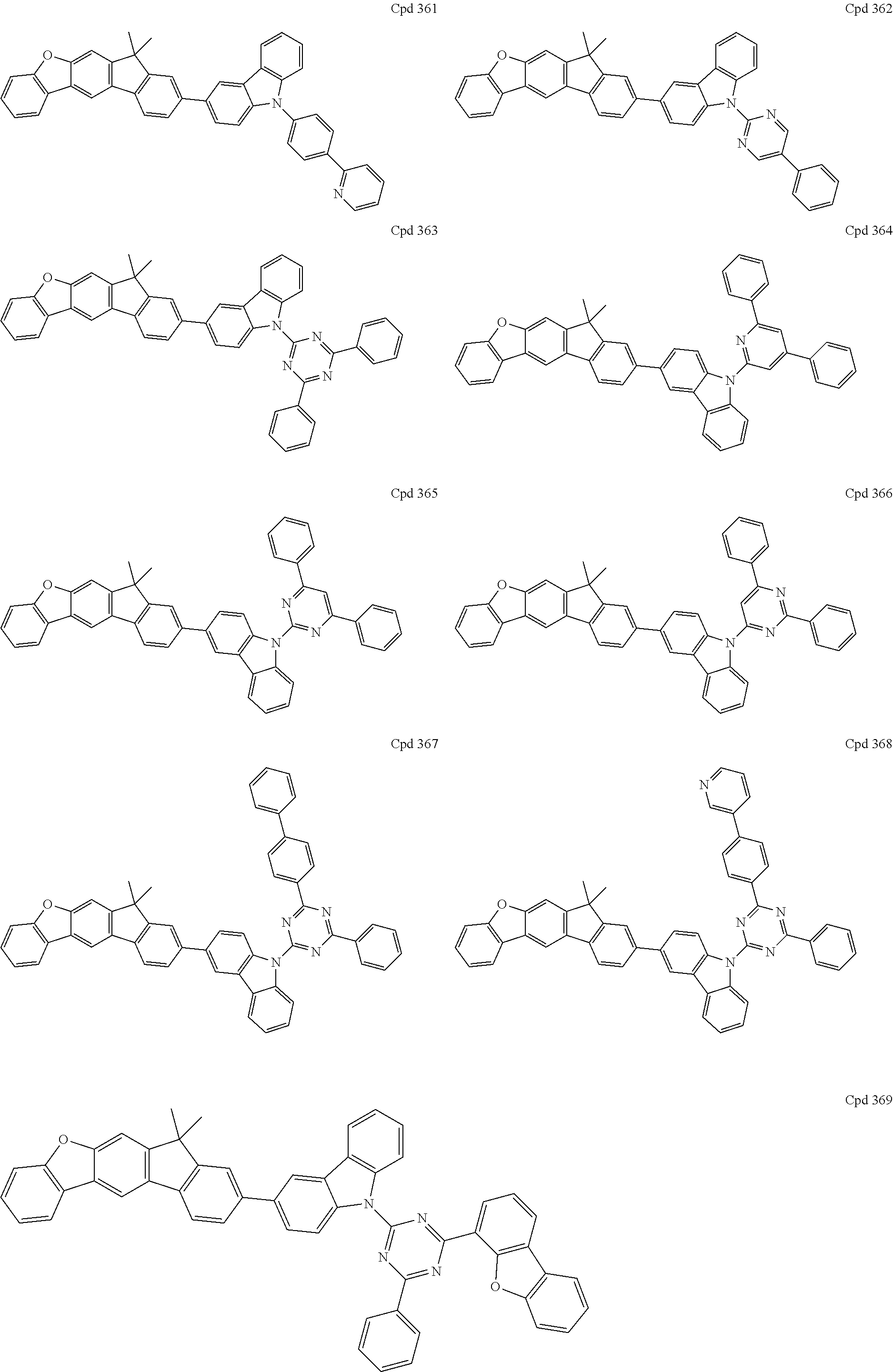

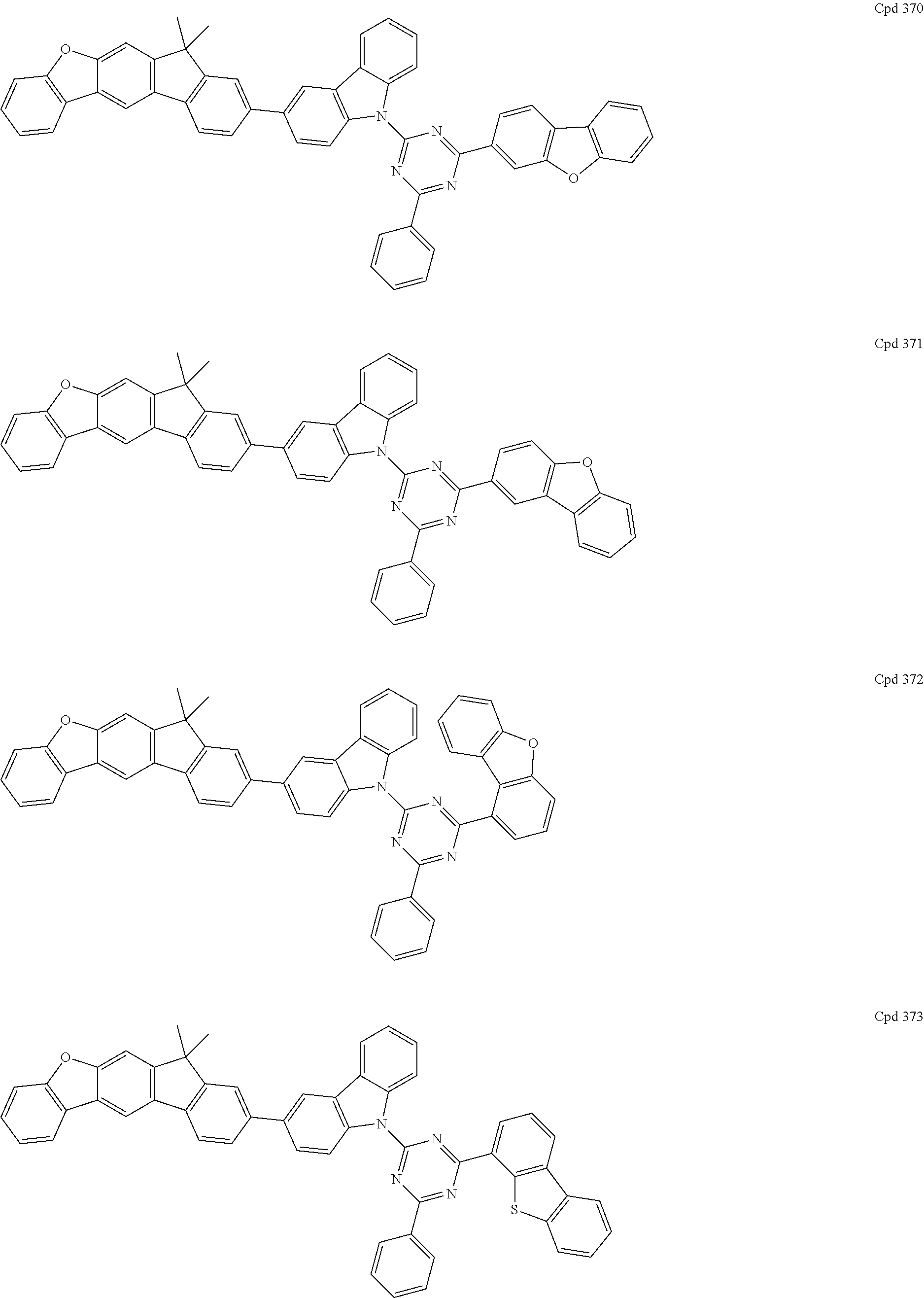 [SMILES: CC1(C)c2cc(-c3ccc4c(c3)c3ccccc3n4-c3nc(-c4ccccc4)nc(-c4ccc5c(c4)oc4ccccc45)n3)ccc2-c2cc3c(cc21)oc1ccccc13 ; CC1(C)c2cc(-c3ccc4c(c3)c3ccccc3n4-c3nc(-c4ccccc4)nc(-c4ccc5oc6ccccc6c5c4)n3)ccc2-c2cc3c(cc21)oc1ccccc13 ; CC1(C)c2cc(-c3ccc4c(c3)c3ccccc3n4-c3nc(-c4ccccc4)nc(-c4cccc5oc6ccccc6c45)n3)ccc2-c2cc3c(cc21)oc1ccccc13 ; CC1(C)c2cc(-c3ccc4c(c3)c3ccccc3n4-c3nc(-c4ccccc4)nc(-c4cccc5c4sc4ccccc45)n3)ccc2-c2cc3c(cc21)oc1ccccc13]

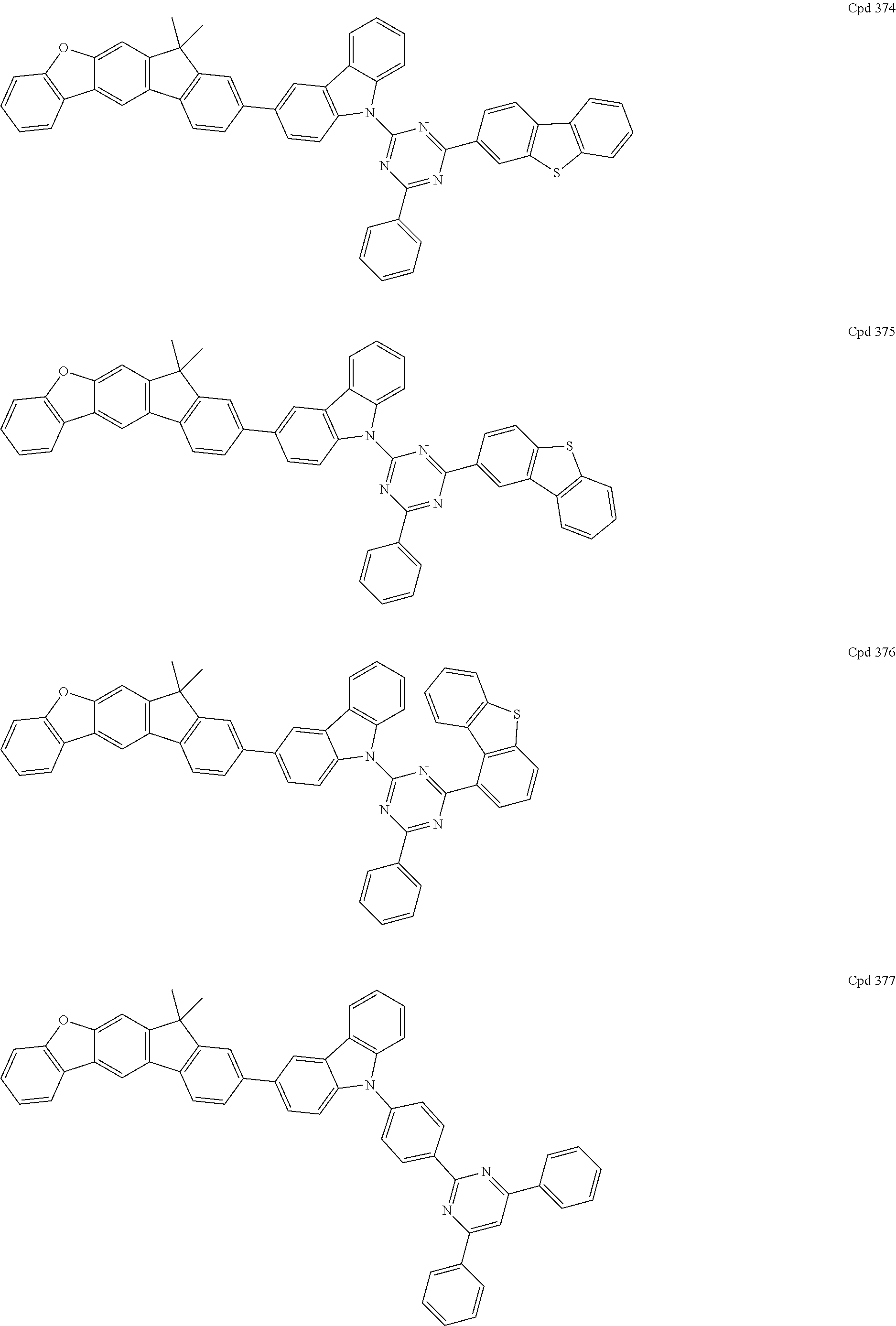 [SMILES: CC1(C)c2cc(-c3ccc4c(c3)c3ccccc3n4-c3nc(-c4ccccc4)nc(-c4ccc5c(c4)sc4ccccc45)n3)ccc2-c2cc3c(cc21)oc1ccccc13 ; CC1(C)c2cc(-c3ccc4c(c3)c3ccccc3n4-c3nc(-c4ccccc4)nc(-c4ccc5sc6ccccc6c5c4)n3)ccc2-c2cc3c(cc21)oc1ccccc13 ; CC1(C)c2cc(-c3ccc4c(c3)c3ccccc3n4-c3nc(-c4ccccc4)nc(-c4cccc5sc6ccccc6c45)n3)ccc2-c2cc3c(cc21)oc1ccccc13 ; CC1(C)c2cc(-c3ccc4c(c3)c3ccccc3n4-c3ccc(-c4nc(-c5ccccc5)cc(-c5ccccc5)n4)cc3)ccc2-c2cc3c(cc21)oc1ccccc13]

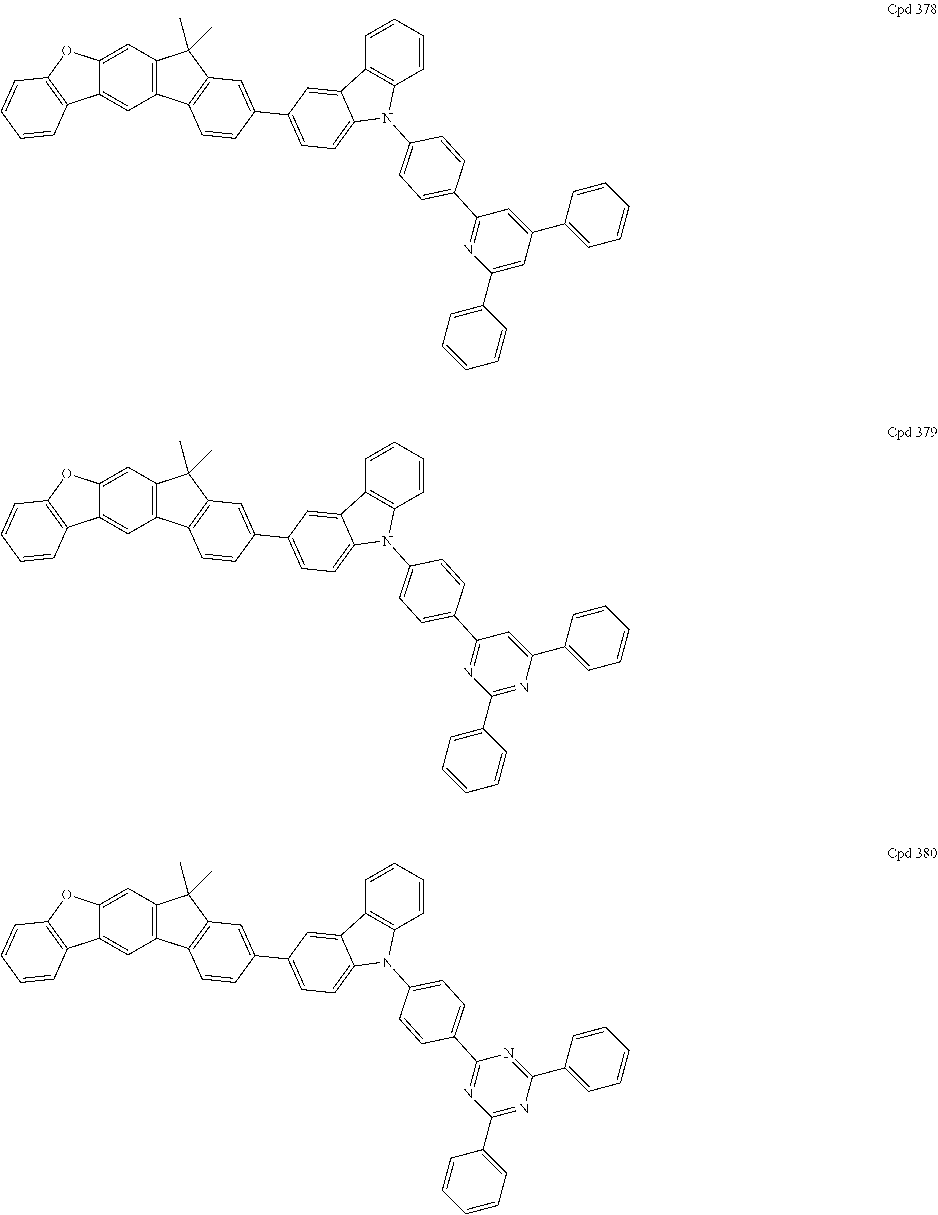 [SMILES: CC1(C)c2cc(-c3ccc4c(c3)c3ccccc3n4-c3ccc(-c4cc(-c5ccccc5)cc(-c5ccccc5)n4)cc3)ccc2-c2cc3c(cc21)oc1ccccc13 ; CC1(C)c2cc(-c3ccc4c(c3)c3ccccc3n4-c3ccc(-c4cc(-c5ccccc5)nc(-c5ccccc5)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5ccccc5)n4)cc3)ccc2-c2cc3c(cc21)oc1ccccc13]

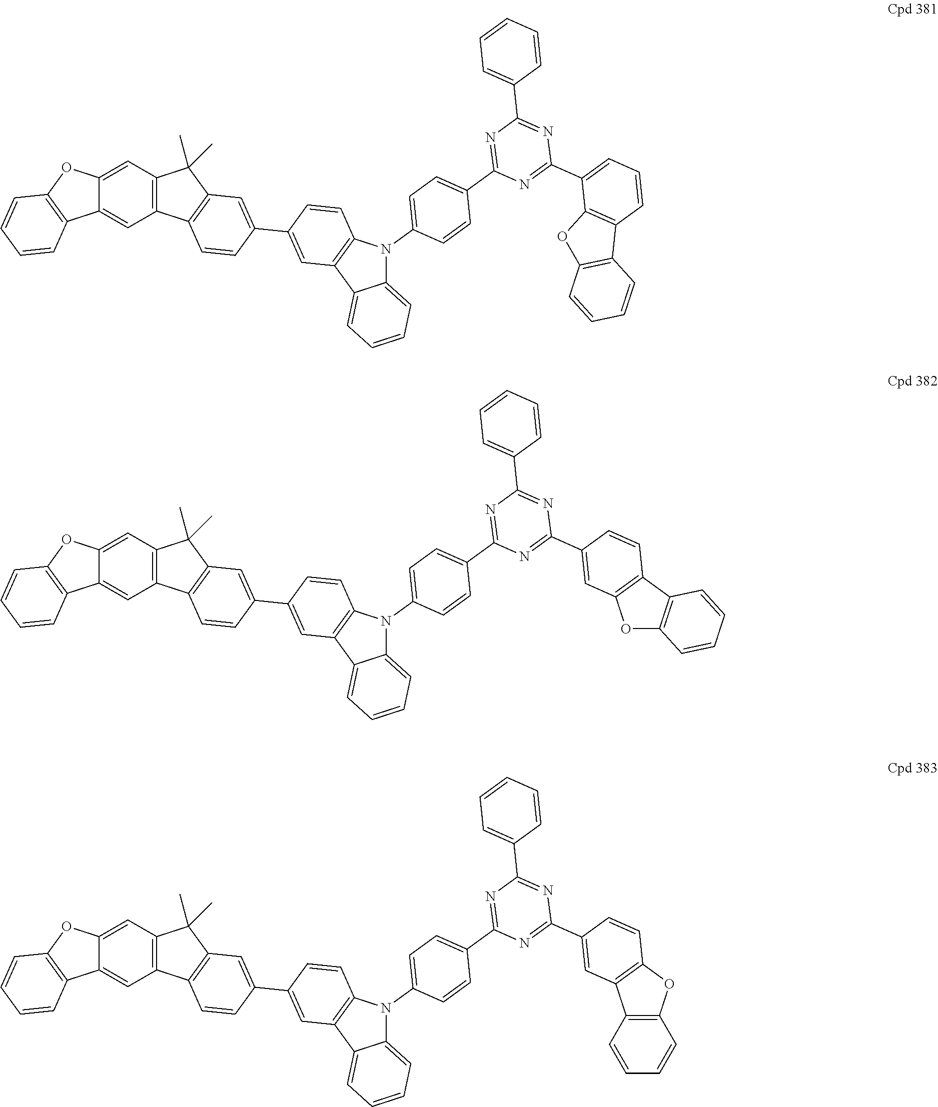 [SMILES: CC1(C)c2cc(-c3ccc4c(c3)c3ccccc3n4-c3ccc(-c4nc(-c5ccccc5)nc(-c5cccc6c5oc5ccccc56)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5ccc6c(c5)oc5ccccc56)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5ccc6oc7ccccc7c6c5)n4)cc3)ccc2-c2cc3c(cc21)oc1ccccc13]

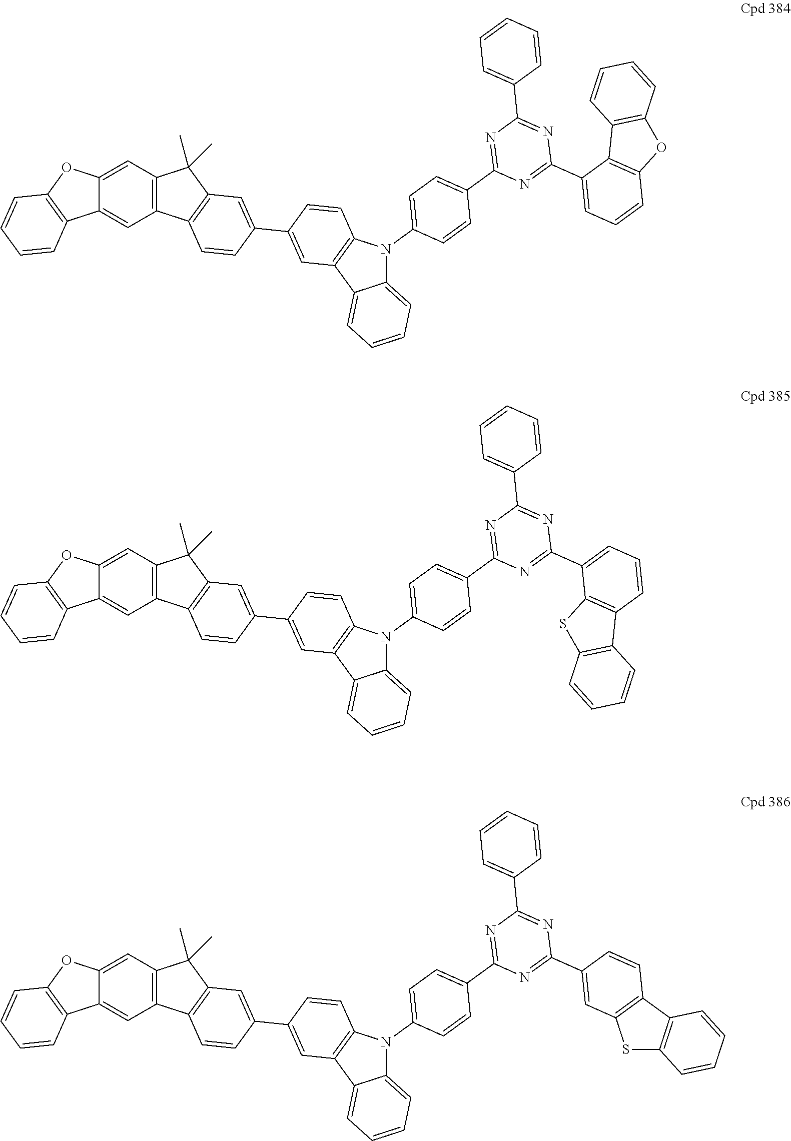 [SMILES: CC1(C)c2cc(-c3ccc4c(c3)c3ccccc3n4-c3ccc(-c4nc(-c5ccccc5)nc(-c5cccc6oc7ccccc7c56)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5cccc6c5sc5ccccc56)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5ccc6c(c5)sc5ccccc56)n4)cc3)ccc2-c2cc3c(cc21)oc1ccccc13]

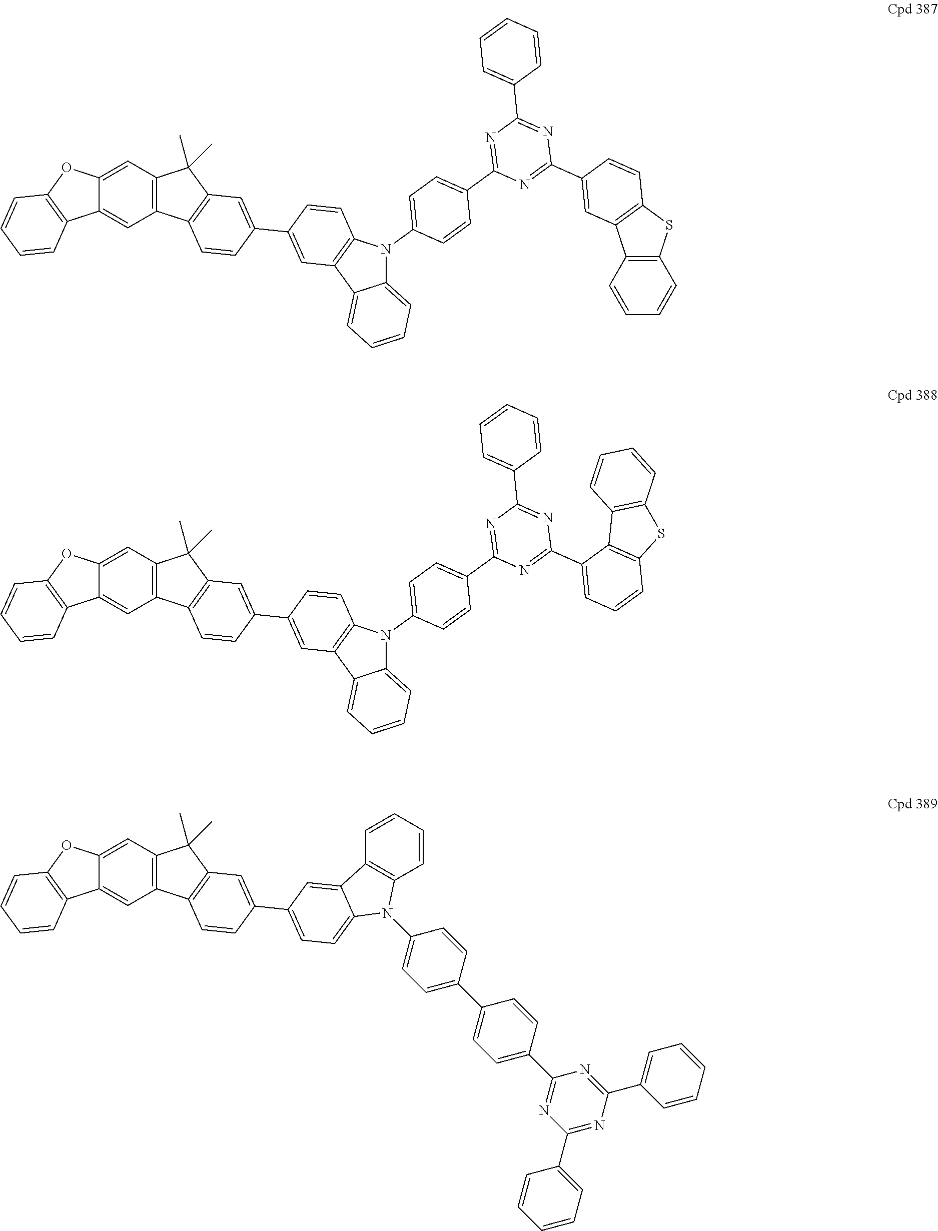 [SMILES: CC1(C)c2cc(-c3ccc4c(c3)c3ccccc3n4-c3ccc(-c4nc(-c5ccccc5)nc(-c5ccc6sc7ccccc7c6c5)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5cccc6sc7ccccc7c56)n4)cc3)ccc2-c2cc3c(cc21)oc1ccccc13 ; CC1(C)c2cc(-c3ccc4c(c3)c3ccccc3n4-c3ccc(-c4ccc(-c5nc(-c6ccccc6)nc(-c6ccccc6)n5)cc4)cc3)ccc2-c2cc3c(cc21)oc1ccccc13]

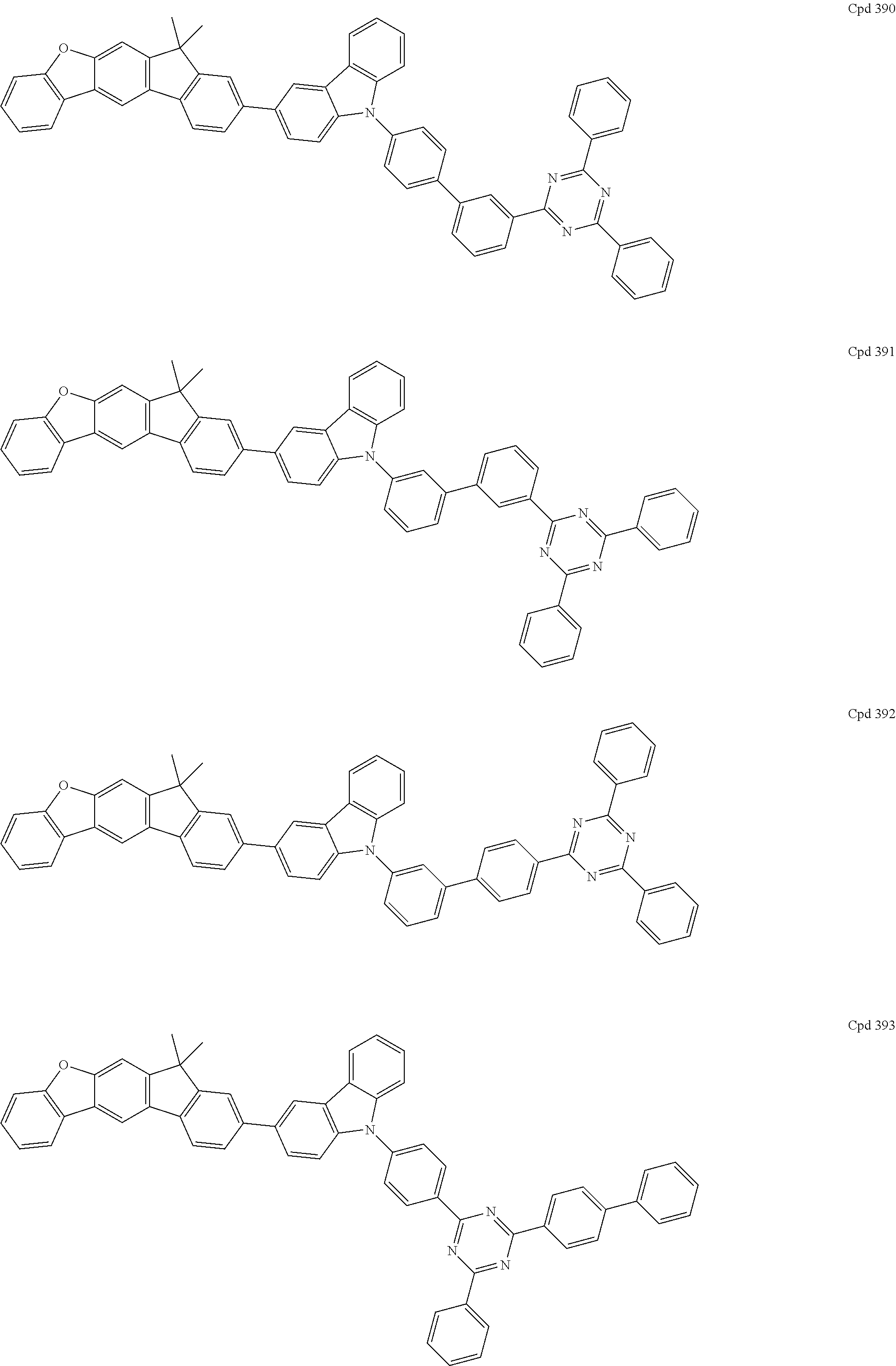 [SMILES: CC1(C)c2cc(-c3ccc4c(c3)c3ccccc3n4-c3ccc(-c4cccc(-c5nc(-c6ccccc6)nc(-c6ccccc6)n5)c4)cc3)ccc2-c2cc3c(cc21)oc1ccccc13 ; CC1(C)c2cc(-c3ccc4c(c3)c3ccccc3n4-c3cccc(-c4cccc(-c5nc(-c6ccccc6)nc(-c6ccccc6)n5)c4)c3)ccc2-c2cc3c(cc21)oc1ccccc13 ; CC1(C)c2cc(-c3ccc4c(c3)c3ccccc3n4-c3cccc(-c4ccc(-c5nc(-c6ccccc6)nc(-c6ccccc6)n5)cc4)c3)ccc2-c2cc3c(cc21)oc1ccccc13 ; CC1(C)c2cc(-c3ccc4c(c3)c3ccccc3n4-c3ccc(-c4nc(-c5ccccc5)nc(-c5ccc(-c6ccccc6)cc5)n4)cc3)ccc2-c2cc3c(cc21)oc1ccccc13]

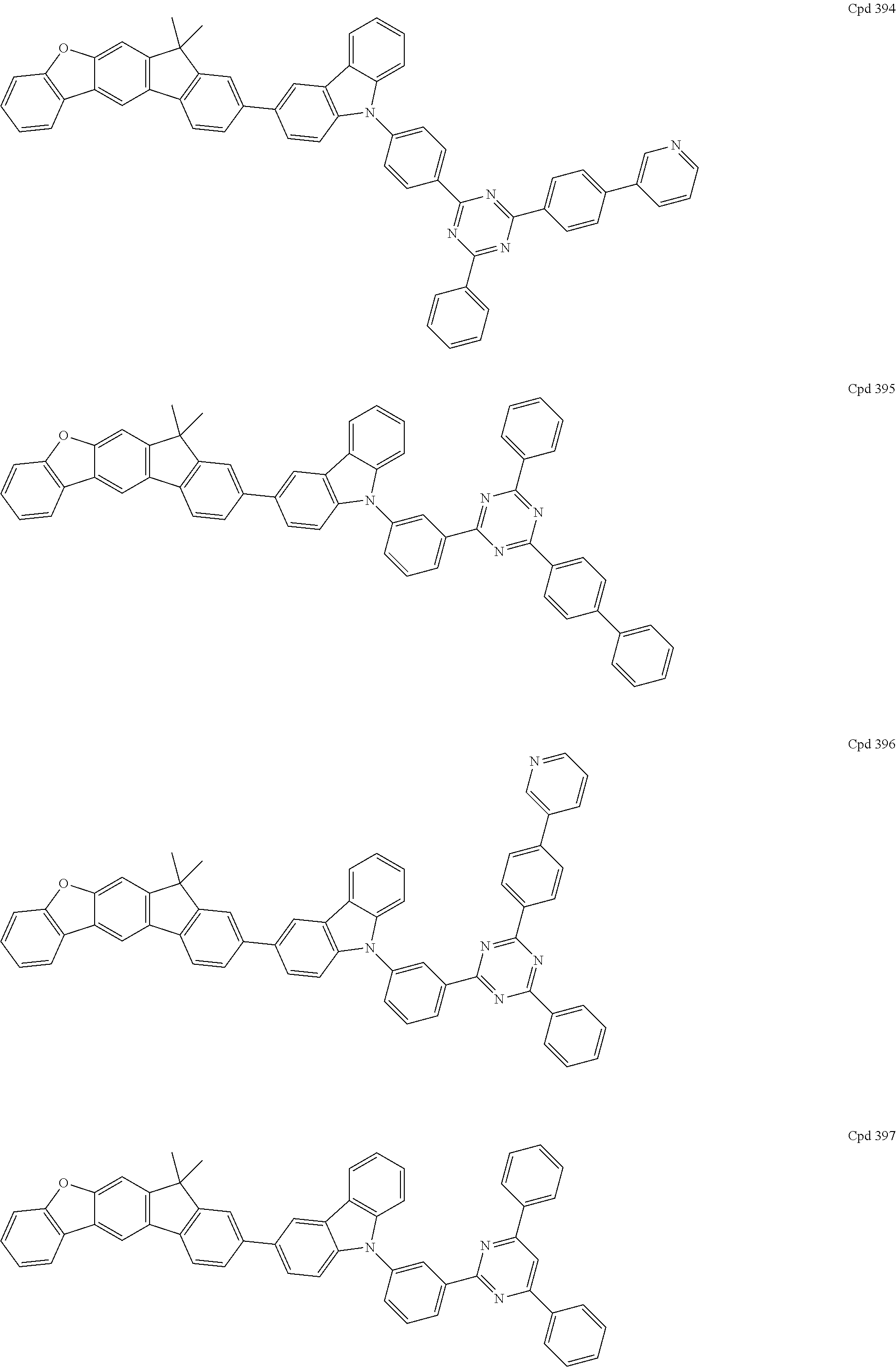 [SMILES: CC1(C)c2cc(-c3ccc4c(c3)c3ccccc3n4-c3ccc(-c4nc(-c5ccccc5)nc(-c5ccc(-c6cccnc6)cc5)n4)cc3)ccc2-c2cc3c(cc21)oc1ccccc13 ; CC1(C)c2cc(-c3ccc4c(c3)c3ccccc3n4-c3cccc(-c4nc(-c5ccccc5)nc(-c5ccc(-c6ccccc6)cc5)n4)c3)ccc2-c2cc3c(cc21)oc1ccccc13 ; CC1(C)c2cc(-c3ccc4c(c3)c3ccccc3n4-c3cccc(-c4nc(-c5ccccc5)nc(-c5ccc(-c6cccnc6)cc5)n4)c3)ccc2-c2cc3c(cc21)oc1ccccc13 ; CC1(C)c2cc(-c3ccc4c(c3)c3ccccc3n4-c3cccc(-c4nc(-c5ccccc5)cc(-c5ccccc5)n4)c3)ccc2-c2cc3c(cc21)oc1ccccc13]

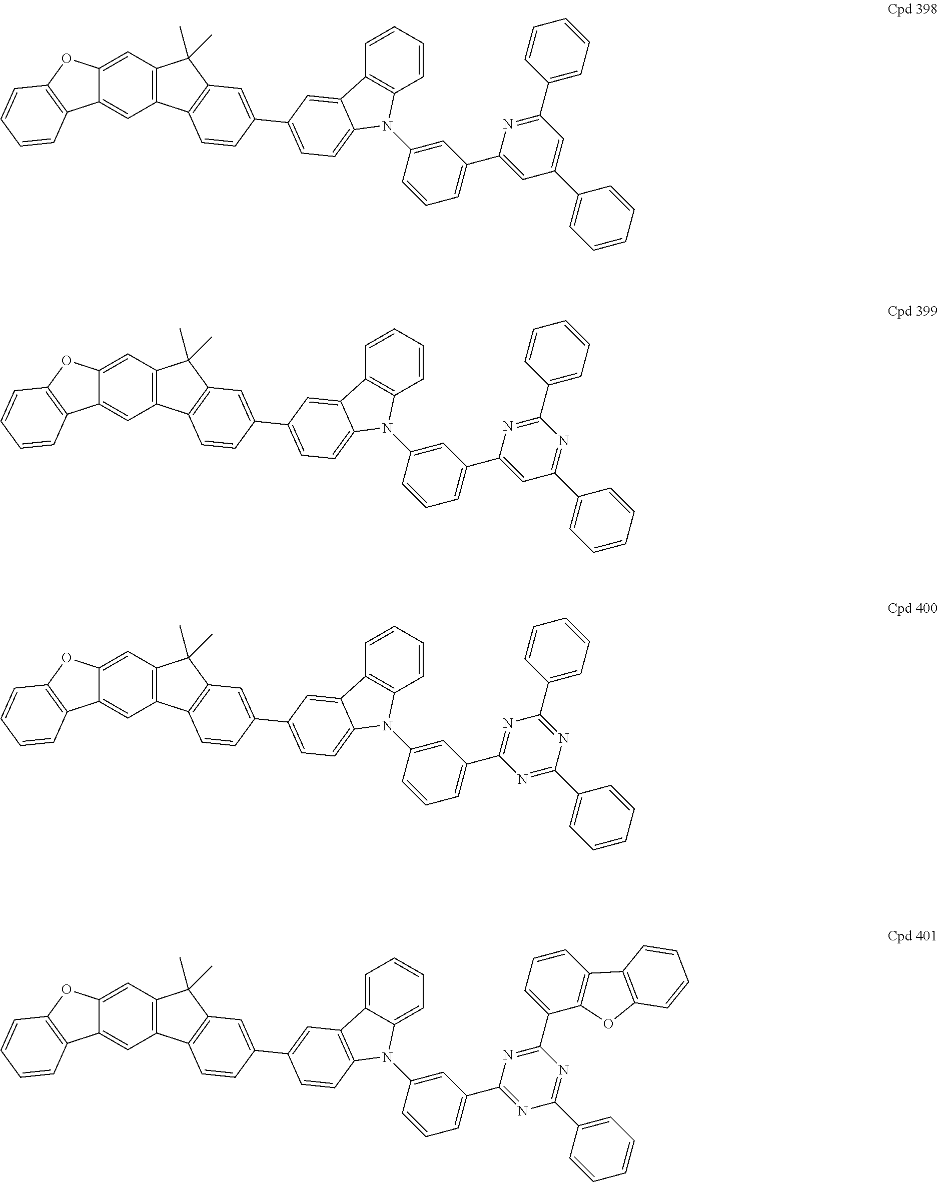 [SMILES: CC1(C)c2cc(-c3ccc4c(c3)c3ccccc3n4-c3cccc(-c4cc(-c5ccccc5)cc(-c5ccccc5)n4)c3)ccc2-c2cc3c(cc21)oc1ccccc13 ; CC1(C)c2cc(-c3ccc4c(c3)c3ccccc3n4-c3cccc(-c4cc(-c5ccccc5)nc(-c5ccccc5)n4)c3)ccc2-c2cc3c(cc21)oc1ccccc13 ; CC1(C)c2cc(-c3ccc4c(c3)c3ccccc3n4-c3cccc(-c4nc(-c5ccccc5)nc(-c5ccccc5)n4)c3)ccc2-c2cc3c(cc21)oc1ccccc13 ; CC1(C)c2cc(-c3ccc4c(c3)c3ccccc3n4-c3cccc(-c4nc(-c5ccccc5)nc(-c5cccc6c5oc5ccccc56)n4)c3)ccc2-c2cc3c(cc21)oc1ccccc13]

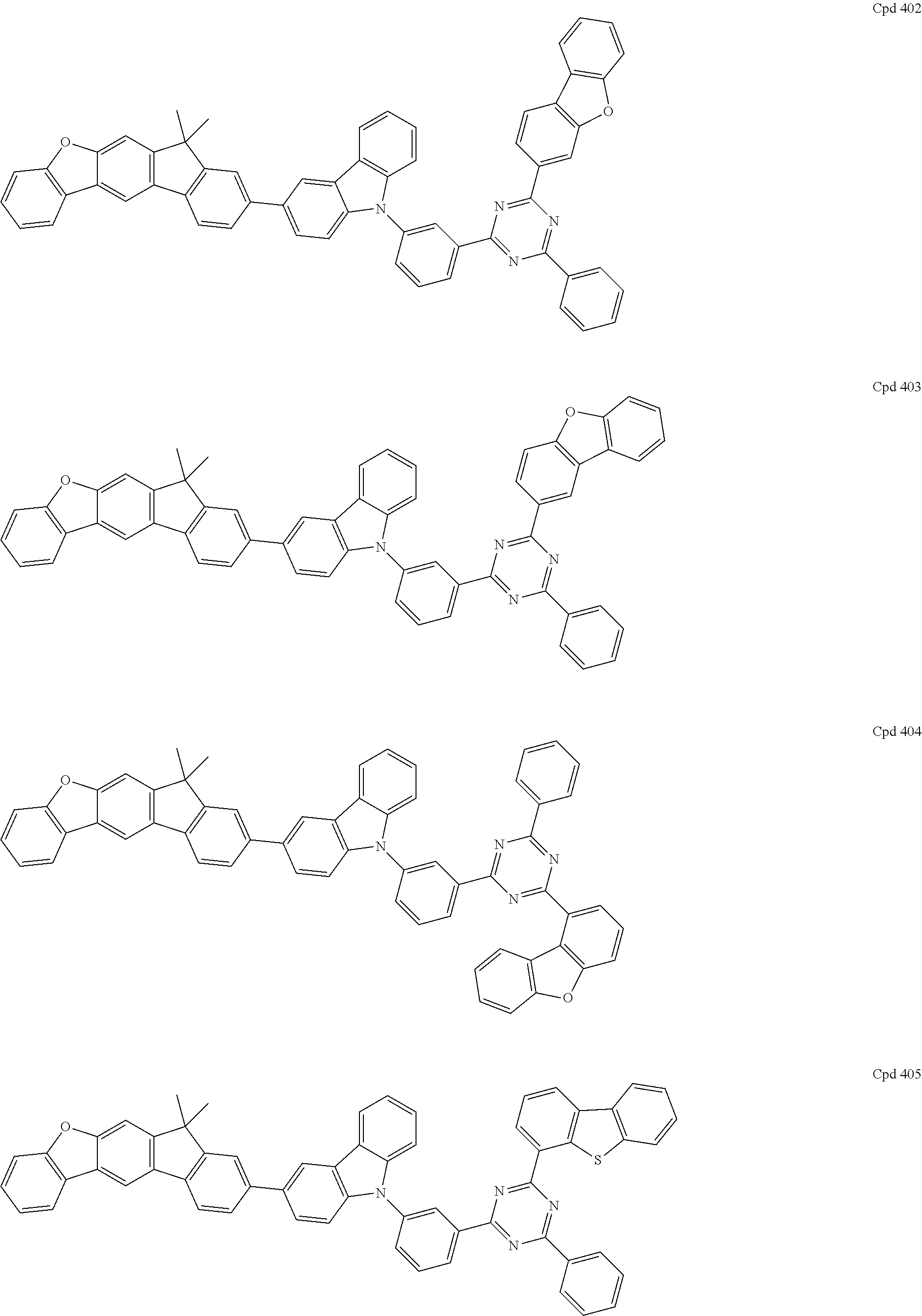 [SMILES: CC1(C)c2cc(-c3ccc4c(c3)c3ccccc3n4-c3cccc(-c4nc(-c5ccccc5)nc(-c5ccc6c(c5)oc5ccccc56)n4)c3)ccc2-c2cc3c(cc21)oc1ccccc13 ; CC1(C)c2cc(-c3ccc4c(c3)c3ccccc3n4-c3cccc(-c4nc(-c5ccccc5)nc(-c5ccc6oc7ccccc7c6c5)n4)c3)ccc2-c2cc3c(cc21)oc1ccccc13 ; CC1(C)c2cc(-c3ccc4c(c3)c3ccccc3n4-c3cccc(-c4nc(-c5ccccc5)nc(-c5cccc6oc7ccccc7c56)n4)c3)ccc2-c2cc3c(cc21)oc1ccccc13 ; CC1(C)c2cc(-c3ccc4c(c3)c3ccccc3n4-c3cccc(-c4nc(-c5ccccc5)nc(-c5cccc6c5sc5ccccc56)n4)c3)ccc2-c2cc3c(cc21)oc1ccccc13]

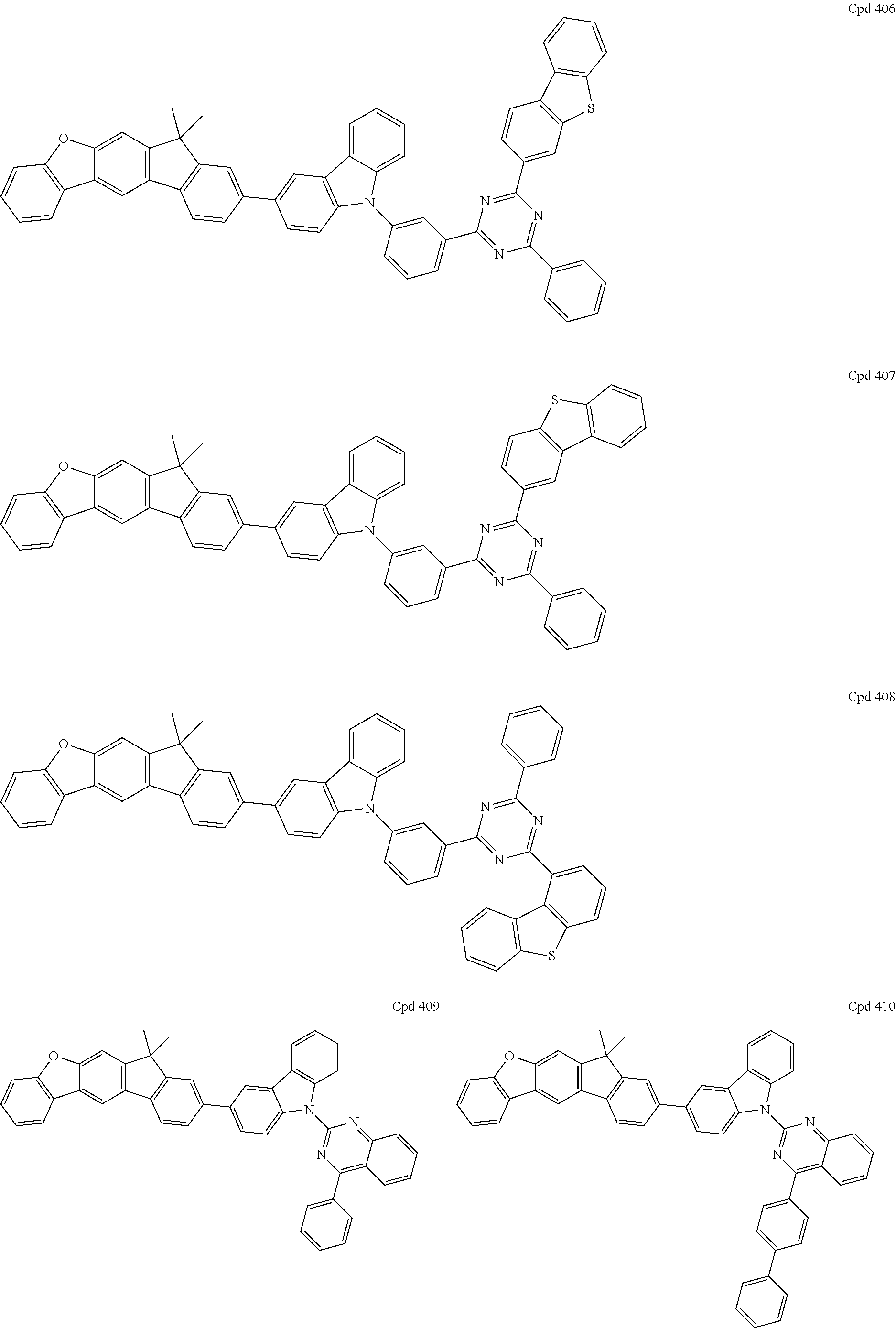

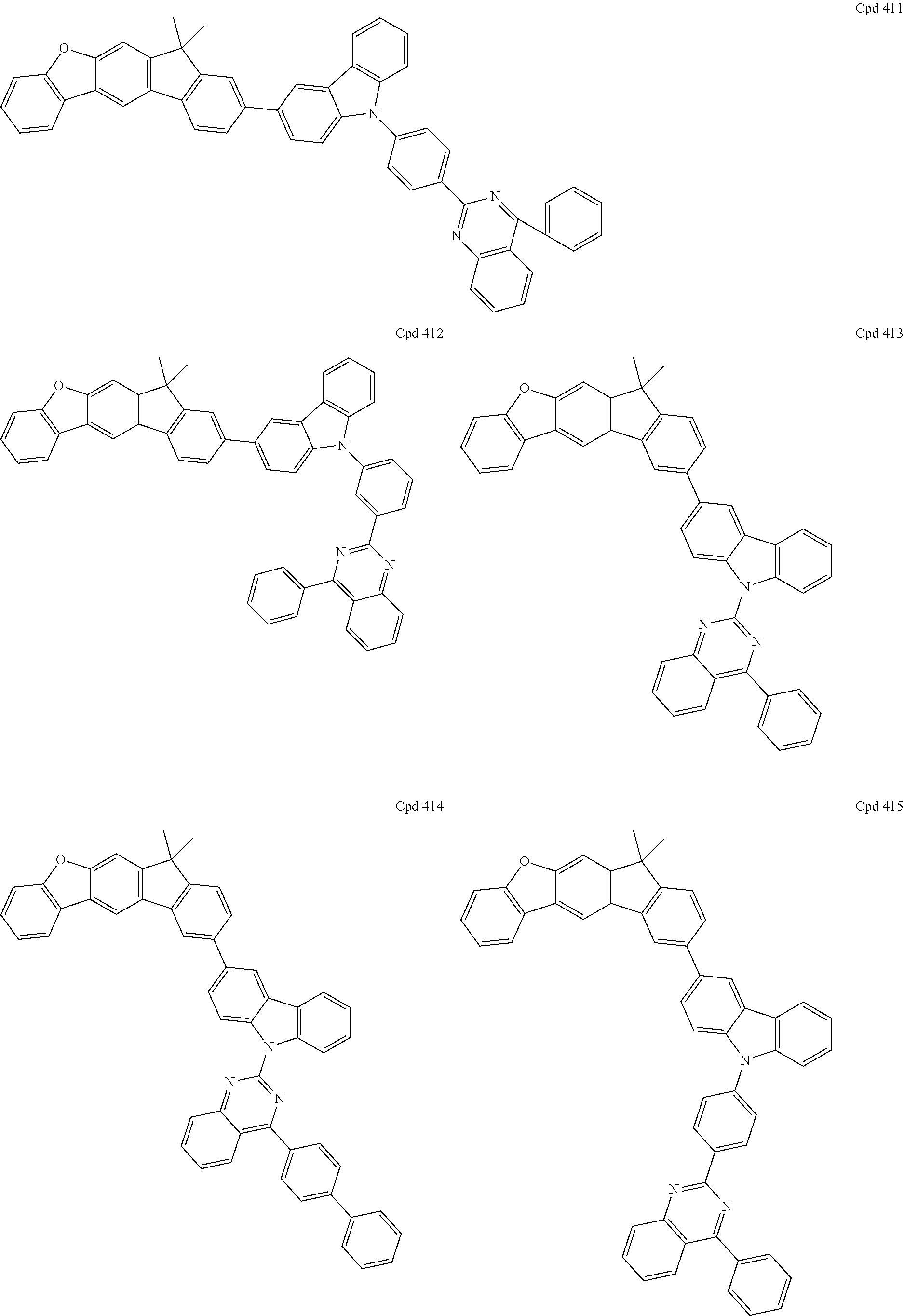 [SMILES: CC1(C)c2cc(-c3ccc4c(c3)c3ccccc3n4-c3ccc(-c4nc(-c5ccccc5)c5ccccc5n4)cc3)ccc2-c2cc3c(cc21)oc1ccccc13 ; CC1(C)c2cc(-c3ccc4c(c3)c3ccccc3n4-c3cccc(-c4nc(-c5ccccc5)c5ccccc5n4)c3)ccc2-c2cc3c(cc21)oc1ccccc13 ; CC1(C)c2ccc(-c3ccc4c(c3)c3ccccc3n4-c3nc(-c4ccccc4)c4ccccc4n3)cc2-c2cc3c(cc21)oc1ccccc13 ; CC1(C)c2ccc(-c3ccc4c(c3)c3ccccc3n4-c3nc(-c4ccc(-c5ccccc5)cc4)c4ccccc4n3)cc2-c2cc3c(cc21)oc1ccccc13 ; CC1(C)c2ccc(-c3ccc4c(c3)c3ccccc3n4-c3ccc(-c4nc(-c5ccccc5)c5ccccc5n4)cc3)cc2-c2cc3c(cc21)oc1ccccc13]

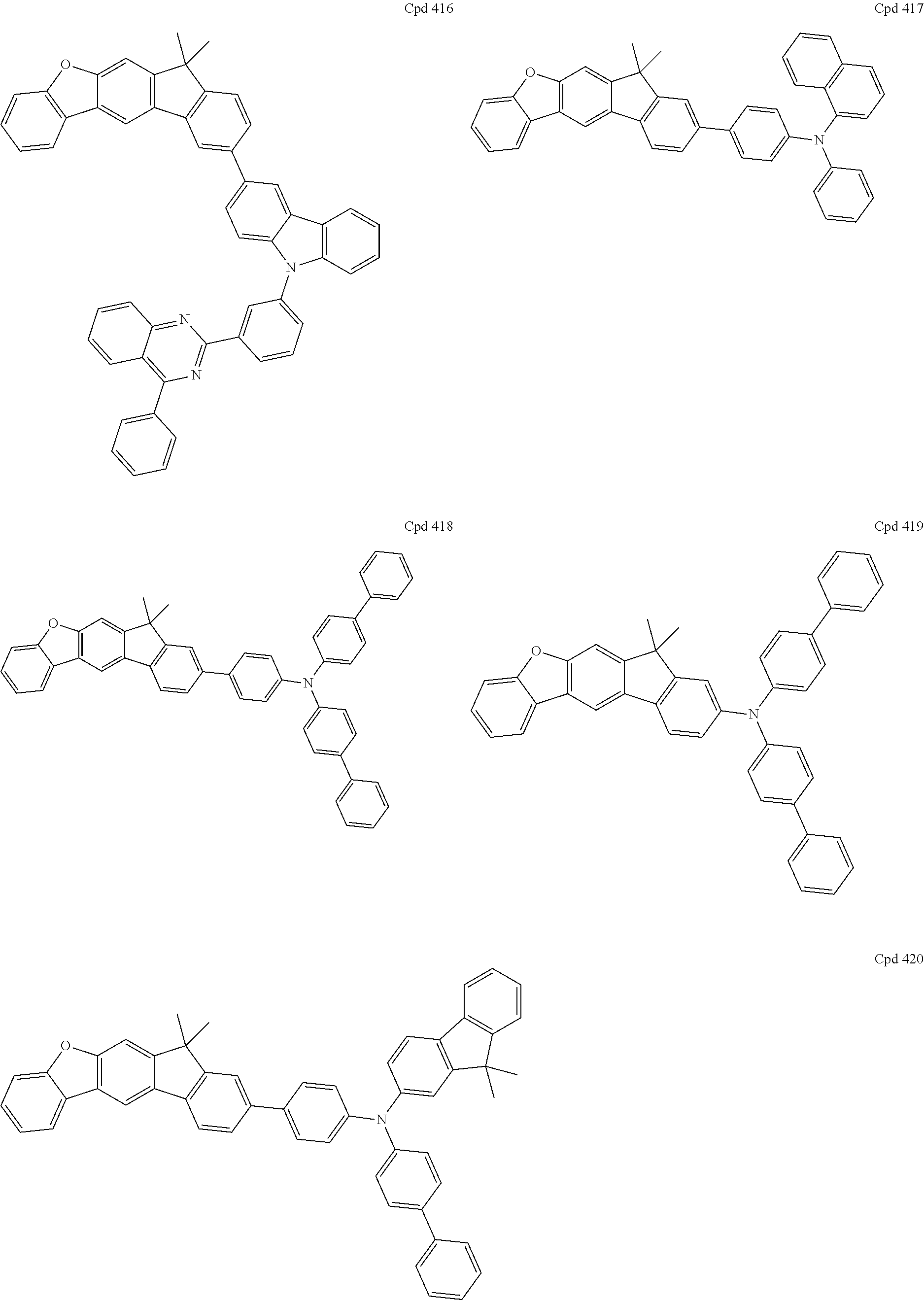 [SMILES: CC1(C)c2ccc(-c3ccc4c(c3)c3ccccc3n4-c3cccc(-c4nc(-c5ccccc5)c5ccccc5n4)c3)cc2-c2cc3c(cc21)oc1ccccc13 ; CC1(C)c2cc(-c3ccc(N(c4ccccc4)c4cccc5ccccc45)cc3)ccc2-c2cc3c(cc21)oc1ccccc13 ; CC1(C)c2cc(-c3ccc(N(c4ccc(-c5ccccc5)cc4)c4ccc(-c5ccccc5)cc4)cc3)ccc2-c2cc3c(cc21)oc1ccccc13 ; CC1(C)c2cc(N(c3ccc(-c4ccccc4)cc3)c3ccc(-c4ccccc4)cc3)ccc2-c2cc3c(cc21)oc1ccccc13 ; CC1(C)c2ccccc2-c2ccc(N(c3ccc(-c4ccccc4)cc3)c3ccc(-c4ccc5c(c4)C(C)(C)c4cc6oc7ccccc7c6cc4-5)cc3)cc21]

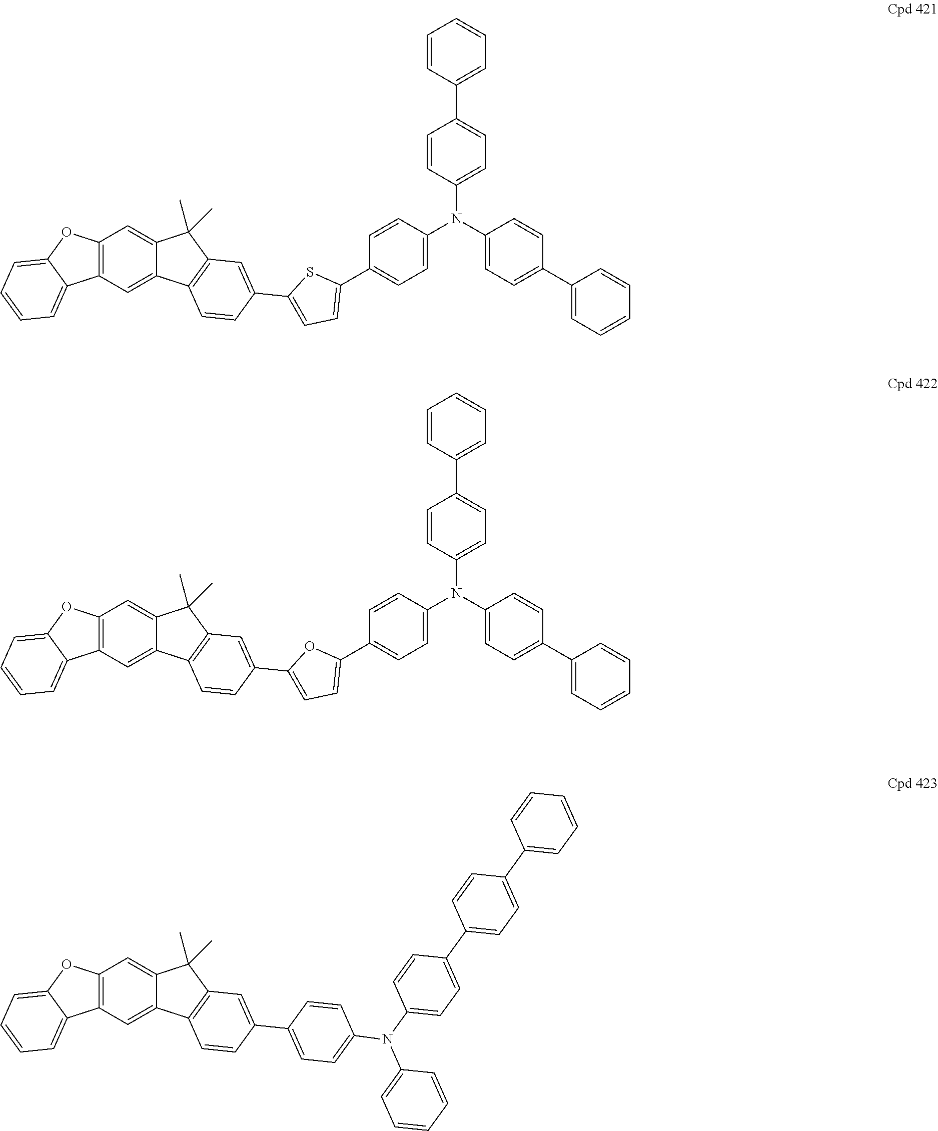 [SMILES: CC1(C)c2cc(-c3ccc(-c4ccc(N(c5ccc(-c6ccccc6)cc5)c5ccc(-c6ccccc6)cc5)cc4)s3)ccc2-c2cc3c(cc21)oc1ccccc13 ; CC1(C)c2cc(-c3ccc(-c4ccc(N(c5ccc(-c6ccccc6)cc5)c5ccc(-c6ccccc6)cc5)cc4)o3)ccc2-c2cc3c(cc21)oc1ccccc13 ; CC1(C)c2cc(-c3ccc(N(c4ccccc4)c4ccc(-c5ccc(-c6ccccc6)cc5)cc4)cc3)ccc2-c2cc3c(cc21)oc1ccccc13]

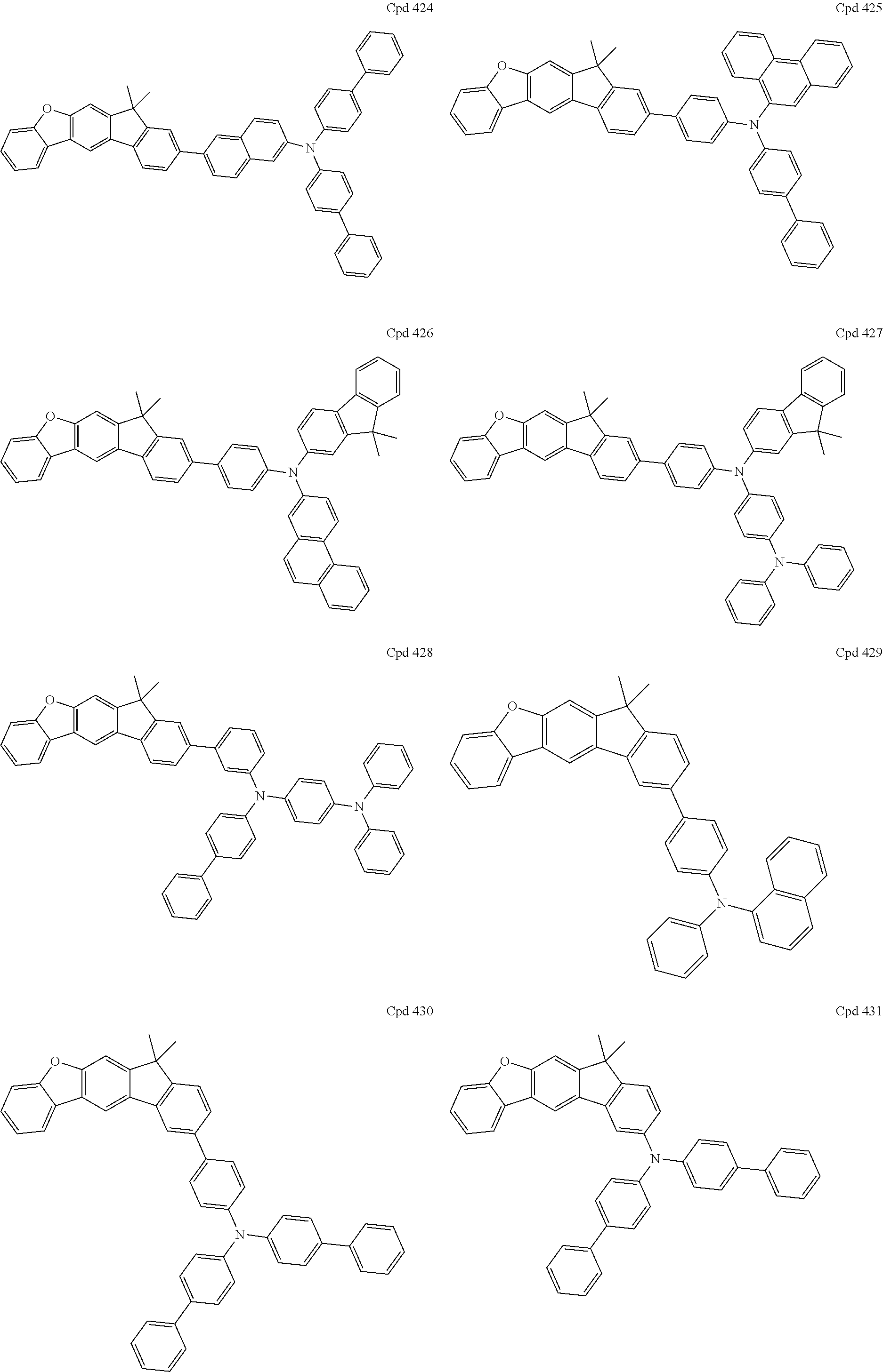

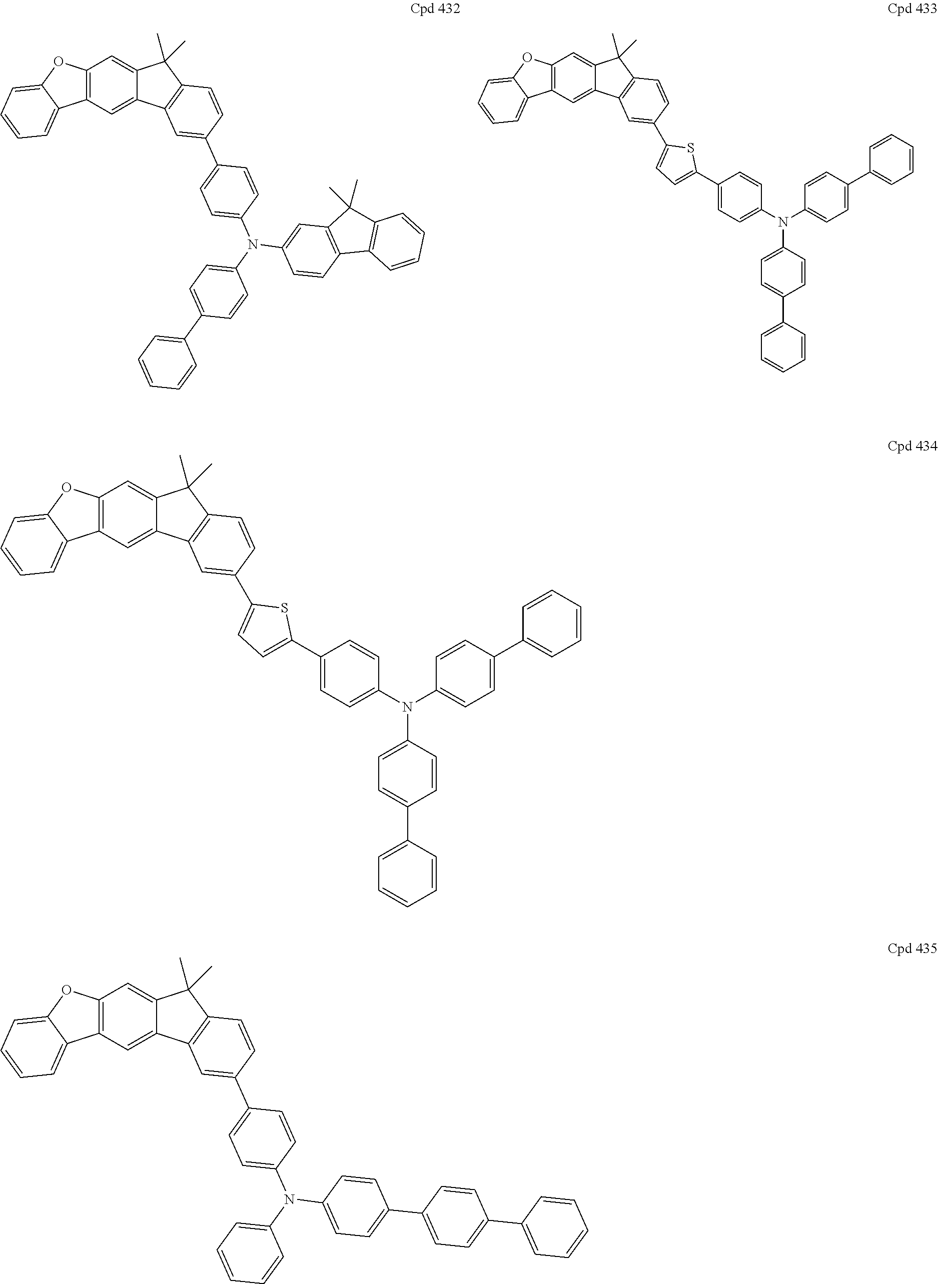 [SMILES: CC1(C)c2ccccc2-c2ccc(N(c3ccc(-c4ccccc4)cc3)c3ccc(-c4ccc5c(c4)-c4cc6c(cc4C5(C)C)oc4ccccc46)cc3)cc21 ; CC1(C)c2ccc(-c3ccc(-c4ccc(N(c5ccc(-c6ccccc6)cc5)c5ccc(-c6ccccc6)cc5)cc4)s3)cc2-c2cc3c(cc21)oc1ccccc13 ; CC1(C)c2ccc(-c3ccc(-c4ccc(N(c5ccc(-c6ccccc6)cc5)c5ccc(-c6ccccc6)cc5)cc4)s3)cc2-c2cc3c(cc21)oc1ccccc13 ; CC1(C)c2ccc(-c3ccc(N(c4ccccc4)c4ccc(-c5ccc(-c6ccccc6)cc5)cc4)cc3)cc2-c2cc3c(cc21)oc1ccccc13]

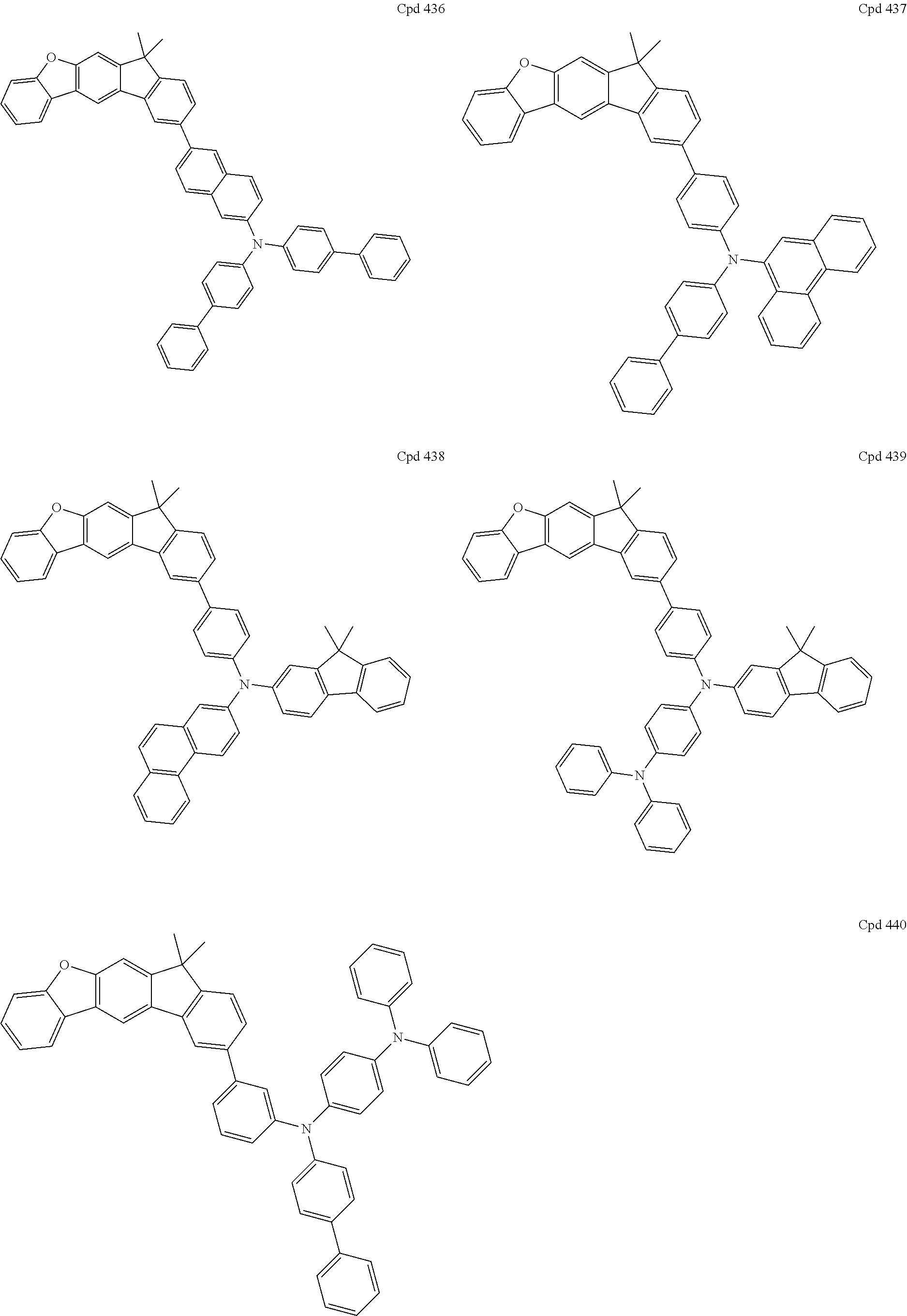

The compound of Chemical Formula 1 of the present invention may be synthesized using general synthesis methods (refer to Chem. Rev., 60:313 (1960); J. Chem. SOC. 4482 (1955); Chem. Rev. 95: 2457 (1995) or the like). Detailed synthesis processes of the compounds of the present invention will be specifically described in synthesis examples to be described later.

2. Organic Electroluminescent Device

Meanwhile, another aspect of the present invention provides an organic electroluminescent device including the compound represented by Chemical Formula 1.

Specifically, the present invention relates to an organic electroluminescent device including (i) an anode, (ii) a cathode and (iii) one or more organic material layers provided between the anode and the cathode, and at least one of the organic material layers includes the compound represented by Chemical Formula 1. Herein, the compound represented by Chemical Formula 1 may be used either alone or as a mixture of two or more types.

According to one embodiment of the present invention, the one or more organic material layers may include any one or more of a hole injection layer, a hole transport layer, a light emitting layer, an electron transport layer and an electron injection layer, and at least one organic material layer thereof may include the compound represented by Chemical Formula 1.

Preferably, the organic material layer including the compound represented by Chemical Formula 1 may be a light emitting layer or a hole transport layer. More preferably, when the compound represented by Chemical Formula 1 is included in a light emitting layer, luminous efficiency, luminance, power efficiency, thermal stability and device lifetime of an organic electroluminescent device may be significantly enhanced.

For example, the compound represented by Chemical Formula 1 may be a phosphorescent host, a fluorescent host or a dopant material of a light emitting layer, and may be preferably a phosphorescent host of a light emitting layer.

According to another embodiment of the present invention, the one or more organic material layers may include any one or more of a hole injection layer, a hole transport layer, a light emitting auxiliary layer, a light emitting layer, an electron transport layer and an electron injection layer, and herein, at least one of the organic material layers may include the compound represented by Chemical Formula 1.

Preferably, the organic material layer including the compound represented by Chemical Formula 1 may be a light emitting auxiliary layer. Particularly, when the compound represented by Chemical Formula 1 is included in a light emitting auxiliary layer material of an organic electroluminescent device, efficiency (luminous efficiency and power efficiency), lifetime and luminance of the organic electroluminescent device may be further enhanced, and driving voltage may be further lowered.

According to another embodiment of the present invention, the one or more organic material layers may include any one or more of a hole injection layer, a hole transport layer, a light emitting layer, a hole blocking layer, an electron transport layer and an electron injection layer, and herein, at least one of the organic material layers may include the compound represented by Chemical Formula 1.

Preferably, the organic material layer including the compound represented by Chemical Formula 1 may be a hole blocking layer. Particularly, when the compound represented by Chemical Formula 1 is included in a hole blocking layer of an organic electroluminescent device, efficiency (luminous efficiency and power efficiency), lifetime and luminance of the organic electroluminescent device may be further enhanced, and driving voltage may be further lowered.

The structure of such an organic electroluminescent device of the present invention is not particularly limited, and examples thereof may include a structure of, as well as consecutively laminating an anode, one or more organic material layers and a cathode on a substrate, inserting an insulating layer or an adhesive layer at an interface of the electrode and the organic material layer.

According to one embodiment, the organic electroluminescent device may have a structure in which an anode, a hole injection layer, a hole transport layer, a light emitting layer, an electron transport layer and a cathode are consecutively laminated on a substrate, and as necessary, a light emitting auxiliary layer may be inserted between the hole transport layer and the light emitting layer, and an electron injection layer may also be disposed on the electron transport layer.

The organic electroluminescent device of the present invention may be manufactured by forming organic material layers and electrodes using materials and methods known in the art except that at least one of the one or more organic material layers, for example, a light emitting layer or a light emitting auxiliary layer, is formed to include the compound represented by Chemical Formula 1.

The organic material layer may be formed using a vacuum deposition method or a solution coating method. Examples of the solution coating method may include spin coating, dip coating, doctor blading, inkjet printing, thermal transfer method or the like, but are not limited thereto.

Examples of the substrate capable of being used when manufacturing the organic electroluminescent device in the present invention may include silicon wafers, quartz, glass plates, metal plates, plastic films, sheets and the like, but are not limited thereto.

In addition, examples of the anode material may include metals such as vanadium, chromium, copper, zinc or gold, or alloys thereof; metal oxides such as zinc oxide, indium oxide, indium tin oxide (ITO) or indium zinc oxide (IZO); combinations of metals and oxides such as ZnO:Al or SnO₂:Sb; conductive polymers such as polythiophene, poly(3-methylthiophene), poly[3,4-(ethylene-1,2-dioxy)thiophene] (PEDT), polypyrrole and polyaniline; carbon black, or the like, but are not limited thereto.

In addition, examples of the cathode material metals such as magnesium, calcium, sodium, potassium, titanium, indium, yttrium, lithium, gadolinium, aluminum, silver, tin or lead, or alloys thereof; multilayer-structured materials such as LiF/Al or LiO₂/Al, or the like, but are not limited thereto.

In addition, materials used as the hole injection layer, the hole transport layer, the electron injection layer and the electron transport layer are not particularly limited, and common materials known in the art may be used without limit.

Hereinafter, the present invention will be described in detail with reference to examples, however, the following examples are for illustrative purposes only, and the present invention is not limited to the following examples.

### PREPARATION EXAMPLE 1

### Synthesis of Compounds Inv 1 and Inv 2

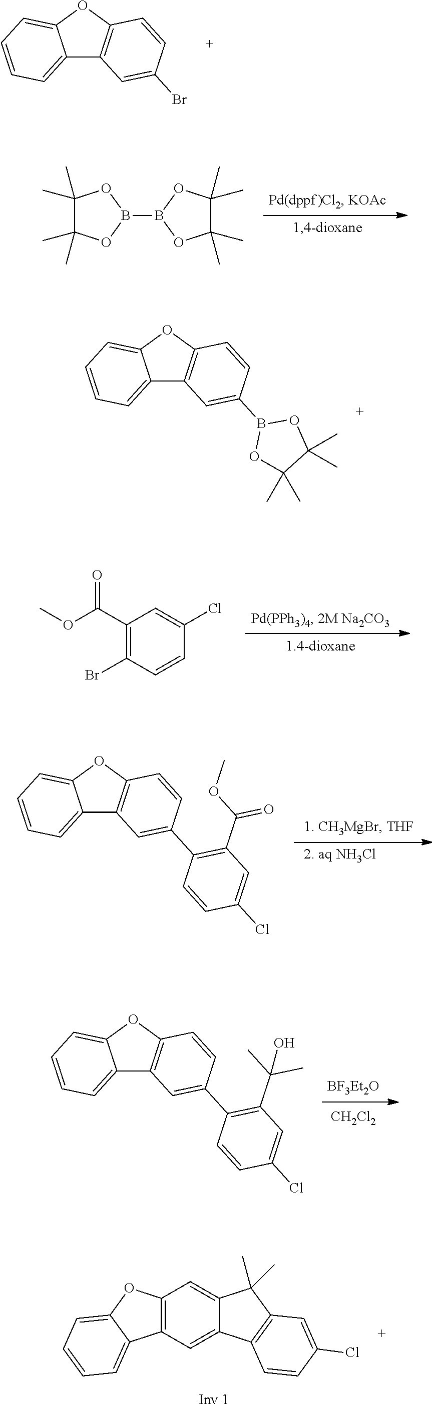

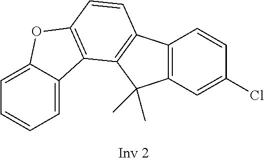 [SMILES: CC1(C)c2cc(Cl)ccc2-c2ccc3oc4ccccc4c3c21]

<Step 1> Synthesis of 2-(dibenzo[b,d]furan-2-yl)-4,4,5,5-tetramethyl-1,3,2-dioxaborolane

2-Bromodibenzo[b,d]furan (94 g, 0.38 mol), bis(pinacolato)diboron (115.8 g, 0.46 mol), Pd(dppf)Cl₂ (31 g, 0.038 mol) and KOAc (111.9 g, 1.14 mol) were introduced to a flask, then dissolved by introducing 1,4-dioxane (2 L) thereto, and the result was stirred for 8 hours while heating. After the reaction was finished, distilled water was introduced thereto, and the organic layer was extracted with ethyl acetate. The obtained organic layer was dried with Na₂SO₄, vacuum distilled, and then purified using column chromatography to obtain compound 2-(dibenzo[b,d]furan-2-yl)-4,4,5,5-tetramethyl-1,3,2-dioxaborolane (73 g, yield 62%).

<Step 2> Synthesis of methyl 5-chloro-2-(dibenzo[b,d]furan-2-yl)benzoate 2-(Dibenzo[b,d]furan-2-yl)-4,4,5,5-tetramethyl-1,3,2-dioxaborolane

(22.6 g, 76.9 mmol) obtained in <Step 1>, methyl 2-bromo-5-chlorobenzoate (21.1 g, 84.57 mmol) and Pd(PPh₃)₄ (0.89 g, 0.769 mmol) were introduced to a flask and dissolved in 1,4-dioxane (257 mL) under the nitrogen atmosphere. An aqueous solution (128 ml) having K₂CO₃ (17 g, 115.3 mmol) dissolved therein was added thereto, and the result was stirred under reflux for 7 hours at 70° C. After the reaction was finished, the result was extracted with ethyl acetate, and then the obtained organic layer was dried with Na₂SO₄, vacuum distilled, and then purified using column chromatography to obtain compound methyl 5-chloro-2-(dibenzo[b,d]furan-2-yl)benzoate (21.8 g, yield 84%).

<Step 3> Synthesis of Compounds Inv 1 and Inv 2

Under the nitrogen atmosphere, methyl 5-chloro-2-(dibenzo[b,d]furan-2-yl)benzoate (21.8 g 64.95 mmol) obtained in <Step 2> was introduced to a three-neck round bottom flask heated and dried under vacuum, then dissolved by adding THF (325 mL) thereto, and the result is was cooled to −10° C. and stirred. 3.0 M CH₃MgBr (54 mL) (in ether, 162.4 mmol) was slowly added thereto over 30 minutes. The reaction solution was warmed to room temperature, and stirred for 12 hours under the nitrogen atmosphere. The reaction solution was cooled to 0° C., and then an aqueous solution dissolving NH₄Cl (10.4 g, 194.85 mmol) in distilled water (100 mL) was slowly added thereto. The reaction solution was extracted with distilled water and ether, the organic layer solution was dried with Na₂SO₄ and filtered, and the filtrate was vacuum concentrated. The dried residue was introduced to a three-neck round bottom flask and dissolved by adding CH₂C₂ (325 mL) thereto under the nitrogen atmosphere, and the result was cooled to 0° C. and stirred. Boron trifluoride dimethyl etherate (4 mL, 32.5 mmol) was slowly added thereto for 10 minutes, and after raising the temperature to room temperature, the result was stirred for 12 hours. When the reaction was finished, an aqueous sodium bicarbonate solution was slowly added thereto at 0° C., and the result was stirred for 30 minutes. The extract obtained by extracting the reaction solution with dichloromethane/distilled water was dried with Na₂SO₄, vacuum distilled, and then purified using column chromatography to obtain Compound Inv 1 (10.3 g, yield 50%) and Compound Inv 2 (9.3 g, yield 45%).

### PREPARATION EXAMPLE 2

### Synthesis of Compounds Inv 3 and Inv 4

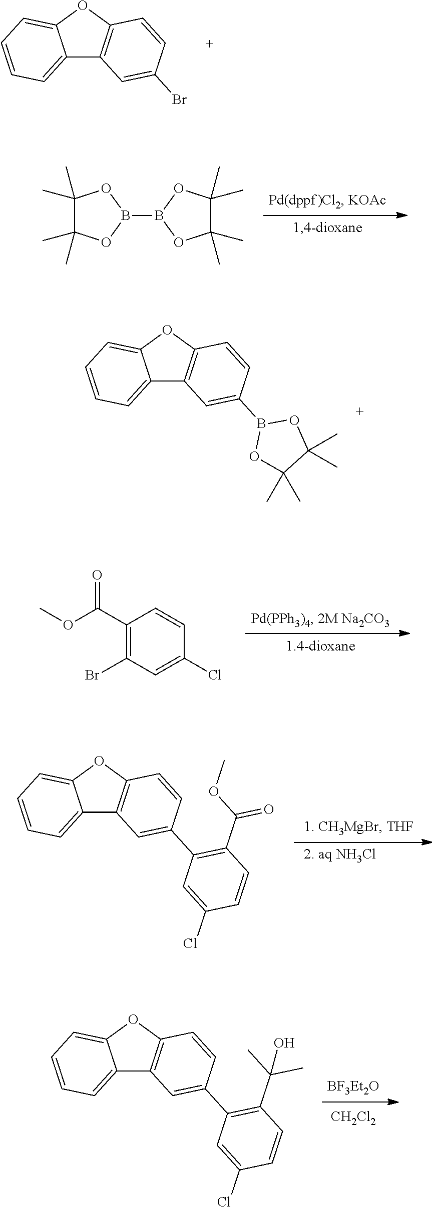

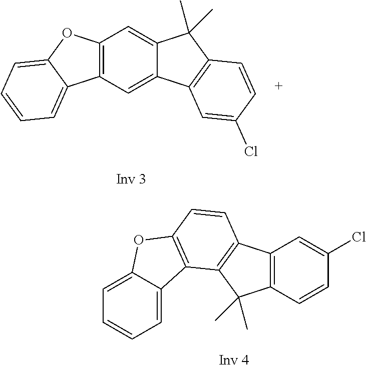 [SMILES: CC1(C)c2ccc(Cl)cc2-c2cc3c(cc21)oc1ccccc13 ; CC1(C)c2ccc(Cl)cc2-c2ccc3oc4ccccc4c3c21]

<Step 1> Synthesis of 2-(dibenzol[b,d]furan-2-yl)-4,4,5,5-tetramethyl-1,3,2-dioxaborolane

2-Bromodibenzo[b,d]furan (94 g, 0.38 mol), bis(pinacolato)diboron (115.8 g, 0.46 mol), Pd(dppf)C₂ (31 g, 0.038 mol) and KOAc(111.9 g 1.14 mol) were introduced to a flask, then dissolved by introducing 1,4-dioxane (2 L) thereto, and the result was stirred for 8 hours while heating. After the reaction was finished, distilled water was introduced thereto, and the organic layer was extracted with ethyl acetate. The obtained organic layer was dried with Na₂SO₄, vacuum distilled, and then purified using column chromatography to obtain compound 2-(dibenzo[b,d]furan-2-yl)-4,4,5,5-tetramethyl-1,3,2-dioxaborolane (73 g, yield 62%).

<Step 2> Synthesis of methyl 4-chloro-2-(dibenzo[b,d]furan-2-yl)benzoate

2-(Dibenzo[b,d]furan-2-yl)-4,4,5,5-tetramethyl-1,3,2-dioxaborolane (69 g, 0.235 mol) obtained in <Step 1>, 2-bromo-4-chloro-1-nitrobenzene (67 g, 0.282 mol) and Pd(PPh₃)₄ (13.5 g, 0.011 mol) were introduced to a flask and dissolved by introducing a saturated aqueous 2 M Na₂CO₃ solution (352 ml) and 1,4-dioxane (2 L) thereto, and the result was stirred for 8 hours while heating. After the reaction was finished, distilled water was introduced thereto, and the organic layer was extracted with ethyl acetate. The obtained organic layer was dried with Na₂SO₄, vacuum distilled, and then purified using column chromatography to obtain compound methyl 4-chloro-2-(dibenzo[b,d]furan-2-yl)benzoate (71 g yield 91%).

<Step 3> Synthesis of Compounds Inv 3 and Inv 4

Under the nitrogen atmosphere, methyl 4-chloro-2-(dibenzo[b,d]furan-2-yl)benzoate (21.8 g 64.95 mmol) obtained in <Step 2> was introduced to a three-neck round bottom flask heated and dried under vacuum, then dissolved by adding THF (325 mL) thereto, and the result was cooled to −10° C. and stirred. 3.0 M CH₃MgBr (54 mL) (in ether, 162.4 mmol) was slowly added thereto over 30 minutes. The reaction solution was warmed to room temperature, and stirred for 12 hours under the nitrogen atmosphere. The reaction solution was cooled to 0° C., and then an aqueous solution dissolving NH₄Cl (10.4 g, 194.85 mmol) in distilled water (100 mL) was slowly added thereto. The reaction solution was extracted with distilled water and ether, the organic layer solution was dried with Na₂SO₄ and filtered, and the filtrate was vacuum concentrated. The dried residue was introduced to a three-neck round bottom flask and dissolved by adding CH₂C₂ (325 mL) thereto under the nitrogen atmosphere, and the result was cooled to 0° C. and stirred. Boron trifluoride dimethyl etherate (4 mL, 32.5 mmol) was slowly added thereto for 10 minutes, and after raising the temperature to room temperature, the result was stirred for 12 hours. When the reaction was finished, an aqueous sodium bicarbonate solution was slowly added thereto at 0° C., and the result was stirred for 30 minutes. The extract obtained by extracting the reaction solution with dichloromethane/distilled water was dried with Na₂SO₄, vacuum distilled, and then purified using column chromatography to obtain Compound Inv 3 (9.3 g, yield 45%) and Compound Inv 4 (8.2 g, yield 39%).

### PREPARATION EXAMPLE 5

### Synthesis of Compounds Inv 5 and Inv 6

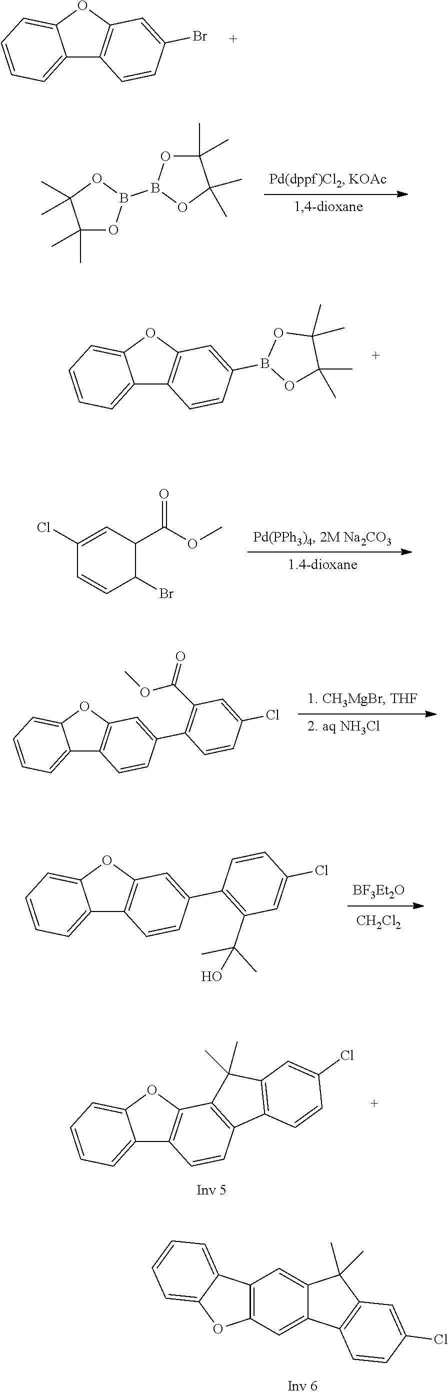

Compound Inv 5 (9.0 g, yield 43%) and Compound Inv 6 (8.6 g, yield 41%) were obtained in the same manner as in Preparation Example 1 except that, in <Step 1>, 3-bromodibenzo[b,d]furan was used instead of 2-bromodibenzo[b,d]furan.

### PREPARATION EXAMPLE 6

### Synthesis of Compounds Inv 7 and Inv 8

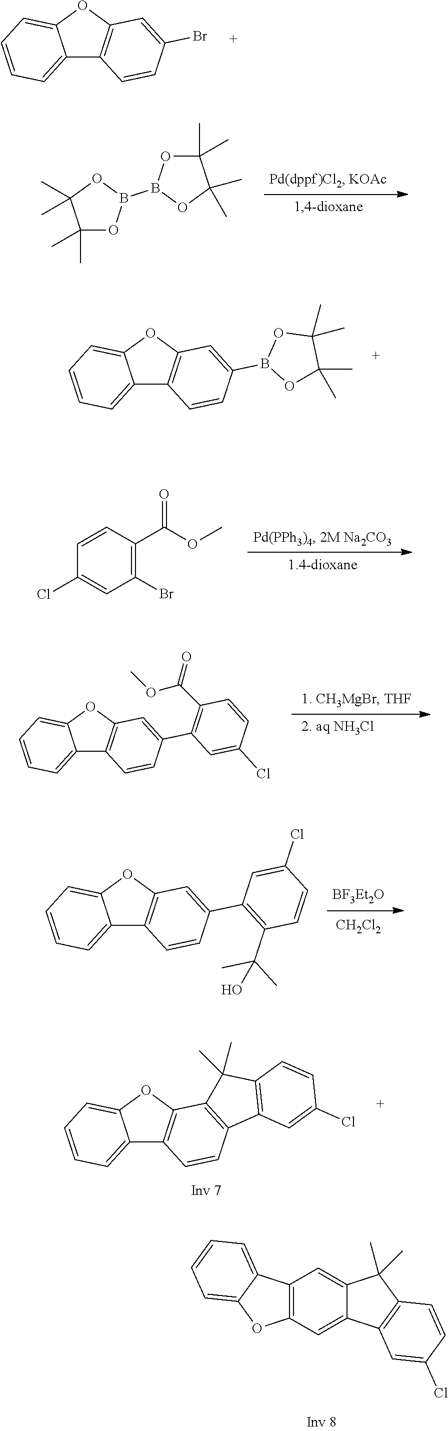

Compound Inv 7 (8.8 g, yield 43%) and Compound Inv 8 (8.9 g, yield 43%) were obtained in the same manner as in Preparation Example 2 except that, in <Step 1>, 3-bromodibenzo[b,d]furan was used instead of 2-bromodibenzo[b,d]furan.

### SYNTHESIS EXAMPLE 1

### Synthesis of Cpd 10

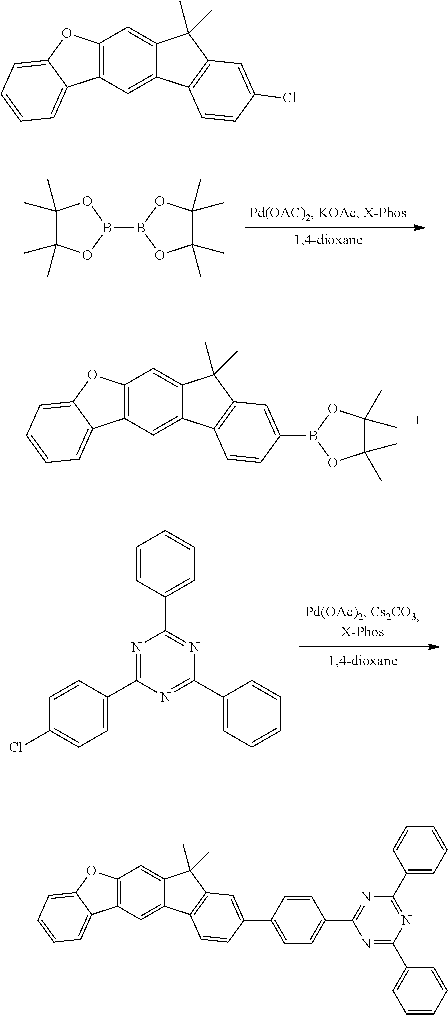

<Step 1> Synthesis of 2-(7,7-dimethyl-7H-fluoreno[2,3-b]benzofuran-9-yl)-4,4,5,5-tetramethyl-1,3,2-dioxaborolane

Inv 1 (100 g, 0.313 mol), 4,4,4′,4′,5,5,5′,5′-octamethyl-2,2′-bi(1,3,2-dioxaborolane) (95.6 g, 0.376 mol), Pd(OAc)₂ (7 g, 0.031 mol), X-Phos (14.9 g, 0.031 mol) and KOAc (61.4 g, 0.626 mol) were introduced to a flask, then dissolved by introducing 1,4-dioxane (2 L) thereto, and the result was stirred for 8 hours while heating. After the reaction was finished, distilled water was introduced thereto, and the organic layer was extracted with ethyl acetate. The obtained organic layer was dried with Na₂SO₄, vacuum distilled, and then purified using column chromatography to obtain compound 2-(7,7-dimethyl-7H-fluoreno[2,3-b]benzofuran-9-yl)-4,4,5,5-tetramethyl-1,3,2-dioxaborolane (91.1 g, yield 71%).

<Step 2> Synthesis of Cpd 10

2-(7,7-Dimethyl-7H-fluoreno[2,3-b]benzofuran-9-yl)-4,4,5,5-tetramethyl-1,3,2-dioxaborolane (8.2 g, 20 mmol) obtained in <Step 1>, 2-(4-chlorophenyl)-4,6-diphenyl-1,3,5-triazine (8.2 g, 24 mmol), Pd(OAc)₂ (0.45 g, 2 mmol), X-Phos (0.95 g 2 mmol) and Cs₂CO₃ (13.0 g, 40 mmol) were introduced to a flask, then dissolved by introducing 1,4-dioxane (2 L) thereto, and the result was stirred for 8 hours while heating. After the reaction was finished, distilled water was introduced thereto, and the organic layer was extracted with ethyl acetate. The obtained organic layer was dried with Na₂SO₄, vacuum distilled, and then purified using column chromatography to obtain Compound Cpd 10 (9.3 g, yield 79%). HRMS M⁺: 591.231

### SYNTHESIS EXAMPLE 2

### Synthesis of Cpd 15

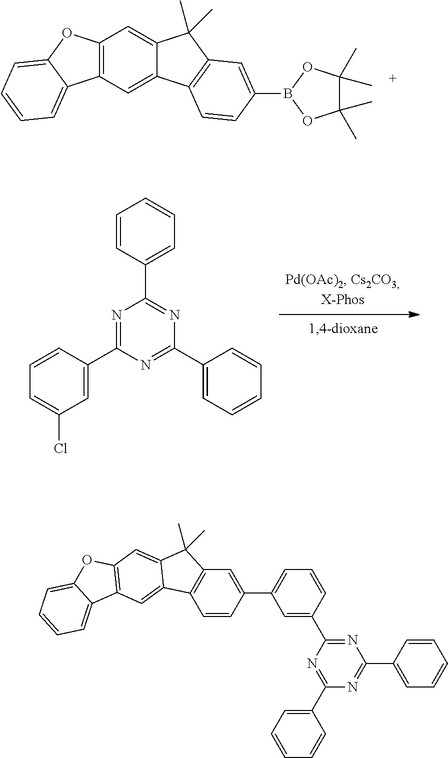 [SMILES: CC1(C)c2cc(B3OC(C)(C)C(C)(C)O3)ccc2-c2cc3c(cc21)oc1ccccc13 ; Clc1cccc(-c2nc(-c3ccccc3)nc(-c3ccccc3)n2)c1 ; CC1(C)c2cc(-c3cccc(-c4nc(-c5ccccc5)nc(-c5ccccc5)n4)c3)ccc2-c2cc3c(cc21)oc1ccccc13]

Compound Cpd 15 (8.7 g, yield 74%) was obtained in the same manner as in Synthesis Example 1 except that, in <Step 2>, 2-(3-chlorophenyl)-4,6-diphenyl-1,3,5-triazine was used instead of 2-(4-chlorophenyl)-4,6-diphenyl-1,3,5-triazine. HRMS[M]⁺: 591.231

### SYNTHESIS EXAMPLE 3

### Synthesis of Cpd 16

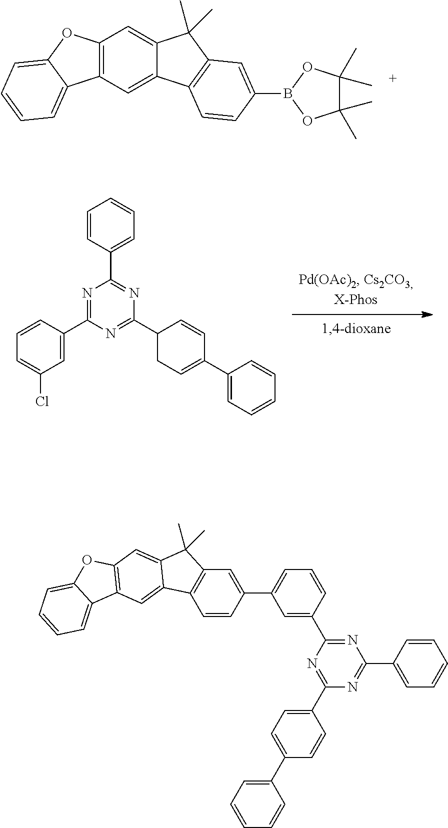 [SMILES: CC1(C)c2cc(B3OC(C)(C)C(C)(C)O3)ccc2-c2cc3c(cc21)oc1ccccc13 ; Clc1cccc(-c2nc(-c3ccccc3)nc(C3C=CC(c4ccccc4)=CC3)n2)c1 ; CC1(C)c2cc(-c3cccc(-c4nc(-c5ccccc5)nc(-c5ccc(-c6ccccc6)cc5)n4)c3)ccc2-c2cc3c(cc21)oc1ccccc13]

Compound Cpd 16 (9.5 g, yield 71%) was obtained in the same manner as in Synthesis Example 1 except that, in <Step 2>, 2-([1,1′-biphenyl]-4-yl)-4-(3-chlorophenyl)-6-phenyl-1,3,5-triazine was used instead of 2-(4-chlorophenyl)-4,6-diphenyl-1,3,5-triazine. HRMS [M]⁺: 667.262

### SYNTHESIS EXAMPLE 4

### Synthesis of Cpd 18

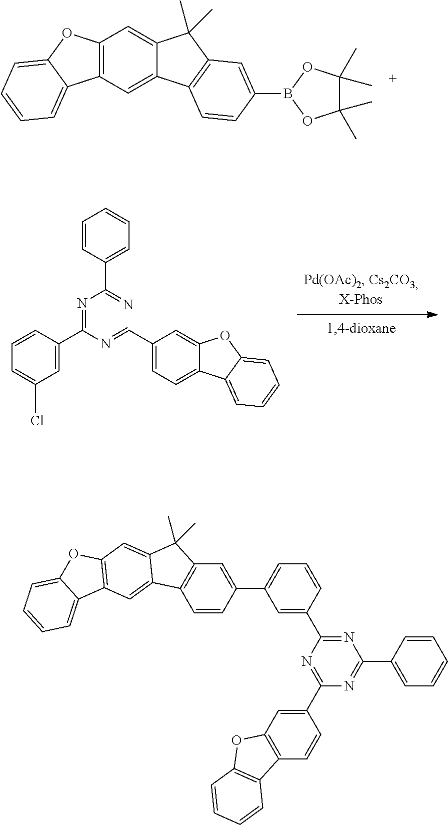 [SMILES: CC1(C)c2cc(B3OC(C)(C)C(C)(C)O3)ccc2-c2cc3c(cc21)oc1ccccc13 ; N=C(/N=C(\N=C\c1ccc2c(c1)oc1ccccc12)c1cccc(Cl)c1)c1ccccc1 ; CC1(C)c2cc(-c3cccc(-c4nc(-c5ccccc5)nc(-c5ccc6c(c5)oc5ccccc56)n4)c3)ccc2-c2cc3c(cc21)oc1ccccc13]

Compound Cpd 18 (10.3 g, yield 76%) was obtained in the same manner as in Synthesis Example 1 except that, in <Step 2>, 2-(3-chlorophenyl)-4-(dibenzo[b,d]furan-3-yl)-6-phenyl-1,3,5-triazine was used instead of 2-(4-chlorophenyl)-4,6-diphenyl-1,3,5-triazine. HRMS [M]⁺: 681.242

### SYNTHESIS EXAMPLE 5

### Synthesis of Cpd 28

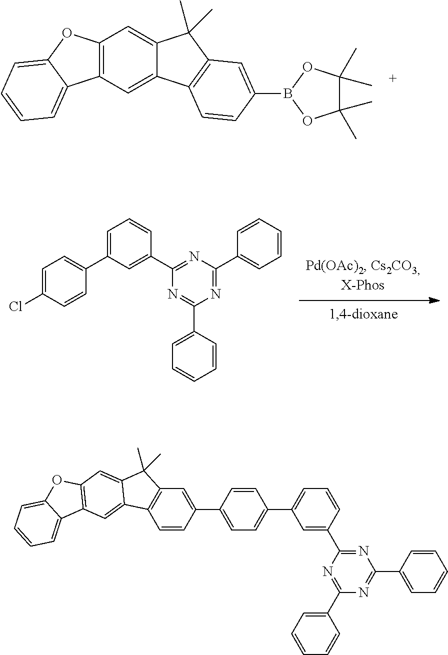 [SMILES: CC1(C)c2cc(B3OC(C)(C)C(C)(C)O3)ccc2-c2cc3c(cc21)oc1ccccc13 ; Clc1ccc(-c2cccc(-c3nc(-c4ccccc4)nc(-c4ccccc4)n3)c2)cc1 ; CC1(C)c2cc(-c3ccc(-c4cccc(-c5nc(-c6ccccc6)nc(-c6ccccc6)n5)c4)cc3)ccc2-c2cc3c(cc21)oc1ccccc13]

Compound Cpd 28 (9.7 g, yield 73%) was obtained in the same manner as in Synthesis Example 1 except that, in <Step 2>, 2-(4′-chloro-[1,1′-biphenyl]-3-yl)-4,6-diphenyl-1,3,5-triazine was used instead of 2-(4-chlorophenyl)-4,6-diphenyl-1,3,5-triazine. HRMS[M]⁺: 667.262

### SYNTHESIS EXAMPLE 6

### Synthesis of Cpd 29

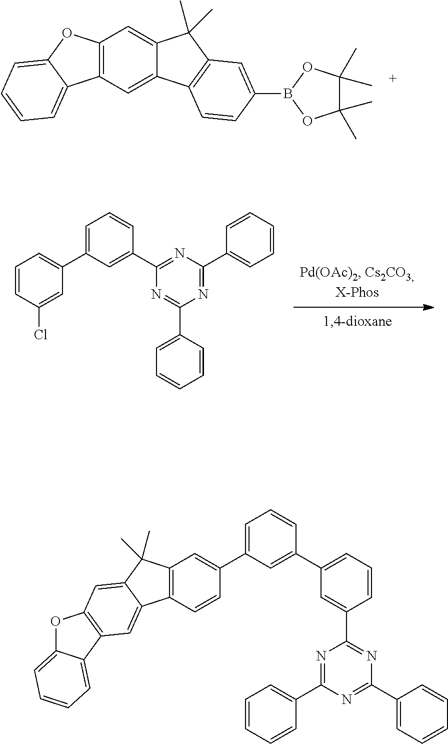 [SMILES: CC1(C)c2cc(B3OC(C)(C)C(C)(C)O3)ccc2-c2cc3c(cc21)oc1ccccc13 ; Clc1cccc(-c2cccc(-c3nc(-c4ccccc4)nc(-c4ccccc4)n3)c2)c1 ; CC1(C)c2cc(-c3cccc(-c4cccc(-c5nc(-c6ccccc6)nc(-c6ccccc6)n5)c4)c3)ccc2-c2cc3c(cc21)oc1ccccc13]

Compound Cpd 29 (10.1 g, yield 76%) was obtained in the same manner as in Synthesis Example 1 except that, in <Step 2>, 2-(3′-chloro-[1,1′-biphenyl]-3-yl)-4,6-diphenyl-1,3,5-triazine was used instead of 2-(4-chlorophenyl)-4,6-diphenyl-1,3,5-triazine. HRMS[M]⁺: 667.262

### SYNTHESIS EXAMPLE 7

### Synthesis of Cpd 30

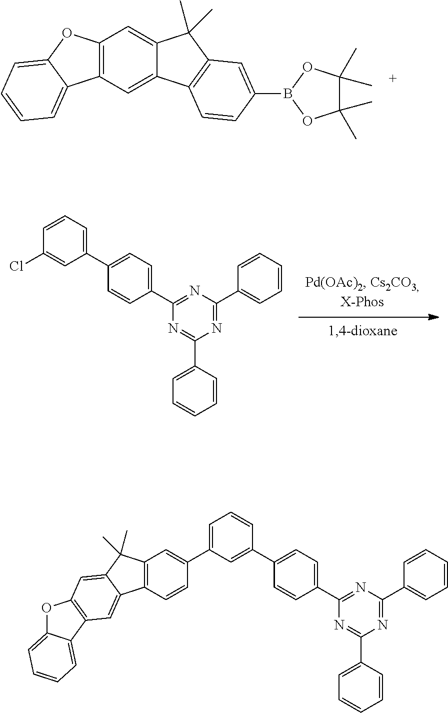 [SMILES: CC1(C)c2cc(B3OC(C)(C)C(C)(C)O3)ccc2-c2cc3c(cc21)oc1ccccc13 ; Clc1cccc(-c2ccc(-c3nc(-c4ccccc4)nc(-c4ccccc4)n3)cc2)c1 ; CC1(C)c2cc(-c3cccc(-c4ccc(-c5nc(-c6ccccc6)nc(-c6ccccc6)n5)cc4)c3)ccc2-c2cc3c(cc21)oc1ccccc13]

Compound Cpd 30 (9.5 g, yield 71%) was obtained in the same manner as in Synthesis Example 1 except that, in <Step 2>, 2-(3′-chloro-[1,1′-biphenyl]-4-yl)-4,6-diphenyl-1,3,5-triazine was used instead of 2-(4-chlorophenyl)-4,6-diphenyl-1,3,5-triazine. HRMS[M]⁺: 667.262

### SYNTHESIS EXAMPLE 8

### Synthesis of Cpd 40

Compound Cpd 40 (9.1 g, yield 77%) was obtained in the same manner as in

Synthesis Example 1 except that, in <Step 1>, Inv 3 was used instead of Inv 1. HRMS[M]⁺: 591.231

### SYNTHESIS EXAMPLE 9

### Synthesis of Cpd 45

Compound Cpd 45 (8.6 g, yield 73%) was obtained in the same manner as in Synthesis Example 2 except that, in <Step 1>, Inv 3 was used instead of Inv 1. HRMS[M]⁺: 591.231

### SYNTHESIS EXAMPLE 10

### Synthesis of Cpd 46

Compound Cpd 46 (9.5 g, yield 71%) was obtained in the same manner as in Synthesis Example 3 except that, in <Step 1>, Inv 3 was used instead of Inv 1. HRMS[M]⁺: 667.262

### SYNTHESIS EXAMPLE 11

### Synthesis of Cpd 48

Compound Cpd 48 (9.8 g, yield 72%) was obtained in the same manner as in Synthesis Example 4 except that, in <Step 1>, Inv 3 was used instead of Inv 1. HRMS[M]⁺: 681.242

### SYNTHESIS EXAMPLE 12

### Synthesis of Cpd 58

Compound Cpd 58 (10.0 g, yield 75%) was obtained in the same manner as in Synthesis Example 5 except that, in <Step 1>, Inv 3 was used instead of Inv 1. HRMS[M]⁺: 667.262

### SYNTHESIS EXAMPLE 13

### Synthesis of Cpd 59

Compound Cpd 59 (9.7 g, yield 73%) was obtained in the same manner as in Synthesis Example 6 except that, in <Step 1>, Inv 3 was used instead of Inv 1. HRMS[M]⁺: 667.262

### SYNTHESIS EXAMPLE 14

### Synthesis of Cpd 60

Compound Cpd 60 (10.1 g, yield 76%) was obtained in the same manner as in Synthesis Example 7 except that, in <Step 1>, Inv 3 was used instead of Inv 1. HRMS[M]⁺: 667.262

### SYNTHESIS EXAMPLE 15

### Synthesis of Cpd 130

Compound Cpd 130 (8.3 g, yield 70%) was obtained in the same manner as in Synthesis Example 1 except that, in <Step 1>, Inv 6 was used instead of Inv 1. HRMS[M]⁺: 591.231

### SYNTHESIS EXAMPLE 16

### Synthesis of Cpd 135

Compound Cpd 135 (9.0 g, yield 76%) was obtained in the same manner as in Synthesis Example 2 except that, in <Step 1>, Inv 6 was used instead of Inv 1. HRMS[M]⁺: 591.231

### SYNTHESIS EXAMPLE 17

### Synthesis of Cpd 136

Compound Cpd 136 (10.3 g, yield 77%) was obtained in the same manner as in Synthesis Example 3 except that, in <Step 1>, Inv 6 was used instead of Inv 1. HRMS[M]⁺: 667.262

### SYNTHESIS EXAMPLE 18

### Synthesis of Cpd 138

Compound Cpd 138 (10.2 g, yield 75%) was obtained in the same manner as in Synthesis Example 4 except that, in <Step 1>, Inv 6 was used instead of Inv 1. HRMS[M]⁺: 681.242

### SYNTHESIS EXAMPLE 19

### Synthesis of Cpd 148

Compound Cpd 148 (9.6 g, yield 72%) was obtained in the same manner as in Synthesis Example 5 except that, in <Step 1>, Inv 6 was used instead of Inv 1. HRMS[M]⁺: 667.262

### SYNTHESIS EXAMPLE 20

### Synthesis of Cpd 149

Compound Cpd 149 (9.7 g, yield 73%) was obtained in the same manner as in Synthesis Example 6 except that, in <Step 1>, Inv 6 was used instead of Inv 1. HRMS[M]⁺: 667.262

### SYNTHESIS EXAMPLE 21

### Synthesis of Cpd 150

Compound Cpd 150 (9.6 g, yield 72%) was obtained in the same manner as in Synthesis Example 7 except that, in <Step 1>, Inv 6 was used instead of Inv 1. HRMS[M]⁺: 667.262

### SYNTHESIS EXAMPLE 22

### Synthesis of Cpd 160

Compound Cpd 160 (9.1 g, yield 77%) was obtained in the same manner as in Synthesis Example 1 except that, in <Step 1>, Inv 8 was used instead of Inv 1. HRMS[M]⁺: 591.231

### SYNTHESIS EXAMPLE 23

### Synthesis of Cpd 165

Compound Cpd 165 (8.6 g, yield 73%) was obtained in the same manner as in Synthesis Example 2 except that, in <Step 1>, Inv 8 was used instead of Inv 1. HRMS[M]⁺: 591.231

### SYNTHESIS EXAMPLE 24

### Synthesis of Cpd 166

Compound Cpd 166 (9.5 g, yield 71%) was obtained in the same manner as in Synthesis Example 3 except that, in <Step 1>, Inv 8 was used instead of Inv 1. HRMS[M]⁺: 667.262

### SYNTHESIS EXAMPLE 25

### Synthesis of Cpd 168

Compound Cpd 168 (9.8 g, yield 72%) was obtained in the same manner as in Synthesis Example 4 except that, in <Step 1>, Inv 8 was used instead of Inv 1. HRMS[M]⁺: 681.242

### SYNTHESIS EXAMPLE 26

### Synthesis of Cpd 178

Compound Cpd 178 (10.0 g, yield 75%) was obtained in the same manner as in Synthesis Example 5 except that, in <Step 1>, Inv 8 was used instead of Inv 1. HRMS[M]⁺: 667.262

### SYNTHESIS EXAMPLE 27

### Synthesis of Cpd 179

Compound Cpd 179 (9.7 g, yield 73%) was obtained in the same manner as in Synthesis Example 6 except that, in <Step 1>, Inv 8 was used instead of Inv 1. HRMS[M]⁺: 667.262

### SYNTHESIS EXAMPLE 28

### Synthesis of Cpd 180

Compound Cpd 180 (10.1 g, yield 76%) was obtained in the same manner as in Synthesis Example 7 except that, in <Step 1>, Inv 8 was used instead of Inv 1. HRMS[M]⁺: 667.262

### SYNTHESIS EXAMPLE 29

### Synthesis of Cpd 190

Compound Cpd 190 (8.8 g, yield 74%) was obtained in the same manner as in Synthesis Example 1 except that, in <Step 1>, Inv 2 was used instead of Inv 1. HRMS[M]⁺: 591.231

### SYNTHESIS EXAMPLE 30

### Synthesis of Cpd 195

Compound Cpd 195 (9.2 g, yield 78%) was obtained in the same manner as in Synthesis Example 2 except that, in <Step 1>, Inv 2 was used instead of Inv 1. HRMS[M]⁺: 591.231

### SYNTHESIS EXAMPLE 31

### Synthesis of Cpd 196

Compound Cpd 196 (10.0 g, yield 75%) was obtained in the same manner as in Synthesis Example 3 except that, in <Step 1>, Inv 2 was used instead of Inv 1. HRMS[M]⁺: 667.262

### SYNTHESIS EXAMPLE 32

### Synthesis of Cpd 198

Compound Cpd 198 (10.4 g, yield 76%) was obtained in the same manner as in Synthesis Example 4 except that, in <Step 1>, Inv 2 was used instead of Inv 1. HRMS[M]⁺: 681.242

### SYNTHESIS EXAMPLE 33

### Synthesis of Cpd 208

Compound Cpd 208 (10.2 g, yield 76%) was obtained in the same manner as in Synthesis Example 5 except that, in <Step 1>, Inv 2 was used instead of Inv 1. HRMS[M]⁺: 667.262

### SYNTHESIS EXAMPLE 34

### Synthesis of Cpd 209

Compound Cpd 209 (9.5 g, yield 71%) was obtained in the same manner as in Synthesis Example 6 except that, in <Step 1>, Inv 2 was used instead of Inv 1. HRMS[M]⁺: 667.262

### SYNTHESIS EXAMPLE 35

### Synthesis of Cpd 210

Compound Cpd 210 (9.9 g, yield 74%) was obtained in the same manner as in Synthesis Example 7 except that, in <Step 1>, Inv 2 was used instead of Inv 1. HRMS[M]⁺: 667.262

### SYNTHESIS EXAMPLE 36

### Synthesis of Cpd 220

Compound Cpd 220 (9.2 g, yield 78%) was obtained in the same manner as in Synthesis Example 1 except that, in <Step 1>, Inv 4 was used instead of Inv 1. HRMS[M]⁺: 591.231

### SYNTHESIS EXAMPLE 37

### Synthesis of Cpd 225

Compound Cpd 225 (9.3 g, yield 79%) was obtained in the same manner as in Synthesis Example 2 except that, in <Step 1>, Inv 4 was used instead of Inv 1. HRMS[M]⁺: 591.231

### SYNTHESIS EXAMPLE 38

### Synthesis of Cpd 226

Compound Cpd 226 (9.8 g, yield 73%) was obtained in the same manner as in Synthesis Example 3 except that, in <Step 1>, Inv 4 was used instead of Inv 1. HRMS[M]⁺: 667.262

### SYNTHESIS EXAMPLE 39

### Synthesis of Cpd 228

Compound Cpd 228 (10.4 g, yield 76%) was obtained in the same manner as in Synthesis Example 4 except that, in <Step 1>, Inv 4 was used instead of Inv 1. HRMS[M]⁺: 681.242

### SYNTHESIS EXAMPLE 40

### Synthesis of Cpd 238

Compound Cpd 238 (10.3 g, yield 77%) was obtained in the same manner as in Synthesis Example 5 except that, in <Step 1>, Inv 4 was used instead of Inv 1. HRMS[M]⁺: 667.262

### SYNTHESIS EXAMPLE 41

### Synthesis of Cpd 239

Compound Cpd 239 (9.9 g, yield 74%) was obtained in the same manner as in Synthesis Example 6 except that, in <Step 1>, Inv 4 was used instead of Inv 1. HRMS[M]⁺: 667.262

### SYNTHESIS EXAMPLE 42

### Synthesis of Cpd 240

Compound Cpd 240 (10.4 g, yield 78%) was obtained in the same manner as in Synthesis Example 7 except that, in <Step 1>, Inv 4 was used instead of Inv 1. HRMS[M]⁺: 667.262

### SYNTHESIS EXAMPLE 43

### Synthesis of Cpd 310

Compound Cpd 310 (8.5 g, yield 72%) was obtained in the same manner as in Synthesis Example 1 except that, in <Step 1>, Inv 5 was used instead of Inv 1. HRMS[M]⁺: 591.231

### SYNTHESIS EXAMPLE 44

### Synthesis of Cpd 315

Compound Cpd 315 (8.9 g, yield 75%) was obtained in the same manner as in Synthesis Example 2 except that, in <Step 1>, Inv 5 was used instead of Inv 1. HRMS[M]⁺: 591.231

### SYNTHESIS EXAMPLE 45

### Synthesis of Cpd 316

Compound Cpd 316 (10.2 g, yield 76%) was obtained in the same manner as in Synthesis Example 3 except that, in <Step 1>, Inv 5 was used instead of Inv 1. HRMS[M]⁺: 667.262

### SYNTHESIS EXAMPLE 46

### Synthesis of Cpd 318

Compound Cpd 318 (10.4 g, yield 76%) was obtained in the same manner as in Synthesis Example 4 except that, in <Step 1>, Inv 5 was used instead of Inv 1. HRMS[M]⁺: 681.242

### SYNTHESIS EXAMPLE 47

### Synthesis of Cpd 328

Compound Cpd 328 (10.3 g, yield 77%) was obtained in the same manner as in Synthesis Example 5 except that, in <Step 1>, Inv 5 was used instead of Inv 1. HRMS[M]⁺: 667.262

### SYNTHESIS EXAMPLE 48

### Synthesis of Cpd 329

Compound Cpd 329 (9.9 g, yield 74%) was obtained in the same manner as in Synthesis Example 6 except that, in <Step 1>, Inv 5 was used instead of Inv 1. HRMS[M]⁺: 667.262

### SYNTHESIS EXAMPLE 49

### Synthesis of Cpd 330

Compound Cpd 330 (10.0 g, yield 75%) was obtained in the same manner as in Synthesis Example 7 except that, in <Step 1>, Inv 5 was used instead of Inv 1. HRMS[M]⁺: 667.262

### SYNTHESIS EXAMPLE 50

### Synthesis of Cpd 340

Compound Cpd 340 (9.3 g, yield 79%) was obtained in the same manner as in Synthesis Example 1 except that, in <Step 1>, Inv 7 was used instead of Inv 1. HRMS[M]⁺: 591.231

### SYNTHESIS EXAMPLE 51

### Synthesis of Cpd 345

Compound Cpd 345 (9.0 g, yield 76%) was obtained in the same manner as in Synthesis Example 2 except that, in <Step 1>, Inv 7 was used instead of Inv 1. HRMS[M]⁺: 591.231

### SYNTHESIS EXAMPLE 52

### Synthesis of Cpd 346

Compound Cpd 346 (10.4 g, yield 78%) was obtained in the same manner as in Synthesis Example 3 except that, in <Step 1>, Inv 7 was used instead of Inv 1. HRMS[M]⁺: 667.262

### SYNTHESIS EXAMPLE 53

### Synthesis of Cpd 348

Compound Cpd 348 (10.2 g, yield 75%) was obtained in the same manner as in Synthesis Example 4 except that, in <Step 1>, Inv 7 was used instead of Inv 1. HRMS[M]⁺: 681.242

### SYNTHESIS EXAMPLE 54

### Synthesis of Cpd 358

Compound Cpd 358 (9.9 g, yield 74%) was obtained in the same manner as in Synthesis Example 5 except that, in <Step 1>, Inv 7 was used instead of Inv 1. HRMS[M]⁺: 667.262

### SYNTHESIS EXAMPLE 55

### Synthesis of Cpd 359

Compound Cpd 359 (9.6 g, yield 72%) was obtained in the same manner as in Synthesis Example 6 except that, in <Step 1>, Inv 7 was used instead of Inv 1. HRMS[M]⁺: 667.262

### SYNTHESIS EXAMPLE 56

### Synthesis of Cpd 360

Compound Cpd 360 (10.0 g, yield 75%) was obtained in the same manner as in Synthesis Example 7 except that, in <Step 1>, Inv 7 was used instead of Inv 1. HRMS[M]⁺: 667.262

### SYNTHESIS EXAMPLE 57

### Synthesis of Cpd 363

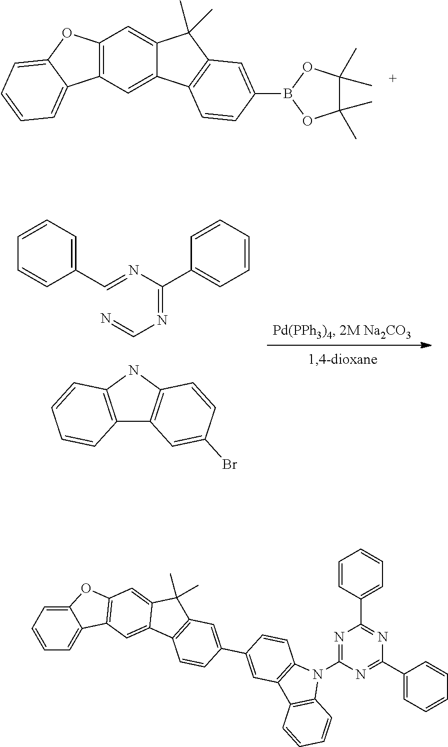

2-(7,7-Dimethyl-7H-fluoreno[2,3-b]benzofuran-9-yl)-4,4,5,5-tetramethyl-1,3,2-dioxaborolane (8.2 g 20 mmol) obtained in Synthesis Example 1, 3-bromo-9-(4,6-diphenyl-1,3,5-triazin-2-yl)-9H-carbazole (11.4 g, 24 mmol) and Pd(PPh₃)₄ (1.2 g, 1 mmol) were introduced to a flask and dissolved by introducing a saturated aqueous 2 M Na₂CO₃ solution (30 ml) and 1,4-dioxane (100 ml) thereto, and the result was stirred for 8 hours while heating. After the reaction was finished, distilled water was introduced thereto, and the organic layer was extracted with ethyl acetate. The obtained organic layer was dried with Na₂SO₄, vacuum distilled, and then purified using column chromatography to obtain Compound Cpd 363 (11.3, yield 83%). HRMS[M]⁺: 680.258

### SYNTHESIS EXAMPLE 58

### Synthesis of Cpd 367

Compound Cpd 367 (12.8 g, yield 85%) was obtained in the same manner as in Synthesis Example 57 except that 9-(4-([1,1′-biphenyl]-4-yl)-6-phenyl-1,3,5-triazin-2-yl)-3-bromo-9H-carbazole was used instead of 3-bromo-9-(4,6-diphenyl-1,3,5-triazin-2-yl)-9H-carbazole. HRMS[M]⁺: 756.289

### SYNTHESIS EXAMPLE 59

### Synthesis of Cpd 370

Compound Cpd 370 (12.3 g, yield 80%) was obtained in the same manner as in Synthesis Example 57 except that 3-bromo-9-(4-(dibenzo[b,d]furan-3-yl)-6-phenyl-1,3,5-triazin-2-yl)-9H-carbazole was used instead of 3-bromo-9-(4,6-diphenyl-1,3,5-triazin-2-yl)-9H-carbazole. HRMS[M]⁺: 770.268

### SYNTHESIS EXAMPLE 60

### Synthesis of Cpd 371

Compound Cpd 371 (12.6 g, yield 82%) was obtained in the same manner as in Synthesis Example 57 except that 3-bromo-9-(4-(dibenzo[b,d]furan-2-yl)-6-phenyl-1,3,5-triazin-2-yl)-9H-carbazole was used instead of 3-bromo-9-(4,6-diphenyl-1,3,5-triazin-2-yl)-9H-carbazole. HRMS[M]⁺: 770.268

### SYNTHESIS EXAMPLE 61

### Synthesis of Cpd 409

Compound Cpd 409 (9.4 g, yield 72%) was obtained in the same manner as in Synthesis Example 57 except that 3-bromo-9-(4-phenylquinazolin-2-yl)-9H-carbazole was used instead of 3-bromo-9-(4,6-diphenyl-1,3,5-triazin-2-yl)-9H-carbazole. HRMS[M]⁺: 653.247

### SYNTHESIS EXAMPLE 62

### Synthesis of Cpd 411

Compound Cpd 411 (10.8 g, yield 74%) was obtained in the same manner as in Synthesis Example 57 except that 3-bromo-9-(4-(4-phenylquinazolin-2-yl)phenyl)-9H-carbazole was used instead of 3-bromo-9-(4,6-diphenyl-1,3,5-triazin-2-yl)-9H-carbazole. HRMS[M]⁺: 729.278

### SYNTHESIS EXAMPLE 63

### Synthesis of Cpd 412

Compound Cpd 412 (10.3 g, yield 71%) was obtained in the same manner as in Synthesis Example 57 except that 3-bromo-9-(3-(4-phenylquinazolin-2-yl)phenyl)-9H-carbazole was used instead of 3-bromo-9-(4,6-diphenyl-1,3,5-triazin-2-yl)-9H-carbazole. HRMS[M]⁺: 729.278

### SYNTHESIS EXAMPLE 64

### Synthesis of Cpd 413

Compound Cpd 413 (9.8 g, yield 75%) was obtained in the same manner as in Synthesis Example 61 except that 2-(7,7-dimethyl-7H-fluoreno[2,3-b]benzofuran-10-yl)-4,4,5,5-tetramethyl-1,3,2-dioxaborolane was used instead of 2-(7,7-dimethyl-7H-fluoreno[2,3-b]benzofuran-9-yl)-4,4,5,5-tetramethyl-1,3,2-dioxaborolane. HRMS[M]⁺: 653.247

### SYNTHESIS EXAMPLE 65

### Synthesis of Cpd 415

Compound Cpd 415 (10.3 g, yield 71%) was obtained in the same manner as in Synthesis Example 62 except that 2-(7,7-dimethyl-7H-fluoreno[2,3-b]benzofuran-10-yl)-4,4,5,5-tetramethyl-1,3,2-dioxaborolane was used instead of 2-(7,7-dimethyl-7H-fluoreno[2,3-b]benzofuran-9-yl)-4,4,5,5-tetramethyl-1,3,2-dioxaborolane. HRMS[M]⁺: 729.278

### SYNTHESIS EXAMPLE 66

### Synthesis of Cpd 416

Compound Cpd 416 (10.5 g, yield 72%) was obtained in the same manner as in Synthesis Example 63 except that 2-(7,7-dimethyl-7H-fluoreno[2,3-b]benzofuran-10-yl)-4,4,5,5-tetramethyl-1,3,2-dioxaborolane was used instead of 2-(7,7-dimethyl-7H-fluoreno[2,3-b]benzofuran-9-yl)-4,4,5,5-tetramethyl-1,3,2-dioxaborolane. HRMS[M]⁺: 729.278

### SYNTHESIS EXAMPLE 67

### Synthesis of Cpd 417

Compound Cpd 417 (10.0 g, yield 87%) was obtained in the same manner as in Synthesis Example 57 except that N-(4-bromophenyl)-N-phenylnaphthalen-1-amine was used instead of 3-bromo-9-(4,6-diphenyl-1,3,5-triazin-2-yl)-9H-carbazole. HRMS[M]⁺: 577.241

### SYNTHESIS EXAMPLE 68

### Synthesis of Cpd 418

Compound Cpd 418 (12.1 g, yield 89%) was obtained in the same manner as in Synthesis Example 57 except that N-([1,1′-biphenyl]-4-yl)-N-(4-bromophenyl)[1,1′-biphenyl]-4-amine was used instead of 3-bromo-9-(4,6-diphenyl-1,3,5-triazin-2-yl)-9H-carbazole. HRMS[M]⁺: 679.288

### SYNTHESIS EXAMPLE 69

### Synthesis of Cpd 420

Compound Cpd 420 (12.5 g, yield 87%) was obtained in the same manner as in Synthesis Example 57 except that N-([1,1′-biphenyl]-4-yl)-N-(4-bromophenyl)-9,9-dimethyl-9H-fluoren-2-amine was used instead of 3-bromo-9-(4,6-diphenyl-1,3,5-triazin-2-yl)-9H-carbazole. HRMS[M]⁺: 719.319

### SYNTHESIS EXAMPLE 70

### Synthesis of Cpd 419

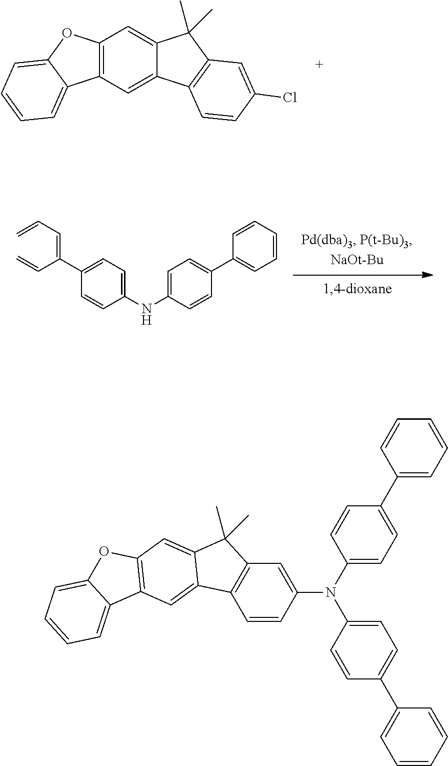 [SMILES: CC1(C)c2cc(Cl)ccc2-c2cc3c(cc21)oc1ccccc13 ; C=C/C=C(\C=C)c1ccc(Nc2ccc(-c3ccccc3)cc2)cc1 ; CC1(C)c2cc(N(c3ccc(-c4ccccc4)cc3)c3ccc(-c4ccccc4)cc3)ccc2-c2cc3c(cc21)oc1ccccc13]

After dissolving Compound Inv 1 (6.4 g, 20.0 mmol) synthesized in Preparation Example 1 and di([1,1′-biphenyl]-4-yl)amine (7.7 g, 24.0 mmol) in toluene (100 ml), Pd₂(dba)₃ (0.9 g, 1.0 mmol) was introduced thereto under nitrogen. After that, NaOtBu (38.4 g, 40 mmol) was introduced thereto, and after introducing (t-Bu)₃P (1.0 ml, 1.0 mmol) to the reaction solution, the mixture was stirred for 5 hours under reflux.

After identifying termination of the reaction by TLC, the result was cooled to room temperature. After the reaction was finished, distilled water was introduced thereto, and the organic layer was extracted with ethyl acetate. The obtained organic layer was dried with Na₂SO₄, vacuum distilled, and then purified using column chromatography to obtain Compound Cpd 419 (10.7 g, yield 89%). HRMS[M]⁺: 603.256

### SYNTHESIS EXAMPLE 71

### Synthesis of Cpd 429

Compound Cpd 429 (10.1 g, yield 88%) was obtained in the same manner as in Synthesis Example 67 except that 2-(7,7-dimethyl-7H-fluoreno[2,3-b]benzofuran-10-yl)-4,4,5,5-tetramethyl-1,3,2-dioxaborolane was used instead of 2-(7,7-dimethyl-7H-fluoreno[2,3-b]benzofuran-9-yl)-4,4,5,5-tetramethyl-1,3,2-dioxaborolane. HRMS[M]⁺: 577.241

### SYNTHESIS EXAMPLE 72

### Synthesis of Cpd 430

Compound Cpd 430 (11.4 g, yield 84%) was obtained in the same manner as in Synthesis Example 68 except that 2-(7,7-dimethyl-7H-fluoreno[2,3-b]benzofuran-10-yl)-4,4,5,5-tetramethyl-1,3,2-dioxaborolane was used instead of 2-(7,7-dimethyl-7H-fluoreno[2,3-b]benzofuran-9-yl)-4,4,5,5-tetramethyl-1,3,2-dioxaborolane. HRMS[M]⁺: 679.288

### SYNTHESIS EXAMPLE 73

### Synthesis of Cpd 432

Compound Cpd 432 (11.8 g, yield 82%) was obtained in the same manner as in Synthesis Example 69 except that 2-(7,7-dimethyl-7H-fluoreno[2,3-b]benzofuran-10-yl)-4,4,5,5-tetramethyl-1,3,2-dioxaborolane was used instead of 2-(7,7-dimethyl-7H-fluoreno[2,3-b]benzofuran-9-yl)-4,4,5,5-tetramethyl-1,3,2-dioxaborolane. HRMS[M]⁺: 719.319

### SYNTHESIS EXAMPLE 74

### Synthesis of Cpd 431

Compound Cpd 431 (10.4 g, yield 86%) was obtained in the same manner as in Synthesis Example 70 except that Inv 3 was used instead of Inv 1. HRMS[M]⁺: 603.256

### EXAMPLE 1

### Manufacture of Green Organic Electroluminescent Device

After high purity sublimation purifying Compound Cpd 417 synthesized in Synthesis Example 67 using a commonly known method, a green organic electroluminescent device was manufactured as follows.

A glass substrate on which indium tin oxide (ITO) was coated as a thin film to a thickness of 1500 Å was ultrasonic cleaned using distilled water. After the cleaning with distilled water was finished, the substrate was ultrasonic cleaned with solvents of isopropyl alcohol, acetone, methanol and the like, dried, then transferred to a UV OZONE washer (Power sonic 405, manufactured by Hwashin Tech. Co., Ltd.), and then, after cleaning the substrate for 5 minutes using UV, the substrate was transferred to a vacuum deposition apparatus.

On the transparent ITO electrode prepared as above, m-MTDATA (60 nm)/TCTA (80 nm)/Compound Cpd 417 (40 nm)/CBP+10% Ir(ppy)₃ (30 nm)/BCP (10 nm)/Alq₃(30 nm)/LiF (1 nm)/Al (200 nm) were laminated in this order to manufacture an organic electroluminescent device.

Structures of the m-MTDATA, the TCTA, the Ir(ppy)₃, the CBP and the BCP used herein are as follows.

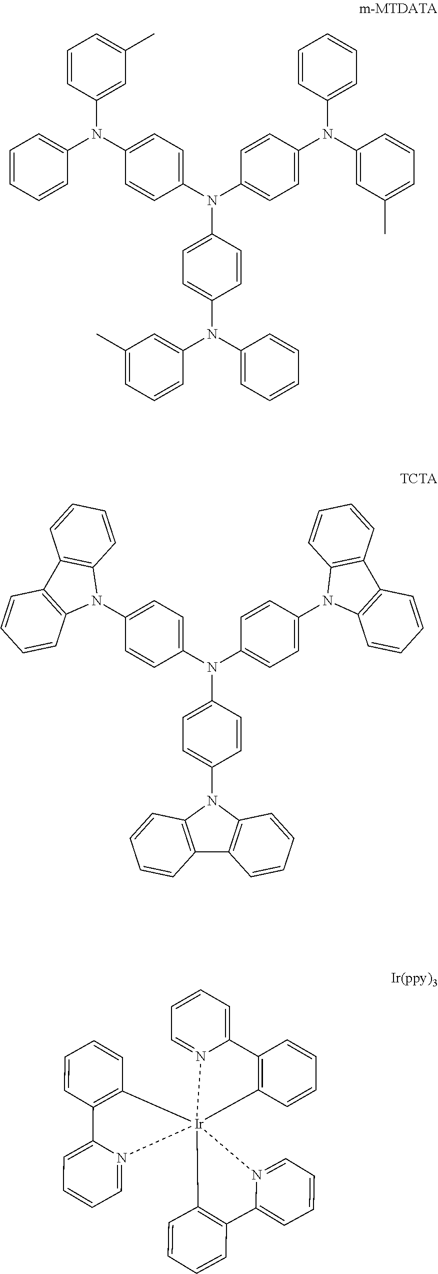

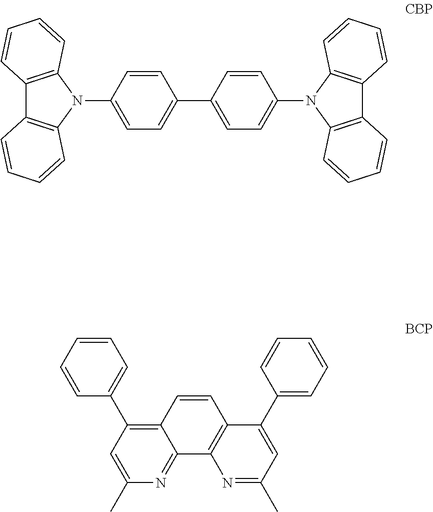 [SMILES: c1ccc2c(c1)c1ccccc1n2-c1ccc(-c2ccc(-n3c4ccccc4c4ccccc43)cc2)cc1 ; Cc1cc(-c2ccccc2)c2ccc3c(-c4ccccc4)cc(C)nc3c2n1]

### EXAMPLES 2 to 8

### Manufacture of Green Organic Electroluminescent Devices

Organic EL devices were manufactured in the same manner as in Example 1 except that compounds described in the following Table 1 were each used instead of Compound Cpd 417 used in Example 1.

### COMPARATIVE EXAMPLE 1

### Manufacture of Green Organic Electroluminescent Device

A green organic electroluminescent device was manufactured in the same manner as in Example 1 except that Compound Cpd 417 used in Example 1 was not used.

### EVALUATION EXAMPLE 1

For the green organic electroluminescent devices each manufactured in Examples 1 to 8 and Comparative Example 1, driving voltage, current efficiency and light emission peak at current density of 10 mA/cm² were measured, and the results are shown in the following Table 1.

TABLE 1

| | Light Emitting | Driving | Light | Current |
|---|---|---|---|---|
| | Auxiliaty Layer | Voltage | Emission | Efficiency |
| | Material | (V) | Peak (nm) | (cd/A) |
| Example 1 | Cpd 417 | 6.80 | 520 | 42.0 |
| Example 2 | Cpd 418 | 6.75 | 519 | 41.9 |
| Example 3 | Cpd 419 | 6.70 | 517 | 41.5 |
| Example 4 | Cpd 420 | 6.73 | 518 | 41.8 |
| Example 5 | Cpd 429 | 6.80 | 520 | 41.5 |
| Example 6 | Cpd 430 | 6.71 | 519 | 41.5 |
| Example 7 | Cpd 431 | 6.85 | 517 | 41.8 |
| Example 8 | Cpd 432 | 6.80 | 520 | 41.8 |
| Comparative | — | 6.93 | 516 | 38.2 |
| Example 1 | | | | |

As shown in Table 1, it was seen that the green organic electroluminescent devices of Examples 1 to 8 using the compound according to the present invention as a light emitting auxiliary layer material had a slightly lower driving voltage compared to the green organic electroluminescent device of Comparative Example 1 using just CBP as a light emitting layer material without a light emitting auxiliary layer, and more superior current efficiency was obtained compared to the green organic electroluminescent device of Comparative Example 1.

### EXAMPLE 9

### Manufacture of Red Organic Electroluminescent Device

After high purity sublimation purifying Compound Cpd 417 synthesized in Synthesis Example 67 using a commonly known method, a red organic electroluminescent device was manufactured as follows.

First, a glass substrate on which indium tin oxide (ITO) was coated as a thin film to a thickness of 1500 Å was ultrasonic cleaned using distilled water. After the cleaning with distilled water was finished, the substrate was ultrasonic cleaned with solvents of isopropyl alcohol, acetone, methanol and the like, dried, then transferred to a UV OZONE washer (Power sonic 405, manufactured by Hwashin Tech. Co., Ltd.), and then, after cleaning the substrate for 5 minutes using UV, the substrate was transferred to a vacuum deposition apparatus.

On the transparent ITO electrode prepared as above, m-MTDATA (60 nm)/TCTA (80 nm)/Compound Cpd 417 (40 nm)/CBP+10% (piq)₂Ir(acac) (30 nm)/BCP (10 nm)/Alq₃ (30 nm)/LiF (1 nm)/Al (200 nm) were laminated in this order to manufacture an organic electroluminescent device.

Structures of the m-MTDATA, the TCTA, the CBP and the BCP used herein are the same as described in Example 1, and a structure of the (piq)₂Ir(acac) is as follows.

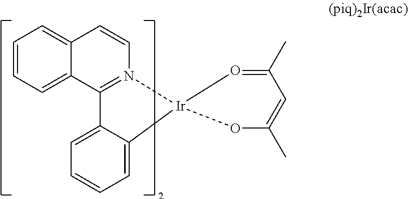

### EXAMPLES 10 to 16

### Manufacture of Red Organic Electroluminescent Devices

Red organic EL devices were manufactured in the same manner as in Example 9 except that compounds described in the following Table 2 were each used instead of Compound Cpd 417 used in Example 9.

### COMPARATIVE EXAMPLE 2

### Manufacture of Red Organic Electroluminescent Device

A red organic electroluminescent device was manufactured in the same manner as in Example 9 except that Compound Cpd 417 used in Example 9 was not used.

### EVALUATION EXAMPLE 2

For each of the red organic electroluminescent devices manufactured in Examples 9 to 16 and Comparative Example 2, driving voltage and current efficiency at current density of 10 mA/cm² were measured, and the results are shown in the following Table 2.

TABLE 2

| | Light Emitting | Driving | Current |
|---|---|---|---|
| | Auxiliaty | Voltage | Efficiency |
| | Layer Material | (V) | (cd/A) |
| Example 9 | Cpd 417 | 5.15 | 11.2 |
| Example 10 | Cpd 418 | 5.10 | 11.0 |
| Example 11 | Cpd 419 | 5.15 | 11.3 |
| Example 12 | Cpd 420 | 5.10 | 11.0 |
| Example 13 | Cpd 429 | 5.14 | 11.3 |
| Example 14 | Cpd 430 | 5.15 | 10.8 |
| Example 15 | Cpd 431 | 5.10 | 11.3 |
| Example 16 | Cpd 432 | 5.15 | 11.0 |
| Comparative | — | 5.25 | 8.2 |
| Example 2 | | | |

As shown in Table 2, it was seen that the red organic electroluminescent devices of Examples 9 to 16 using the compound according to the present invention as alight emitting auxiliary layer material had more superior current efficiency as well as a slightly lower driving voltage compared to the red organic electroluminescent device of Comparative Example 2 using just CBP as a light emitting layer material without a light emitting auxiliary layer.

### EXAMPLE 17

### Manufacture of Green Organic EL Device

After high purity sublimation purifying Compound Cpd 10 synthesized in Synthesis Example 1 using a commonly known method, a green organic electroluminescent device was manufactured using the following procedure.

First, a glass substrate on which indium tin oxide (ITO) was coated as a thin film to a thickness of 1500 Å was ultrasonic cleaned using distilled water. After the cleaning with distilled water was finished, the substrate was ultrasonic cleaned with solvents of isopropyl alcohol, acetone, methanol and the like, dried, then transferred to a UV OZONE washer (Power sonic 405, manufactured by Hwashin Tech. Co., Ltd.), and then, after cleaning the substrate for 5 minutes using UV, the substrate was transferred to a vacuum deposition apparatus.

On the transparent ITO electrode prepared as above, m-MTDATA (60 nm)/TCTA (80 nm)/Compound Cpd 10+10% Ir(ppy)₃ (30nm)/BCP (10 nm)/Alq₃ (30 nm)/LiF (1 nm)/Al (200 nm) were laminated in this order to manufacture an organic electroluminescent device.

### EXAMPLES 18 to 75

### Manufacture of Green Organic Electroluminescent Devices

Green organic EL devices were manufactured in the same manner as in Example 17 except that compounds described in the following Table 3 were each used instead of Compound Cpd 10 used in Example 17.

### COMPARATIVE EXAMPLE 3

### Manufacture of Green Organic EL Device

A green organic EL device was manufactured in the same manner as in Example 17 except that CBP was used instead of Compound Cpd 10 used as a light emitting host material when forming the light emitting layer in Example 17.

### EVALUATION EXAMPLE 3

For each of the green organic EL devices manufactured in Examples 17 to 75 and Comparative Example 3, driving voltage, current efficiency and light emission peak at current density of 10 mA/cm² were measured, and the results are shown in the following Table 3.

TABLE 3

| | | Driving | EL | Current |
|---|---|---|---|---|
| | | Voltage | Peak | Efficiency |
| Sample | Host | (V) | (nm) | (cd/A) |
| Example 17 | Cpd 10 | 6.63 | 517 | 40.8 |
| Example 18 | Cpd 15 | 6.78 | 518 | 41.1 |
| Example 19 | Cpd 16 | 6.81 | 517 | 40.8 |
| Example 20 | Cpd 18 | 6.79 | 517 | 40.8 |
| Example 21 | Cpd 28 | 6.81 | 518 | 41.1 |
| Example 22 | Cpd 29 | 6.79 | 515 | 42.4 |
| Example 23 | Cpd 30 | 6.78 | 518 | 41.1 |
| Example 24 | Cpd 40 | 6.81 | 517 | 40.8 |
| Example 25 | Cpd 45 | 6.79 | 518 | 41.1 |
| Example 26 | Cpd 46 | 6.81 | 518 | 41.1 |
| Example 27 | Cpd 48 | 6.79 | 517 | 40.8 |
| Example 28 | Cpd 58 | 6.79 | 517 | 40.8 |
| Example 29 | Cpd 59 | 6.81 | 518 | 41.1 |
| Example 30 | Cpd 60 | 6.79 | 517 | 40.8 |
| Example 31 | Cpd 130 | 6.81 | 518 | 41.1 |
| Example 32 | Cpd 135 | 6.78 | 515 | 42.4 |
| Example 33 | Cpd 136 | 6.79 | 517 | 40.8 |
| Example 34 | Cpd 138 | 6.79 | 517 | 40.8 |
| Example 35 | Cpd 148 | 6.81 | 518 | 41.1 |
| Example 36 | Cpd 149 | 6.79 | 517 | 40.8 |
| Example 37 | Cpd 150 | 6.79 | 517 | 40.8 |
| Example 38 | Cpd 160 | 6.81 | 518 | 41.1 |
| Example 39 | Cpd 165 | 6.79 | 517 | 40.8 |
| Example 40 | Cpd 166 | 6.79 | 517 | 40.8 |
| Example 41 | Cpd 168 | 6.81 | 518 | 41.1 |
| Example 42 | Cpd 178 | 6.79 | 517 | 40.8 |
| Example 43 | Cpd 179 | 6.81 | 518 | 41.1 |
| Example 44 | Cpd 180 | 6.78 | 515 | 42.4 |
| Example 45 | Cpd 190 | 6.81 | 518 | 41.1 |
| Example 46 | Cpd 195 | 6.79 | 517 | 40.8 |
| Example 47 | Cpd 196 | 6.79 | 517 | 40.8 |
| Example 48 | Cpd 198 | 6.81 | 518 | 41.1 |
| Example 49 | Cpd 208 | 6.79 | 517 | 40.8 |
| Example 50 | Cpd 209 | 6.79 | 517 | 40.8 |
| Example 51 | Cpd 210 | 6.81 | 518 | 41.1 |
| Example 52 | Cpd 220 | 6.78 | 515 | 42.4 |
| Example 53 | Cpd 225 | 6.81 | 518 | 41.1 |
| Example 54 | Cpd 226 | 6.79 | 517 | 40.8 |
| Example 55 | Cpd 228 | 6.81 | 518 | 41.1 |
| Example 56 | Cpd 238 | 6.79 | 517 | 40.8 |
| Example 57 | Cpd 239 | 6.79 | 517 | 40.8 |
| Example 58 | Cpd 240 | 6.81 | 518 | 41.1 |
| Example 59 | Cpd 310 | 6.78 | 515 | 42.4 |
| Example 60 | Cpd 315 | 6.81 | 518 | 41.1 |
| Example 61 | Cpd 316 | 6.79 | 517 | 40.8 |
| Example 62 | Cpd 318 | 6.81 | 518 | 41.1 |
| Example 63 | Cpd 328 | 6.81 | 518 | 41.1 |
| Example 64 | Cpd 329 | 6.79 | 517 | 40.8 |
| Example 65 | Cpd 330 | 6.79 | 517 | 40.8 |
| Example 66 | Cpd 340 | 6.81 | 518 | 41.1 |
| Example 67 | Cpd 345 | 6.79 | 517 | 40.8 |
| Example 68 | Cpd 346 | 6.81 | 518 | 41.1 |
| Example 69 | Cpd 348 | 6.78 | 515 | 42.4 |
| Example 70 | Cpd 358 | 6.81 | 518 | 41.1 |
| Example 71 | Cpd 359 | 6.79 | 517 | 40.8 |
| Example 72 | Cpd 360 | 6.81 | 518 | 41.1 |
| Example 73 | Cpd 363 | 6.79 | 517 | 40.8 |
| Example 74 | Cpd 367 | 6.79 | 517 | 40.8 |
| Example 75 | Cpd 370 | 6.81 | 518 | 41.1 |
| Comparative | CBP | 6.93 | 516 | 38.2 |
| Example 4 | | | | |

As shown in Table 3, it was seen that the green organic electroluminescent devices of Examples 17 to 75 each using the compound according to the present invention as a light emitting layer material had more superior performance in terms of current efficiency and driving voltage compared to the green organic EL device of Comparative Example 3 using existing CBP.

### EXAMPLE 76

### Manufacture of Red Organic EL Device

After high purity sublimation purifying Compound Cpd 409 synthesized in Synthesis Example 61 using a commonly known method, a red organic electroluminescent device was manufactured using the following procedure.

First, a glass substrate on which indium tin oxide (ITO) was coated as a thin film to a thickness of 1500 Å was ultrasonic cleaned using distilled water. After the cleaning with distilled water was finished, the substrate was ultrasonic cleaned with solvents of isopropyl alcohol, acetone, methanol and the like, dried, then transferred to a UV OZONE washer (Power sonic 405, manufactured by Hwashin Tech. Co., Ltd.), and then, after cleaning the substrate for 5 minutes using UV, the substrate was transferred to a vacuum deposition apparatus.

On the transparent ITO electrode prepared as above, m-MTDATA (60 nm)/TCTA (80 nm)/Compound Cpd 409+10% (piq)₂Ir(acac) (30 nm)/BCP (10 nm)/Alq₃ (30 nm)/LiF (1 nm)/Al (200 nm) were laminated in this order to manufacture an organic electroluminescent device.

### COMPARATIVE EXAMPLE 4

A red organic electroluminescent device was manufactured in the same manner as in Example 76 except that CBP was used instead of Compound Cpd 409 used as a light emitting host material when forming the light emitting layer.

### EXAMPLES 77 to 81

### Manufacture of Red Organic Electroluminescent Devices

Red organic EL devices were manufactured in the same manner as in Example 76 except that compounds described in the following Table 4 were each used instead of Compound Cpd 409 used in Example 76.

### EVALUATION EXAMPLE 4

For each of the organic electroluminescent devices manufactured in Examples 76 to 81 and Comparative Example 4, driving voltage and current efficiency at current density of 10 mA/cm² were measured, and the results are shown in the following Table 4.

TABLE 4

| | | Driving | Current |
|---|---|---|---|
| | | Voltage | Efficiency |
| Sample | Host | (V) | (cd/A) |
| Example 76 | Cpd 409 | 4.77 | 11.5 |
| Example 77 | Cpd 411 | 4.72 | 10.2 |
| Example 78 | Cpd 412 | 4.80 | 11.0 |
| Example 79 | Cpd 413 | 4.59 | 12.8 |
| Example 80 | Cpd 415 | 4.80 | 10.4 |
| Example 81 | Cpd 416 | 4.54 | 12.1 |
| Comparative | CBP | 5.25 | 8.2 |
| Example 4 | | | |

As shown in Table 4, it was seen that, when using the compound according to the present invention as a light emitting layer material of the red organic electroluminescent device (Examples 76 to 81), more superior performance was obtained in terms of efficiency and driving voltage compared to the red organic electroluminescent device (Comparative Example 4) using existing CBP as a light emitting layer material.

### EXAMPLE 82

### Manufacture of Blue Organic EL Device

After high purity sublimation purifying Compound Cpd 10 synthesized in Synthesis Example 1 using a commonly known method, a blue organic electroluminescent device was manufactured as follows.

A glass substrate on which indium tin oxide (ITO) was coated as a thin film to a thickness of 1500 Å was ultrasonic cleaned using distilled water. After the cleaning with distilled water was finished, the substrate was ultrasonic cleaned with solvents of isopropyl alcohol, acetone, methanol and the like, dried, then transferred to a UV OZONE washer (Power sonic 405, manufactured by Hwashin Tech. Co., Ltd.), and then, after cleaning the substrate for 5 minutes using UV, the substrate was transferred to a vacuum deposition apparatus.

On the transparent ITO electrode prepared as above, DS-205 (80 nm)/NPB (15 nm)/AND+5% DS-405 (30 nm)/Compound Cpd10 (5 nm)/Alq₃ (25 nm)/LiF (1 nm)/Al (200 nm) were laminated in this order to manufacture an organic electroluminescent device.

### EXAMPLES 83 to 137

### Manufacture of Blue Organic EL Devices

Blue organic EL devices were manufactured in the same manner as in Example 82 except that each compound described in the following Table 5 was used instead of Compound Cpd 10 used as a hole blocking layer material in Example 82.

### COMPARATIVE EXAMPLE 5

### Manufacture of Blue Organic Electroluminescent Device

A blue organic electroluminescent device was manufactured in the same manner as in Example 82 except that Compound Cpd 10 used as a hole blocking layer material in Example 82 was not used, and Alq3, the electron transport layer material, was deposited to 30 nm instead of 25 nm.

### COMPARATIVE EXAMPLE 6

### Manufacture of Blue Organic Electroluminescent Device

An organic electroluminescent device was manufactured in the same manner as in Example 83 except that BCP was used instead of not using Compound Cpd 10 used as a hole blocking layer material.

### EVALUATION EXAMPLE 5

For the organic electroluminescent devices each manufactured in Examples 82 to 137 and Comparative Examples 5 and 6, driving voltage, current efficiency, light emission peak and lifetime (T97) at current density of 10 mA/cm² were measured, and the results are shown in the following Table 5.

TABLE 5

| | Hole | Driving | Current | Light | |
|---|---|---|---|---|---|
| | Blocking | Voltage | Efficiency | Emission | Lifetime |
| Sample | Layer | (V) | (cd/A) | Peak (nm) | (hr, T₉₇) |
| Example 82 | Cpd 10 | 4.1 | 6.0 | 458 | 75 |
| Example 83 | Cpd 15 | 4.7 | 6.3 | 458 | 62 |
| Example 84 | Cpd 16 | 4.7 | 6.4 | 457 | 60 |
| Example 85 | Cpd 18 | 4.5 | 6.2 | 458 | 64 |
| Example 86 | Cpd 28 | 4.4 | 6.0 | 458 | 50 |
| Example 87 | Cpd 29 | 4.1 | 6.4 | 457 | 85 |
| Example 88 | Cpd 30 | 4.7 | 6.1 | 458 | 55 |
| Example 89 | Cpd 40 | 4.7 | 6.0 | 458 | 75 |
| Example 90 | Cpd 45 | 4.5 | 6.3 | 458 | 62 |
| Example 91 | Cpd 46 | 4.4 | 6.6 | 458 | 55 |
| Example 92 | Cpd 48 | 4.1 | 6.2 | 458 | 75 |
| Example 93 | Cpd 58 | 4.2 | 6.4 | 457 | 60 |
| Example 94 | Cpd 59 | 4.5 | 6.2 | 458 | 64 |
| Example 95 | Cpd 60 | 4.3 | 6.0 | 458 | 50 |
| Example 96 | Cpd 130 | 4.5 | 6.2 | 458 | 75 |
| Example 97 | Cpd 135 | 4.3 | 6.4 | 457 | 60 |
| Example 98 | Cpd 136 | 4.4 | 6.2 | 458 | 64 |
| Example 99 | Cpd 138 | 4.1 | 6.0 | 458 | 50 |
| Example 100 | Cpd 148 | 4.2 | 6.4 | 457 | 85 |
| Example 101 | Cpd 149 | 4.4 | 6.1 | 458 | 55 |
| Example 102 | Cpd 150 | 4.1 | 6.0 | 458 | 75 |
| Example 103 | Cpd 160 | 4.7 | 6.3 | 458 | 62 |
| Example 104 | Cpd 165 | 4.7 | 6.4 | 457 | 60 |
| Example 105 | Cpd 166 | 4.5 | 6.2 | 458 | 64 |
| Example 106 | Cpd 168 | 4.4 | 6.0 | 458 | 50 |
| Example 107 | Cpd 178 | 4.1 | 6.4 | 457 | 85 |
| Example 108 | Cpd 179 | 4.7 | 6.1 | 458 | 55 |
| Example 109 | Cpd 180 | 4.7 | 6.0 | 458 | 75 |
| Example 110 | Cpd 190 | 4.5 | 6.3 | 458 | 62 |
| Example 111 | Cpd 195 | 4.4 | 6.6 | 458 | 55 |
| Example 112 | Cpd 196 | 4.1 | 6.2 | 458 | 75 |
| Example 113 | Cpd 198 | 4.2 | 6.4 | 457 | 60 |
| Example 114 | Cpd 208 | 4.5 | 6.2 | 458 | 64 |
| Example 115 | Cpd 209 | 4.3 | 6.0 | 458 | 50 |
| Example 116 | Cpd 210 | 4.1 | 6.4 | 457 | 85 |
| Example 117 | Cpd 220 | 4.2 | 6.1 | 458 | 55 |
| Example 118 | Cpd 225 | 4.5 | 6.0 | 458 | 75 |
| Example 119 | Cpd 226 | 4.3 | 6.3 | 458 | 62 |
| Example 120 | Cpd 228 | 4.1 | 6.2 | 458 | 75 |
| Example 121 | Cpd 238 | 4.2 | 6.4 | 457 | 60 |
| Example 122 | Cpd 239 | 4.5 | 6.2 | 458 | 64 |
| Example 123 | Cpd 240 | 4.3 | 6.0 | 458 | 50 |
| Example 124 | Cpd 310 | 4.5 | 6.2 | 458 | 75 |
| Example 125 | Cpd 315 | 4.3 | 6.4 | 457 | 60 |
| Example 126 | Cpd 316 | 4.4 | 6.2 | 458 | 64 |
| Example 127 | Cpd 318 | 4.1 | 6.0 | 458 | 50 |
| Example 128 | Cpd 328 | 4.1 | 6.3 | 458 | 82 |
| Example 129 | Cpd 329 | 4.2 | 6.2 | 458 | 75 |
| Example 130 | Cpd 330 | 4.5 | 6.4 | 457 | 60 |
| Example 131 | Cpd 340 | 4.3 | 6.2 | 458 | 64 |
| Example 132 | Cpd 345 | 4.5 | 6.0 | 458 | 50 |
| Example 133 | Cpd 346 | 4.3 | 6.4 | 457 | 85 |
| Example 134 | Cpd 348 | 4.2 | 6.1 | 458 | 55 |
| Example 135 | Cpd 358 | 4.5 | 6.0 | 458 | 75 |
| Example 136 | Cpd 359 | 4.1 | 6.4 | 458 | 79 |
| Example 137 | Cpd 360 | 4.2 | 6.3 | 458 | 83 |
| Comparative | — | 4.7 | 5.6 | 458 | 32 |
| Example 5 | | | | | |
| Comparative | BCP | 5.3 | 5.9 | 458 | 28 |
| Example 6 | | | | | |

As shown in Table 5, it was seen that the blue organic EL devices of Examples 82 to 137 using the compound according to the present invention as a hole blocking layer material had a similar or slightly superior driving voltage compared to the blue organic EL device of Comparative Example 5 not using the hole blocking layer, but had significantly enhanced current efficiency and lifetime.

In addition, compared to the blue organic EL device of Comparative Example 6 using existing CBP as a hole blocking layer material instead of the hole blocking layer, a significantly enhanced lifetime was obtained as well as excellent driving voltage and current efficiency.

1. A compound represented by the following Chemical Formula 1: 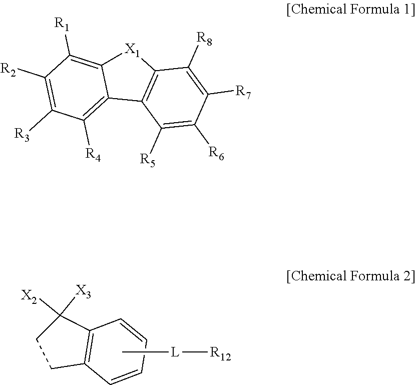
wherein,
X₁ is selected from the group consisting of O, S, C(R₉)(R₁₀) and N(R₁₁);
at least one of R₁ and R₂, R₂ and R₃, R₃ and R₄, R₄ and R₅, R₅ and R₆, R₆ and R₇, and R₇ and R₈ is fused with the ring represented by Chemical Formula 2 to form a fused ring;
R₁, R₂, R₃, R₄, R₅, R₆, R₇ and R₈ not forming a fused ring with the ring represented by Chemical Formula 2 are the same as or different from each other, and each independently selected from the group consisting of hydrogen, deuterium, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group, or bond to adjacent groups to form a fused ring;
R₉ to R₁₁ are the same as or different from each other, and each independently selected from the group consisting of hydrogen, deuterium, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group;
a dotted line of Chemical Formula 2 is a part fused to Chemical Formula 1;
X₂ and X₃ are the same as or different from each other, and each independently selected from the group consisting of hydrogen, deuterium, a C₁˜C₂ alkyl group and a C₆˜C₆₀ aryl group;
L is selected from the group consisting of a single bond, a C₆˜C₁₈ arylene group and a heteroarylene group having 5 to 18 nuclear atoms;
R₁₂ is selected from the group consisting of a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylamine group; and
the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the arylphosphine group, the arylphosphine oxide group, the mono or diarylphosphinyl group and the arylamine group of R₁ to R₁₂, X₂ and X₃ are each independently unsubstituted or substituted with one or more types of substituents selected from the group consisting of a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylamine group, and when substituted with a plurality of the substituents, these are the same as or different from each other.

2. The compound of claim 1, wherein the compound represented by Chemical Formula 1 is represented by any one of the following Chemical Formula 3 to Chemical Formula 8: 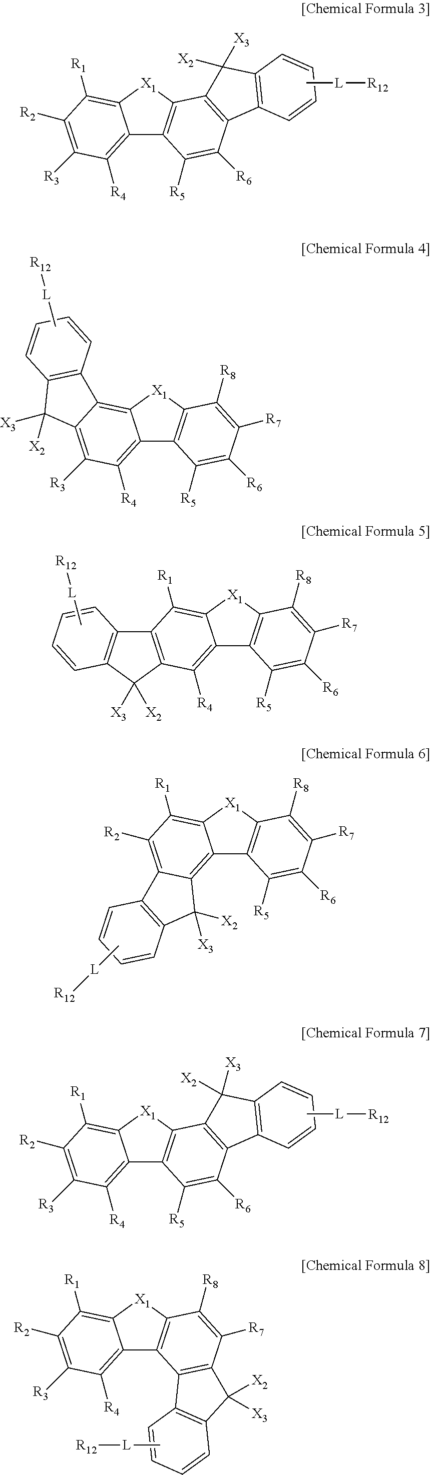
wherein,
X₁ to X₃, L and R₁ to R₈ each have the same definition as in claim 1.

3. The compound of claim 1, wherein X₁ is O.

4. The compound of claim 1, wherein X₂ and X₃ are a methyl group.

5. The compound of claim 1, wherein R₁₂ is a substituent represented by any one of the following Chemical Formula 9 to Chemical Formula 11: 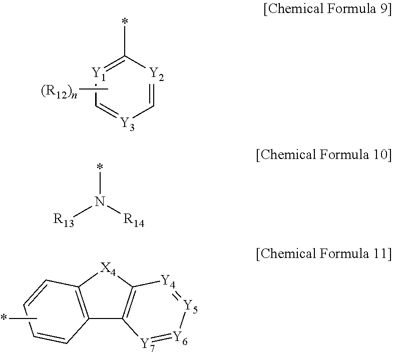
wherein,
* means a part forming a bond;
n is an integer of 0 to 2;
Y₁ to Y₃ are the same as or different from each other and each independently C(R₁₅) or N, and at least one or more are N;
Y₄ to Y₇ are the same as or different from each other, and each independently C(R₁₆) or N;
X₄ is selected from the group consisting of O, S, C(R₁₇)(R₁₈), Si(R₁₉)(R₂₀) and N(R₂₁);
R₁₂ to R₁₄ are the same as or different from each other, and each independently selected from the group consisting of hydrogen, deuterium, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group;
R₁₅ and R₁₆ are the same as or different from each other, and each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group, or bond to adjacent groups to form a fused ring;
R₁₇ to R21 are the same as or different from each other, and each independently selected from the group consisting of a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group, or bond to adjacent groups to form a fused ring; and
the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the arylphosphine group, the arylphosphine oxide group, the mono or diarylphosphinyl group and the arylamine group of R₁₂ to R₂₁ are each independently unsubstituted or substituted with one or more types of substituents selected from the group consisting of a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylamine group, and when substituted with a plurality of the substituents, these are the same as or different from each other.

6. The compound of claim 1, wherein L is a single bond, a phenylene group or a biphenylene group.

7. The compound of claim 1, wherein the compound represented by Chemical Formula 1 is selected from the group consisting of the following compounds:

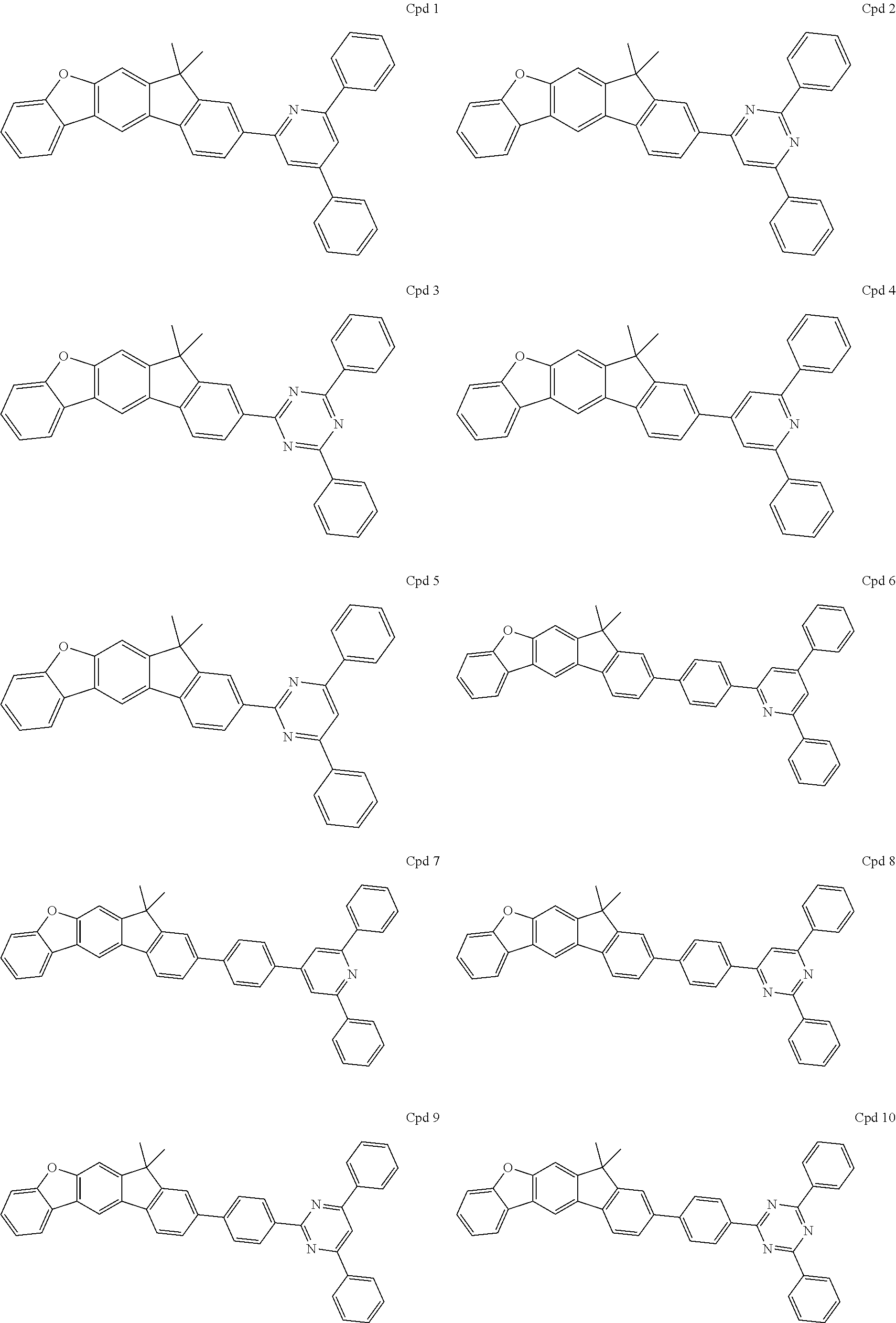

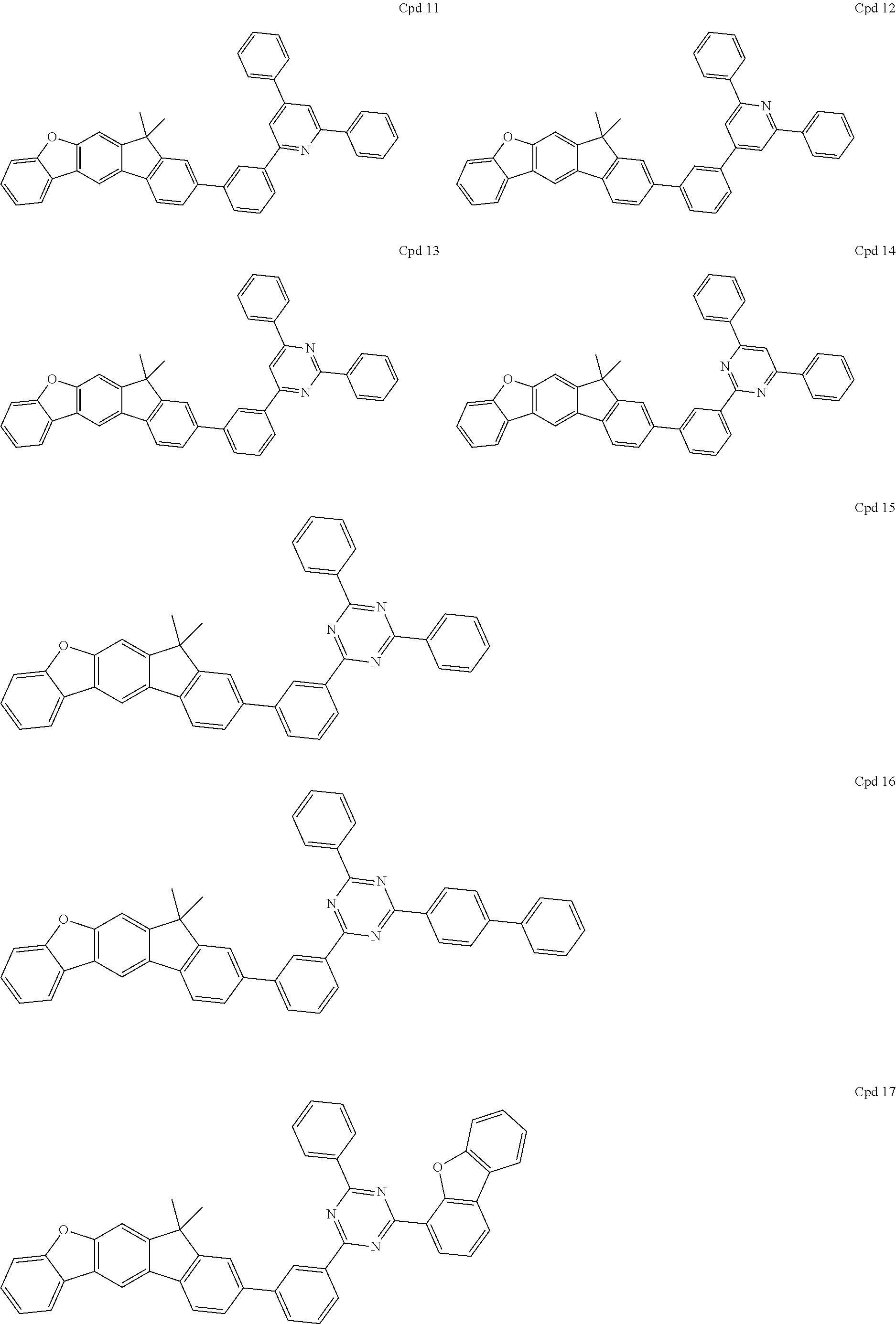

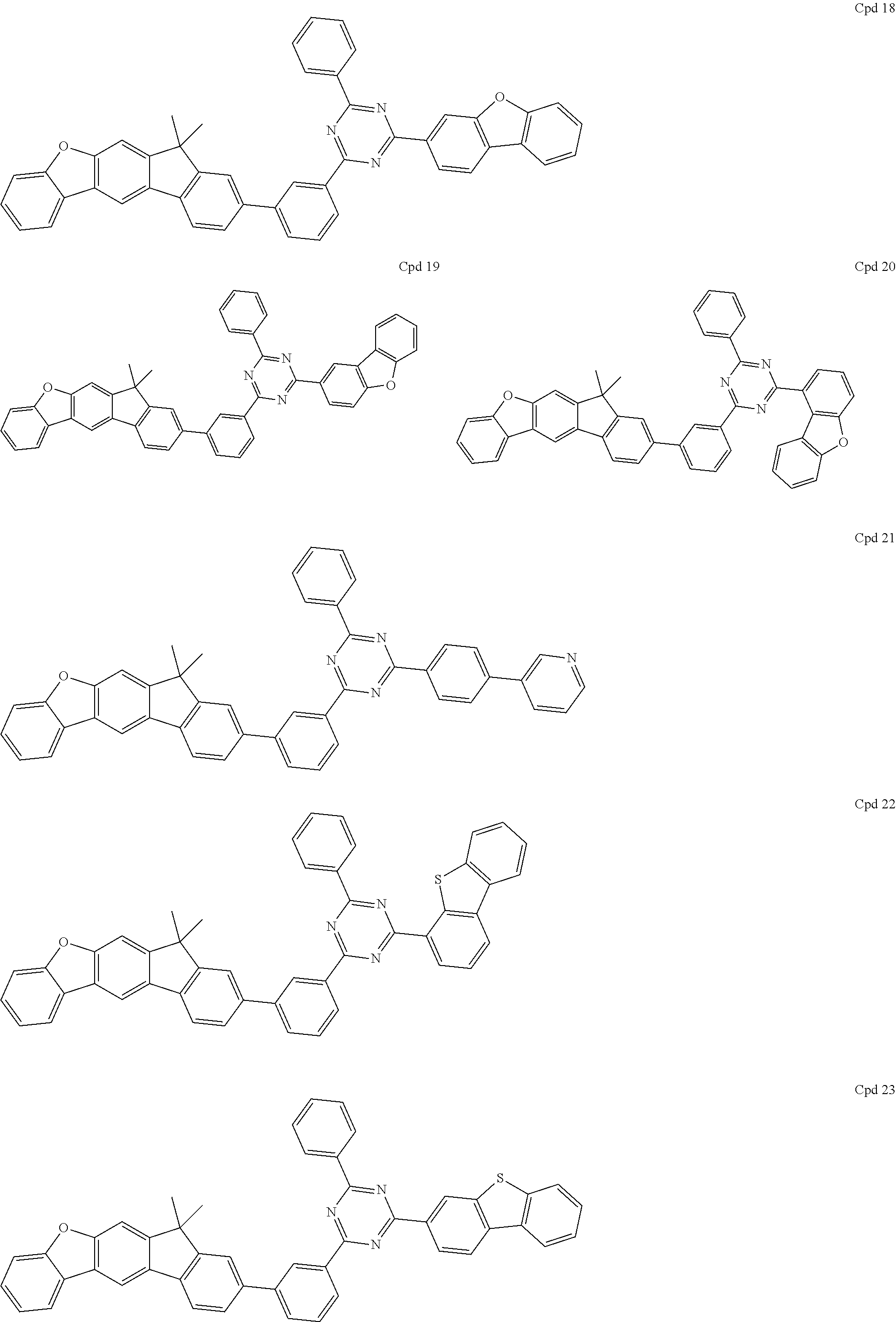

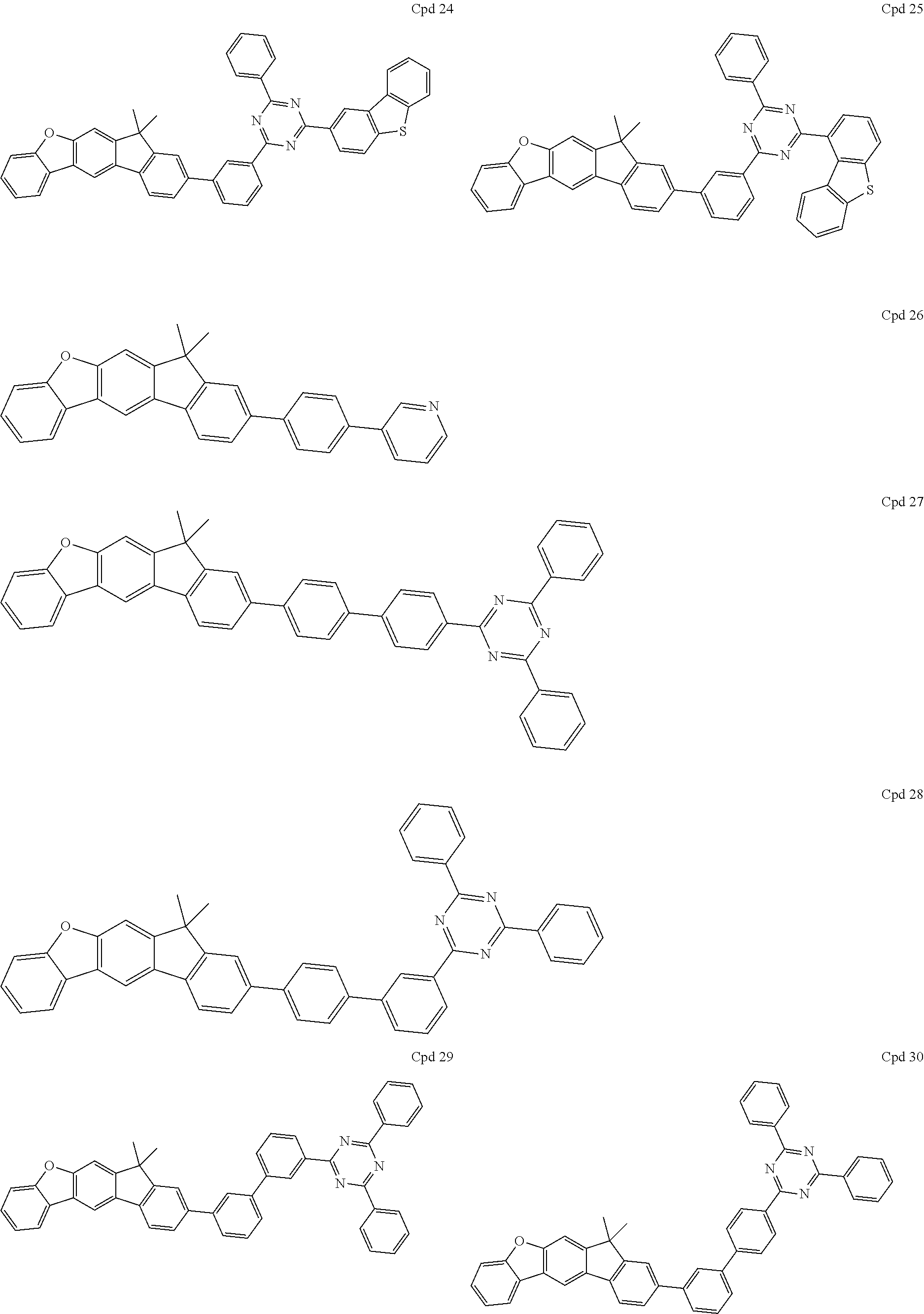

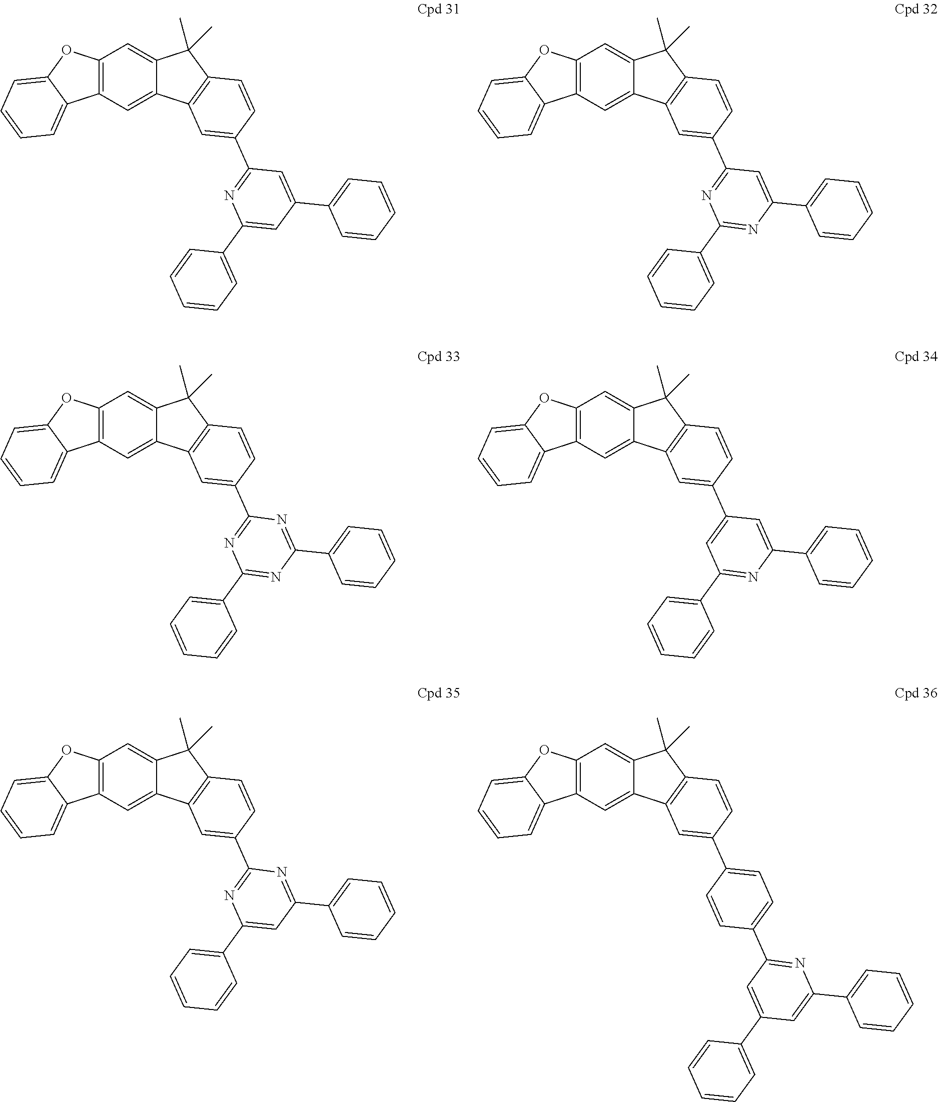 [SMILES: CC1(C)c2ccc(-c3cc(-c4ccccc4)cc(-c4ccccc4)n3)cc2-c2cc3c(cc21)oc1ccccc13 ; CC1(C)c2ccc(-c3cc(-c4ccccc4)nc(-c4ccccc4)n3)cc2-c2cc3c(cc21)oc1ccccc13 ; CC1(C)c2ccc(-c3nc(-c4ccccc4)nc(-c4ccccc4)n3)cc2-c2cc3c(cc21)oc1ccccc13 ; CC1(C)c2ccc(-c3cc(-c4ccccc4)nc(-c4ccccc4)c3)cc2-c2cc3c(cc21)oc1ccccc13 ; CC1(C)c2ccc(-c3nc(-c4ccccc4)cc(-c4ccccc4)n3)cc2-c2cc3c(cc21)oc1ccccc13 ; CC1(C)c2ccc(-c3ccc(-c4cc(-c5ccccc5)cc(-c5ccccc5)n4)cc3)cc2-c2cc3c(cc21)oc1ccccc13]

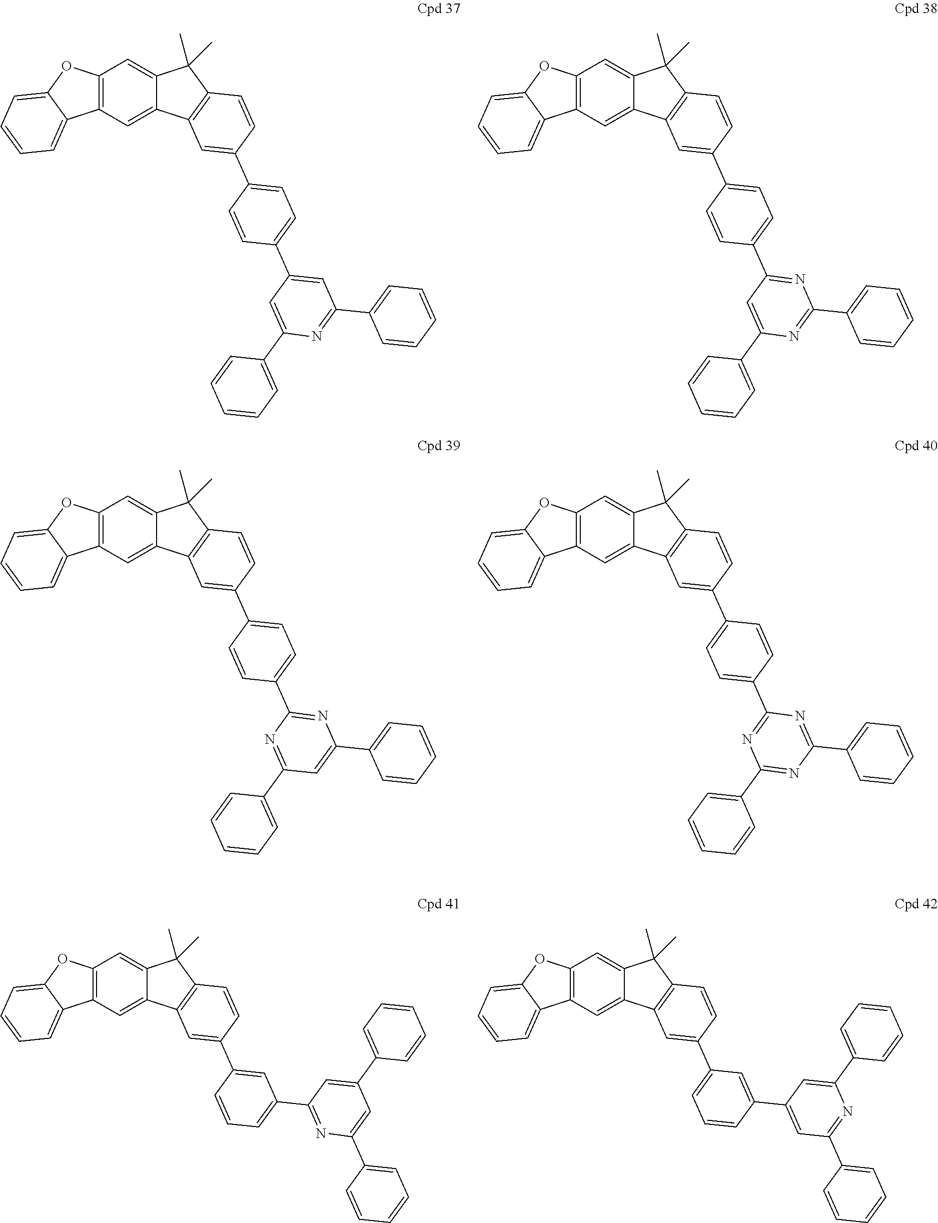

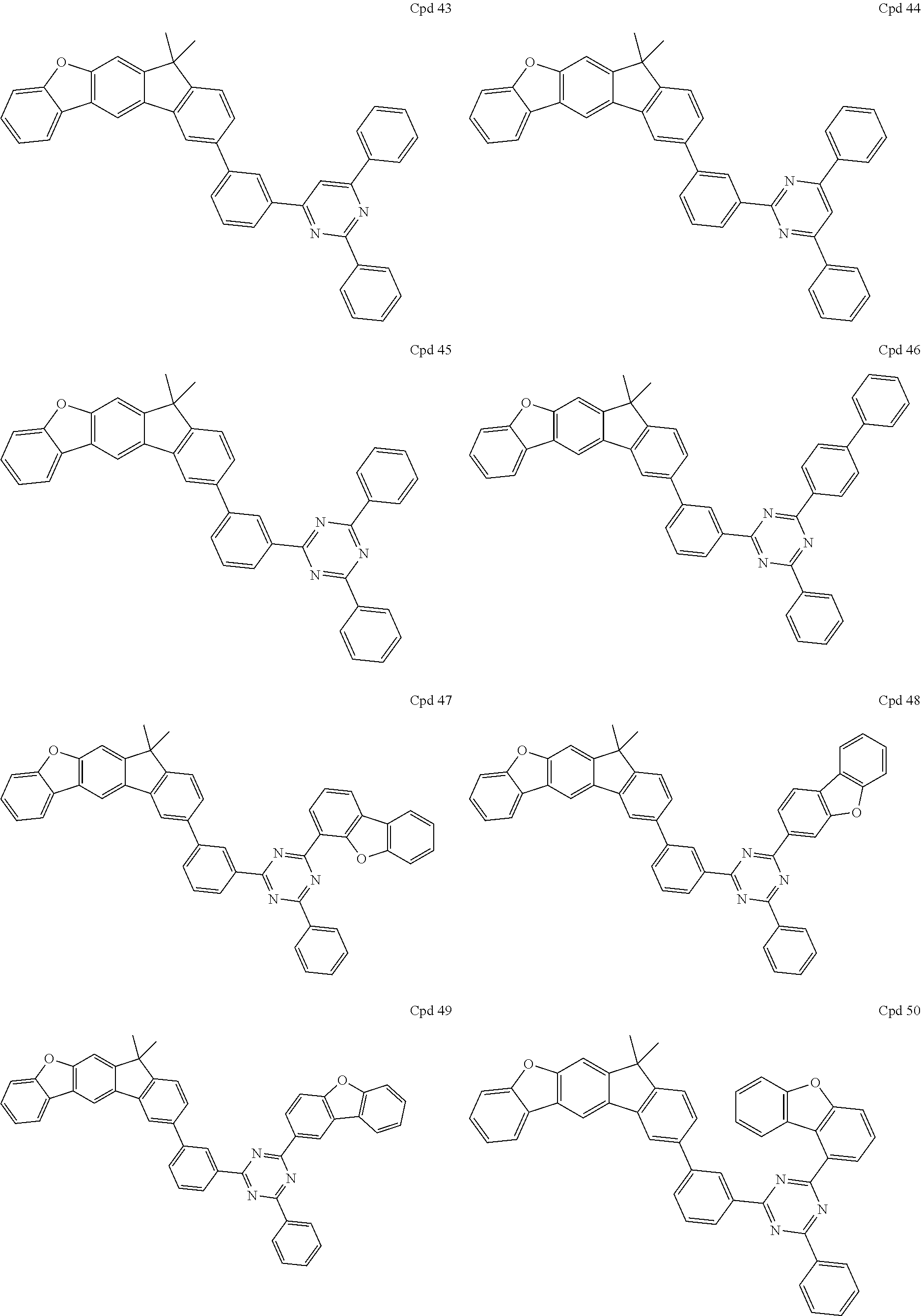

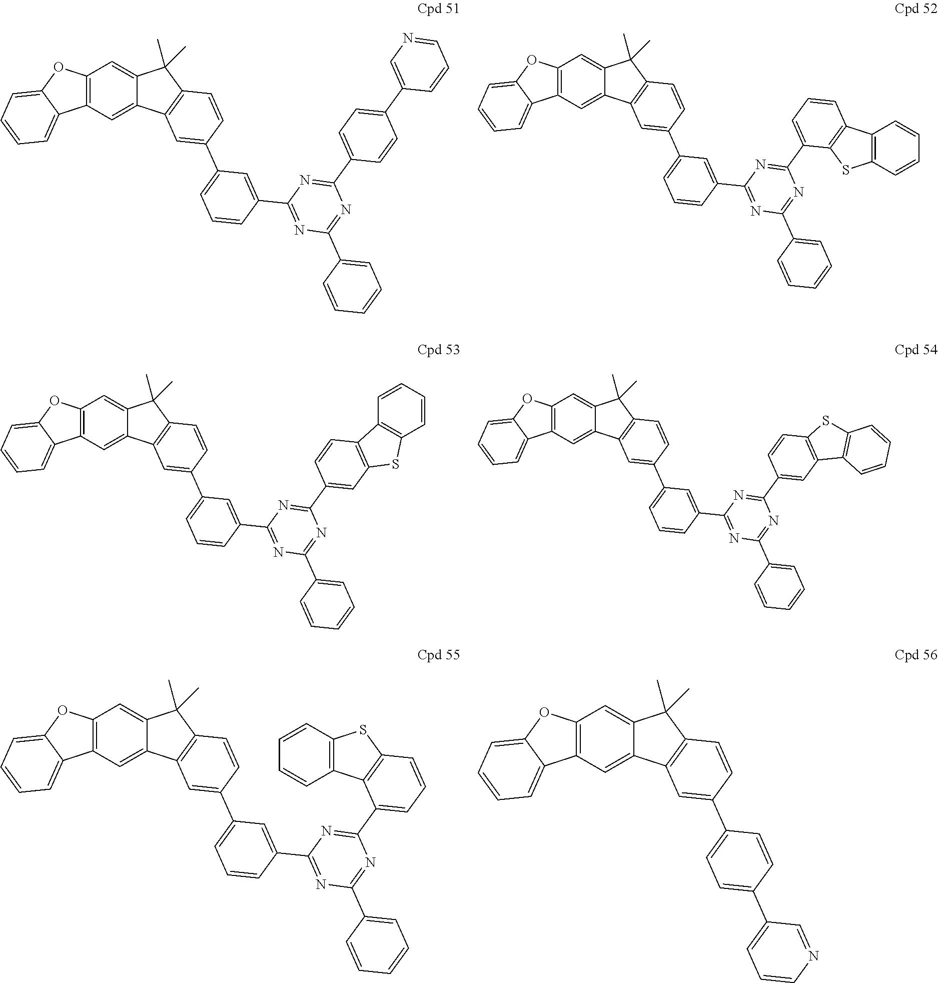

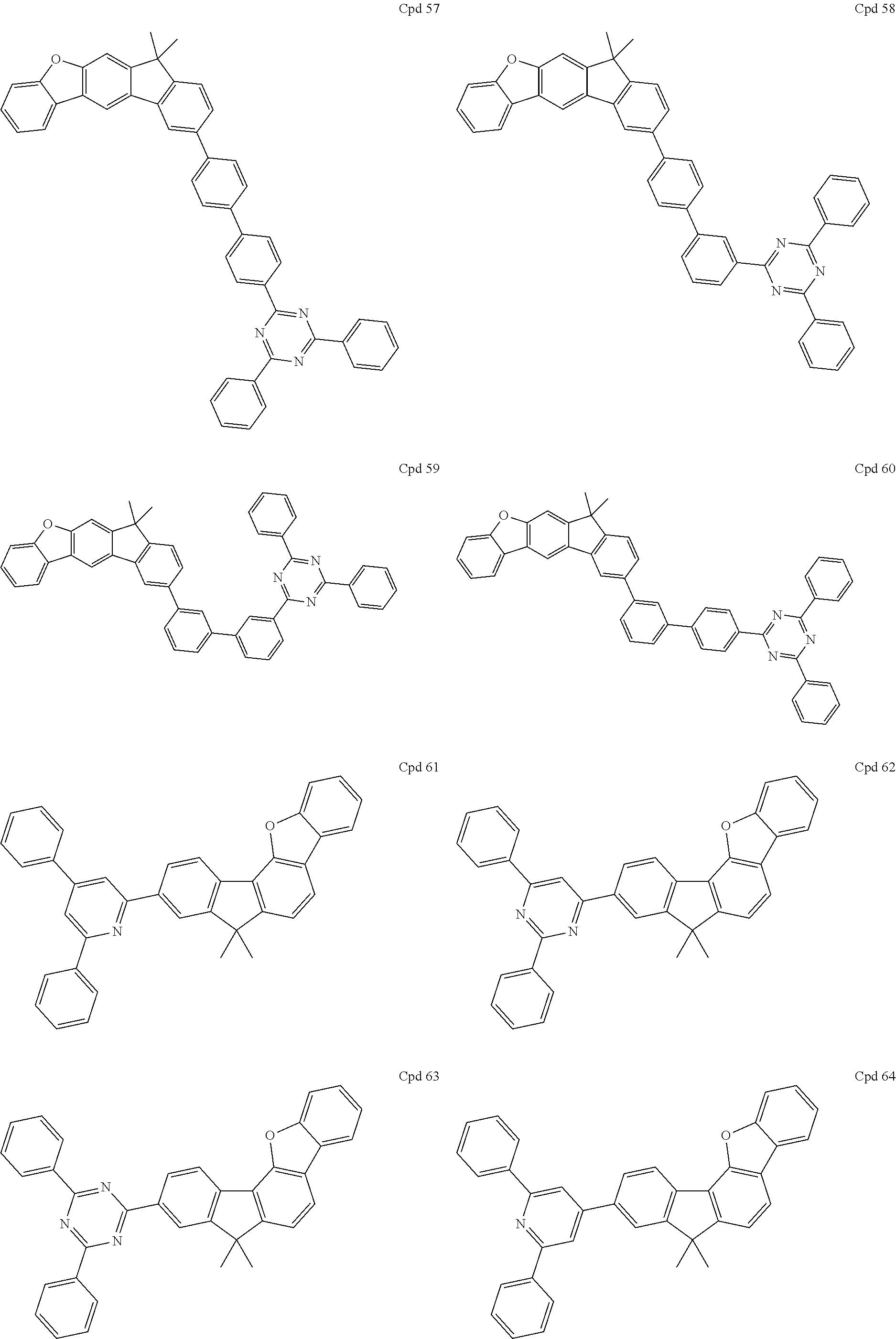

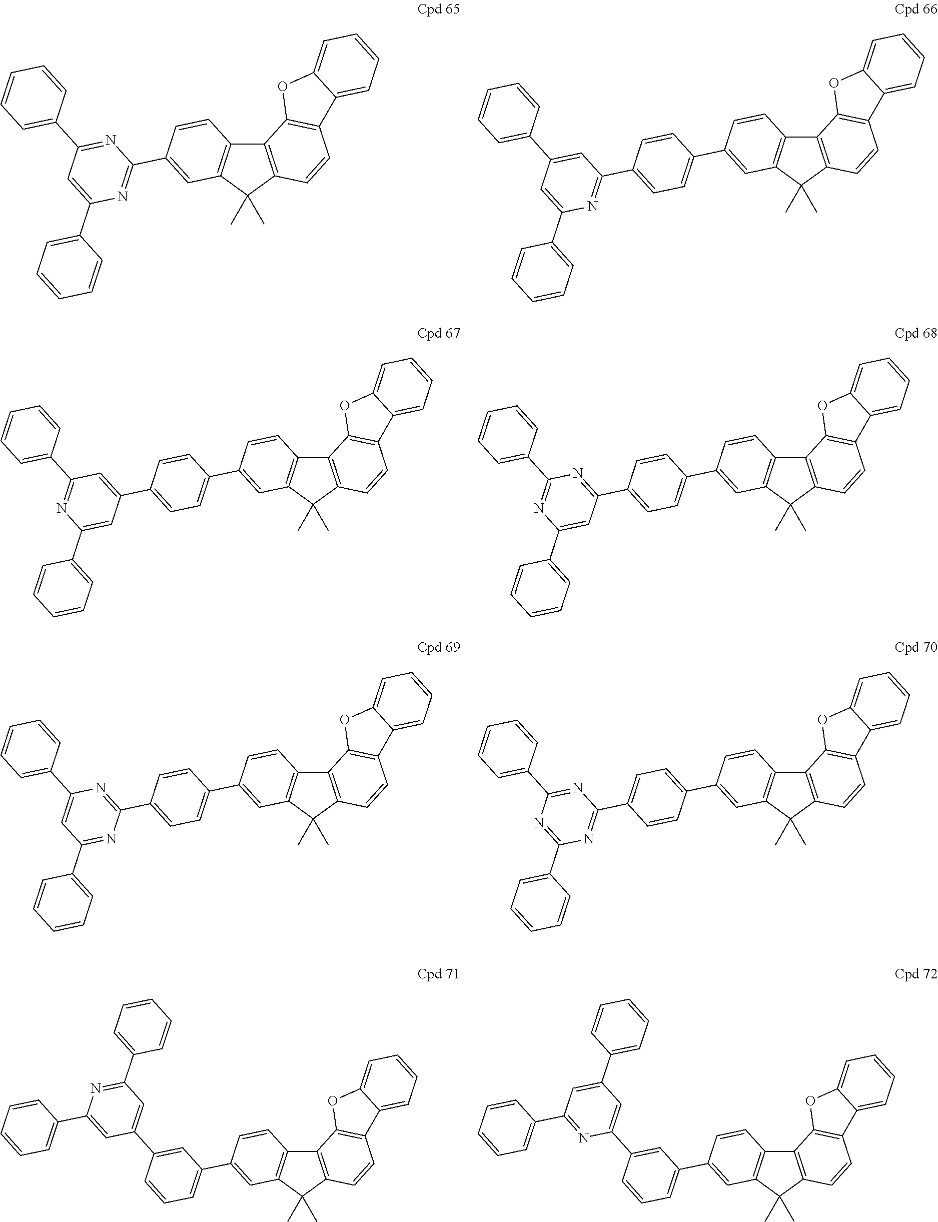

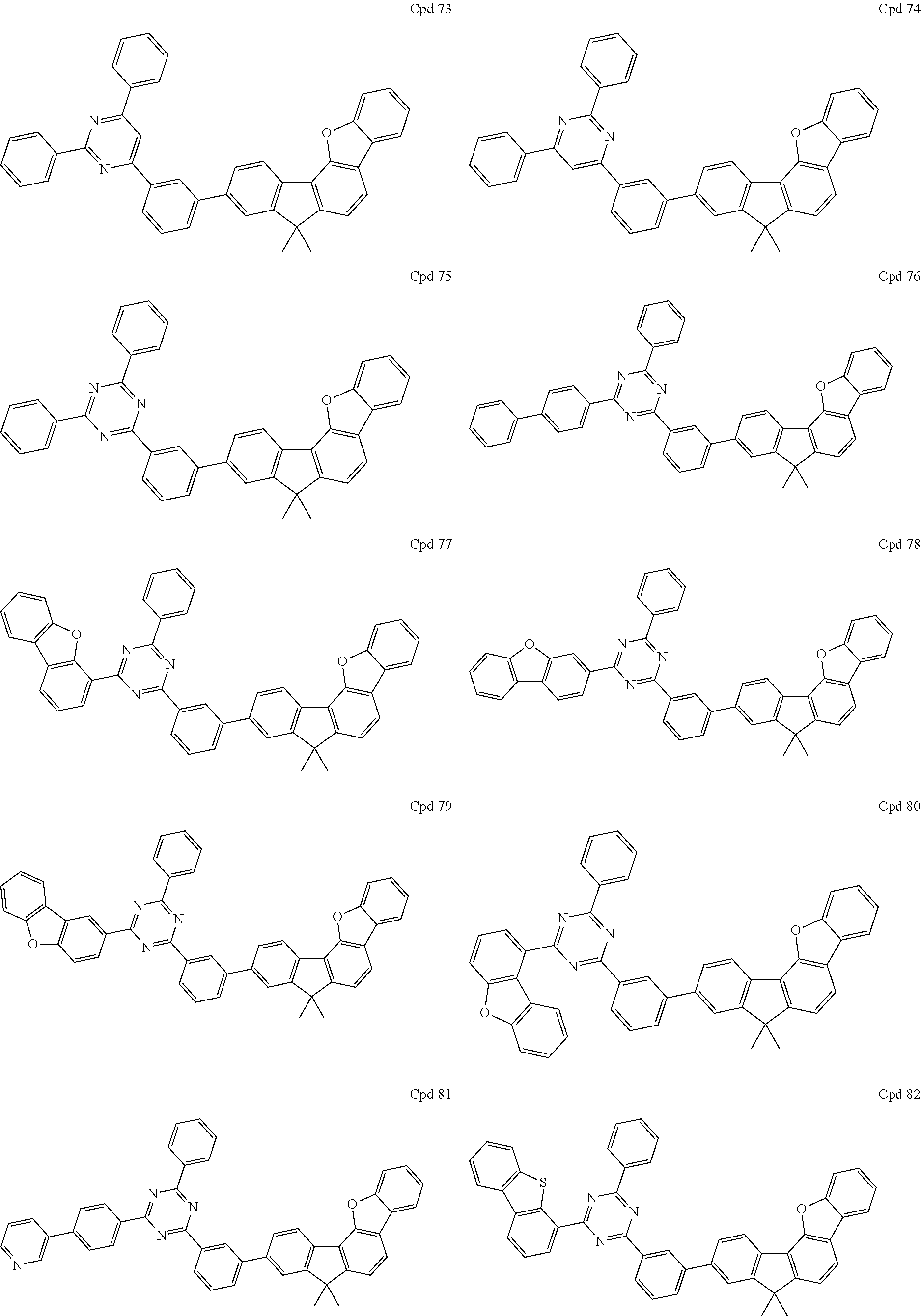

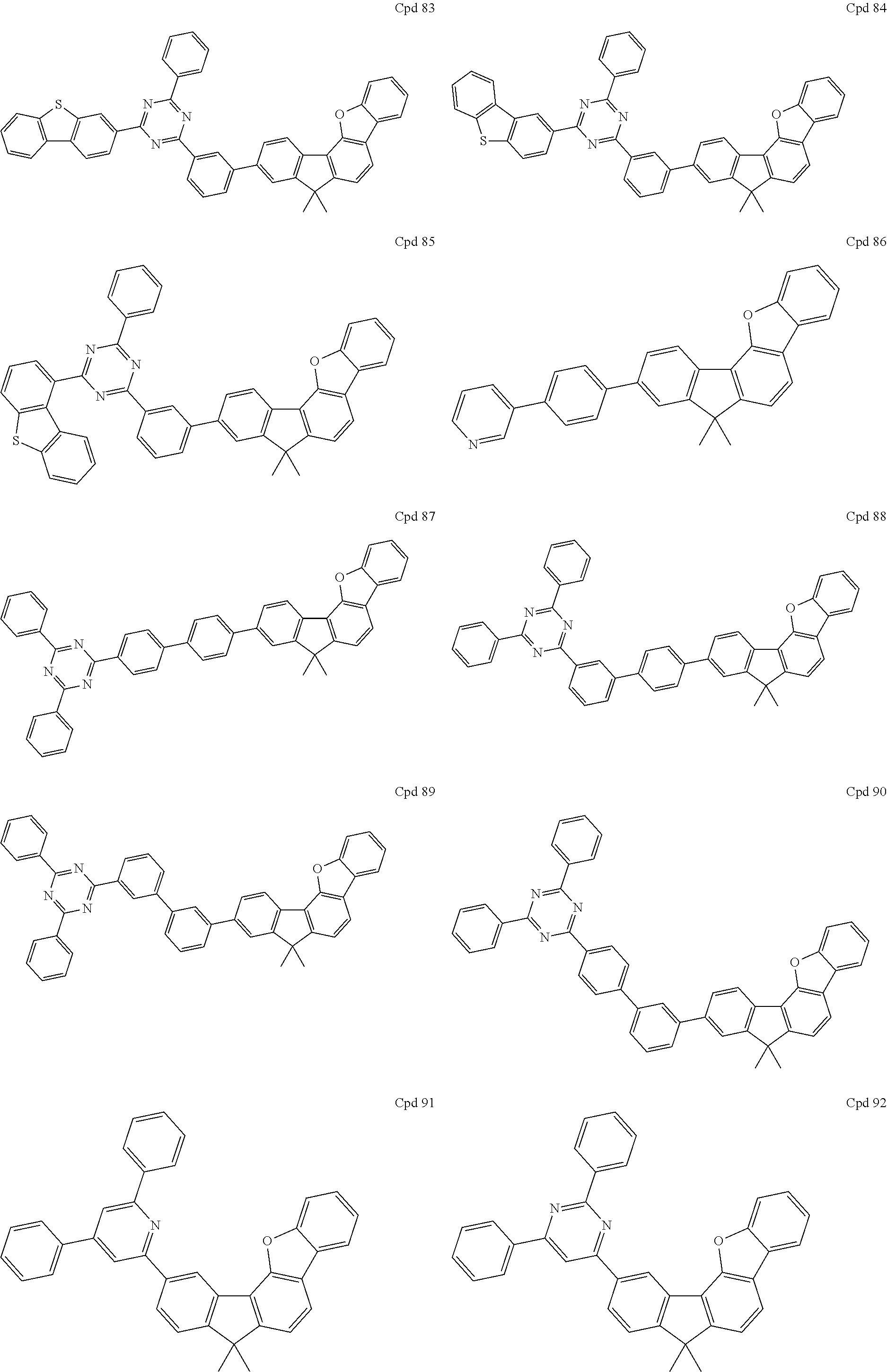

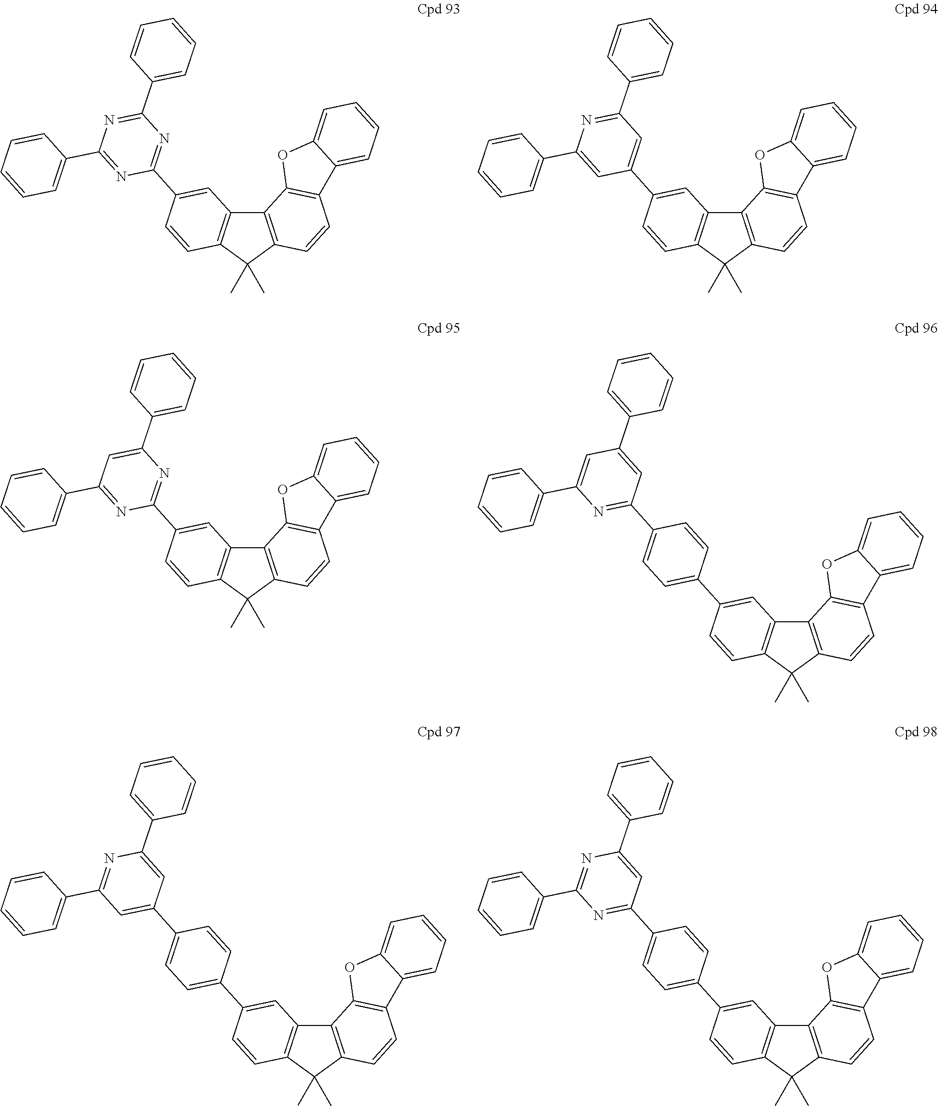

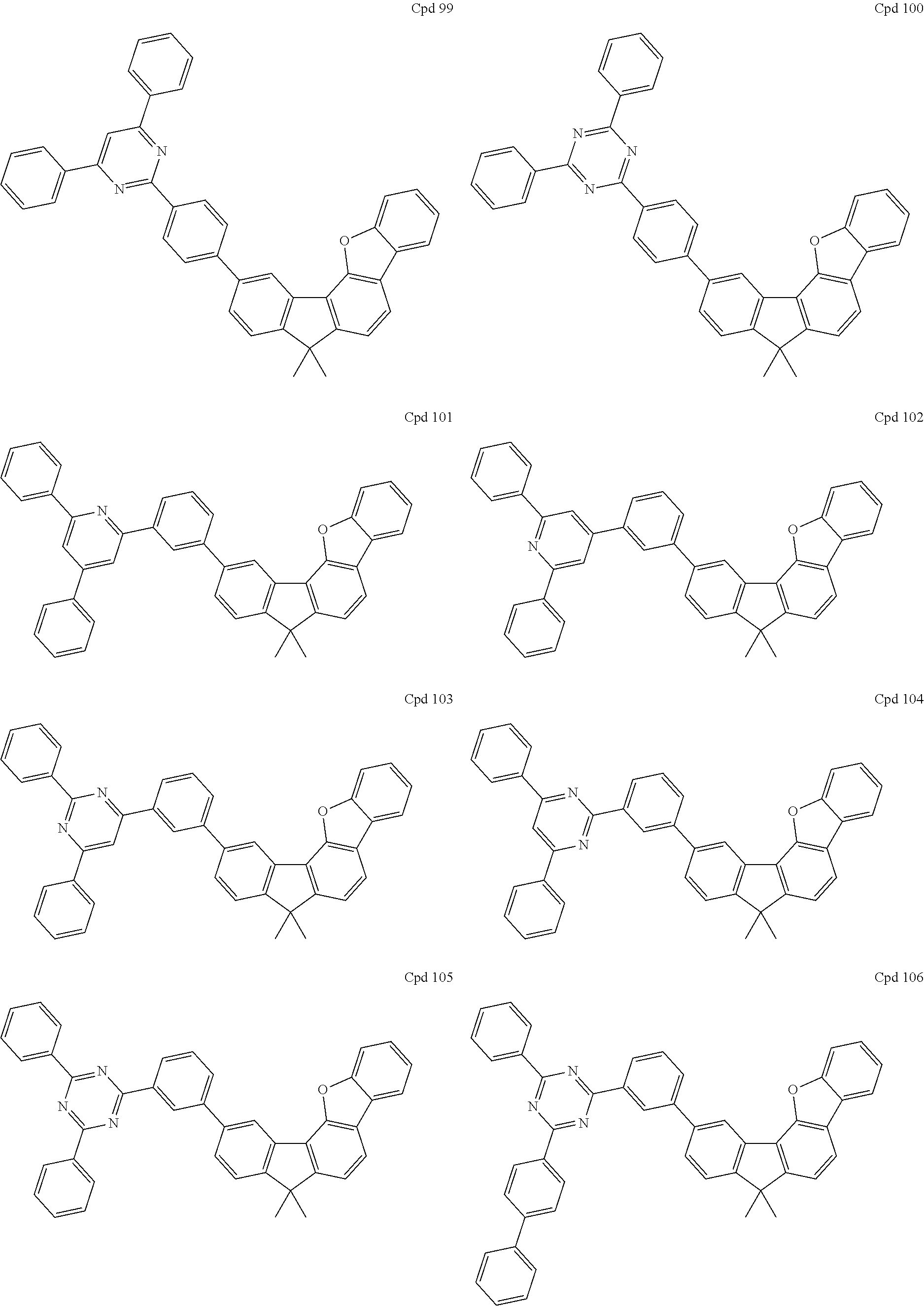

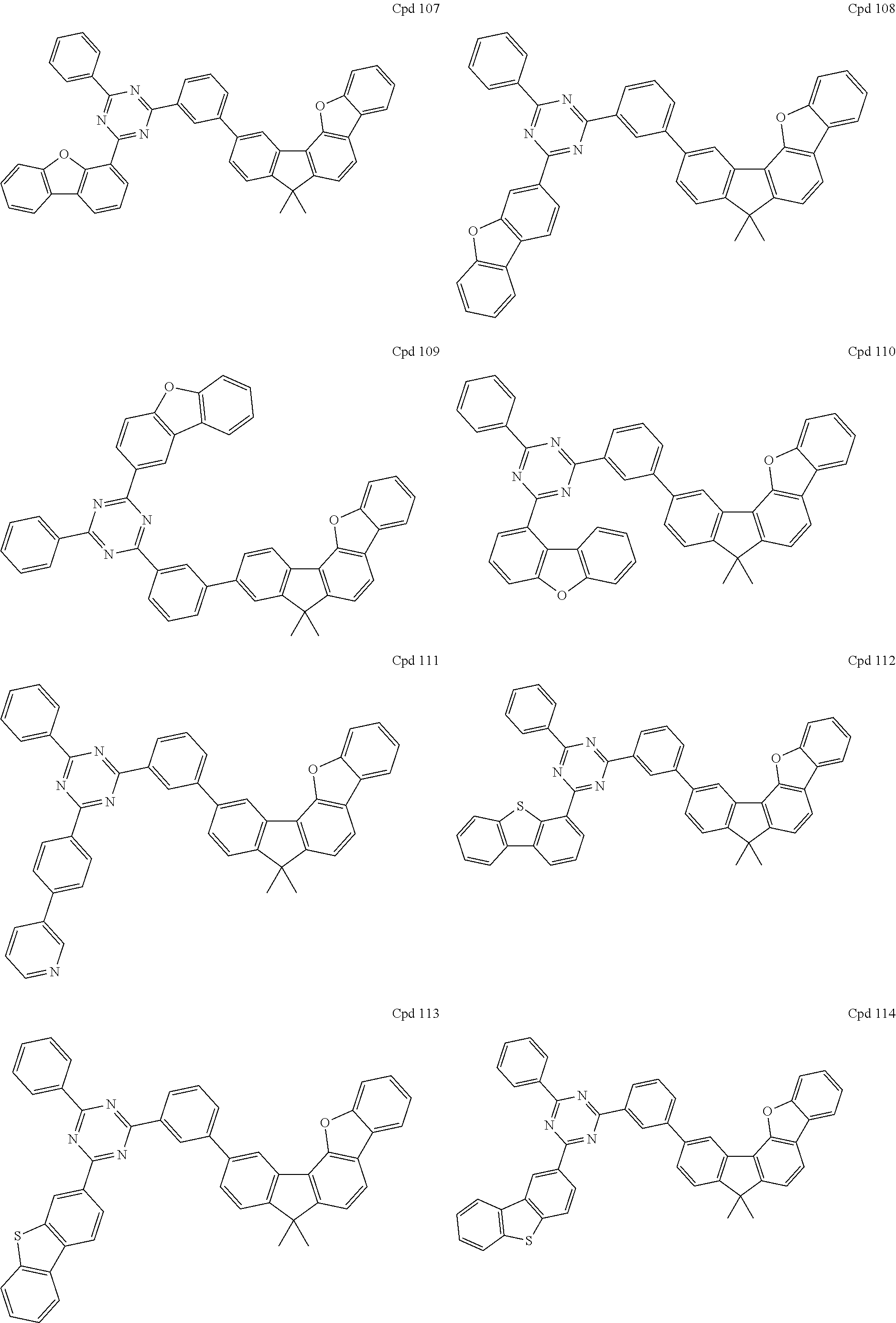

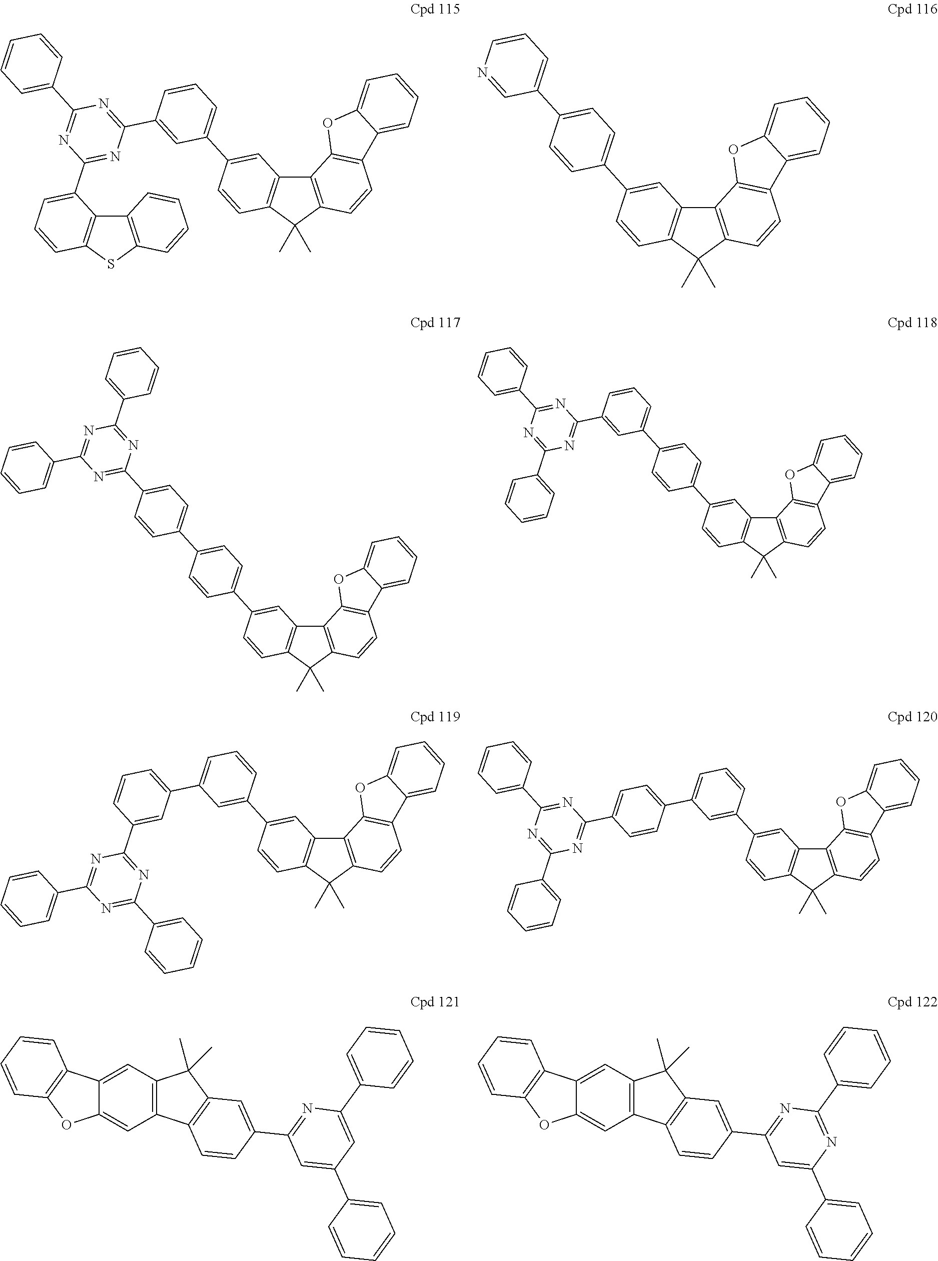

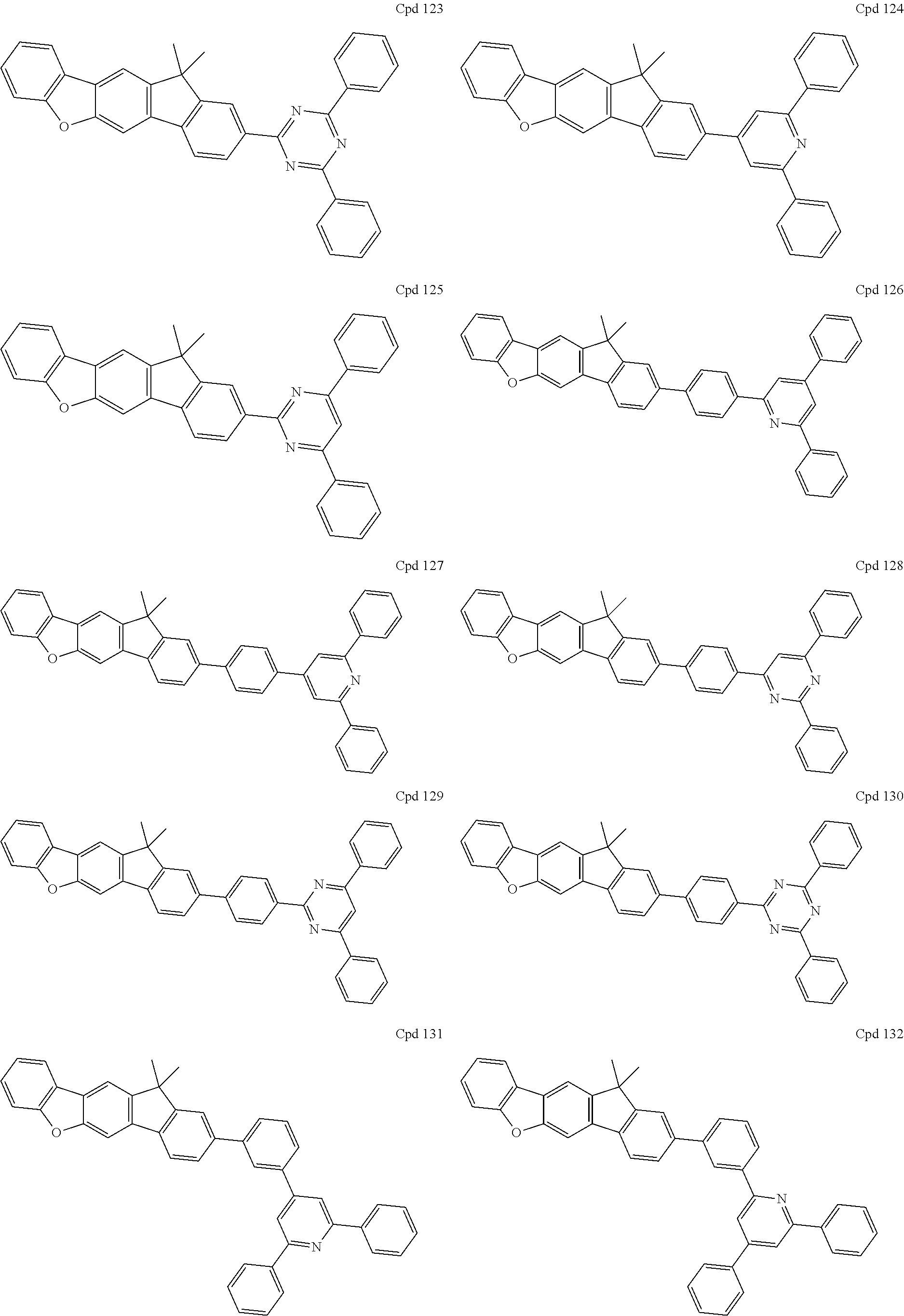

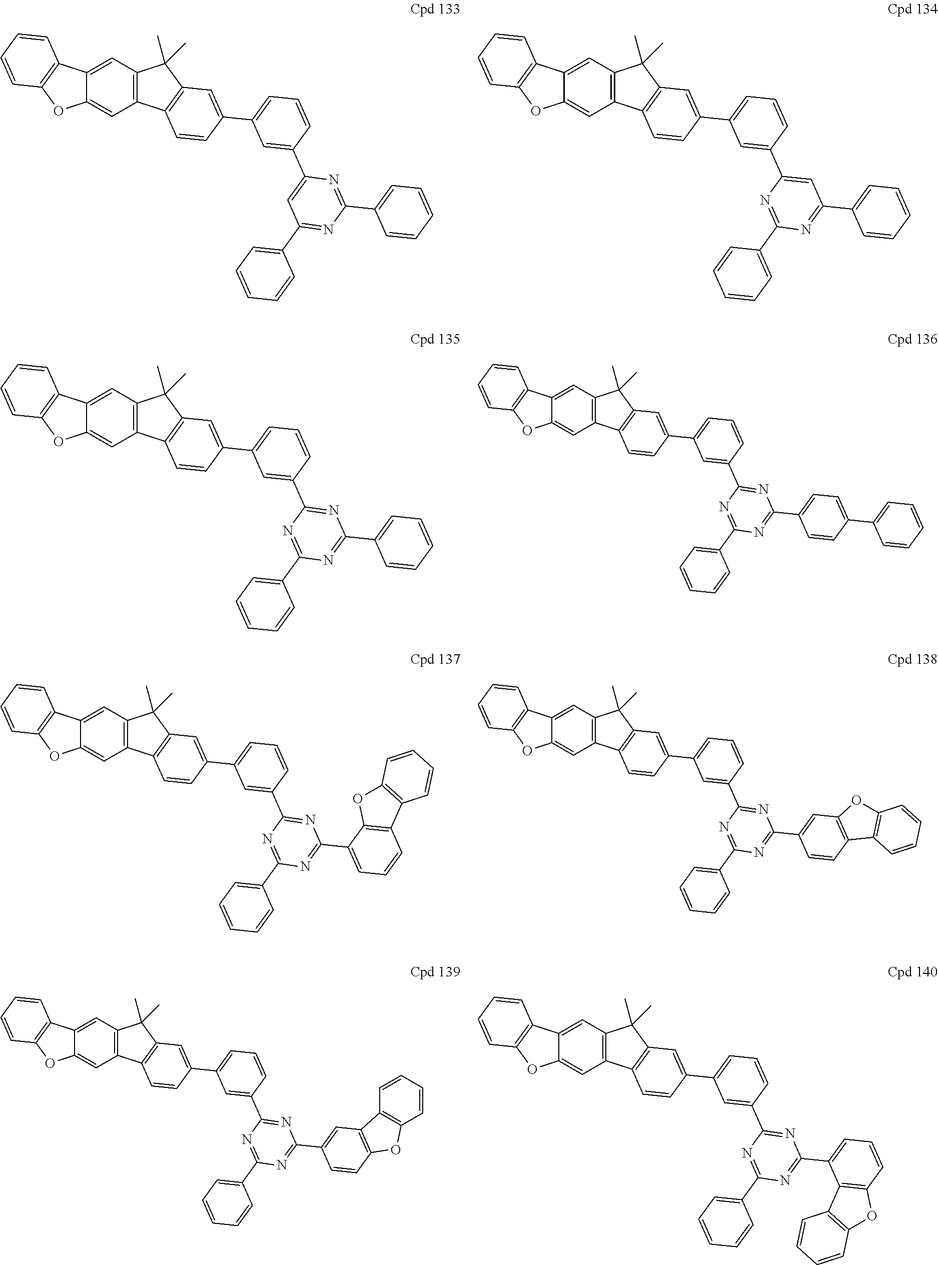

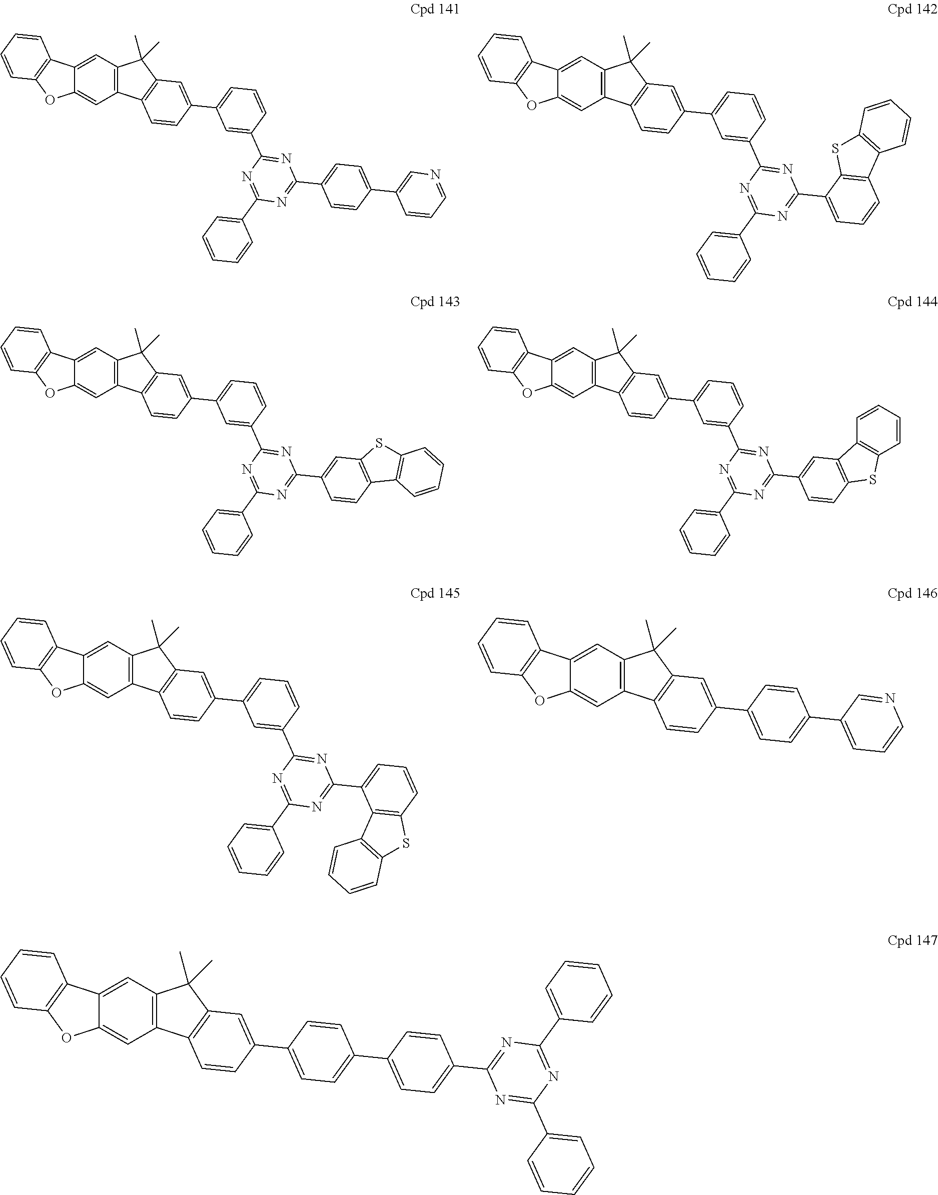

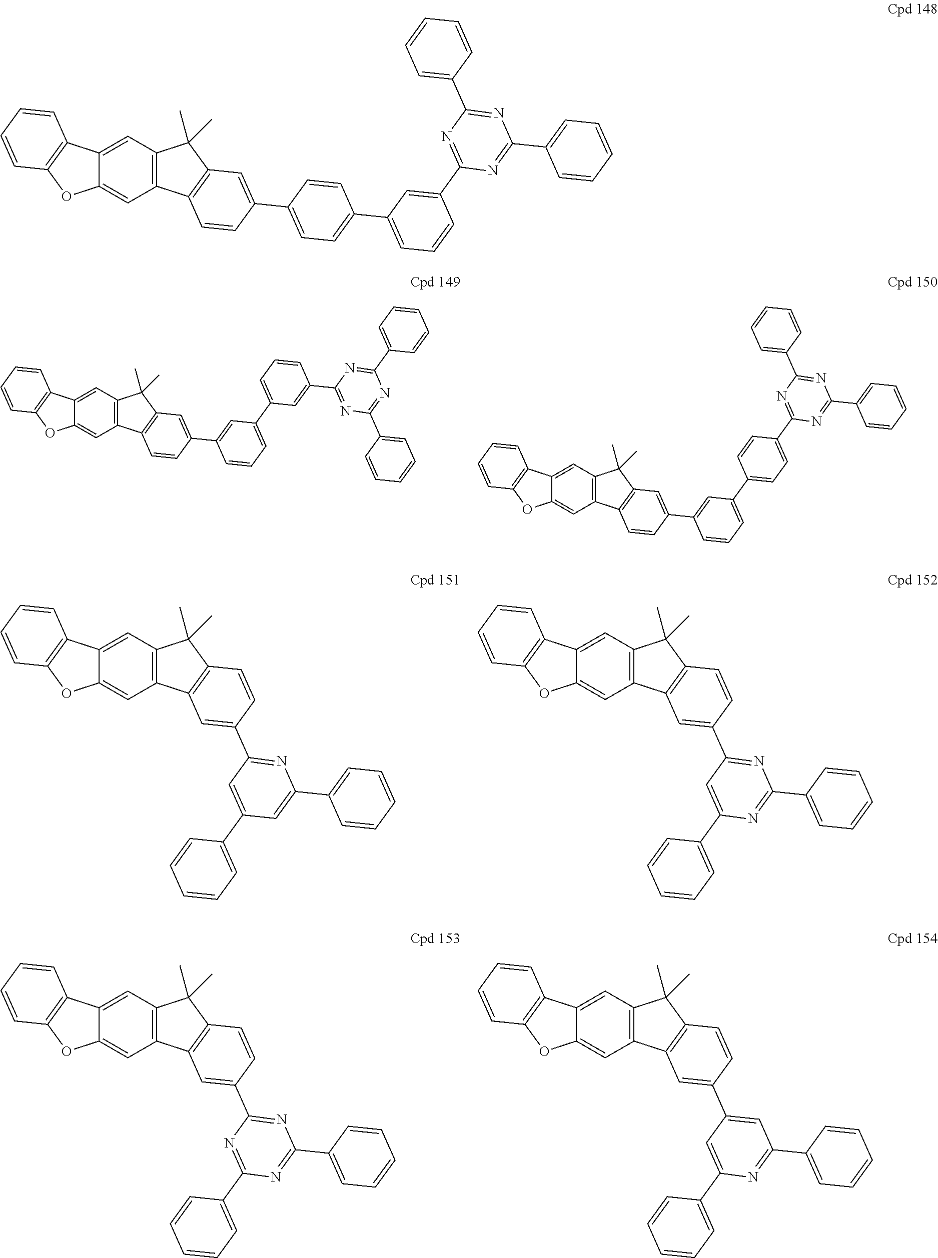

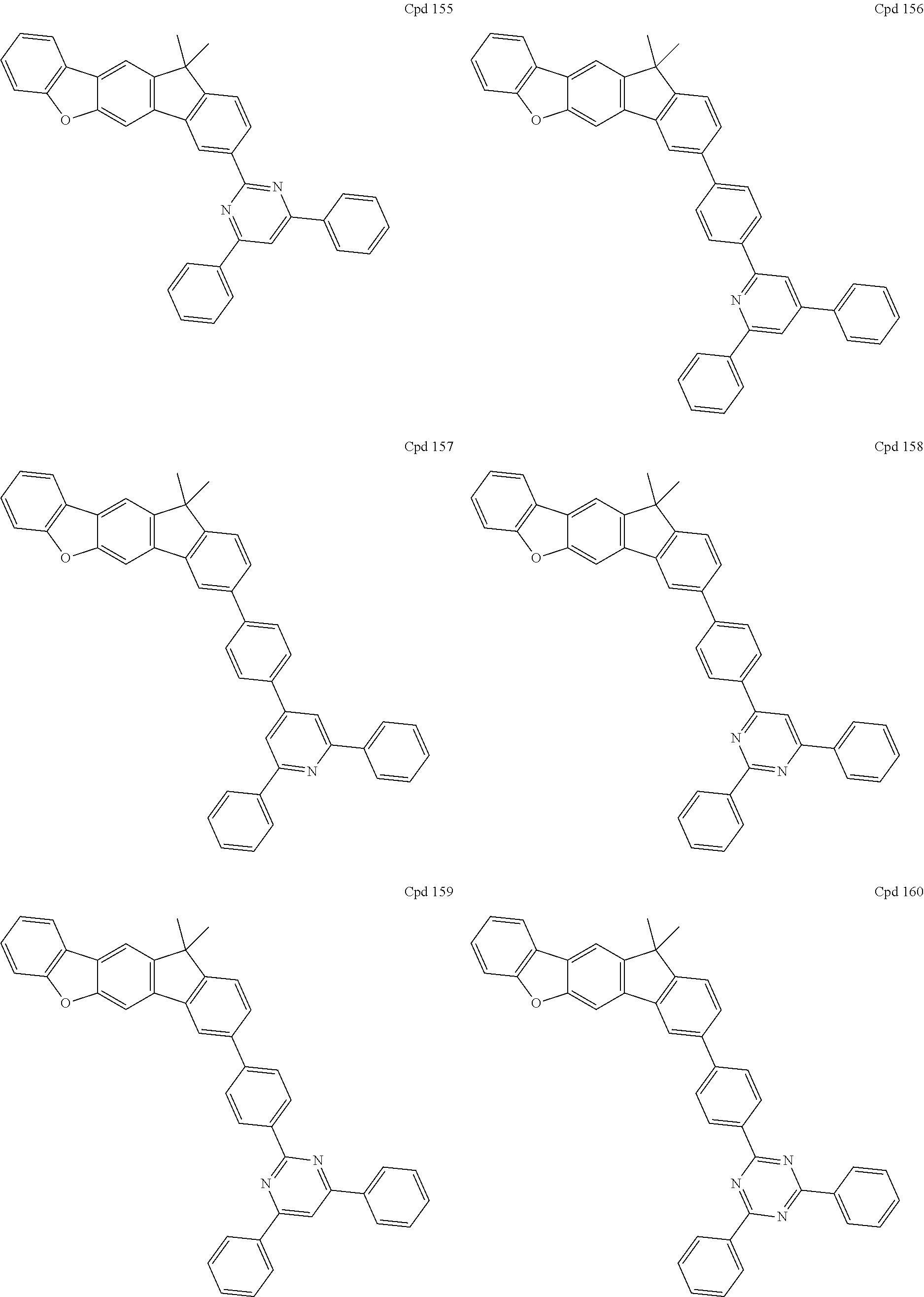 [SMILES: CC1(C)c2ccc(-c3nc(-c4ccccc4)cc(-c4ccccc4)n3)cc2-c2cc3oc4ccccc4c3cc21 ; CC1(C)c2ccc(-c3ccc(-c4cc(-c5ccccc5)cc(-c5ccccc5)n4)cc3)cc2-c2cc3oc4ccccc4c3cc21 ; CC1(C)c2ccc(-c3ccc(-c4cc(-c5ccccc5)nc(-c5ccccc5)c4)cc3)cc2-c2cc3oc4ccccc4c3cc21 ; CC1(C)c2ccc(-c3ccc(-c4cc(-c5ccccc5)nc(-c5ccccc5)n4)cc3)cc2-c2cc3oc4ccccc4c3cc21 ; CC1(C)c2ccc(-c3ccc(-c4nc(-c5ccccc5)cc(-c5ccccc5)n4)cc3)cc2-c2cc3oc4ccccc4c3cc21 ; CC1(C)c2ccc(-c3ccc(-c4nc(-c5ccccc5)nc(-c5ccccc5)n4)cc3)cc2-c2cc3oc4ccccc4c3cc21]

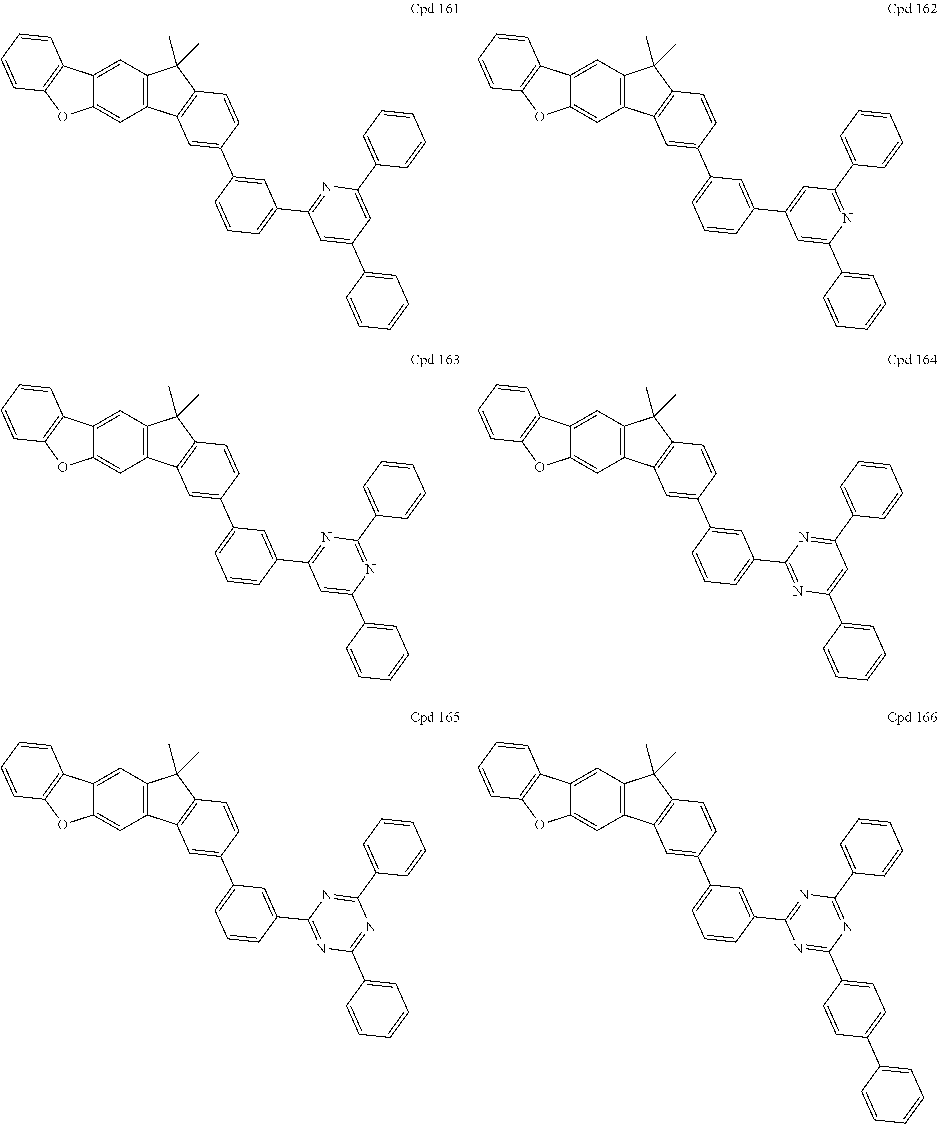 [SMILES: CC1(C)c2ccc(-c3cccc(-c4cc(-c5ccccc5)cc(-c5ccccc5)n4)c3)cc2-c2cc3oc4ccccc4c3cc21 ; CC1(C)c2ccc(-c3cccc(-c4cc(-c5ccccc5)nc(-c5ccccc5)c4)c3)cc2-c2cc3oc4ccccc4c3cc21 ; CC1(C)c2ccc(-c3cccc(-c4cc(-c5ccccc5)nc(-c5ccccc5)n4)c3)cc2-c2cc3oc4ccccc4c3cc21 ; CC1(C)c2ccc(-c3cccc(-c4nc(-c5ccccc5)cc(-c5ccccc5)n4)c3)cc2-c2cc3oc4ccccc4c3cc21 ; CC1(C)c2ccc(-c3cccc(-c4nc(-c5ccccc5)nc(-c5ccccc5)n4)c3)cc2-c2cc3oc4ccccc4c3cc21 ; CC1(C)c2ccc(-c3cccc(-c4nc(-c5ccccc5)nc(-c5ccc(-c6ccccc6)cc5)n4)c3)cc2-c2cc3oc4ccccc4c3cc21]

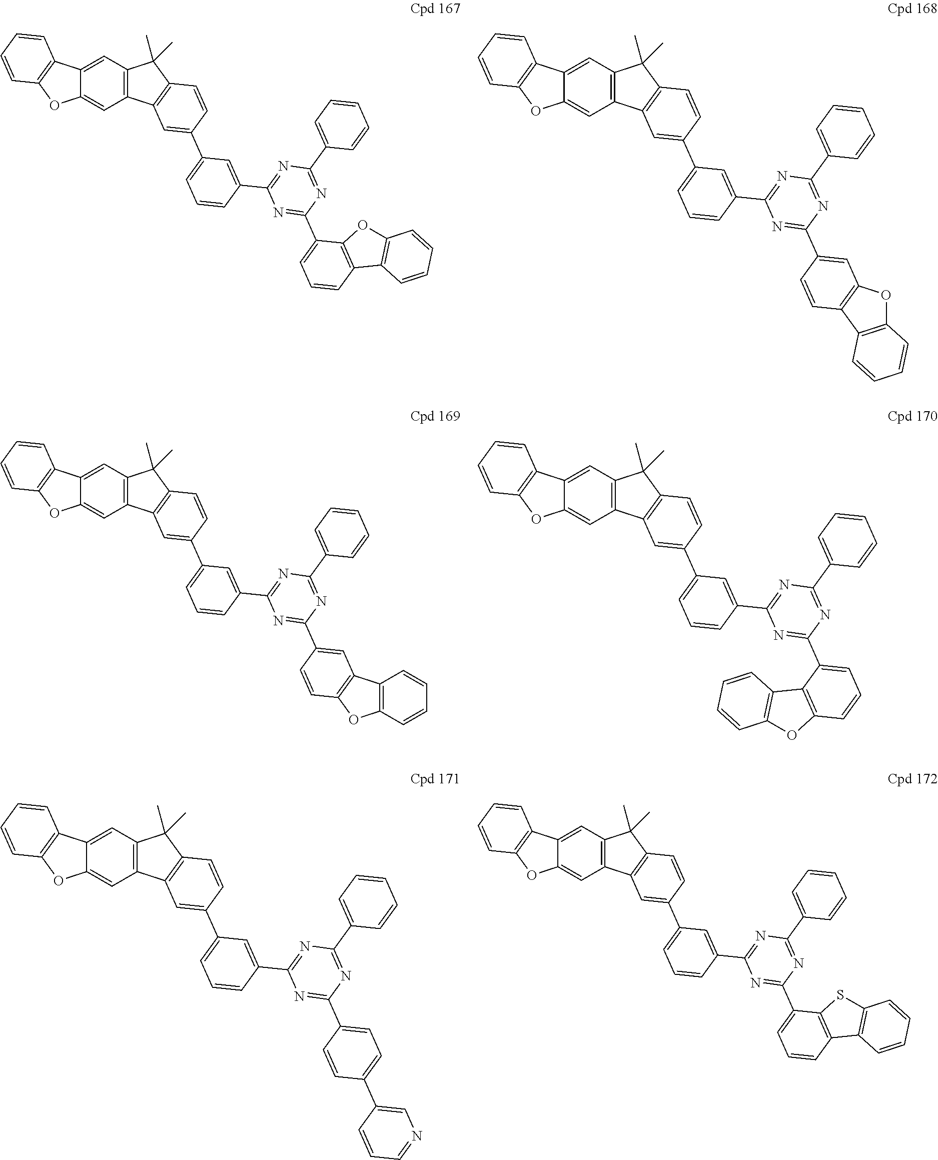

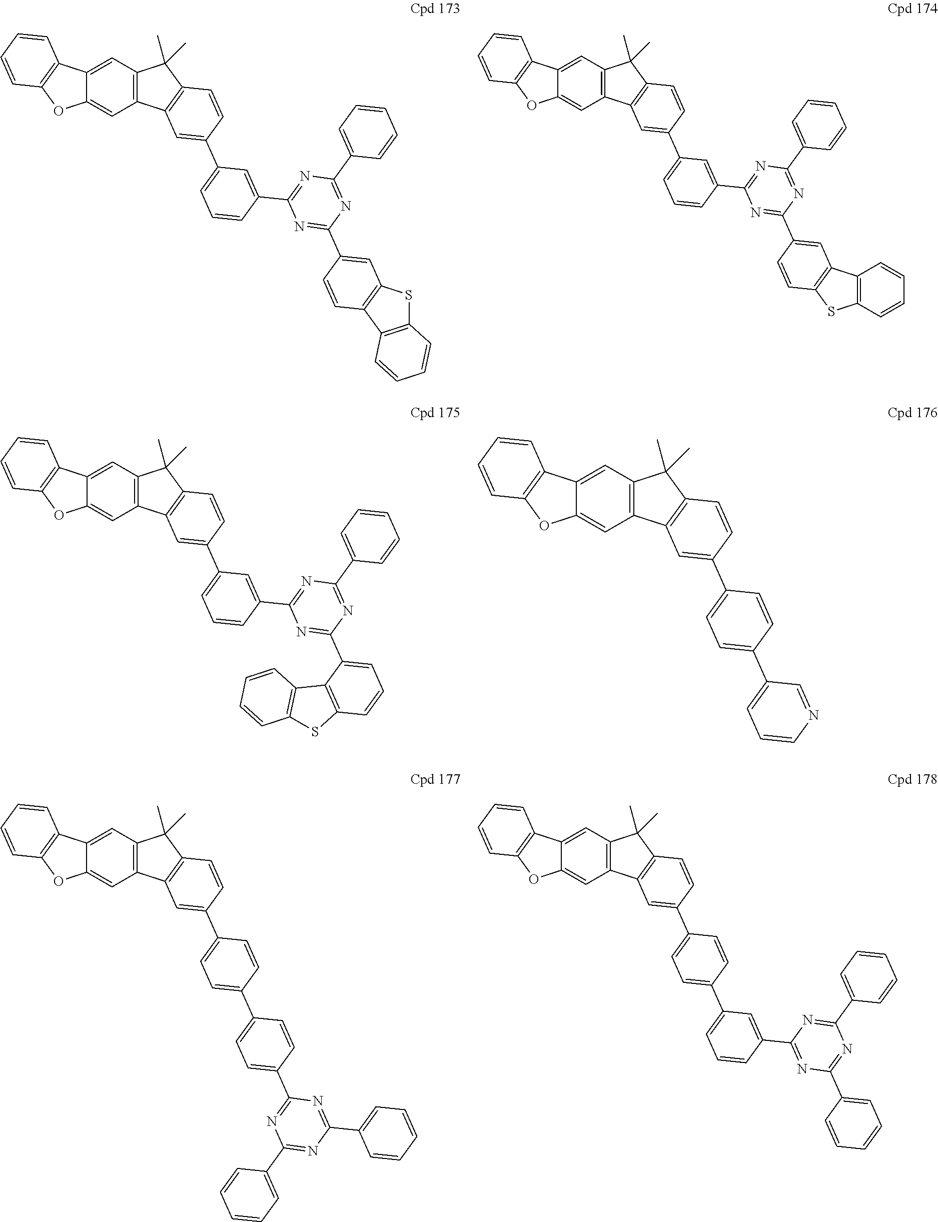

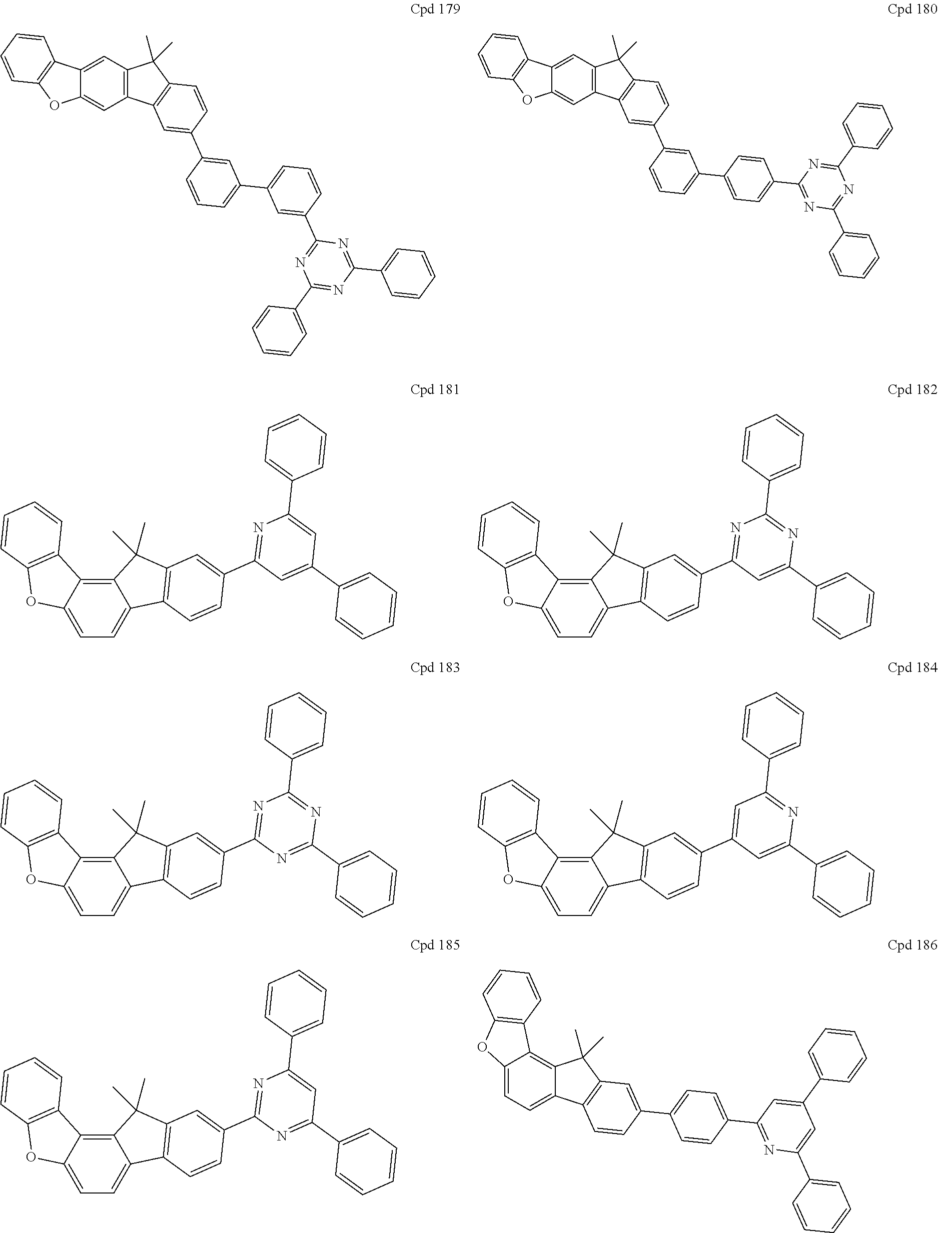

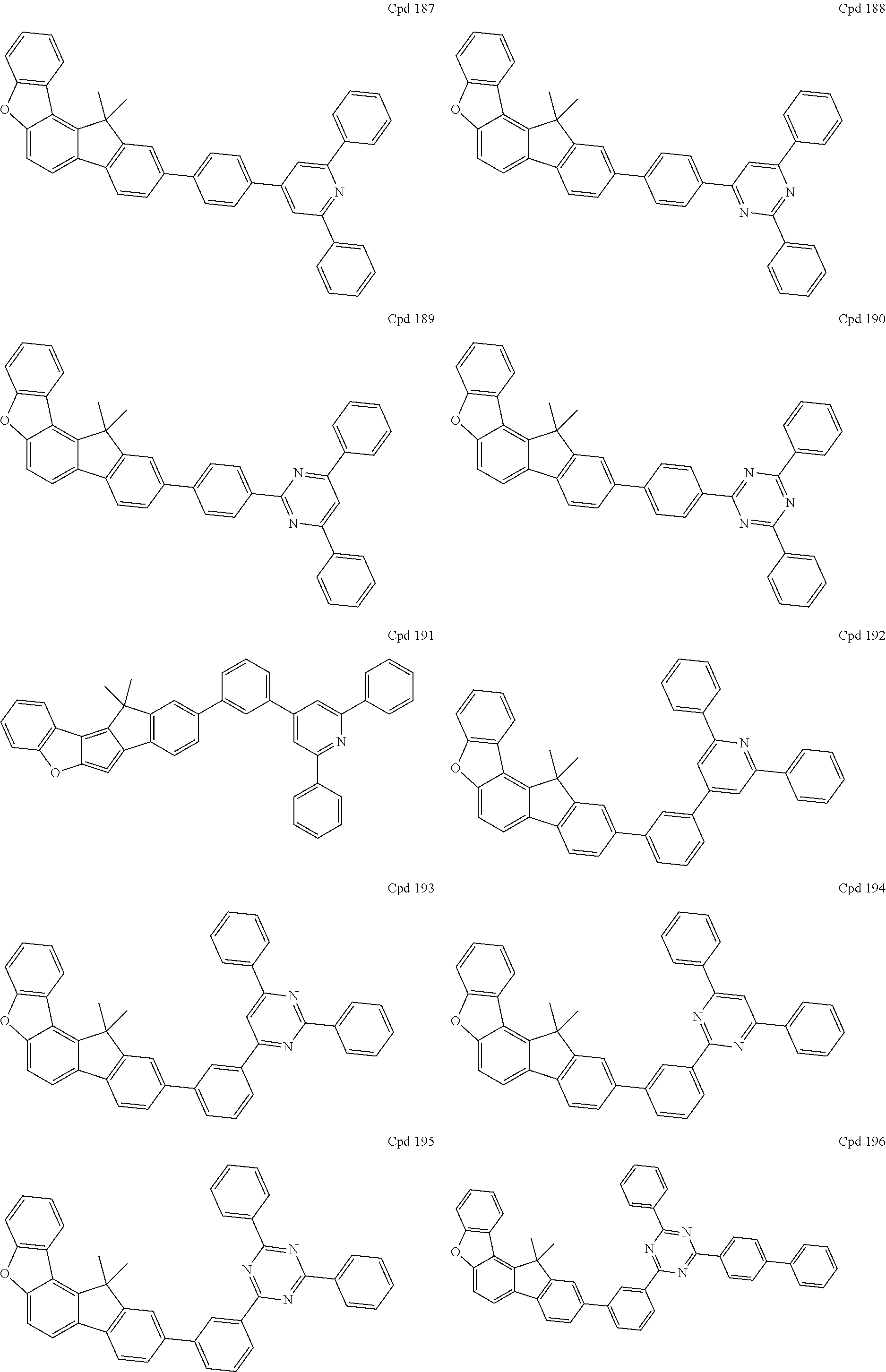

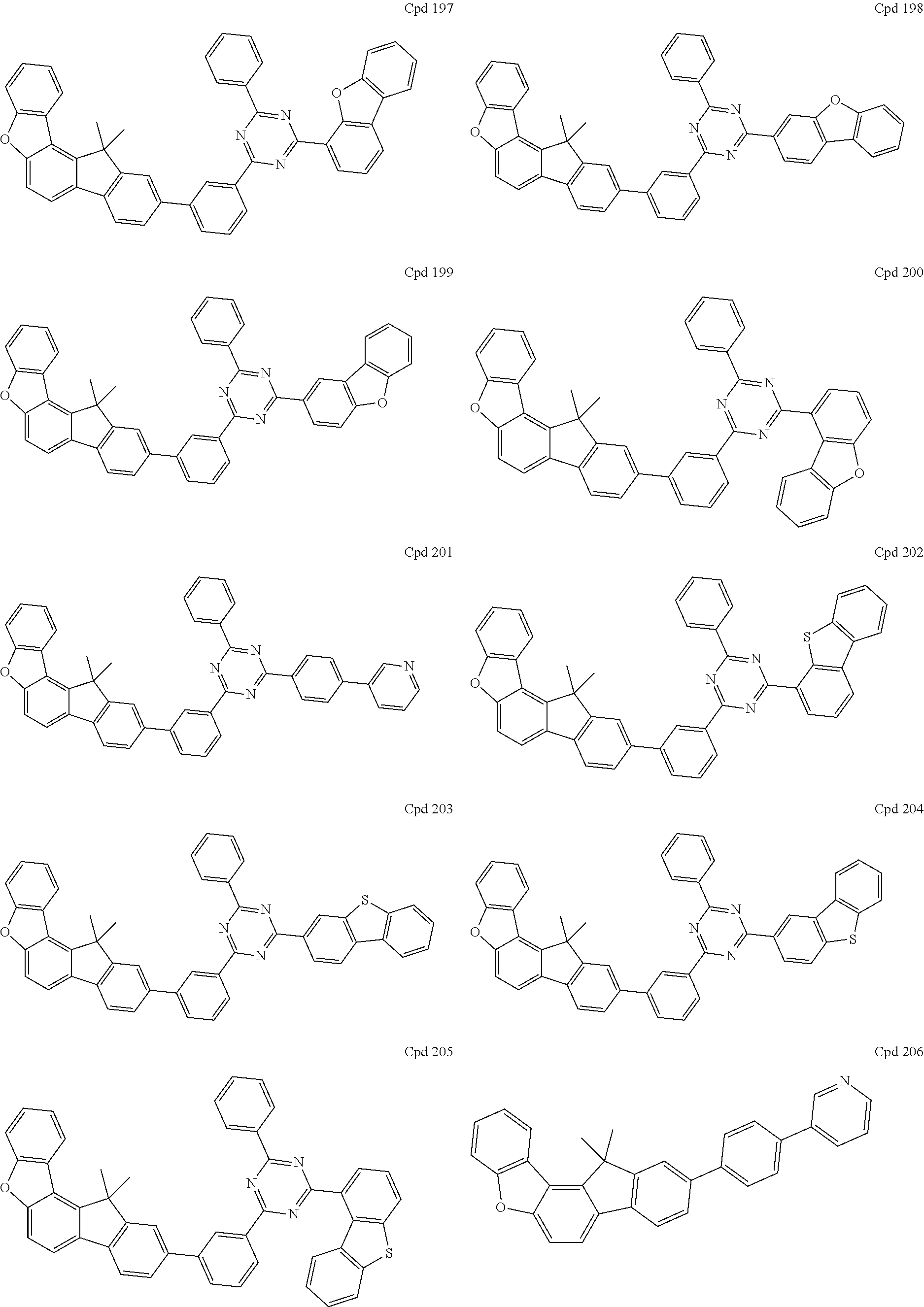

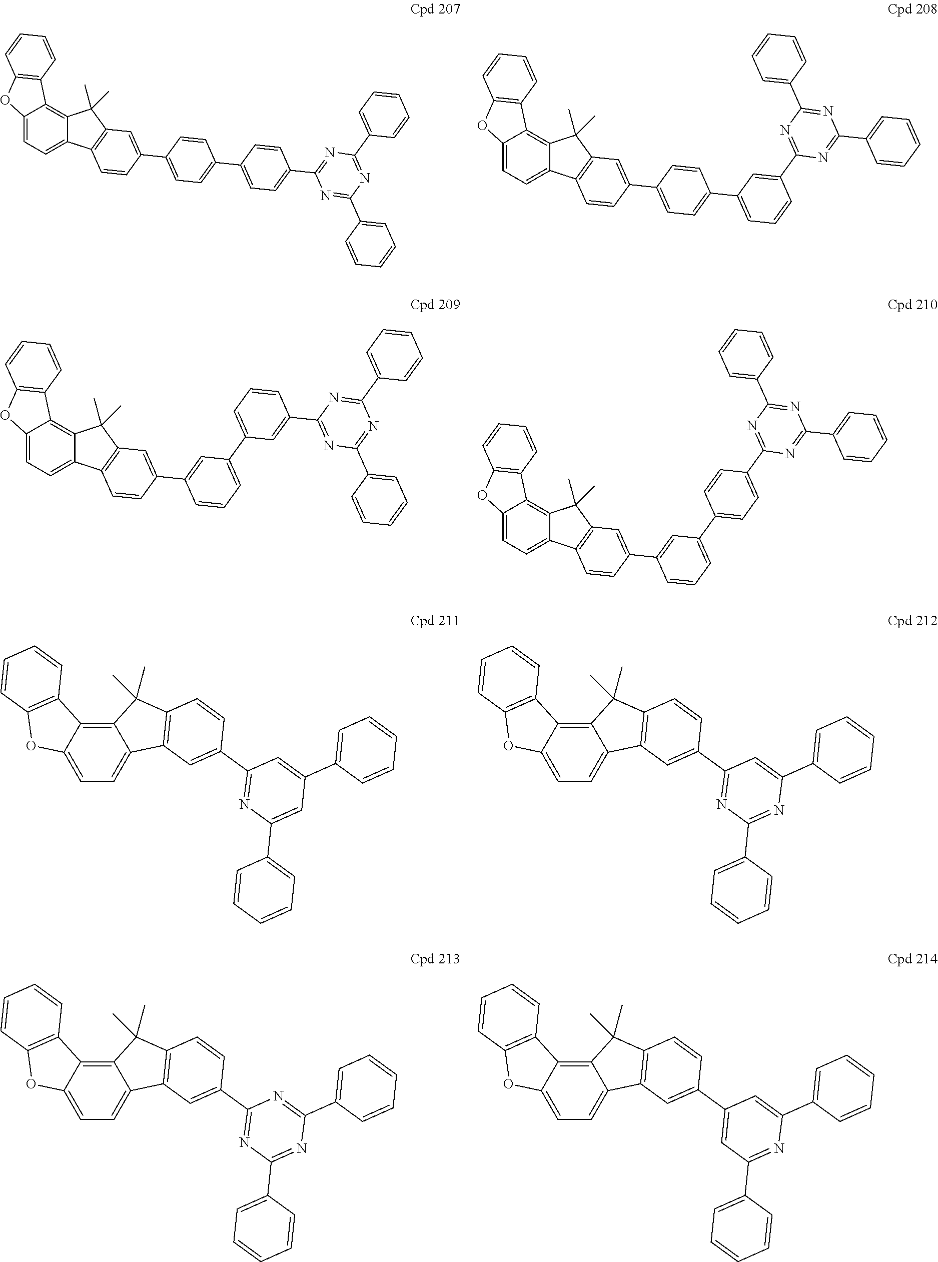

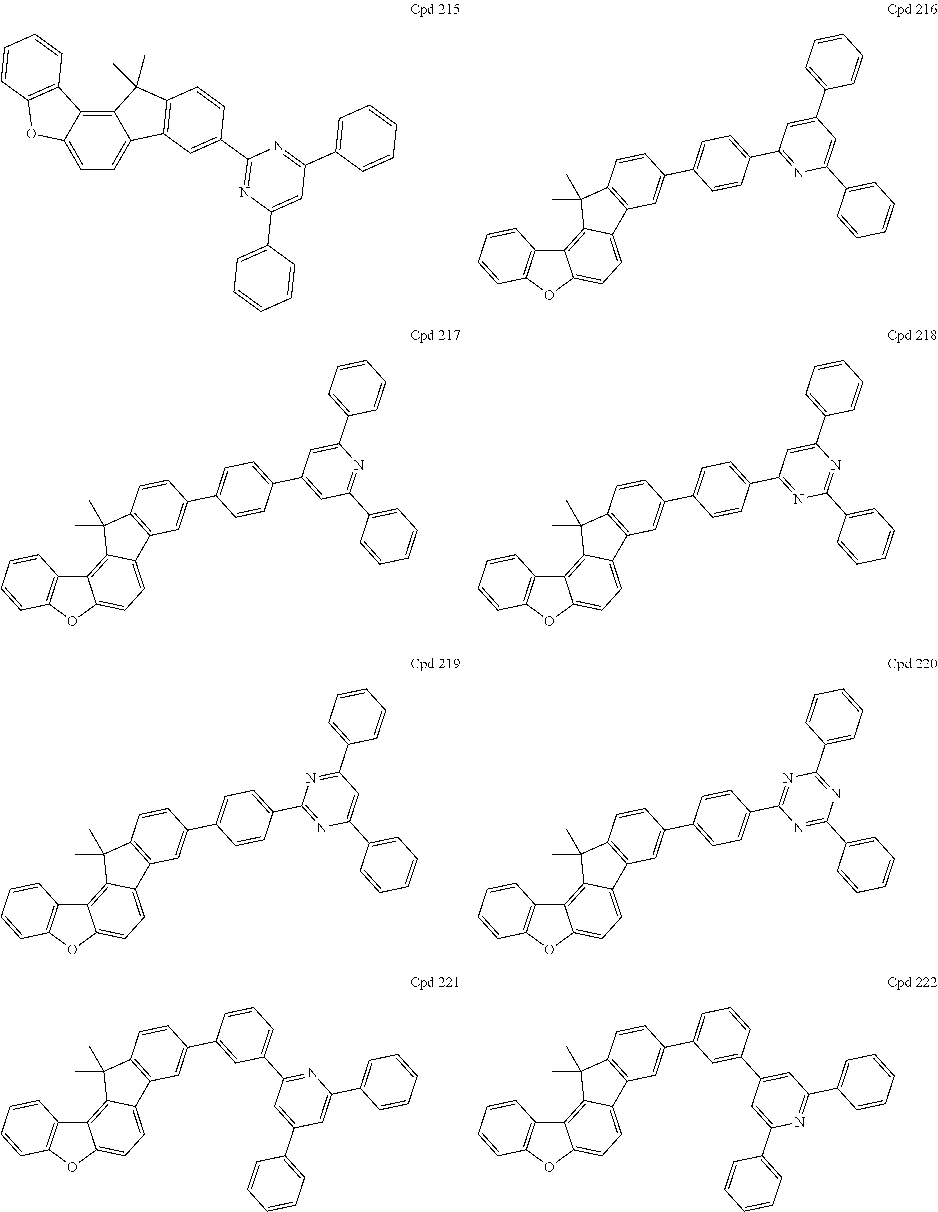

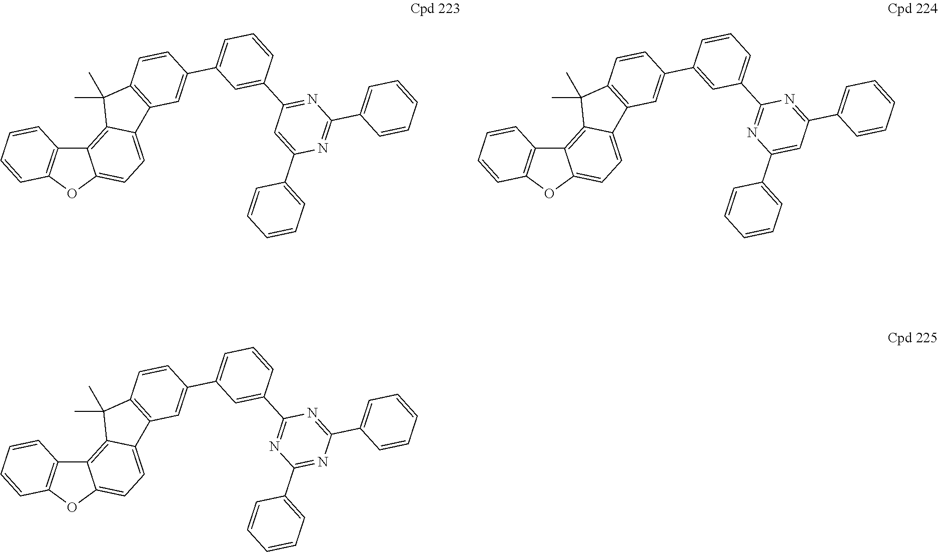 [SMILES: CC1(C)c2ccc(-c3cccc(-c4cc(-c5ccccc5)nc(-c5ccccc5)n4)c3)cc2-c2ccc3oc4ccccc4c3c21 ; CC1(C)c2ccc(-c3cccc(-c4nc(-c5ccccc5)cc(-c5ccccc5)n4)c3)cc2-c2ccc3oc4ccccc4c3c21 ; CC1(C)c2ccc(-c3cccc(-c4nc(-c5ccccc5)nc(-c5ccccc5)n4)c3)cc2-c2ccc3oc4ccccc4c3c21]

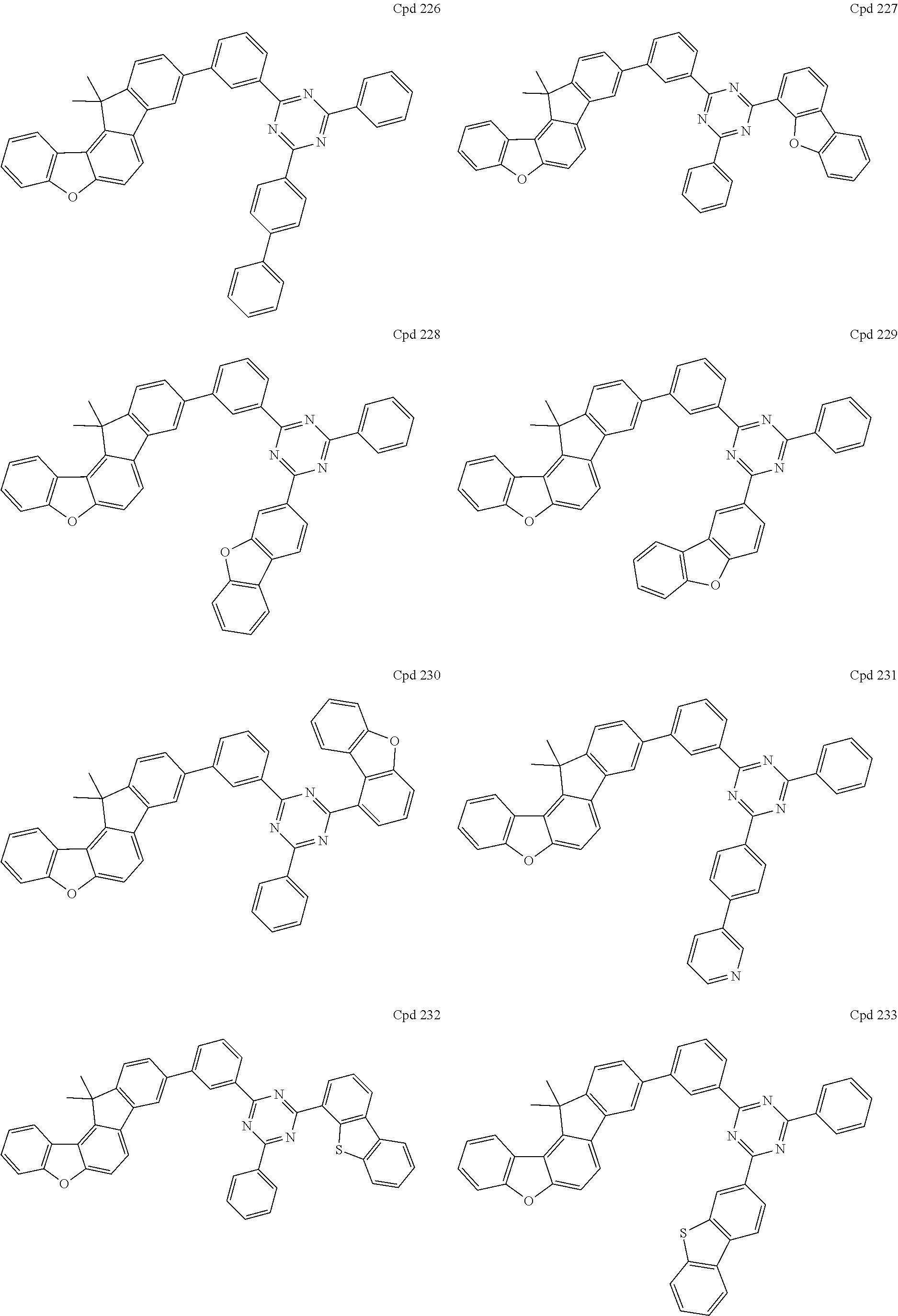

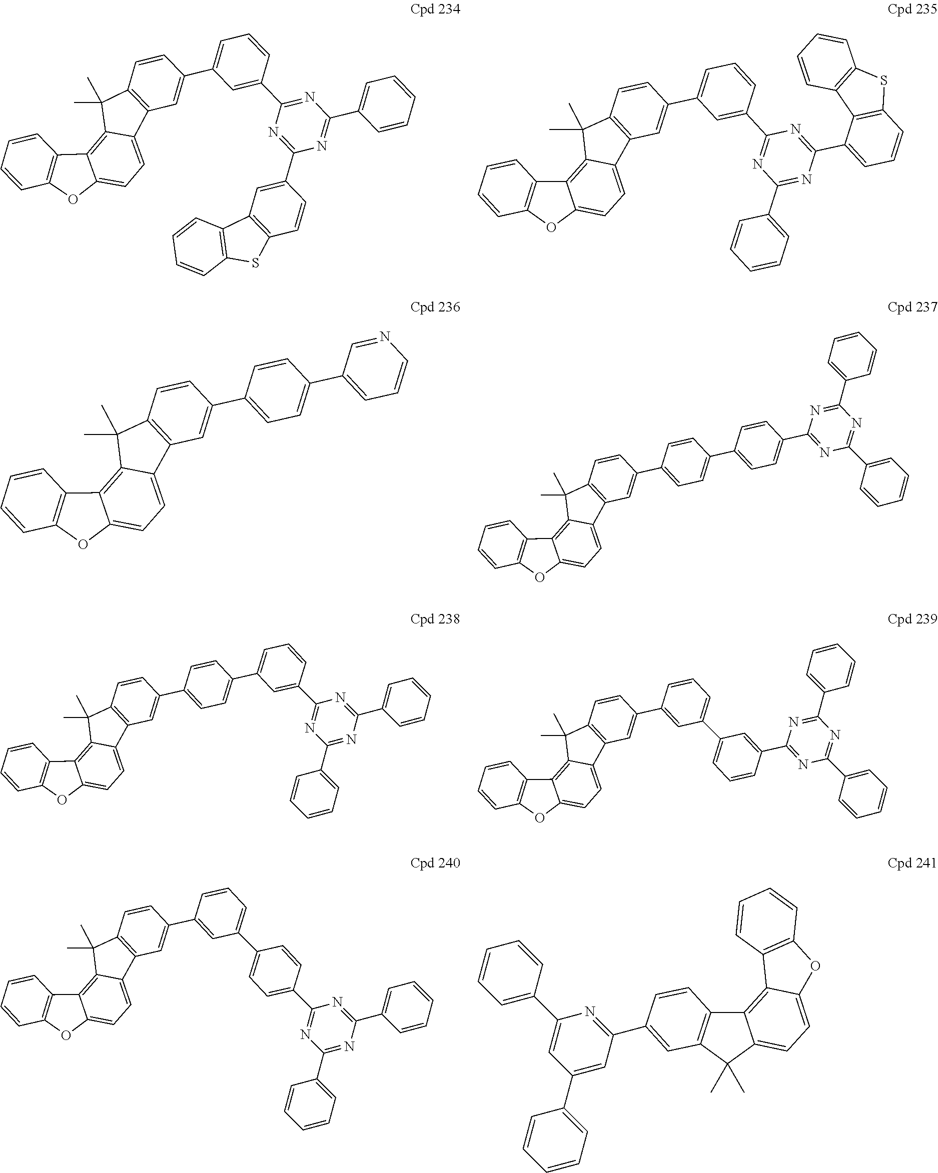

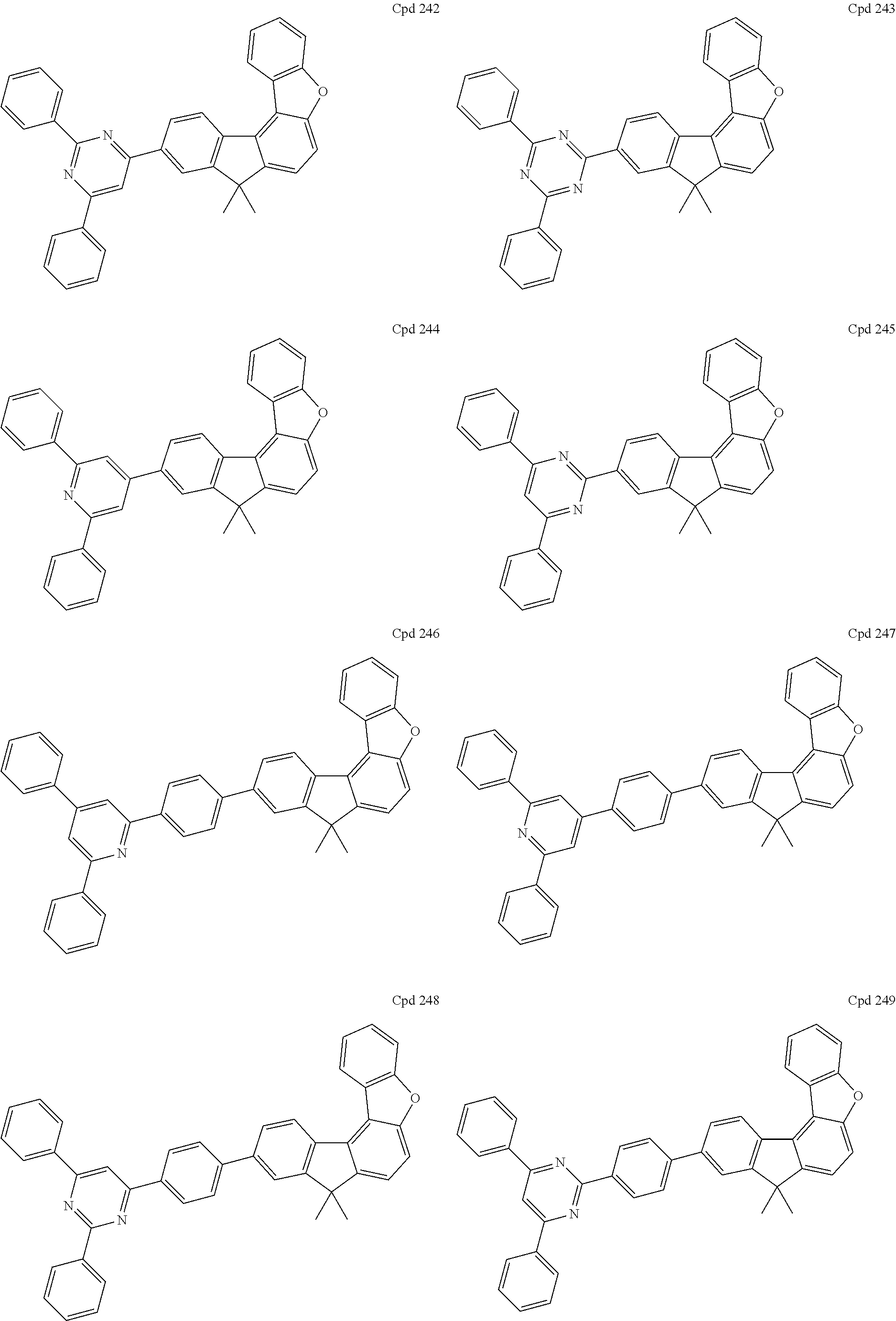

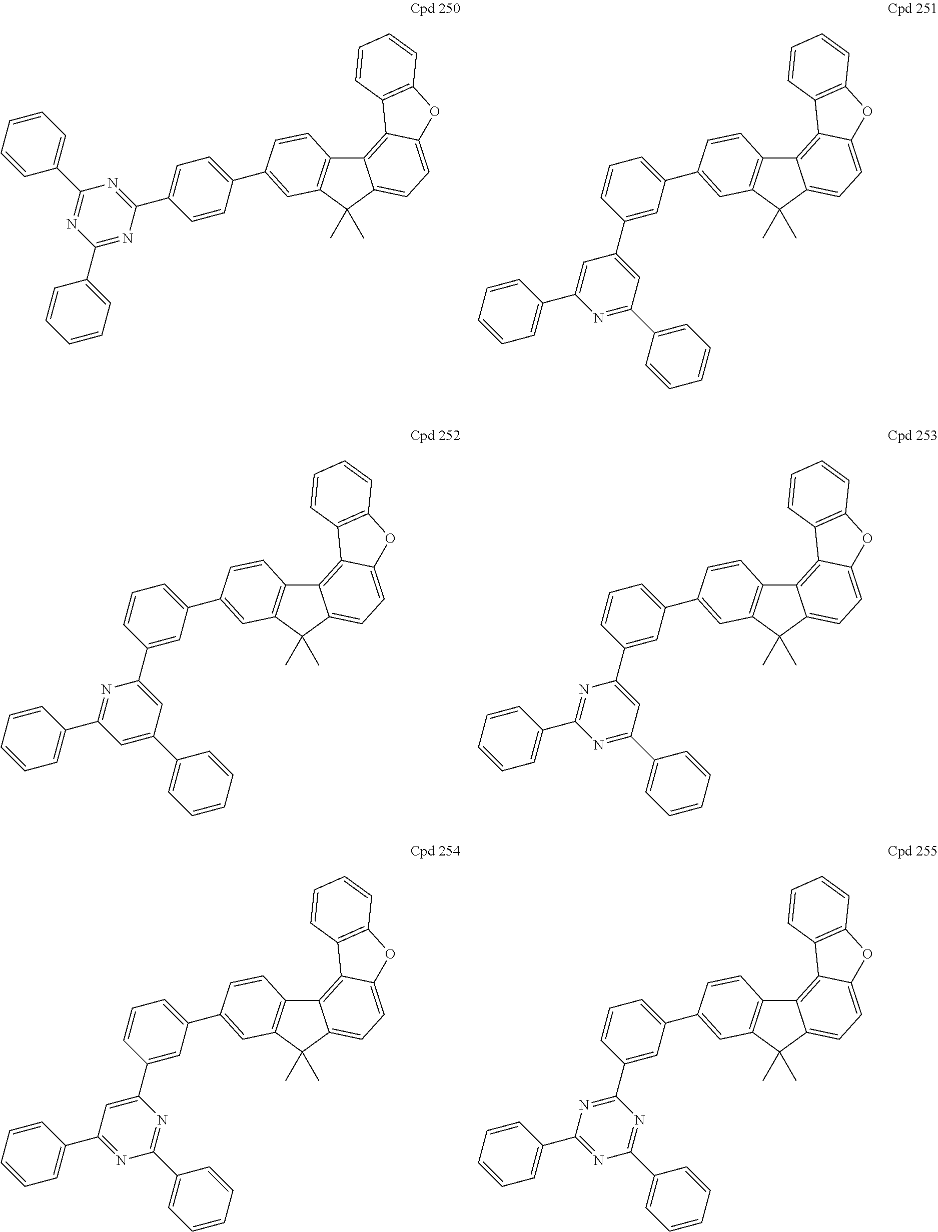 [SMILES: CC1(C)c2cc(-c3ccc(-c4nc(-c5ccccc5)nc(-c5ccccc5)n4)cc3)ccc2-c2c1ccc1oc3ccccc3c21 ; CC1(C)c2cc(-c3cccc(-c4cc(-c5ccccc5)nc(-c5ccccc5)c4)c3)ccc2-c2c1ccc1oc3ccccc3c21 ; CC1(C)c2cc(-c3cccc(-c4cc(-c5ccccc5)cc(-c5ccccc5)n4)c3)ccc2-c2c1ccc1oc3ccccc3c21 ; CC1(C)c2cc(-c3cccc(-c4cc(-c5ccccc5)nc(-c5ccccc5)n4)c3)ccc2-c2c1ccc1oc3ccccc3c21 ; CC1(C)c2cc(-c3cccc(-c4cc(-c5ccccc5)nc(-c5ccccc5)n4)c3)ccc2-c2c1ccc1oc3ccccc3c21 ; CC1(C)c2cc(-c3cccc(-c4nc(-c5ccccc5)nc(-c5ccccc5)n4)c3)ccc2-c2c1ccc1oc3ccccc3c21]

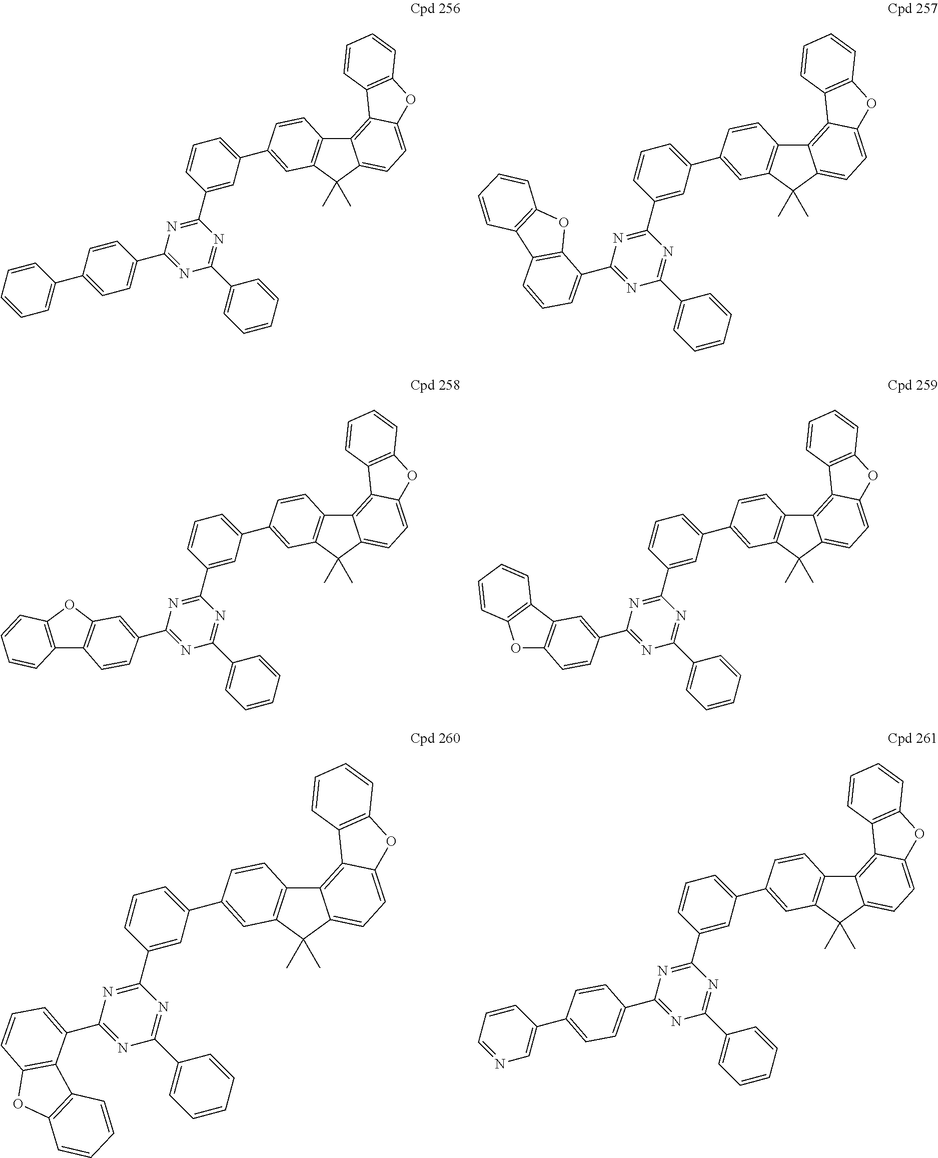

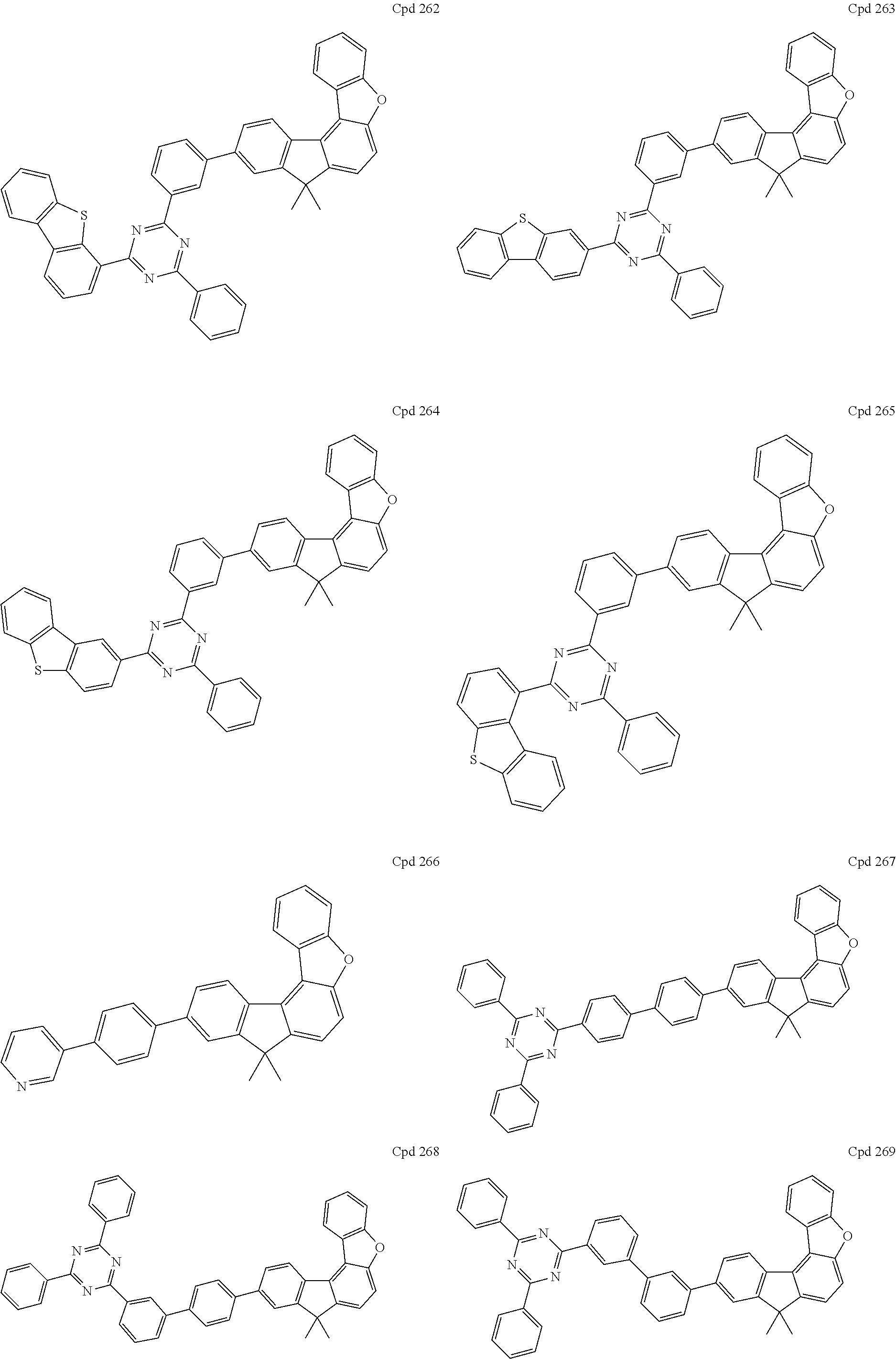

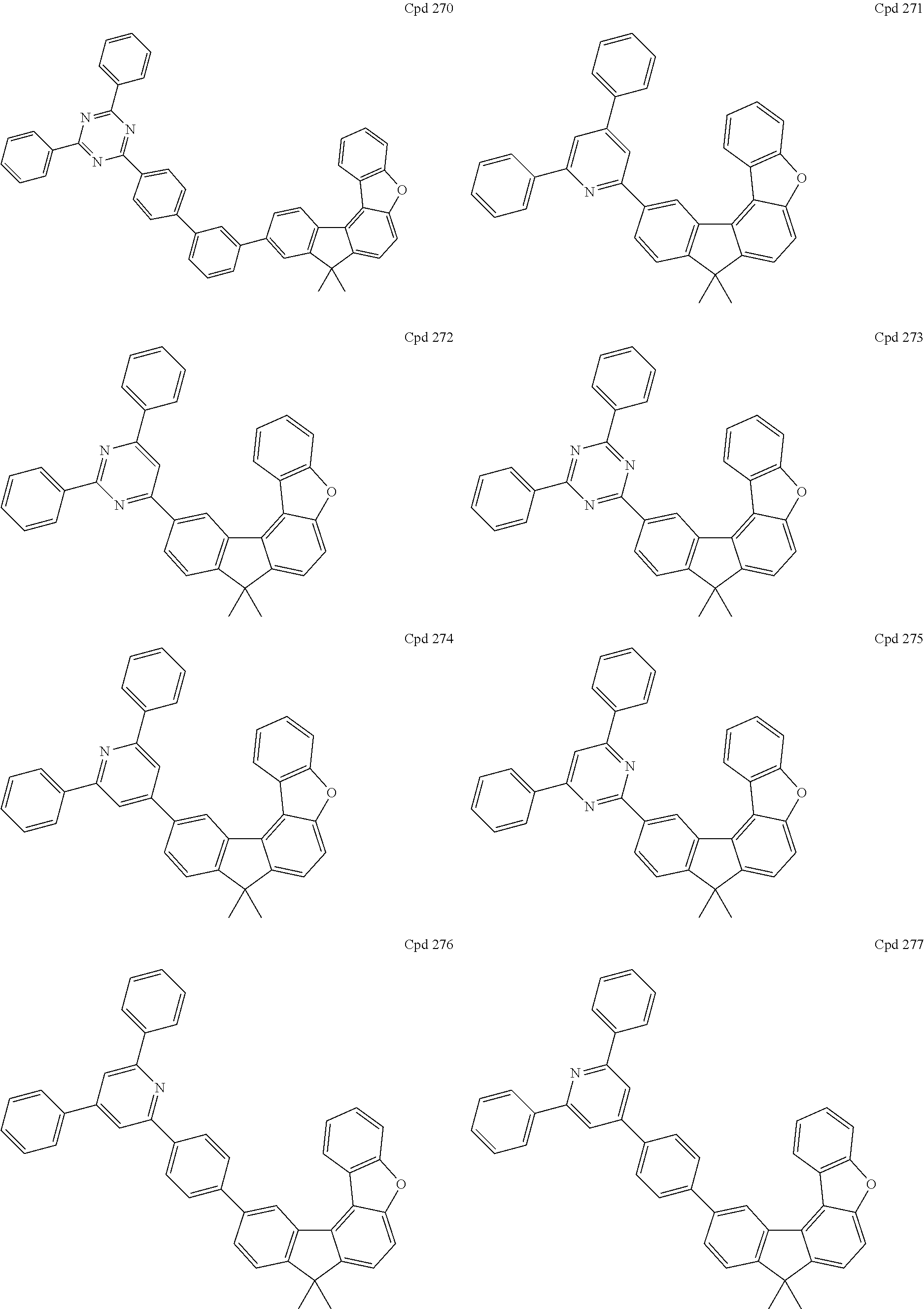

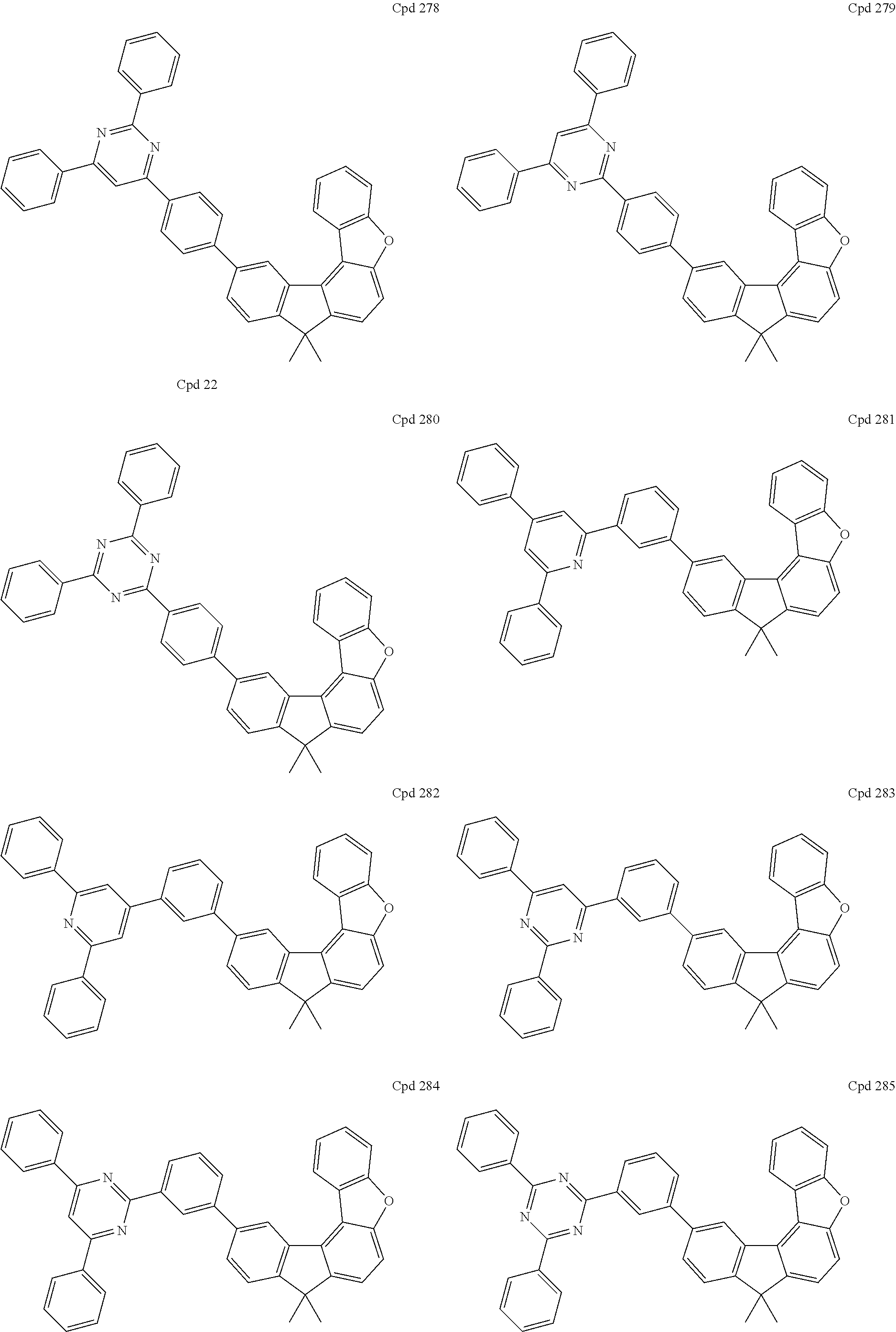

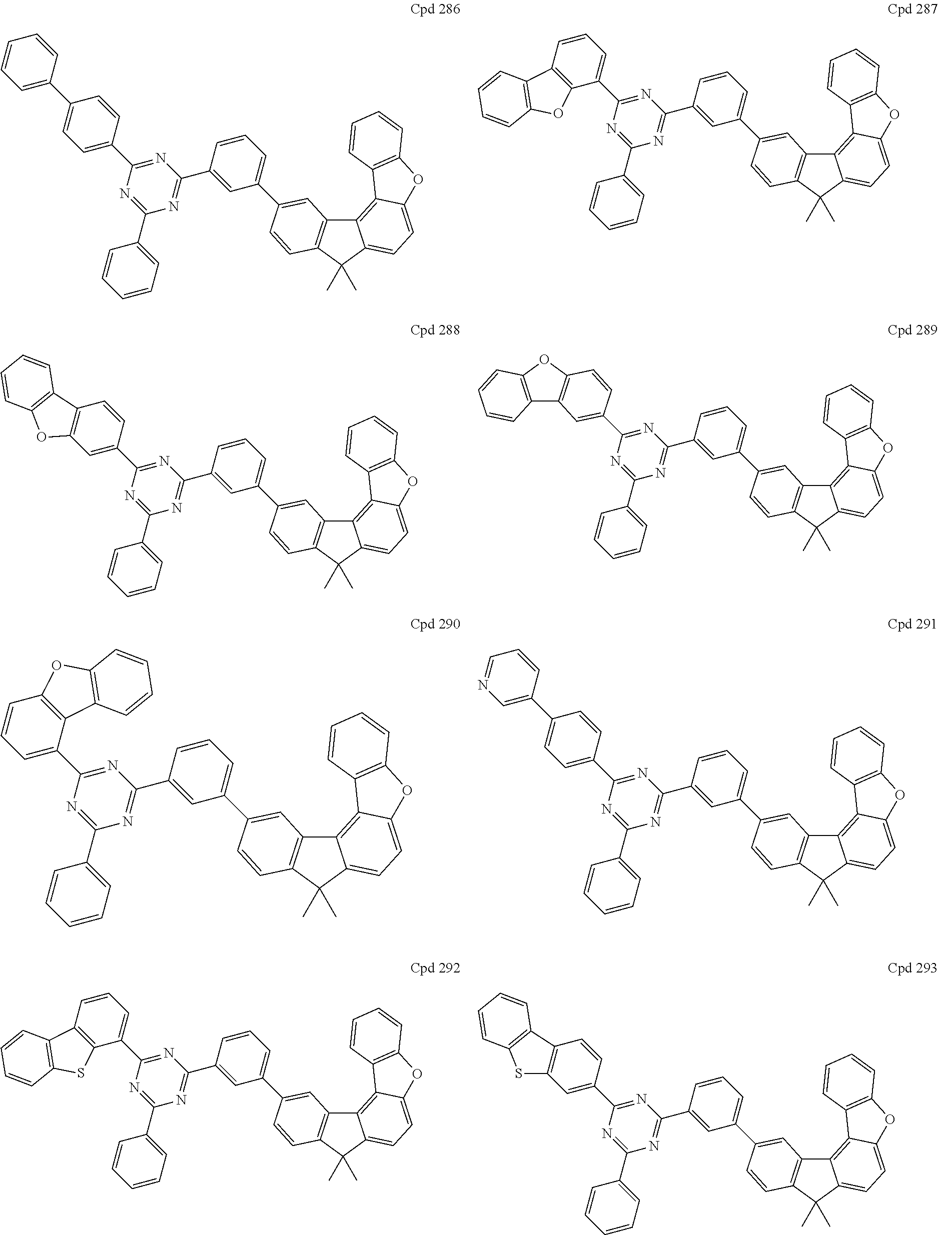

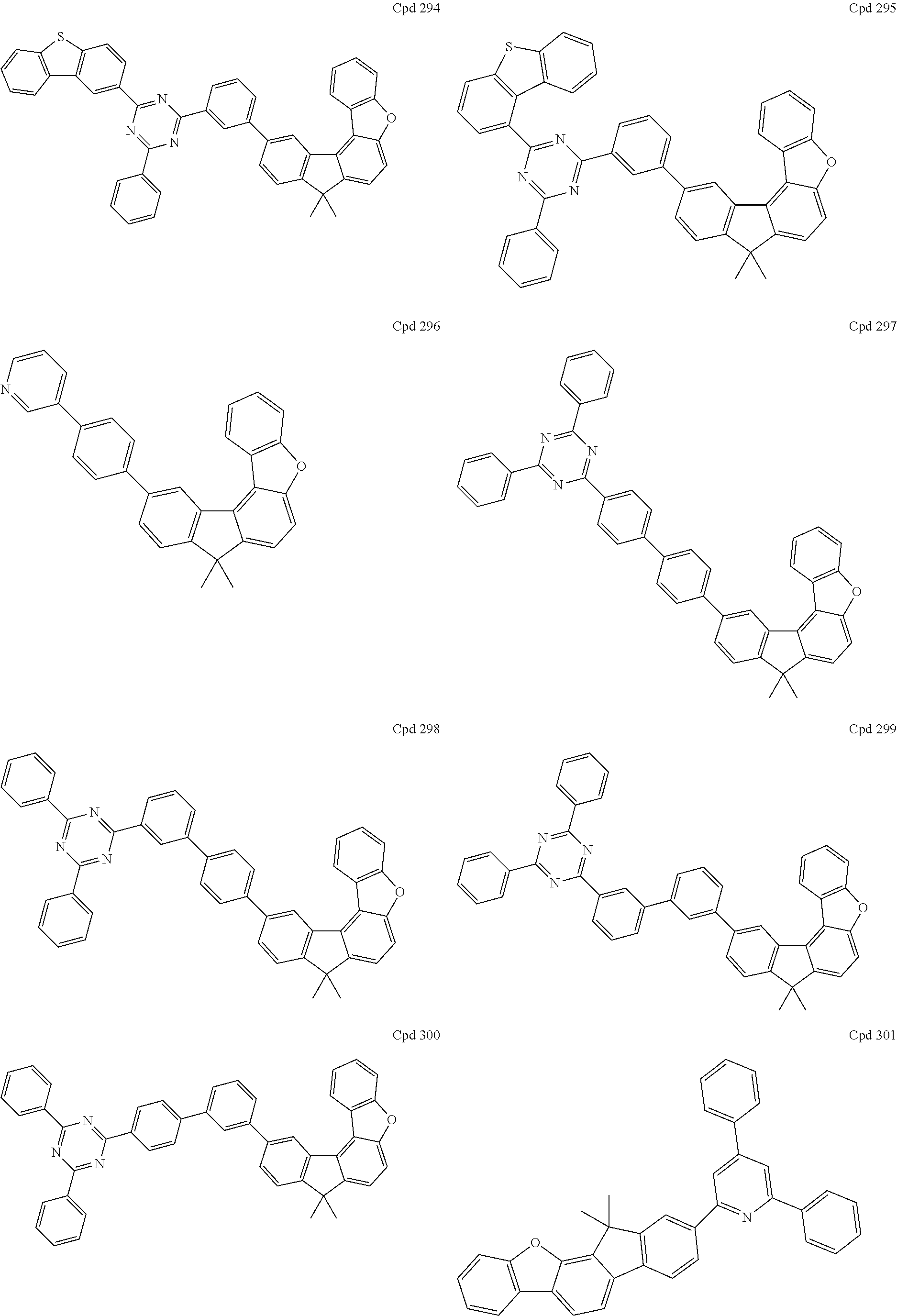

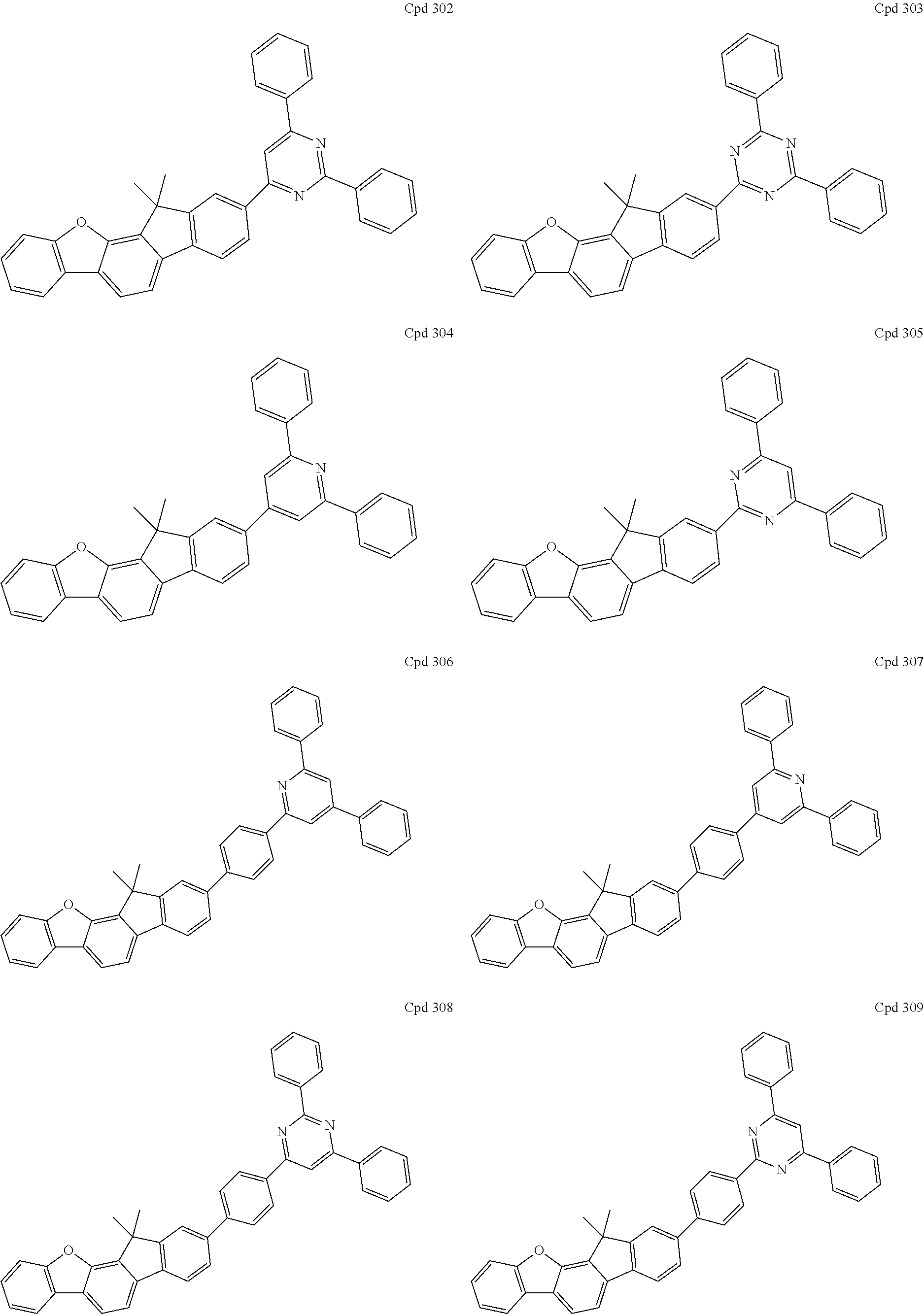

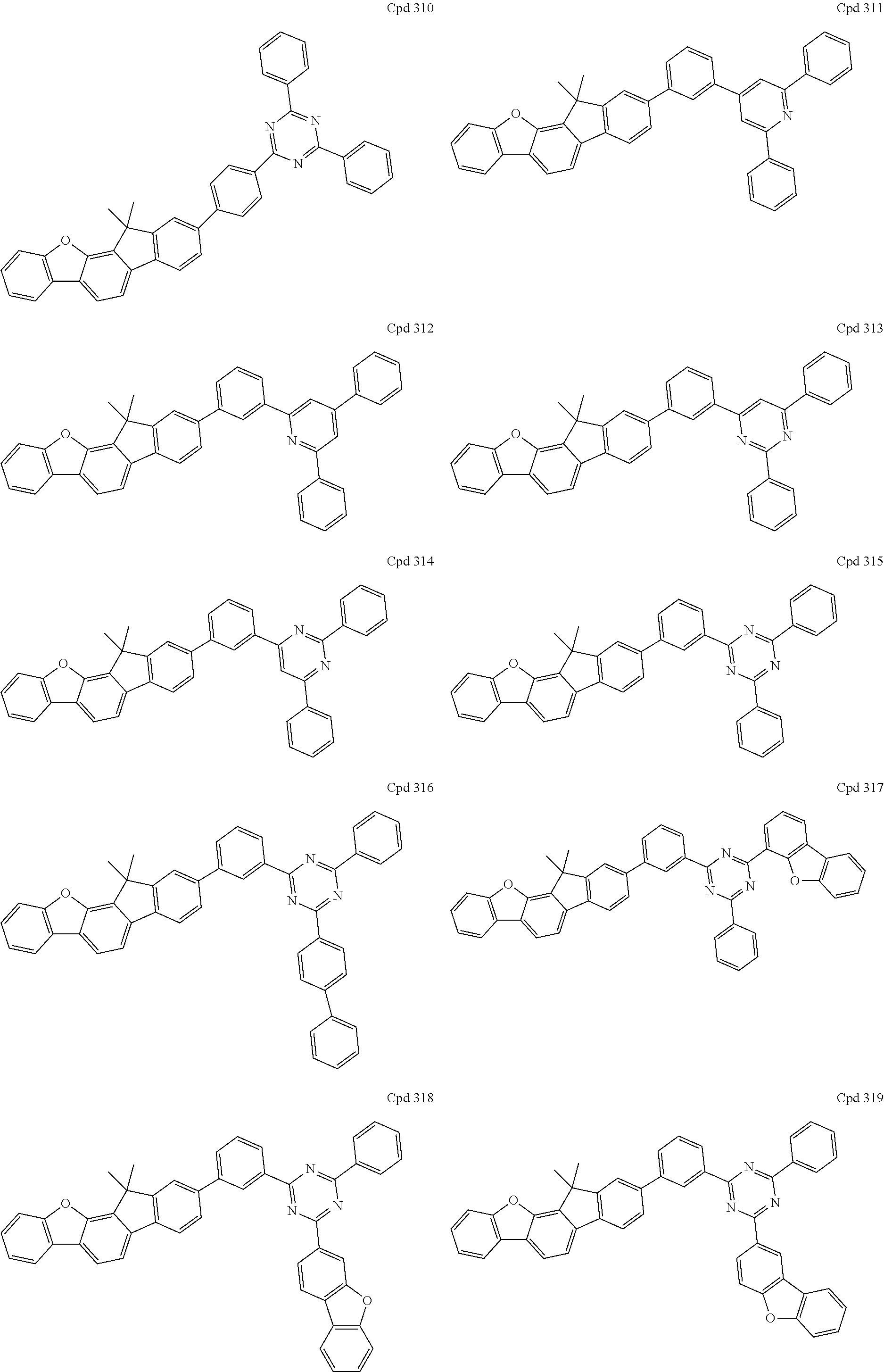

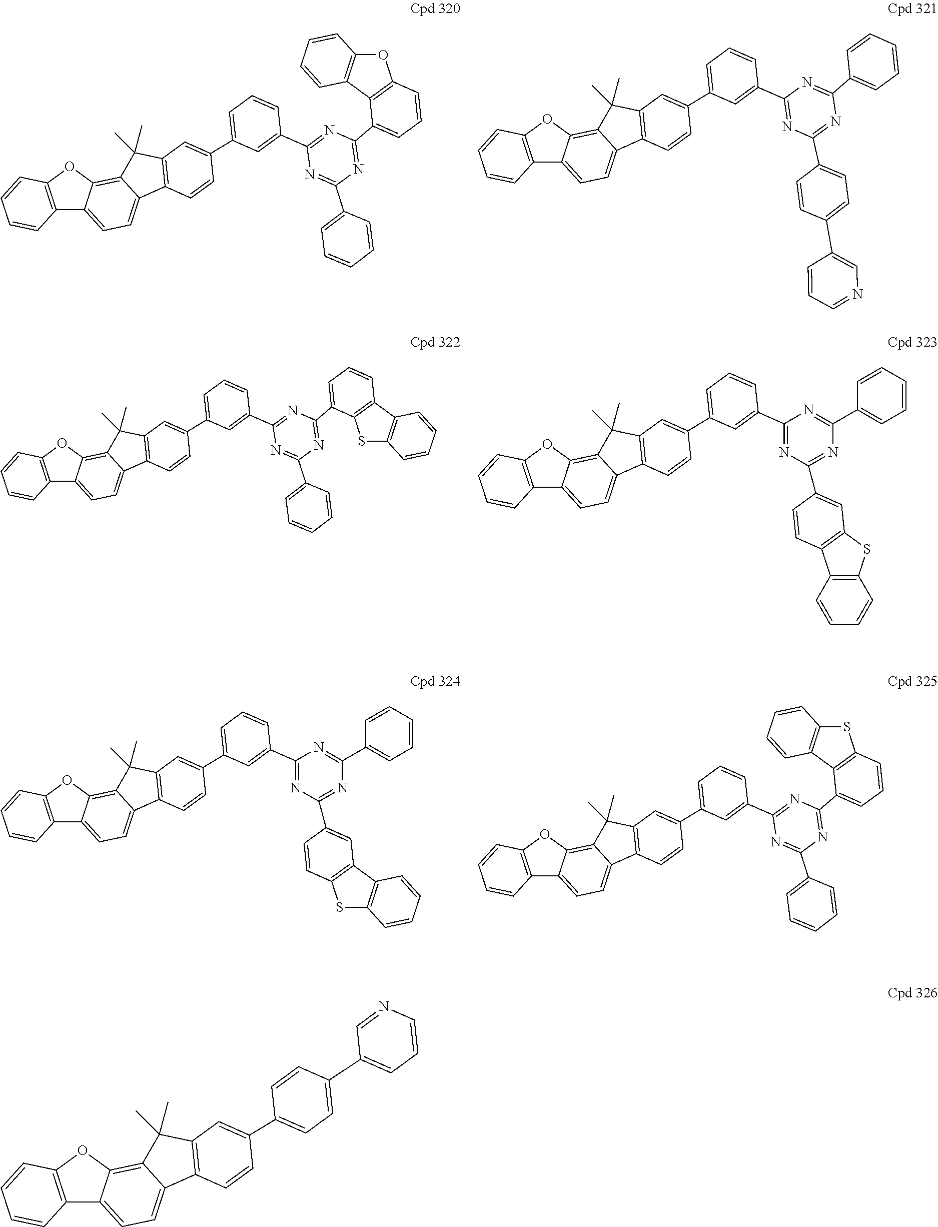

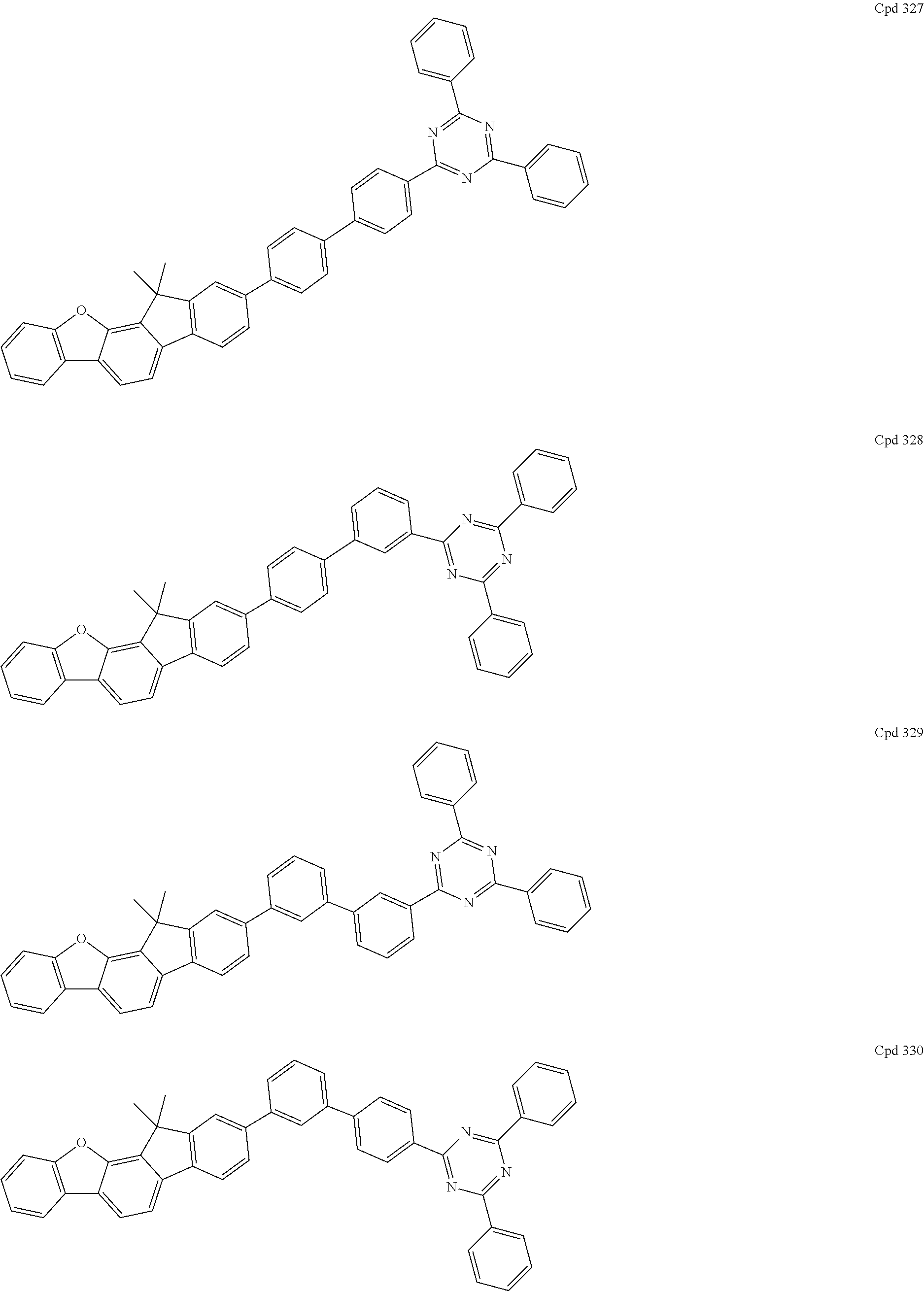 [SMILES: CC1(C)c2cc(-c3ccc(-c4ccc(-c5nc(-c6ccccc6)nc(-c6ccccc6)n5)cc4)cc3)ccc2-c2ccc3c(oc4ccccc43)c21 ; CC1(C)c2cc(-c3ccc(-c4cccc(-c5nc(-c6ccccc6)nc(-c6ccccc6)n5)c4)cc3)ccc2-c2ccc3c(oc4ccccc43)c21 ; CC1(C)c2cc(-c3cccc(-c4cccc(-c5nc(-c6ccccc6)nc(-c6ccccc6)n5)c4)c3)ccc2-c2ccc3c(oc4ccccc43)c21 ; CC1(C)c2cc(-c3cccc(-c4ccc(-c5nc(-c6ccccc6)nc(-c6ccccc6)n5)cc4)c3)ccc2-c2ccc3c(oc4ccccc43)c21]

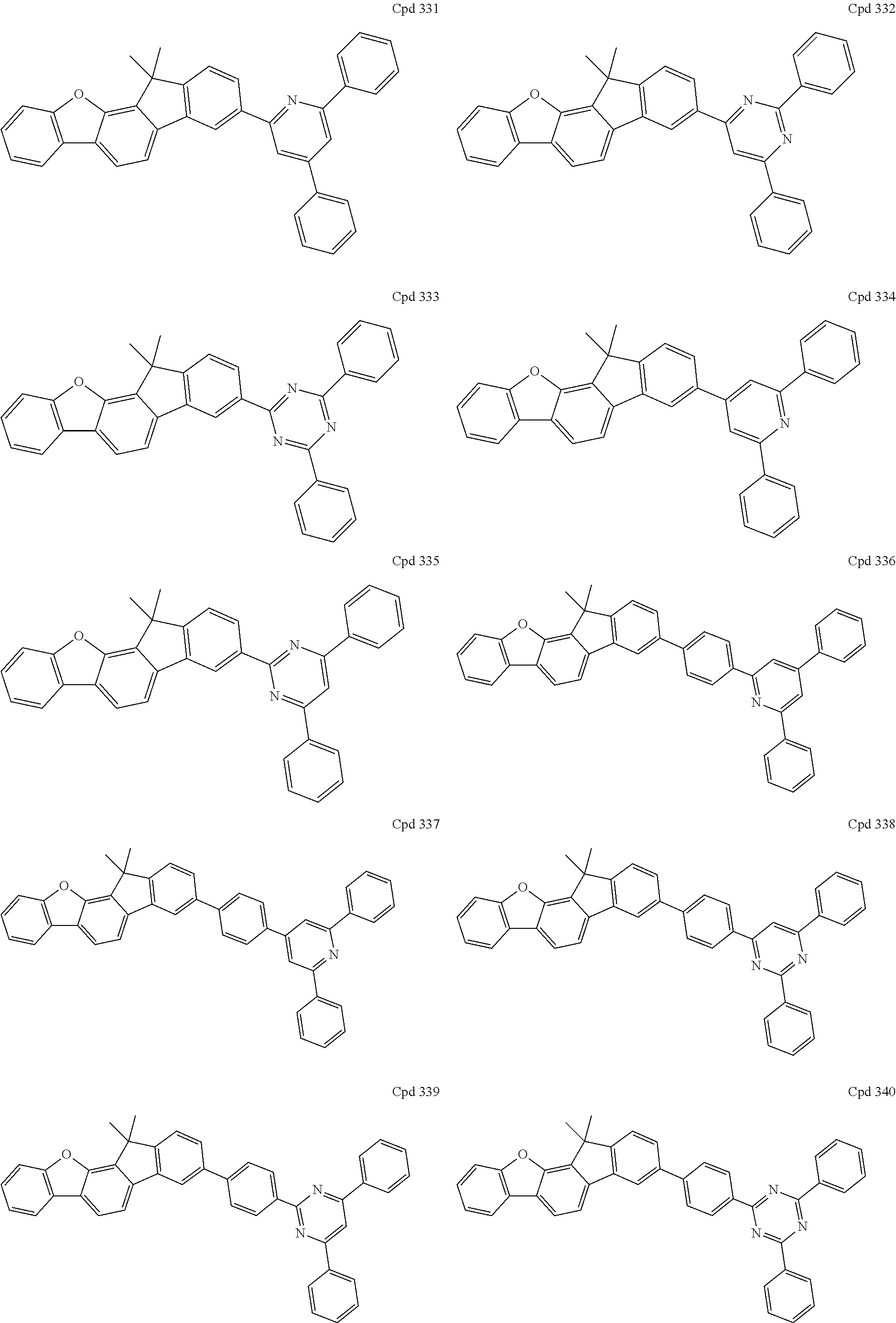

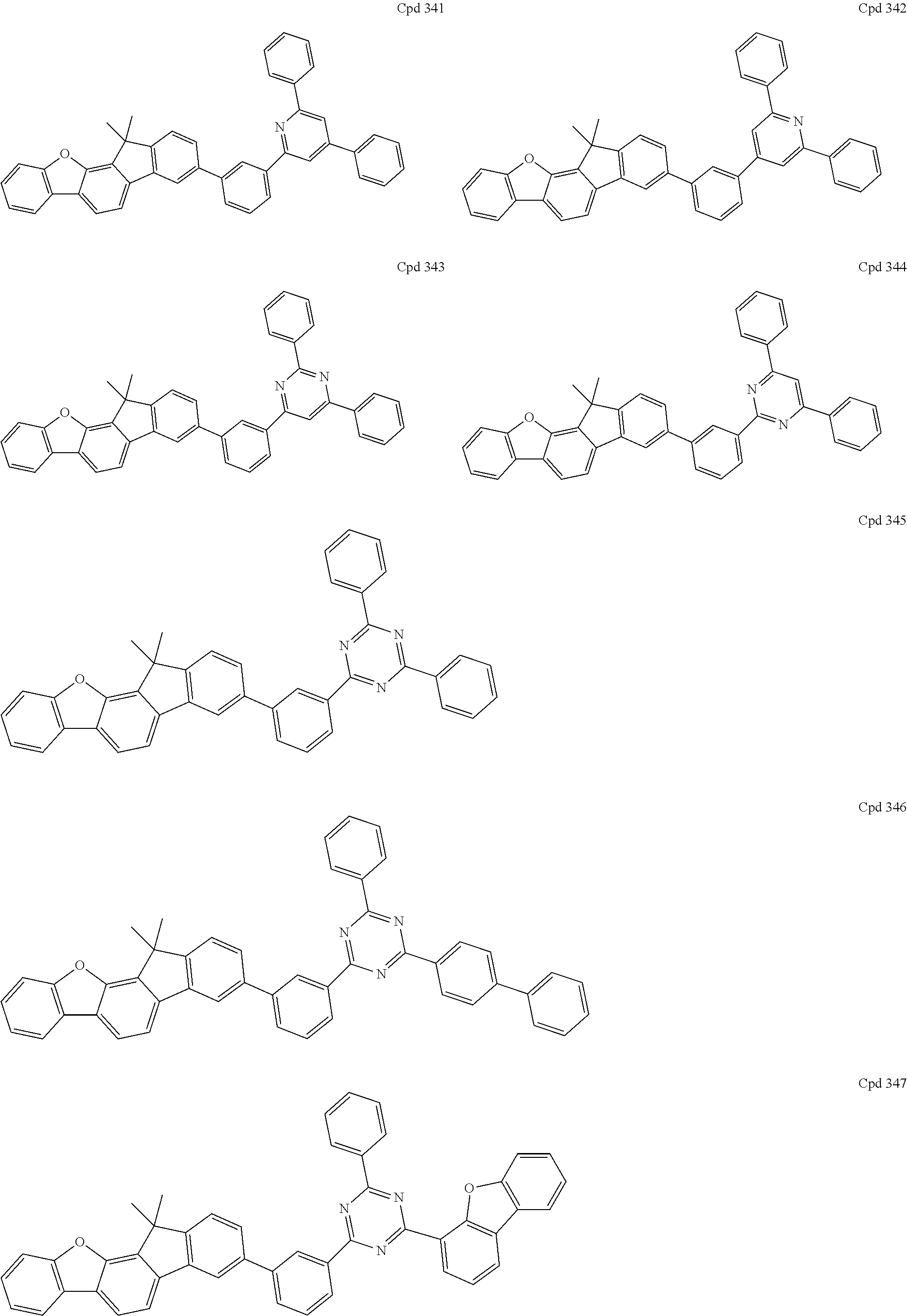

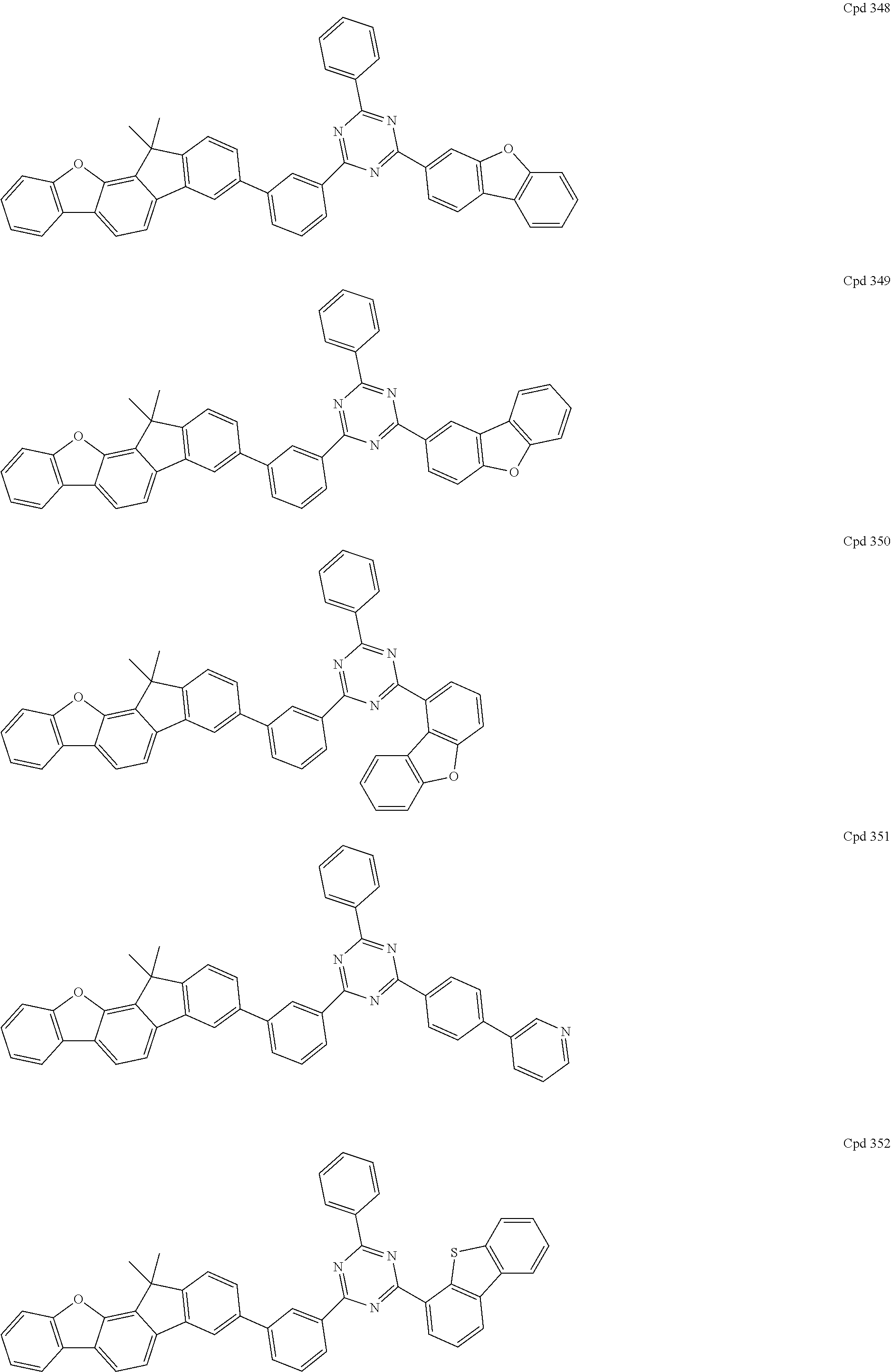 [SMILES: CC1(C)c2ccc(-c3cccc(-c4nc(-c5ccccc5)nc(-c5ccc6c(c5)oc5ccccc56)n4)c3)cc2-c2ccc3c(oc4ccccc43)c21 ; CC1(C)c2ccc(-c3cccc(-c4nc(-c5ccccc5)nc(-c5ccc6oc7ccccc7c6c5)n4)c3)cc2-c2ccc3c(oc4ccccc43)c21 ; CC1(C)c2ccc(-c3cccc(-c4nc(-c5ccccc5)nc(-c5cccc6oc7ccccc7c56)n4)c3)cc2-c2ccc3c(oc4ccccc43)c21 ; CC1(C)c2ccc(-c3cccc(-c4nc(-c5ccccc5)nc(-c5ccc(-c6cccnc6)cc5)n4)c3)cc2-c2ccc3c(oc4ccccc43)c21 ; CC1(C)c2ccc(-c3cccc(-c4nc(-c5ccccc5)nc(-c5cccc6c5sc5ccccc56)n4)c3)cc2-c2ccc3c(oc4ccccc43)c21]

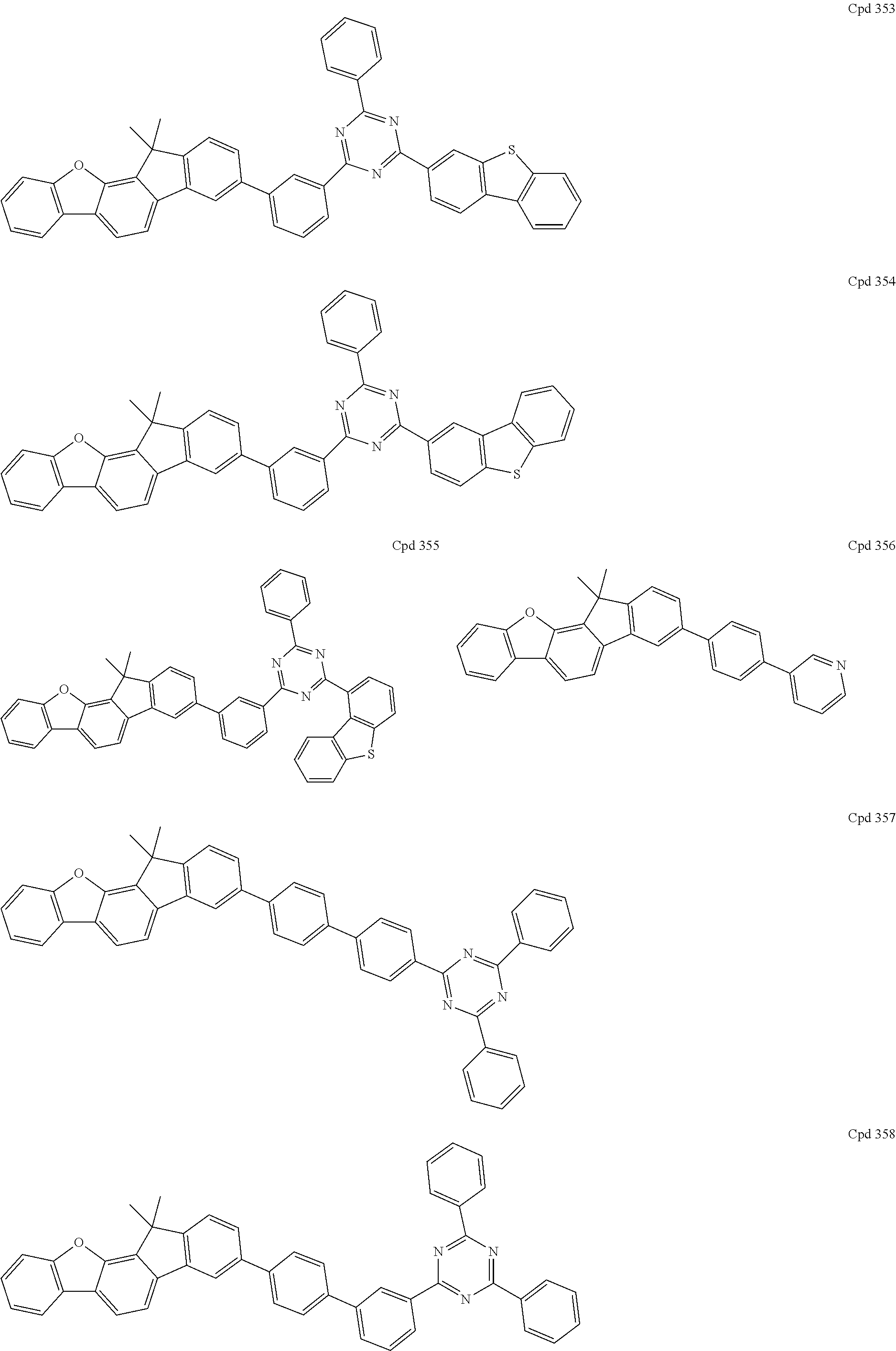

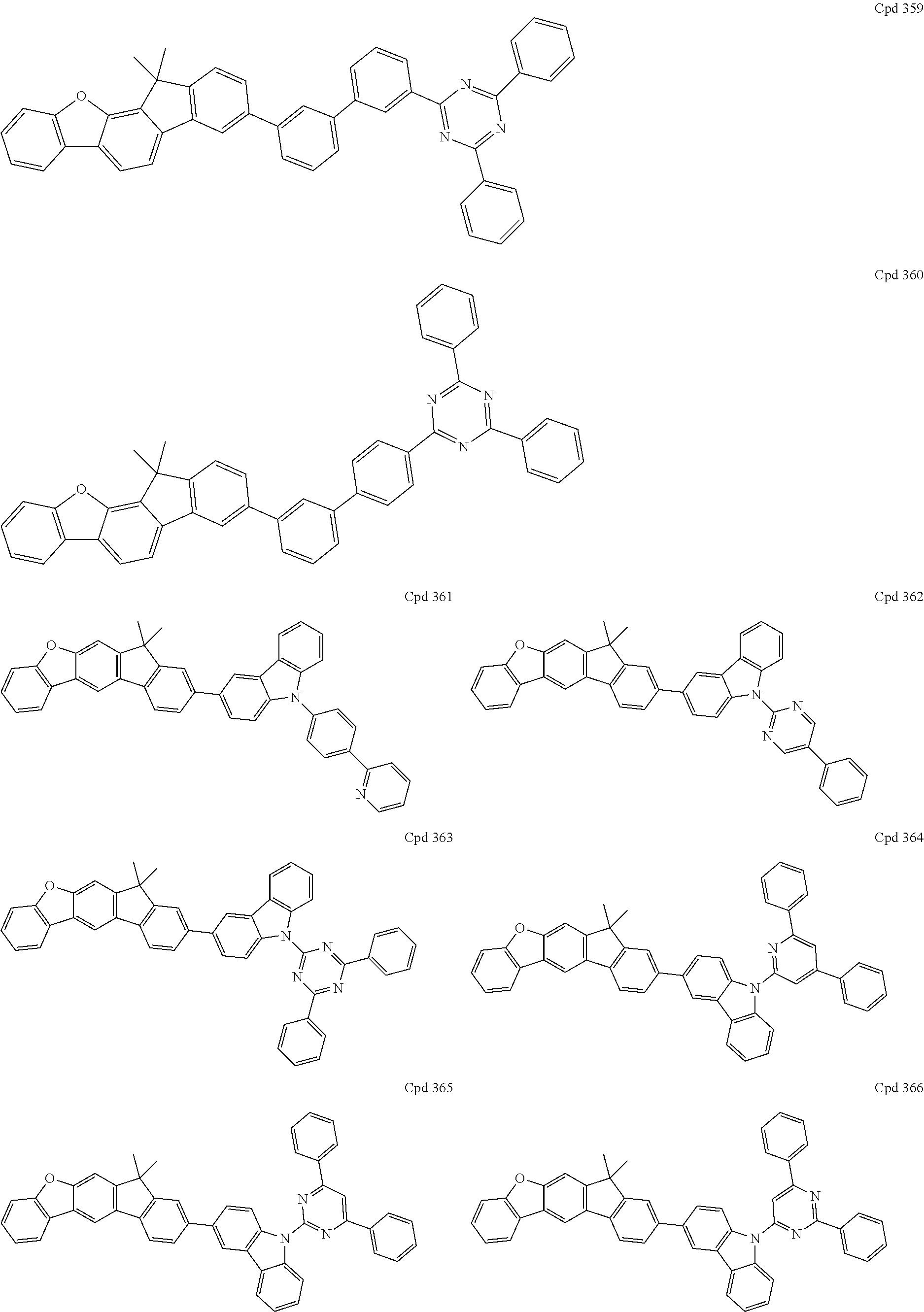

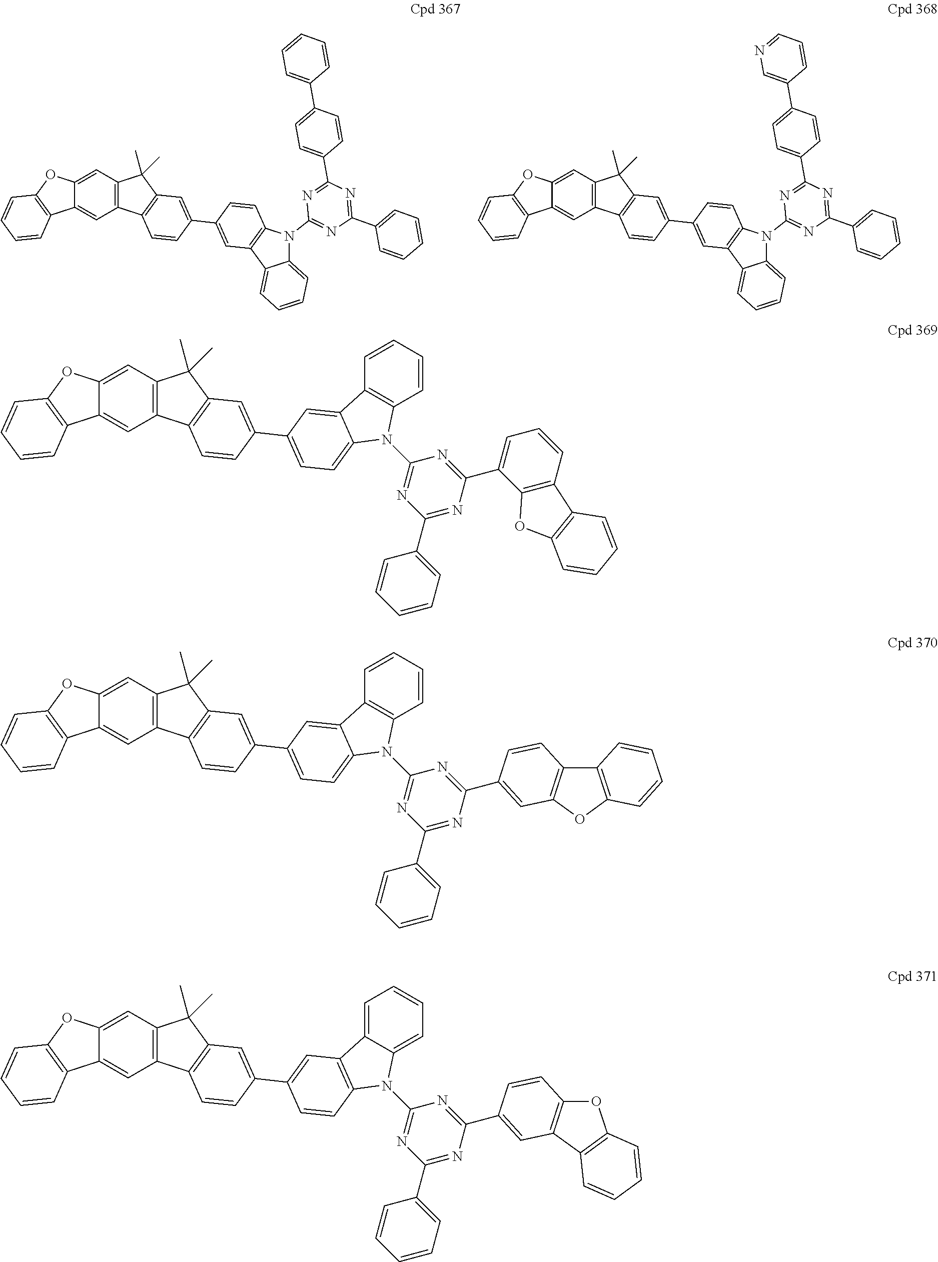

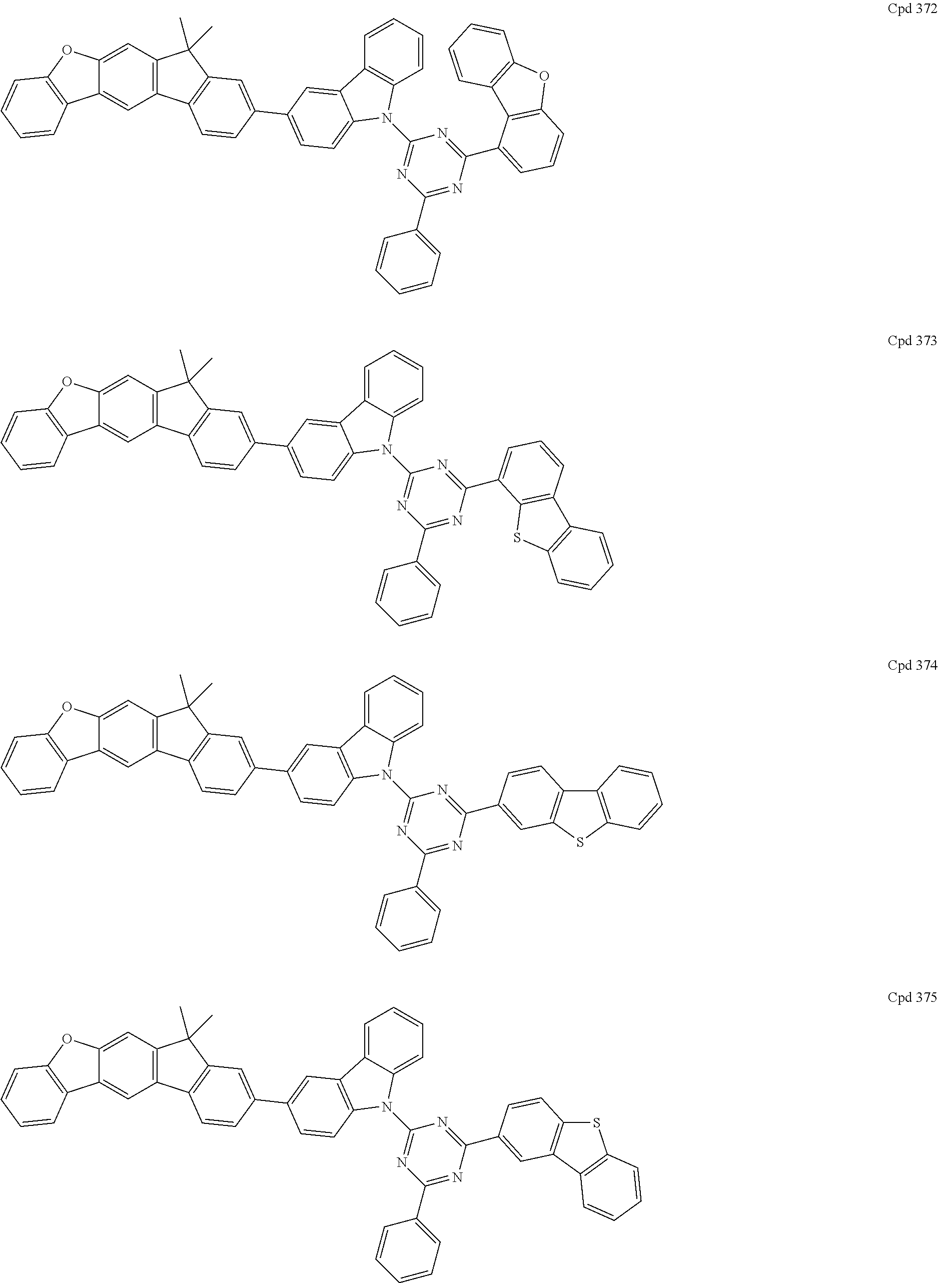 [SMILES: CC1(C)c2cc(-c3ccc4c(c3)c3ccccc3n4-c3nc(-c4ccccc4)nc(-c4cccc5oc6ccccc6c45)n3)ccc2-c2cc3c(cc21)oc1ccccc13 ; CC1(C)c2cc(-c3ccc4c(c3)c3ccccc3n4-c3nc(-c4ccccc4)nc(-c4cccc5c4sc4ccccc45)n3)ccc2-c2cc3c(cc21)oc1ccccc13 ; CC1(C)c2cc(-c3ccc4c(c3)c3ccccc3n4-c3nc(-c4ccccc4)nc(-c4ccc5c(c4)sc4ccccc45)n3)ccc2-c2cc3c(cc21)oc1ccccc13 ; CC1(C)c2cc(-c3ccc4c(c3)c3ccccc3n4-c3nc(-c4ccccc4)nc(-c4ccc5sc6ccccc6c5c4)n3)ccc2-c2cc3c(cc21)oc1ccccc13]

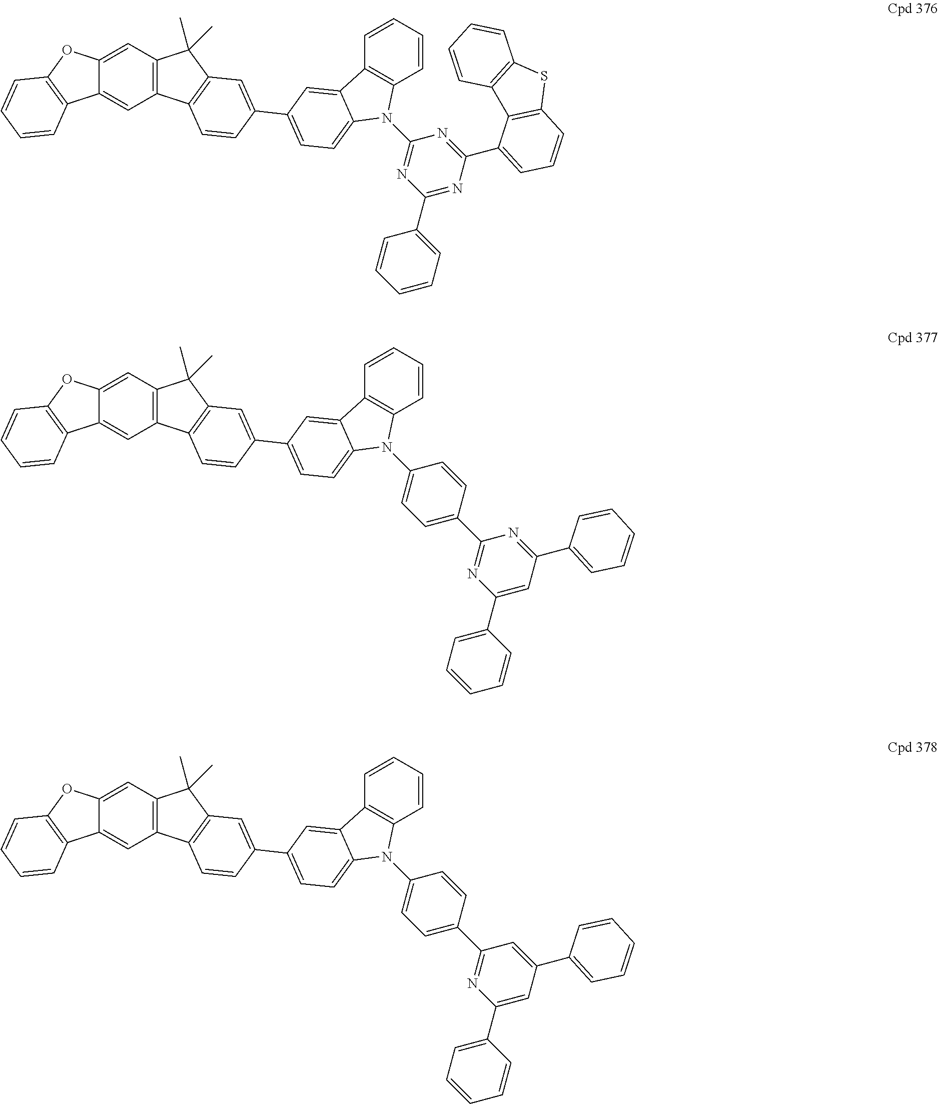 [SMILES: CC1(C)c2cc(-c3ccc4c(c3)c3ccccc3n4-c3nc(-c4ccccc4)nc(-c4cccc5sc6ccccc6c45)n3)ccc2-c2cc3c(cc21)oc1ccccc13 ; CC1(C)c2cc(-c3ccc4c(c3)c3ccccc3n4-c3ccc(-c4nc(-c5ccccc5)cc(-c5ccccc5)n4)cc3)ccc2-c2cc3c(cc21)oc1ccccc13 ; CC1(C)c2cc(-c3ccc4c(c3)c3ccccc3n4-c3ccc(-c4cc(-c5ccccc5)cc(-c5ccccc5)n4)cc3)ccc2-c2cc3c(cc21)oc1ccccc13]

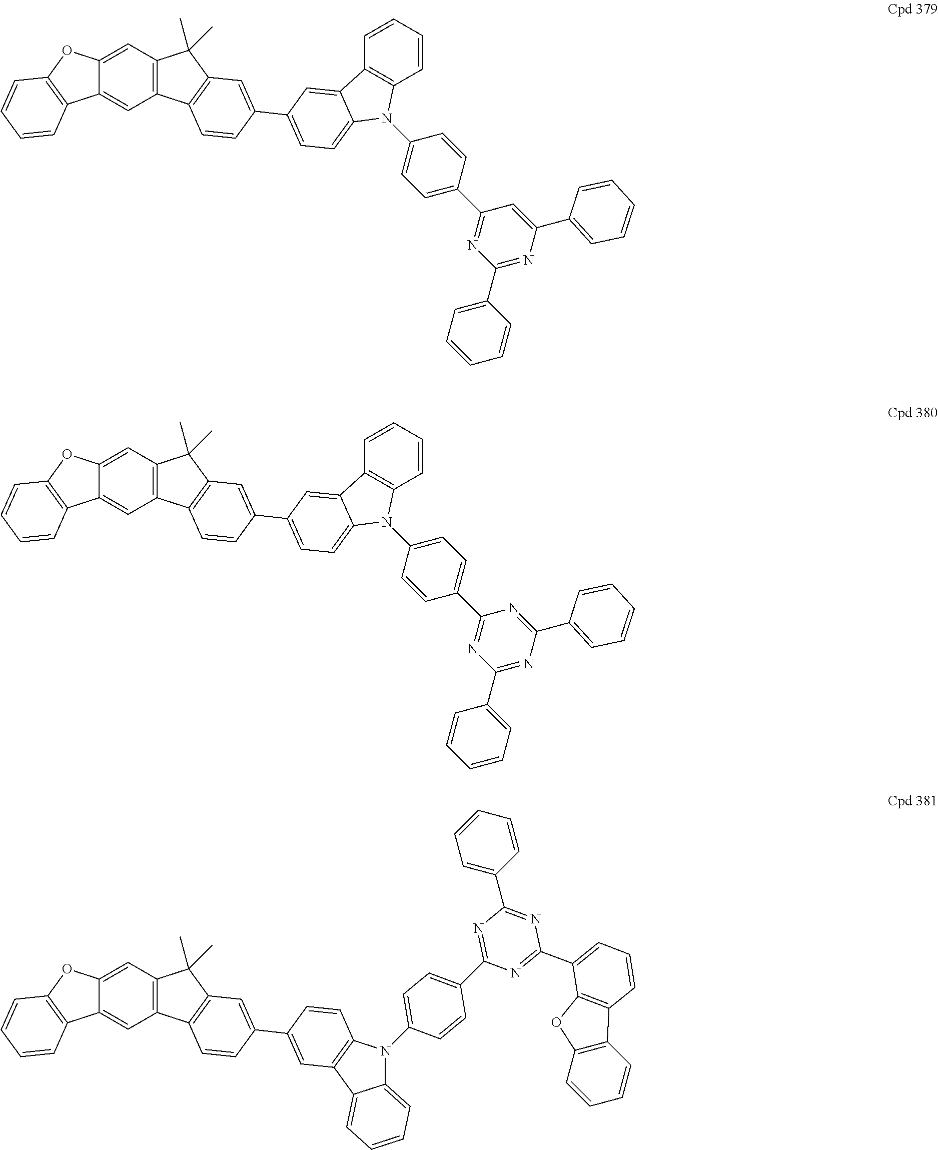

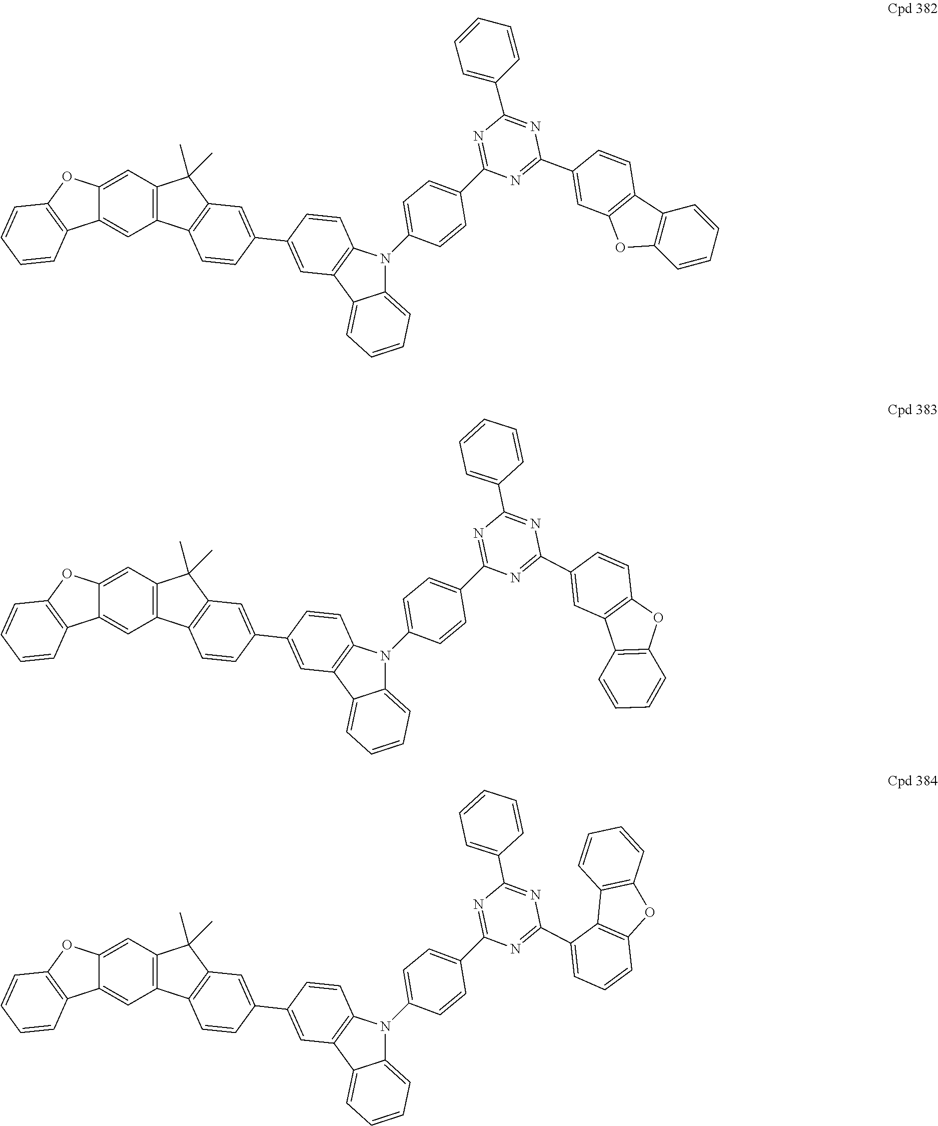 [SMILES: CC1(C)c2cc(-c3ccc4c(c3)c3ccccc3n4-c3ccc(-c4nc(-c5ccccc5)nc(-c5ccc6c(c5)oc5ccccc56)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5ccc6oc7ccccc7c6c5)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5cccc6oc7ccccc7c56)n4)cc3)ccc2-c2cc3c(cc21)oc1ccccc13]

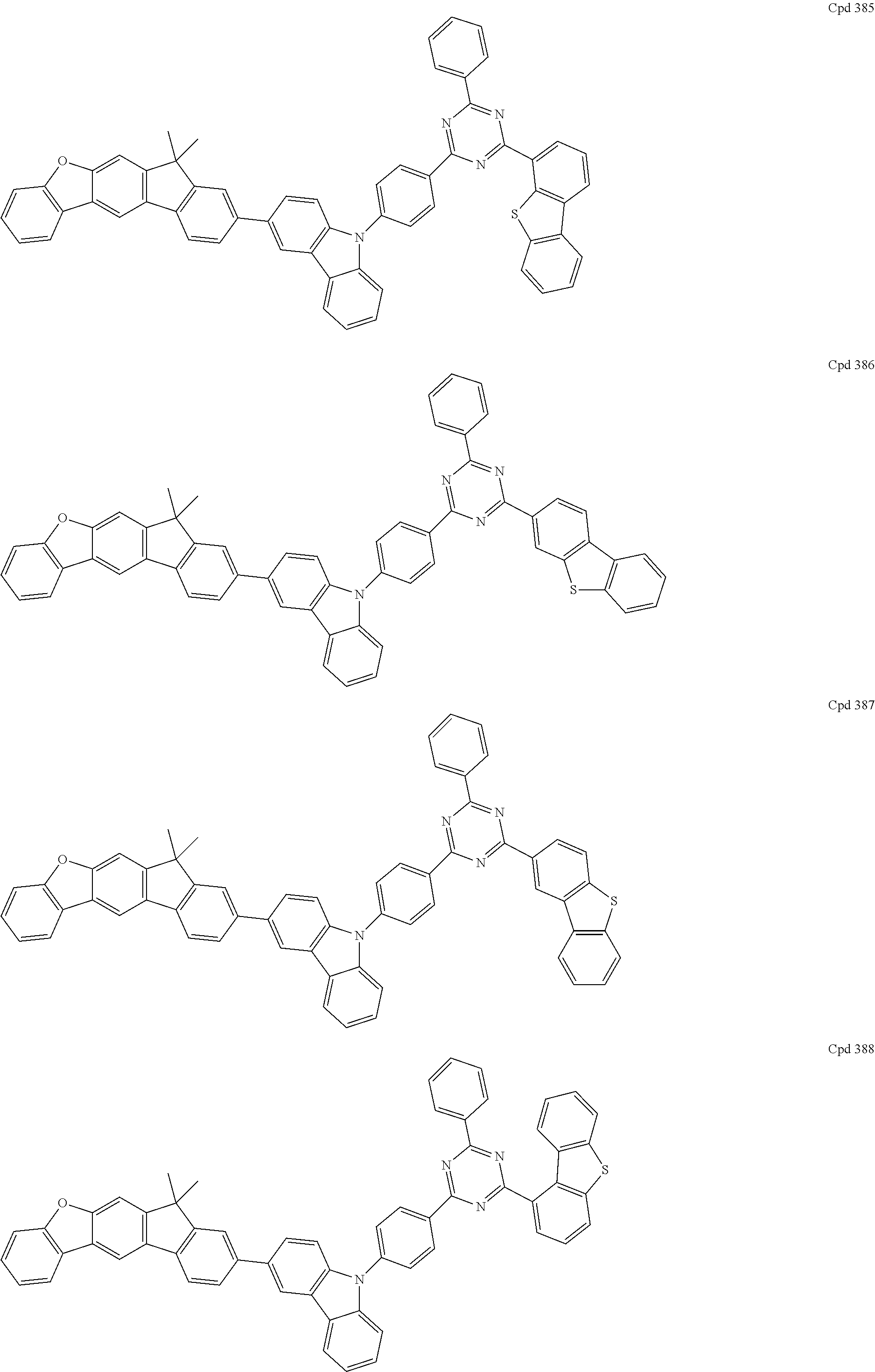 [SMILES: CC1(C)c2cc(-c3ccc4c(c3)c3ccccc3n4-c3ccc(-c4nc(-c5ccccc5)nc(-c5cccc6c5sc5ccccc56)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5ccc6c(c5)sc5ccccc56)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5ccc6sc7ccccc7c6c5)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5cccc6sc7ccccc7c56)n4)cc3)ccc2-c2cc3c(cc21)oc1ccccc13]

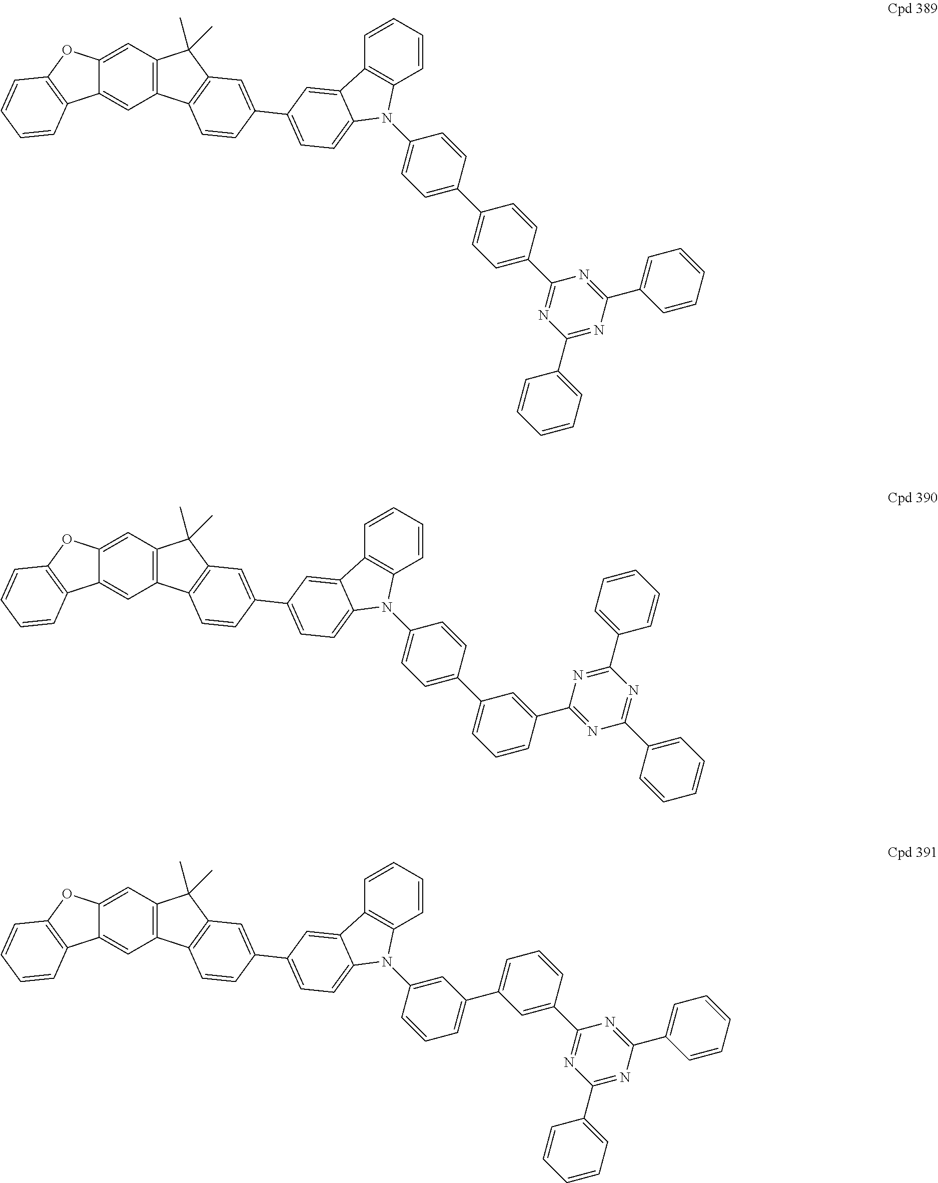 [SMILES: CC1(C)c2cc(-c3ccc4c(c3)c3ccccc3n4-c3ccc(-c4ccc(-c5nc(-c6ccccc6)nc(-c6ccccc6)n5)cc4)cc3)ccc2-c2cc3c(cc21)oc1ccccc13 ; CC1(C)c2cc(-c3ccc4c(c3)c3ccccc3n4-c3ccc(-c4cccc(-c5nc(-c6ccccc6)nc(-c6ccccc6)n5)c4)cc3)ccc2-c2cc3c(cc21)oc1ccccc13 ; CC1(C)c2cc(-c3ccc4c(c3)c3ccccc3n4-c3cccc(-c4cccc(-c5nc(-c6ccccc6)nc(-c6ccccc6)n5)c4)c3)ccc2-c2cc3c(cc21)oc1ccccc13]

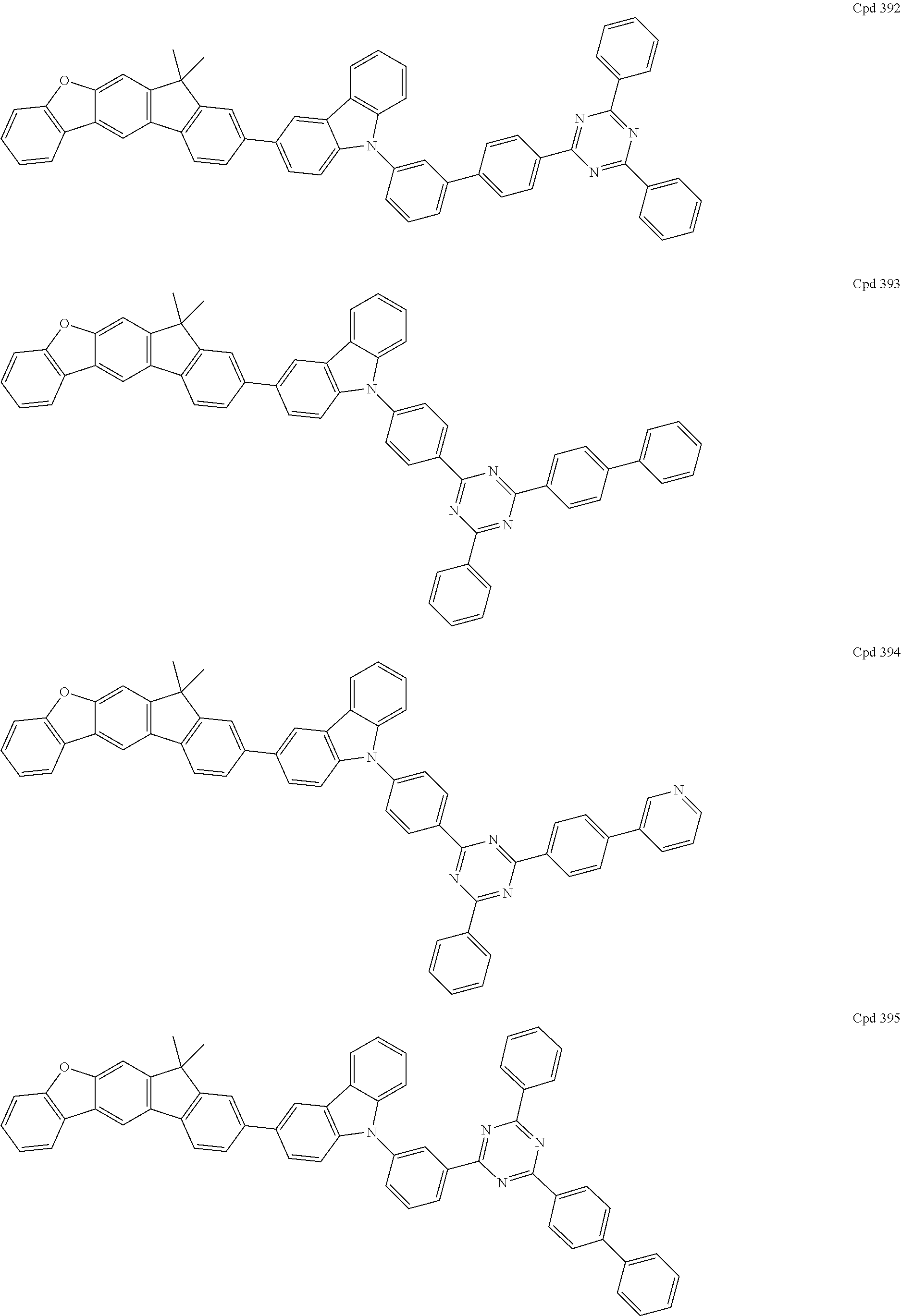 [SMILES: CC1(C)c2cc(-c3ccc4c(c3)c3ccccc3n4-c3cccc(-c4ccc(-c5nc(-c6ccccc6)nc(-c6ccccc6)n5)cc4)c3)ccc2-c2cc3c(cc21)oc1ccccc13 ; CC1(C)c2cc(-c3ccc4c(c3)c3ccccc3n4-c3ccc(-c4nc(-c5ccccc5)nc(-c5ccc(-c6ccccc6)cc5)n4)cc3)ccc2-c2cc3c(cc21)oc1ccccc13 ; CC1(C)c2cc(-c3ccc4c(c3)c3ccccc3n4-c3ccc(-c4nc(-c5ccccc5)nc(-c5ccc(-c6cccnc6)cc5)n4)cc3)ccc2-c2cc3c(cc21)oc1ccccc13 ; CC1(C)c2cc(-c3ccc4c(c3)c3ccccc3n4-c3cccc(-c4nc(-c5ccccc5)nc(-c5ccc(-c6ccccc6)cc5)n4)c3)ccc2-c2cc3c(cc21)oc1ccccc13]

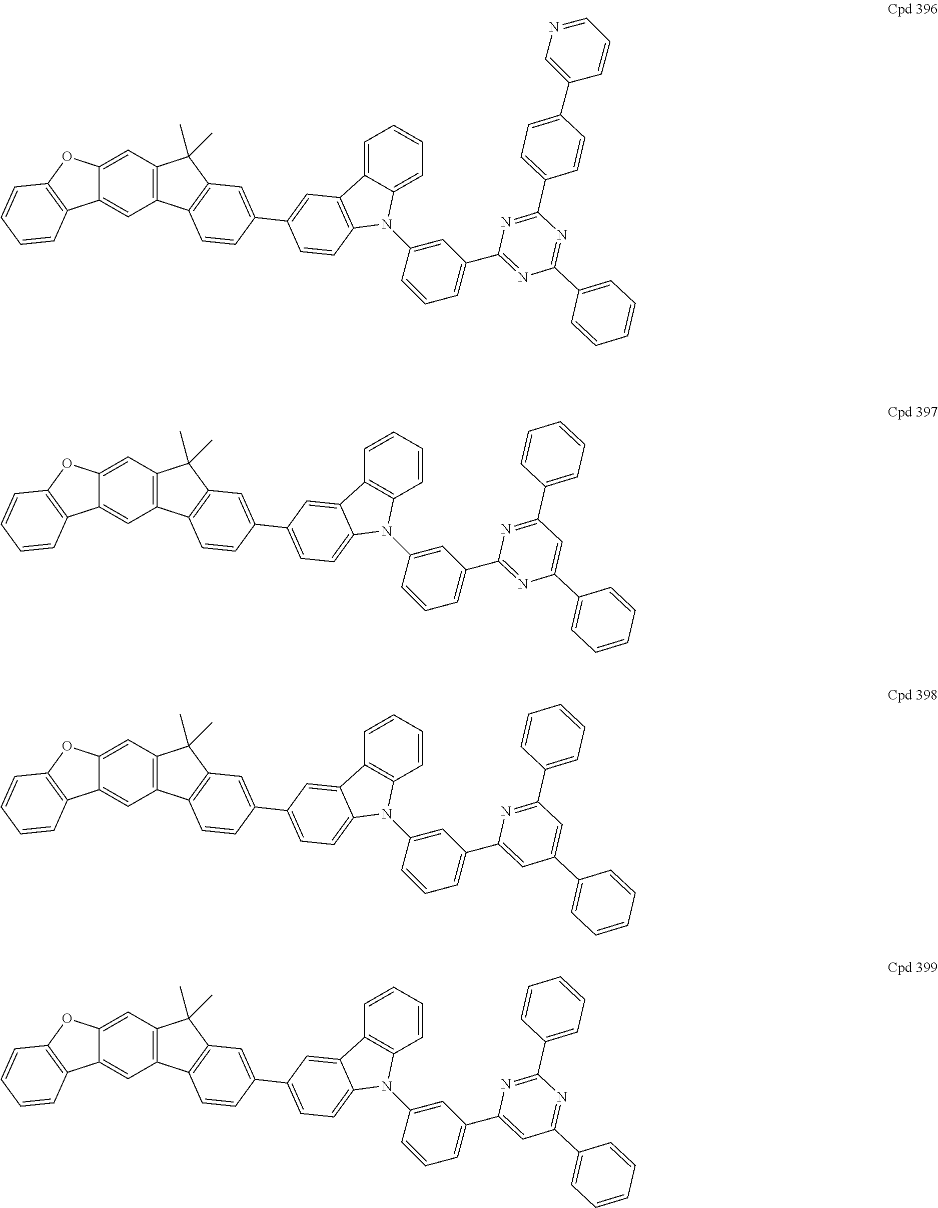 [SMILES: CC1(C)c2cc(-c3ccc4c(c3)c3ccccc3n4-c3cccc(-c4nc(-c5ccccc5)nc(-c5ccc(-c6cccnc6)cc5)n4)c3)ccc2-c2cc3c(cc21)oc1ccccc13 ; CC1(C)c2cc(-c3ccc4c(c3)c3ccccc3n4-c3cccc(-c4nc(-c5ccccc5)cc(-c5ccccc5)n4)c3)ccc2-c2cc3c(cc21)oc1ccccc13 ; CC1(C)c2cc(-c3ccc4c(c3)c3ccccc3n4-c3cccc(-c4cc(-c5ccccc5)cc(-c5ccccc5)n4)c3)ccc2-c2cc3c(cc21)oc1ccccc13 ; CC1(C)c2cc(-c3ccc4c(c3)c3ccccc3n4-c3cccc(-c4cc(-c5ccccc5)nc(-c5ccccc5)n4)c3)ccc2-c2cc3c(cc21)oc1ccccc13]

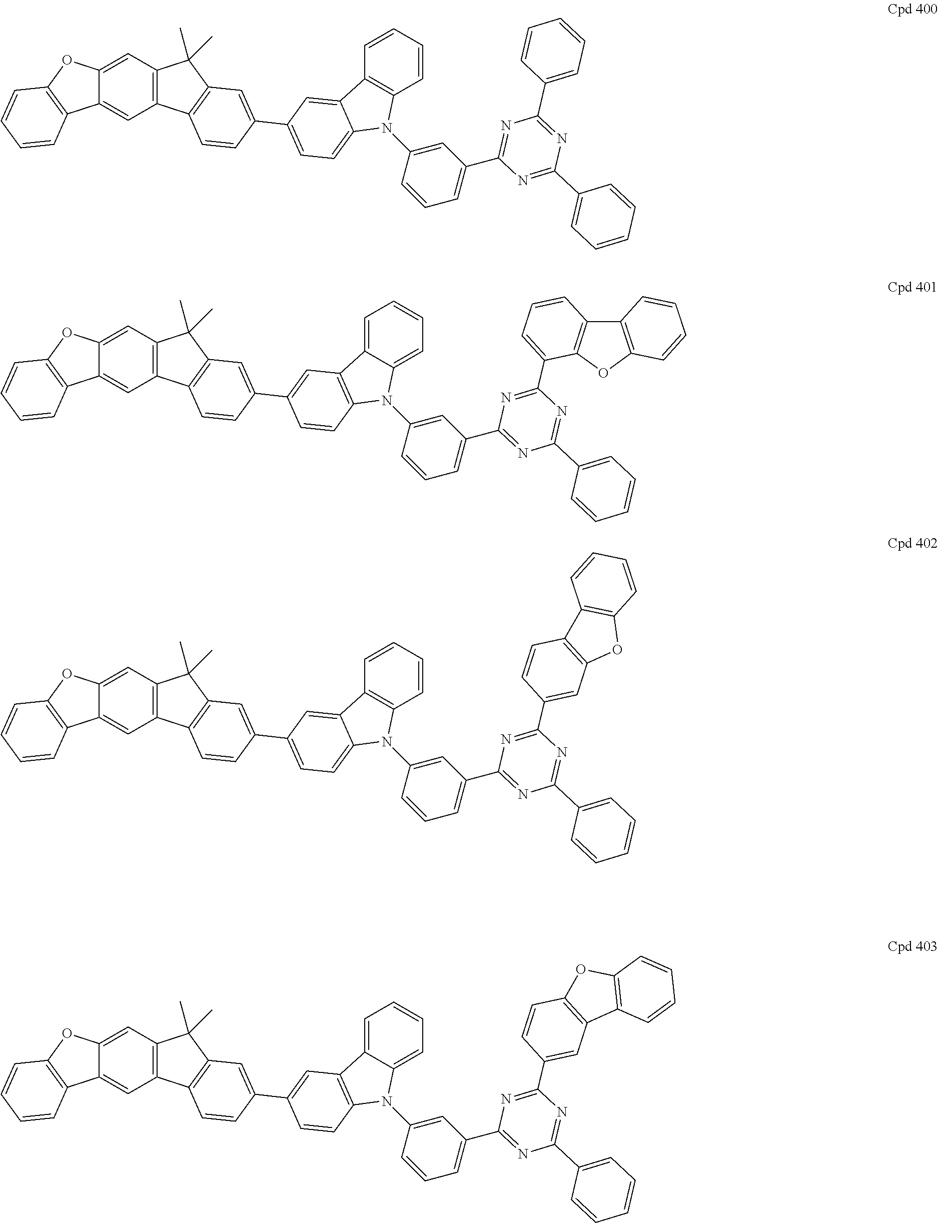

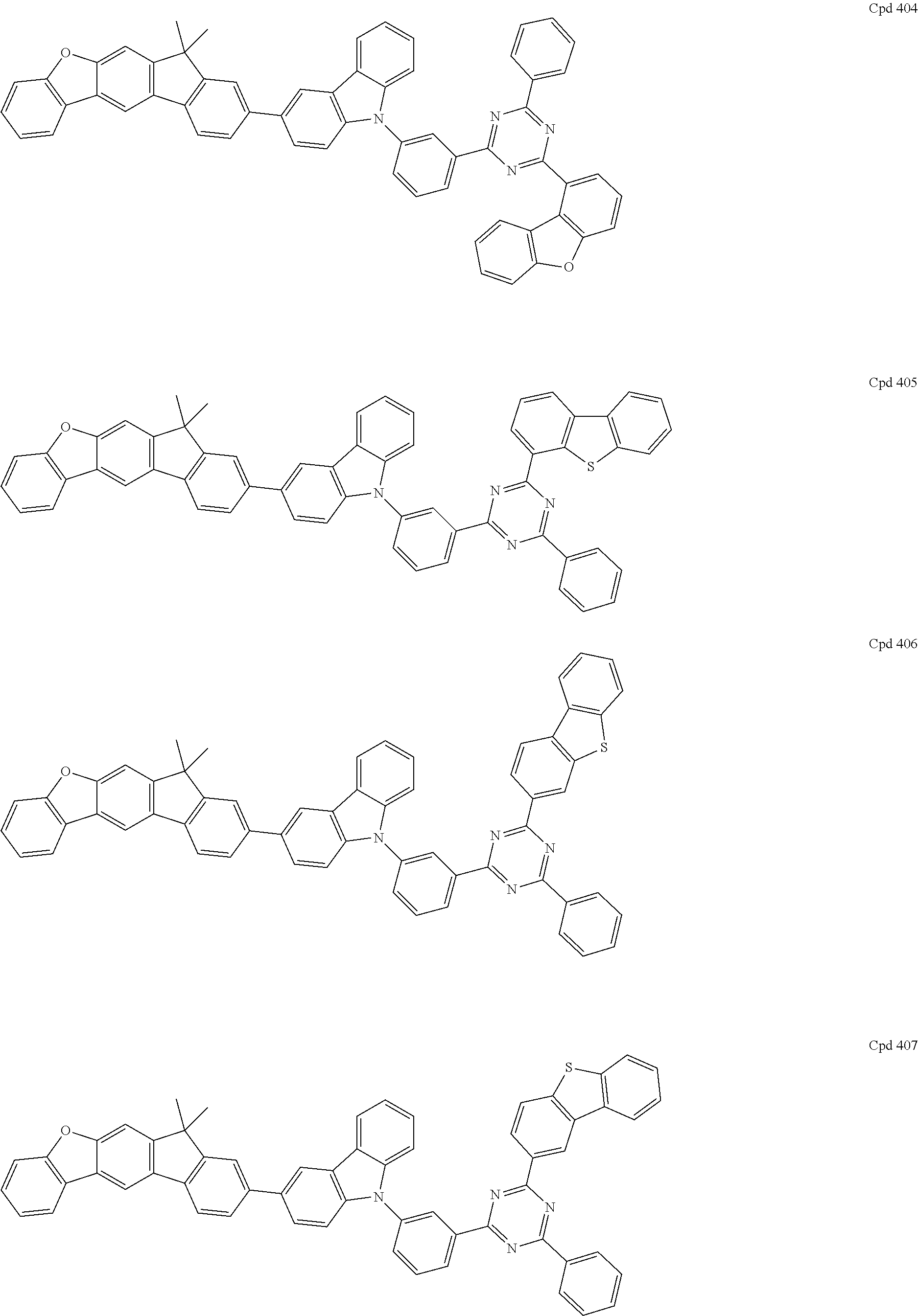

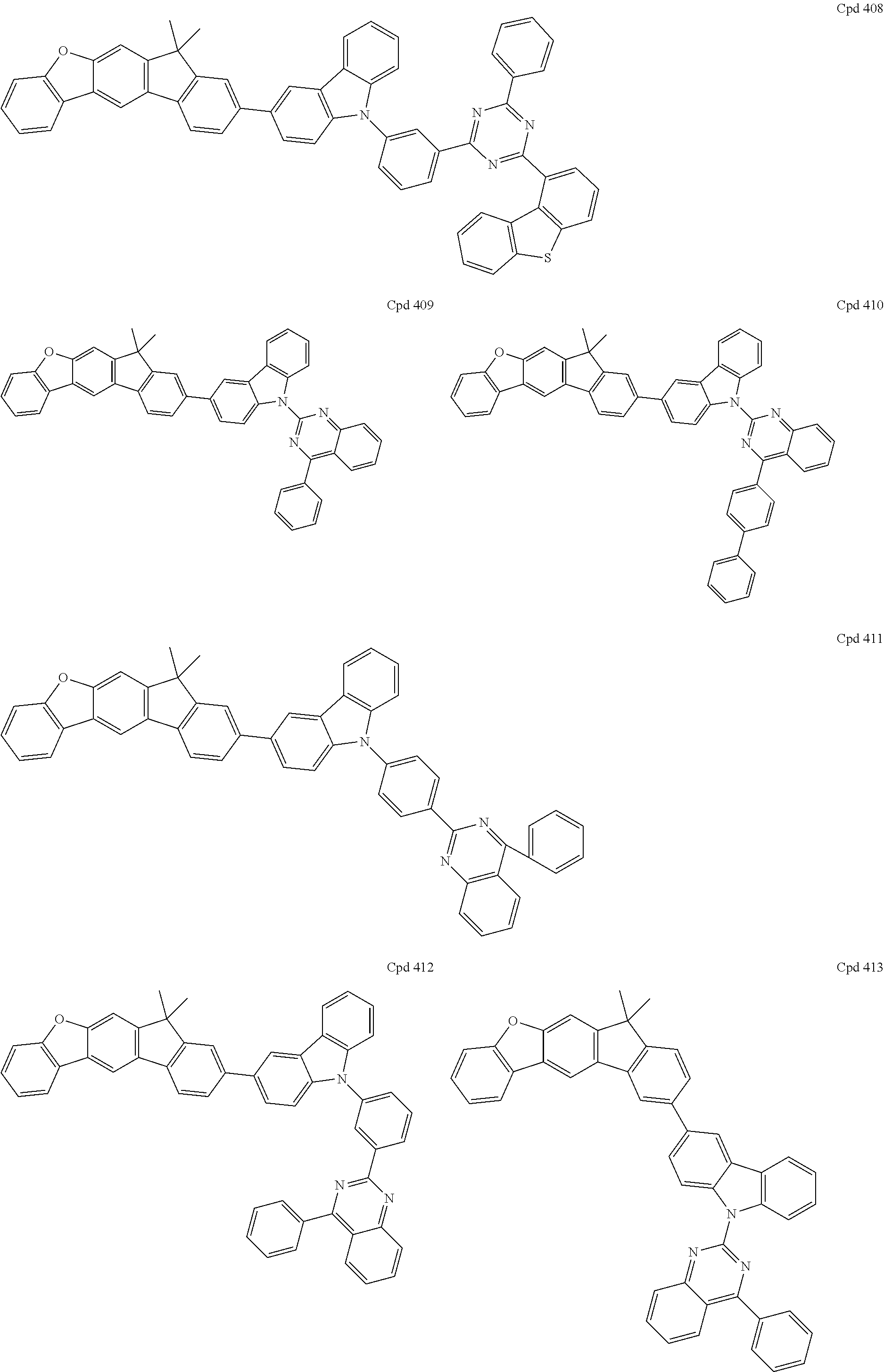

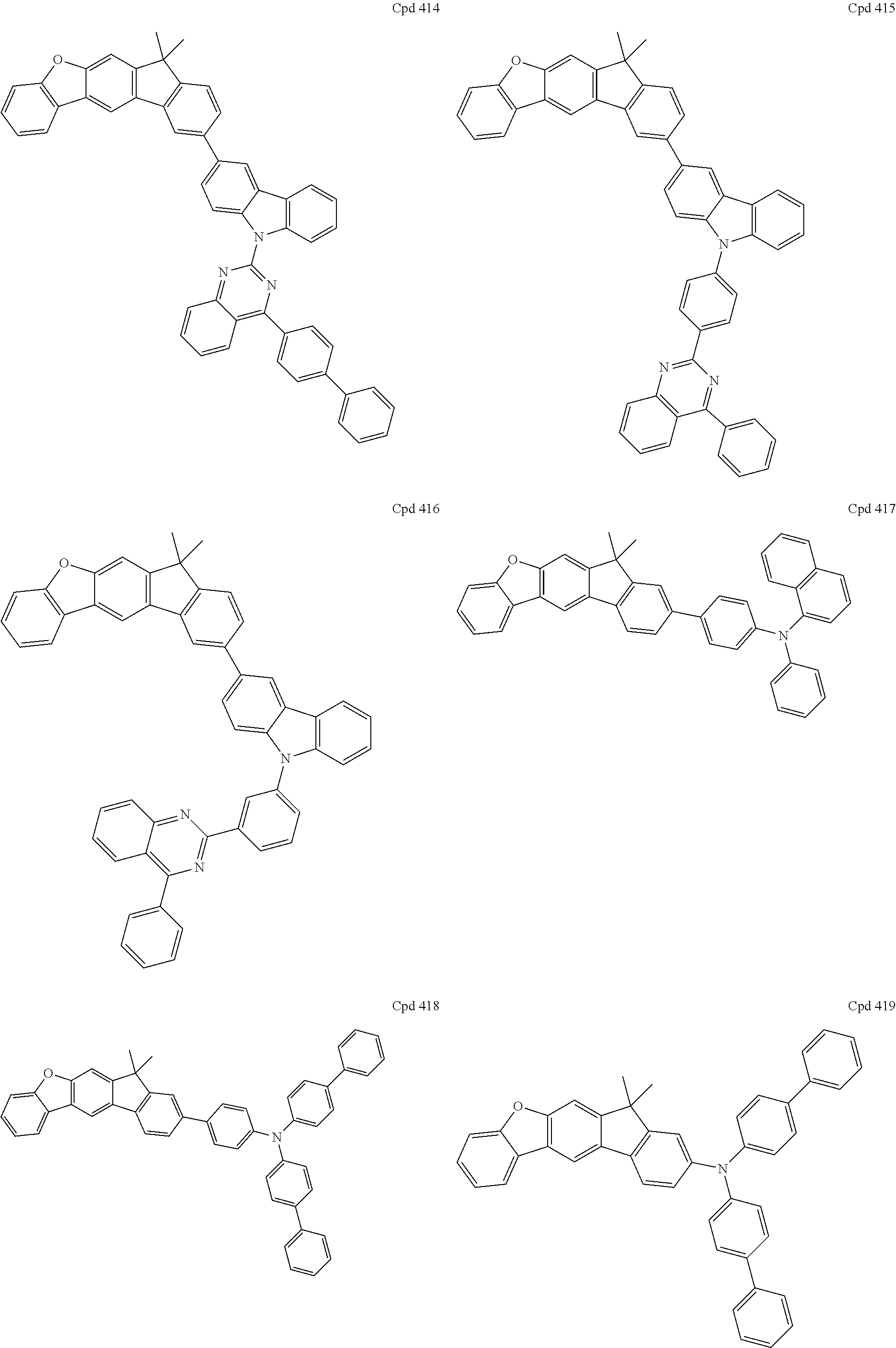

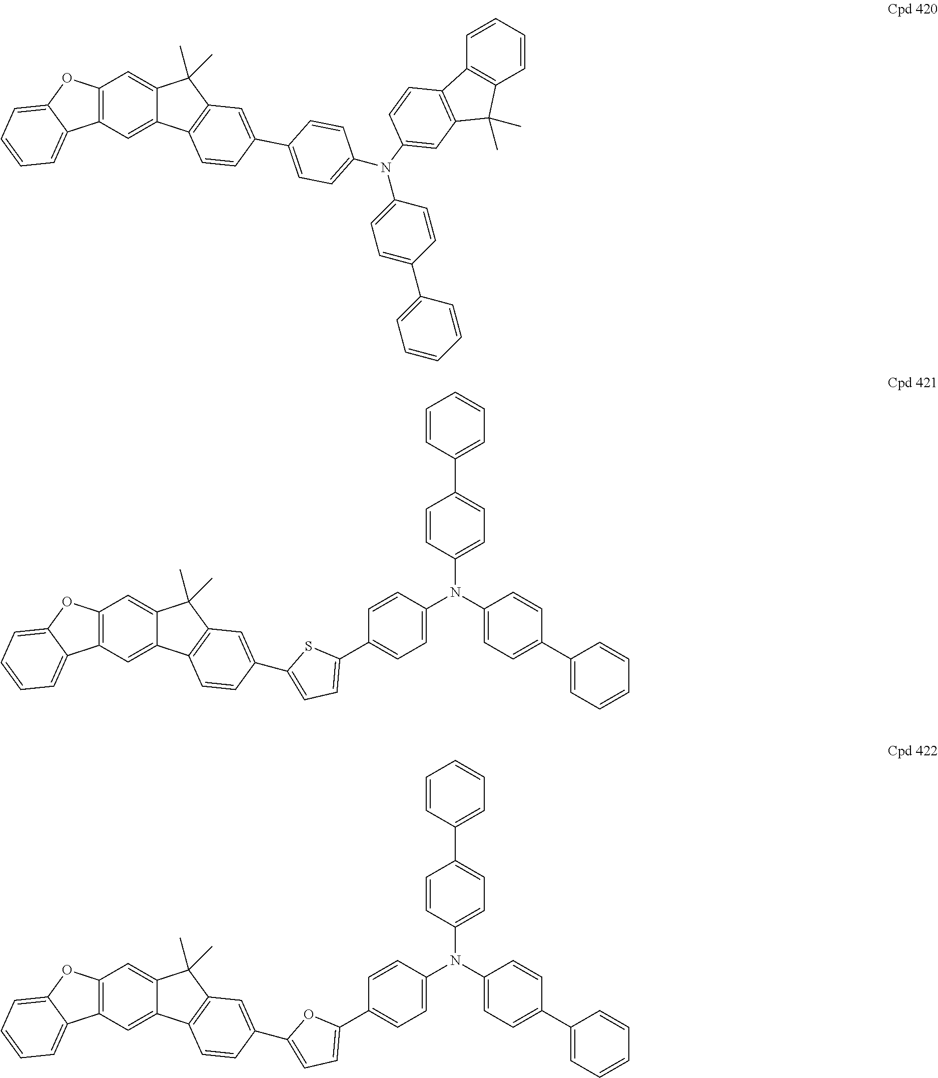

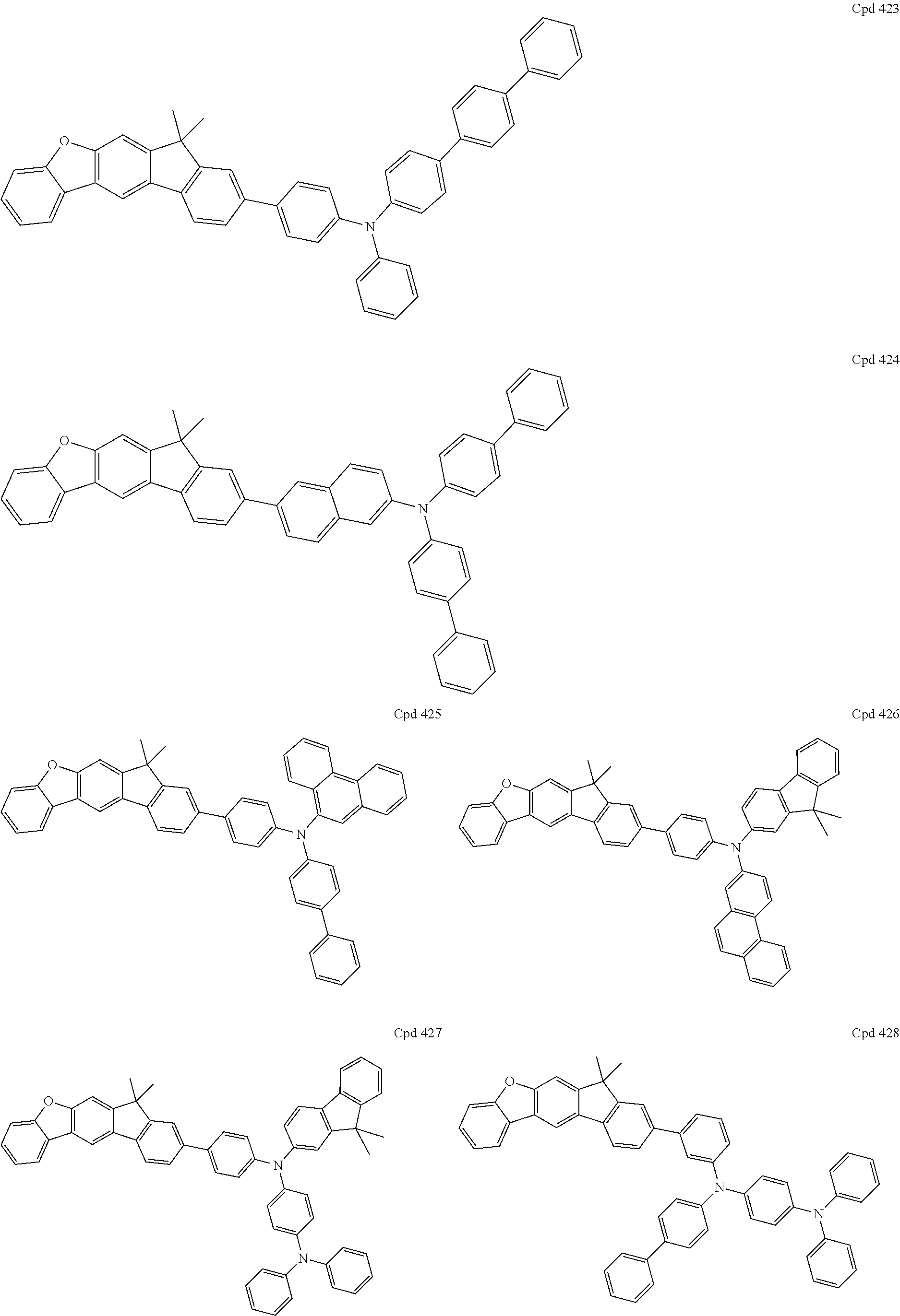

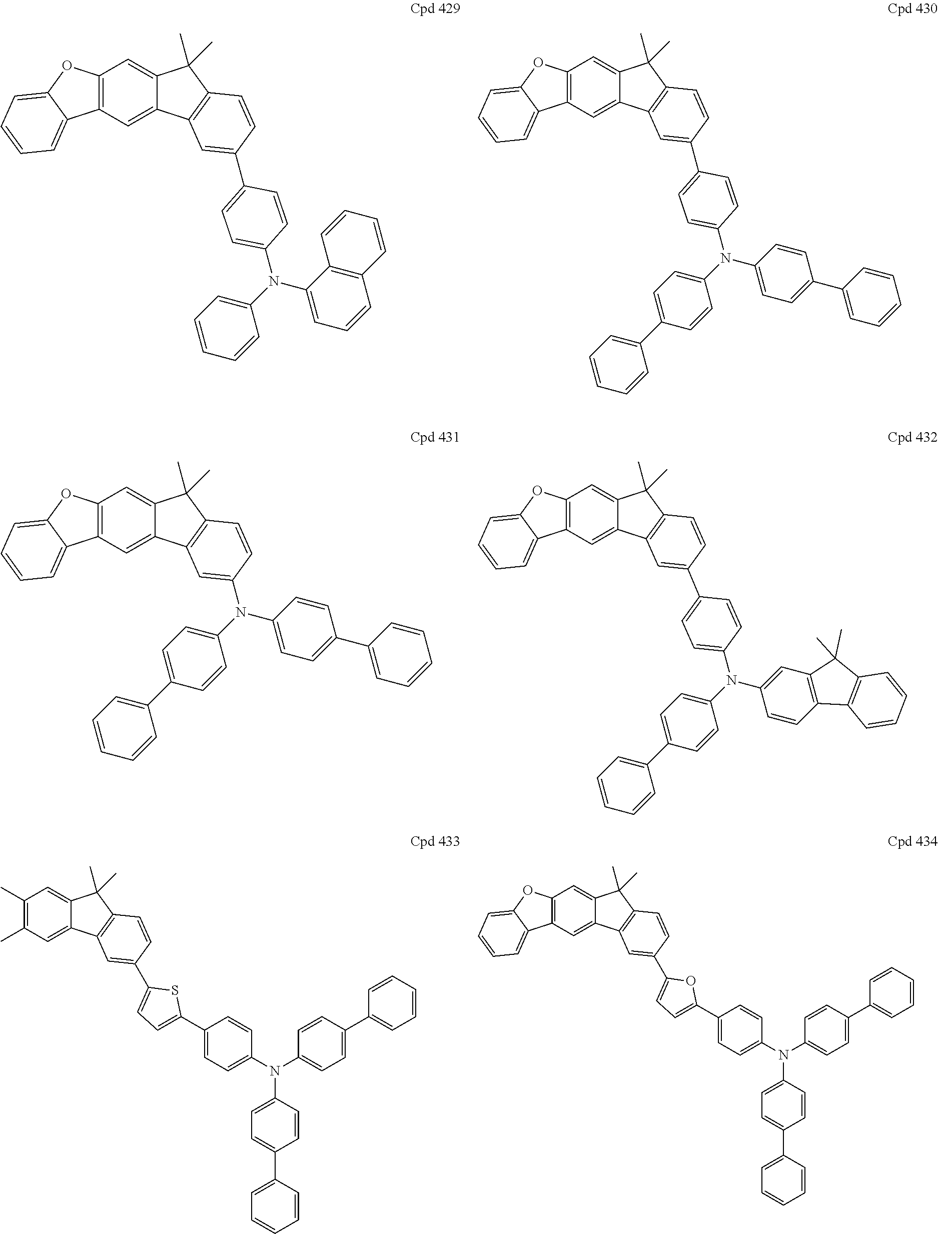

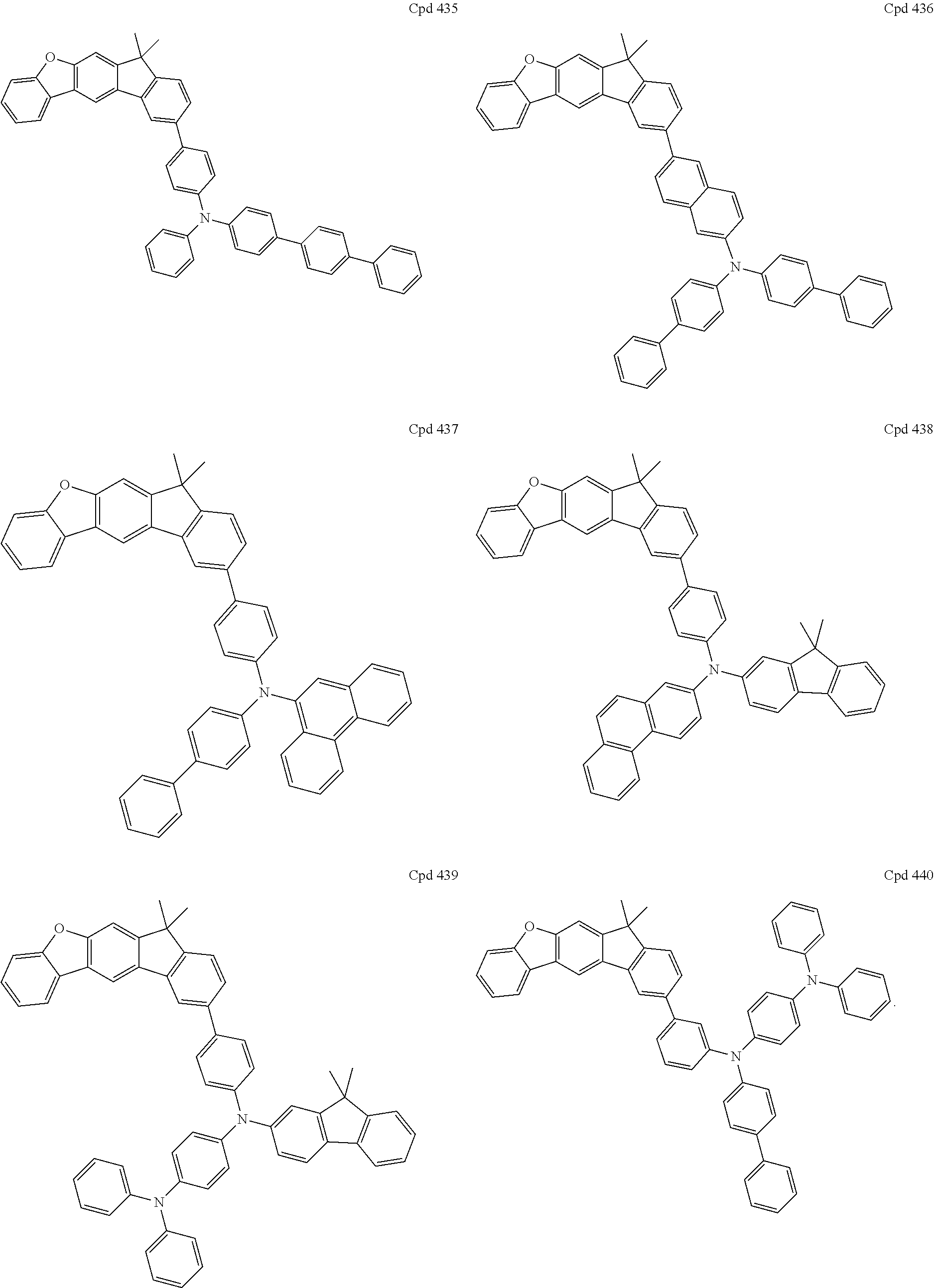

8. An organic electroluminescent device comprising:
(i) an anode;
(ii) a cathode; and
(iii) one or more organic material layers provided between the anode and the cathode,
wherein at least one of the one or more organic material layers includes the compound of claim 1.

9. The organic electroluminescent device of claim 8, wherein the organic material layer is selected from the group consisting of a light emitting layer, a light emitting auxiliary layer, a hole transport layer, a hole injection layer, an electron transport layer, a hole blocking layer and an electron injection layer.

10. The organic electroluminescent device of claim 8, wherein the organic material layer is a light emitting layer or a hole transport layer.

11. The compound of claim 2 wherein X₁ is O.

12. The compound of claim 2, wherein X₂ and X₃ are a methyl group.

13. The compound of claim 2, wherein R₁₂ is a substituent represented by any one of the following Chemical Formula 9 to Chemical Formula 11: 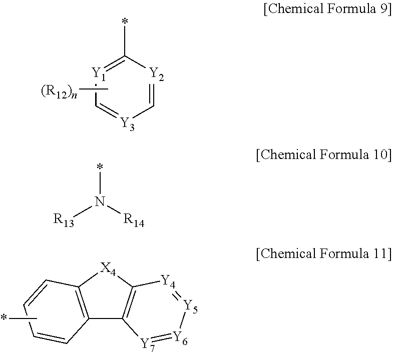
wherein,
* means a part forming a bond;
n is an integer of 0 to 2;
Y₁ to Y₃ are the same as or different from each other and each independently C(R₁₅) or N, and at least one or more are N;
Y₄ to Y₇ are the same as or different from each other, and each independently C(R₁₆) or N;
X₄ is selected from the group consisting of O, S, C(R₁₇)(R₁₈), Si(R₁₉)(R₂₀) and N(R₂₁);
R₁₂ to R₁₄ are the same as or different from each other, and each independently selected from the group consisting of hydrogen, deuterium, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group;
R₁₅ and R₁₆ are the same as or different from each other, and each independently selected from the group consisting of hydrogen, deuterium, halogen, a cyano group, a nitro group, a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group, or bond to adjacent groups to form a fused ring;
R₁₇ to R₂₁ are the same as or different from each other, and each independently selected from the group consisting of a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₁˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ arylphosphine oxide group and a C₆˜C₆₀ arylamine group, or bond to adjacent groups to form a fused ring; and
the alkyl group, the alkenyl group, the alkynyl group, the cycloalkyl group, the heterocycloalkyl group, the aryl group, the heteroaryl group, the alkyloxy group, the aryloxy group, the alkylsilyl group, the arylsilyl group, the alkylboron group, the arylboron group, the arylphosphine group, the arylphosphine oxide group, the mono or diarylphosphinyl group and the arylamine group of R₁₂ to R₂₁ are each independently unsubstituted or substituted with one or more types of substituents selected from the group consisting of a C₁˜C₄₀ alkyl group, a C₂˜C₄₀ alkenyl group, a C₂˜C₄₀ alkynyl group, a C₃˜C₄₀ cycloalkyl group, a heterocycloalkyl group having 3 to 40 nuclear atoms, a C₆˜C₆₀ aryl group, a heteroaryl group having 5 to 60 nuclear atoms, a C₁˜C₄₀ alkyloxy group, a C₆˜C₆₀ aryloxy group, a C₃˜C₄₀ alkylsilyl group, a C₆˜C₆₀ arylsilyl group, a C₁˜C₄₀ alkylboron group, a C₆˜C₆₀ arylboron group, a C₆˜C₆₀ arylphosphine group, a C₆˜C₆₀ mono or diarylphosphinyl group and a C₆˜C₆₀ arylamine group, and when substituted with a plurality of the substituents, these are the same as or different from each other.

14. The compound of claim 2, wherein L is a single bond, a phenylene group or a biphenylene group.
